(19) United States
(12) Patent Application Publication
(10) Pub. No.: US 2024/0018559 A1
(43) Pub. Date: Jan. 18, 2024

(54) GENETICALLY ENGINEERED BACTERIUM CAPABLE OF PRODUCING CYTOKININS WITH ISOPRENOID SIDE CHAINS

(71) Applicant: Acies Bio d.o.o., Ljubljana (SI)

(72) Inventors: Tina Kogej, Ljubljana (SI); Gregor Kosec, Ljubljana (SI); Stefan Fujs, Ljubljana (SI); Marko Blazic, Ljubljana (SI); Jaka Horvat, Ljubljana (SI)

(21) Appl. No.: 18/037,918

(22) PCT Filed: Nov. 26, 2021
(86) PCT No.: PCT/EP2021/083188

§ 371 Date: May 19, 2023

(30) Foreign Application Priority Data
Nov. 26, 2020 (EP) 20075015.6

(51) Int. Cl. C12P 17/16; C12N 9/10; C12N 9/24

(52) U.S. Cl. CPC C12P 17/165; C12N 9/1085; C12Y 205/01027; C12N 9/1022; C12Y 202/01007; C12N 9/2497

(57) ABSTRACT

The present invention generally relates to the biotechnology engineering, and specifically to a genetically engineered bacterium capable of producing cytokinins with isoprenoid side chains (isoprenoid cytokinins), and the preparation and application thereof.

### TECHNICAL FIELD OF THE INVENTION

The present invention generally relates to biotechnology engineering, and specifically to a genetically engineered bacterium capable of producing cytokinins with isoprenoid side chains (isoprenoid cytokinins), and the preparation and application thereof.

### BACKGROUND OF THE INVENTION

Cytokinins are essential plant hormones that control numerous plant growth and developmental processes from seed germination to plant and leaf senescence, characterized by their ability for induction of cell division (Mok, Martin, and Mok 2000). Naturally occurring cytokinins are adenine derivatives with either an aromatic side chain (aromatic cytokinins) or an isoprene-derived side chain at the N⁶-terminus of adenine (isoprenoid cytokinins). The N⁶-side-chain structure and configuration determine both the cytokinin type and its activity. The isoprenoid cytokinins are widespread and are produced naturally in plants and algae, and also in many species of bacteria, fungi, nematodes, and parasitic insects (Stirk and van Staden 2010). Natural isoprenoid cytokinins are: N⁶-(D2-isopentenyl)adenine (iP), trans-zeatin (tZ), cis-zeatin (cZ) and dihydrozeatin (DZ) (FIG. 1).

Isoprenoid cytokinins are derivatives of adenine or adenosine in which the exocyclic amino group at 6 has been modified by attachment of a dimethylallyl side chain, resulting in the parent compound N⁶-(D2-isopentenyl)adenine (iP) and its riboside, N⁶-(D2-isopentenyl)adenosine (iPR). The dimethylallyl side chain can be hydroxylated at 9′ to give trans-zeatin (tZ) or its riboside, trans-ribosylzeatin (tZR). Also, the exocyclic double bond can be reduced to give dihydrozeatin (DZ) or its riboside, ribosyl dihydrozeatin (DZR) (FIG. 1). All six compounds, iP, iPR, tZ, tZR, DZ, and DZR are biologically active and occur naturally.

The majority of naturally occurring cytokinins exist in distinctive structural derivatives or forms, such as free bases, ribosides, and nucleotides, or conjugates with glucose, xylose, or amino acid residues. In plants, the cytokinin free bases are considered the most biologically active forms while glucose conjugates are considered to be either permanently inactive or reversible storage forms, depending on the location of glycosylation.

Zeatin is the predominant form of cytokinin in plants. Zeatin is an adenine derivative with a hydroxylated isoprene-derived side chain at the N⁶ position of adenine, which can exist in cis- or trans-configuration (FIG. 1). Trans-zeatin is one of the most effective naturally occurring cytokinins. In many previous studies, the analytical methods failed to distinguish cis- and trans-zeatin, thereby obscuring the understanding of the presence and role of both compounds in the observed physiological processes.

Despite the structural similarity, cis-zeatin is synthesized through the tRNA pathway, whereas in contrast, trans-zeatin, and biosynthetically related compounds iP and DZ are synthesized in plant cells through the de novo (or AMP) biosynthesis pathway.

In the tRNA pathway, cis-zeatin is a recycled product of degradation of isopentenylated tRNAs. Cis-zeatin is synthesized in almost all organisms except Archaea (Schafer et al. 2015) at extremely low rates by tRNA-isopentenyltransferases (tRNA-IPTs) that catalyze the prenylation of adenine 37 on specific (UNN-)tRNAs leading to the formation of isopentenyl adenine (IP)-containing tRNA. In the de novo cytokinin biosynthesis pathway, the first step is N-prenylation of adenosine 5′-phosphates (AMP, ADP, or ATP) with dimethylallyl diphosphate (DMAPP) catalyzed by adenylate isopentenyltransferase (IPT, EC 2.5.1.27), which produces isopentenyladenine nucleotide (iP). iP, produced by IPT in plants, then undergoes hydroxylation at the prenyl side chain to result in tZ-nucleotides. In Arabidopsis, two cytochrome P450 monooxygenases, CYP735A1, and CYP735A2, catalyze the hydroxylation reaction. The CYP735As preferentially utilize iP-nucleotides rather than the iP-nucleoside and iP. Since this reaction is stereo-specific, the CYP735As produce tZ-nucleotides (Takei, Yamaya, and Sakakibara 2004). In the final step of both pathways, the de novo and the tRNA pathway, the cytokinin-activating enzyme cytokinin riboside 5-monophosphate phosphoribohydrolase ‘Lonely guy’ (LOG, EC 3.2.2.n1) removes the ribosyl moiety and converts cytokinin nucleotides to their active nucleobases (Kurakawa et al. 2007). All four cytokinin nucleoside monophosphates, iPRMP, tZRMP, DZRMP, and cZRMP are utilized by LOG.

In the case of plant infection by phytopathogenic bacteria, such as Agrobacterium tumefaciens, biosynthesis of tZ is initiated to facilitate the infection. During the infection process, tZ or iP biosynthesis can occur inside the bacterial cells or bacterial IPT gene homologs can be integrated into the host's nuclear genome and expressed in the infected plant cells.

Importantly, substrate specificities differ between IPTs of bacteria, and higher plants (Kakimoto 2001; Sakakibara 2005). In the first aspect, bacterial IPTs, such as Tmr and Tzs from A. tumefaciens, use only AMP as an acceptor (whereas plant IPT enzymes preferentially use ADP and ATP) to form trans-zeatin riboside 5′-monophosphate (tZRMP) (Sakakibara 2006; Kamada-Nobusada and Sakakibara 2009). Besides, Agrobacterium IPTs Tzs and Tmr are capable of using either DMAPP or 1-hydroxy-2-methyl-2-butenyl 4-diphosphate (HMBDP), the hydroxylated precursor of DMAPP in the methylerythritol phosphate (MEP) pathway, as the side chain donor. When DMAPP is used as a substrate, the primary product is iPRMP, whereas tZRMP is formed when IPT utilizes HMBDP. Therefore, the expression of Agrobacterium IPT Tmr in the chloroplasts of the plant cells during the infection creates a metabolic bypass for direct synthesis of tZRMP, sequestering the efficient substrate supply of isoprenoid precursors HMBDP in plastids of the plant cells. As in the native pathway, LOG phosphoribohydrolase finally releases the ribose monophosphate moiety from either iP- or tZ-nucleoside monophosphate (iPRMP, tZRMP), creating the biologically active molecule iP and tZ.

The tumor-inducing plant pathogenic bacterium A. tumefaciens has 2 IPTs: Tmr and Tzs. Tmr and Tzs are homologous proteins, both are DMAPP:AMP isopentenyltransferases, but their amino acids crucial for substrate recognition differ from those of plant adenylate IPTs (Chu et al., 2010). The tmr (ipt) gene is located in the T-region of the Ti-plasmid, which mediates infection in host plants. The tmr gene is transferred from the bacterium to the plant genome to force the host plants to produce cytokinins, which results in tumor formation. In vitro experiments demonstrated that Tmr transferred both DMAPP and HMBDP to AMP with similar Km values (Sakakibara et al. 2005). Nopaline-producing strains of A. tumefaciens possess another gene for DMAPP:AMP isopentenyltransferase, tzs, which is present in the vir region of Ti-plasmid and is not translocated to the plant cells. Tzs enables the high level of cytokinin production and secretion by these A. tumefaciens strains (Morris et al. 1993), and can also use HMBDP or DMAPP to produce iPRMP and tZRMP. Tzs has 51.3% protein sequence identity to Tmr. Genes coding for DMAPP:AMP isopentenyltransferases homologous to tmr/tzs are present in other bacteria of the genus Agrobacterium, A. vitis and A. rhizogenes, as well as in other plant-pathogenic bacteria, such as Pseudomonas syringae pv. savastanoi, Pseudomonas solanacearum, Pantoea agglomerans, and Rhodococcus fascians (Kakimoto, 2003).

Isopentenyl donor, dimethylallyl diphosphate (DMAPP), and its precursor 1-hydroxy-2-methyl-2-butenyl 4-diphosphate (HMBDP) are produced by the methylerythritol phosphate (MEP) pathway in chloroplasts of plants and bacteria, also in Bacillus subtilis. In the MEP pathway, pyruvate and glyceraldehyde-3-phosphate are condensed by the enzyme 1-deoxy-D-xylulose-5-phosphate synthase (Dxs, EC 2.2.1.7) into a metabolic cascade ultimately producing HMBDP, DMAPP, and isoprene or larger terpenoid compounds. The first step in the MEP pathway, mediated by DXS, is the rate-limiting step for isoprenoid production in plants and bacteria (Julsing et al. 2007).

### SUMMARY OF THE INVENTION

The natural biosynthesis rate of cytokinins in plant-pathogenic bacteria through the de novo pathway during infection is very low. It depends on the expression of the IPT enzyme of bacterial origin, the LOG enzyme, and the building blocks, supplied by the metabolism of the host cells. Such an infection-based system is not readily transferable to industrial-scale production. Due to the high activity and potential of using cytokinin hormones in agricultural applications, there is thus a need for efficient and sustainable production of cytokinins such as iP, tZ, and ribosides tZR and iPR in genetically and biotechnologically amenable host strains.

The object of the present invention is to provide means allowing more efficient production of cytokinins with isoprenoid side chains (isoprenoid cytokinins), such as tZ and iP, and their ribosides tZR and iPR. More particularly, it is an object of the present invention to provide means allowing the production of cytokinins with isoprenoid side chains (isoprenoid cytokinins), such as tZ and iP, and their ribosides tZR and iPR, at higher nominal yield.

This is achieved by the present inventors who have engineered bacterial strains, which a) express a heterologous polypeptide having adenylate isopentenyltransferase activity and optionally b) have been modified to have an increased protein expression of a polypeptide having cytokinin riboside 5-monophosphate phosphoribohydrolase activity. As shown in the Examples, such engineered bacterial strains surprisingly show unusually high titers of isoprenoid cytokinins of over 10 mg/L in the supernatant. In consequence, this means that the context of plant cell infection is no longer required and the biosynthetic substrates and cofactors for efficient biosynthesis of isoprenoid cytokinins such as tZ, and iP, and their ribosides tZR and iPR are effectively supplied by the engineered bacterial cell.

The present invention thus provides in a first aspect a bacterium expressing a heterologous polypeptide having adenylate isopentenyltransferase activity. More particularly, the present invention provides a bacterium, which a) expresses a heterologous polypeptide having adenylate isopentenyltransferase activity and optionally b) has been modified to have an increased protein expression of a polypeptide having cytokinin riboside 5-monophosphate phosphoribohydrolase activity compared to an otherwise identical bacterium that does not carry said modification.

The present invention further provides in a second aspect a method for producing a cytokinin or riboside derivative thereof, particularly an isoprenoid cytokinin or riboside derivative thereof, comprising cultivating a bacterium according to the present invention under suitable culture conditions in a suitable culture medium.

The present invention may be summarized by the following items:
1. A bacterium, which expresses a heterologous polypeptide having adenylate isopentenyltransferase activity.
2. The bacterium according to item 1, wherein the polypeptide having adenylate isopentenyltransferase activity is selected from the group consisting of: i) a polypeptide comprising an amino acid sequence of any one of SEQ ID NOs: 1 to 33; and ii) a polypeptide comprising an amino acid sequence, which has at least about 50%, such as at least about 55%, at least about 60%, at least about 65%, at least about 70%, at least about 75%, at least about 80%, at least about 85%, at least about 90%, at least about 93%, at least 95%, at least 96%, at least 97%, at least 98%, or at least 99%, sequence identity to the amino acid sequence of any one of SEQ ID NOs: 1 to 33.
3. The bacterium according to item 1, wherein the polypeptide having adenylate isopentenyltransferase activity is selected from the group consisting of: i) a polypeptide comprising the amino acid sequence of any one of SEQ ID NOs: 1 to 10; and ii) a polypeptide comprising an amino acid sequence, which has at least about 50%, such as at least about 55%, at least about 60%, at least about 65%, at least about 70%, at least about 75%, at least about 80%, at least about 85%, at least about 90%, at least about 93%, at least 95%, at least 96%, at least 97%, at least 98%, or at least 99%, sequence identity to the amino acid sequence of any one of SEQ ID NOs: 1 to 10.
4. The bacterium according to item 1, wherein the polypeptide having adenylate isopentenyltransferase activity is selected from the group consisting of: i) a polypeptide comprising the amino acid sequence of SEQ ID NO: 1; and ii) a polypeptide comprising an amino acid sequence, which has at least about 50%, such as at least about 55%, at least about 60%, at least about 65%, at least about 70%, at least about 75%, at least about 80%, at least about 85%, at least about 90%, at least about 93%, at least 95%, at least 96%, at least 97%, at least 98%, or at least 99%, sequence identity to the amino acid sequence of SEQ ID NO: 1.
5. The bacterium according to any one of items 1 to 4, wherein the bacterium comprises an exogenous nucleic acid molecule comprising a nucleotide sequence encoding said heterologous polypeptide.
6. The bacterium according to item 5, wherein the exogenous nucleic acid molecule further comprises a promoter that is functional in the bacterium to cause the production of an mRNA molecule and that is operably linked to the nucleotide sequence encoding said heterologous polypeptide.
7. The bacterium according to item 5 or 6, wherein the exogenous nucleic acid molecule is a vector.
8. The bacterium according to item 5 or 6, wherein the exogenous nucleic acid molecule is stably integrated into the genome of the bacterium.
9. The bacterium according to any one of items 1 to 8, which has been modified to have an increased protein expression of a polypeptide having cytokinin riboside 5-monophosphate phosphoribohydrolase activity compared to an otherwise identical bacterium that does not carry said modification.
10. The bacterium according to item 9, wherein the increase in protein expression of the polypeptide having cytokinin riboside 5-monophosphate phosphoribohydrolase activity is achieved by increasing the number of copies of a gene encoding said polypeptide.
11. The bacterium according to item 10, wherein the increase in the number of copies of the gene is achieved by introducing into the bacterium one or more exogenous nucleic acid molecules (such as one or more vectors) comprising the gene operably linked to a promoter that is functional in the bacterium to cause the production of an mRNA molecule.
12. The bacterium according to any one of items 9 to 11, wherein the bacterium comprises an exogenous nucleic acid molecule (such as a vector) comprising a nucleotide sequence encoding the polypeptide having cytokinin riboside 5-monophosphate phosphoribohydrolase activity.
13. The bacterium according to item 12, wherein the exogenous nucleic acid molecule further comprises a promoter that is functional in the bacterium to cause the production of an mRNA molecule and that is operably linked to the nucleotide sequence encoding the polypeptide.
14. The bacterium according to any one of items 11 to 13, wherein the exogenous nucleic acid molecule is a vector.
15. The bacterium according to any one of items 11 to 13, wherein the exogenous nucleic acid molecule is stably integrated into the genome of the bacterium.
16. The bacterium according to any one of items 9 to 15, wherein the increase in protein expression of the polypeptide having cytokinin riboside 5-monophosphate phosphoribohydrolase activity is achieved by modifying the ribosome binding site.
17. The bacterium according to any one of items 9 to 16, wherein the increase in protein expression of the polypeptide having cytokinin riboside 5-monophosphate phosphoribohydrolase activity is achieved by increasing the strength of the promoter operably linked to the gene encoding the polypeptide.
18. The bacterium according to any one of items 9 to 17, wherein the polypeptide having cytokinin riboside 5-monophosphate phosphoribohydrolase activity is selected from the group consisting of: i) a polypeptide comprising an amino acid sequence of any one of SEQ ID NOs: 34 to 62 and ii) a polypeptide comprising an amino acid sequence, which has at least about 70%, such as at least about 75%, at least about 80%, at least about 85%, at least about 90%, at least about 93%, at least 95%, at least 96%, at least 97%, at least 98%, or at least 99%, sequence identity to the amino acid sequence of any one of SEQ ID NOs: 34 to 62.
19. The bacterium according to any one of items 9 to 18, wherein the polypeptide having cytokinin riboside 5-monophosphate phosphoribohydrolase activity is selected from the group consisting of: i) a polypeptide comprising an amino acid sequence of any one of SEQ ID NOs: 34 to 44; and ii) a polypeptide comprising an amino acid sequence, which has at least about 70%, such as at least about 75%, at least about 80%, at least about 85%, at least about 90%, at least about 93%, at least 95%, at least 96%, at least 97%, at least 98%, or at least 99%, sequence identity to the amino acid sequence of any one of SEQ ID NOs: 34 to 44.
20. The bacterium according to any one of items 9 to 18, wherein the polypeptide having cytokinin riboside 5-monophosphate phosphoribohydrolase activity is selected from the group consisting of: i) a polypeptide comprising the amino acid sequence of SEQ ID NO: 34; and ii) a polypeptide comprising an amino acid sequence, which has at least about 70%, such as at least about 75%, at least about 80%, at least about 85%, at least about 90%, at least about 93%, at least 95%, at least 96%, at least 97%, at least 98%, or at least 99%, sequence identity to the amino acid sequence of SEQ ID NO: 34.
21. The bacterium according to any one of items 9 to 20, wherein the polypeptide having cytokinin riboside 5-monophosphate phosphoribohydrolase activity is a bacterial polypeptide having cytokinin riboside 5-monophosphate phosphoribohydrolase activity.
22. The bacterium according to any one of items 1 to 21, wherein the bacterium has been further modified to have an increased protein expression of a polypeptide having 1-deoxy-D-xylulose-5-phosphate synthase activity compared to an otherwise identical bacterium that does not carry said modification.
23. The bacterium according to item 22, wherein the increase in protein expression of the polypeptide having 1-deoxy-D-xylulose-5-phosphate synthase activity is achieved by increasing the number of copies of a gene encoding said polypeptide.
24. The bacterium according to item 23, wherein the increase in the number of copies of the gene is achieved by introducing into the bacterium one or more exogenous nucleic acid molecules (such as one or more vectors) comprising the gene operably linked to a promoter that is functional in the bacterium to cause the production of an mRNA molecule.
25. The bacterium according to any one of items 22 to 24, wherein the bacterium comprises an exogenous nucleic acid molecule (such as a vector) comprising a nucleotide sequence encoding the polypeptide having 1-deoxy-D-xylulose-5-phosphate synthase activity.
26. The bacterium according to item 25, wherein the exogenous nucleic acid molecule further comprises a promoter that is functional in the bacterium to cause the production of an mRNA molecule and that is operably linked to the nucleotide sequence encoding the polypeptide.
27. The bacterium according to any one of items 24 to 26, wherein the exogenous nucleic acid molecule is a vector.
28. The bacterium according to any one of items 24 to 26, wherein the exogenous nucleic acid molecule is stably integrated into the genome of the bacterium.
29. The bacterium according to any one of items 22 to 28, wherein the increase in protein expression of the polypeptide having 1-deoxy-D-xylulose-5-phosphate synthase activity is achieved by modifying the ribosome binding site.
30. The bacterium according to any one of items 22 to 29, wherein the increase in protein expression of the polypeptide having 1-deoxy-D-xylulose-5-phosphate synthase activity is achieved by increasing the strength of the promoter operably linked to the gene encoding the polypeptide.
31. The bacterium according to any one of items 22 to 30, wherein the polypeptide having 1-deoxy-D-xylulose-5-phosphate synthase activity is selected from the group consisting of: i) a polypeptide comprising an amino acid sequence of any one of SEQ ID NOs: 63 to 70; and ii) a polypeptide comprising an amino acid sequence, which has at least about 70%, such as at least about 75%, at least about 80%, at least about 85%, at least about 90%, at least about 93%, at least 95%, at least 96%, at least 97%, at least 98%, or at least 99%, sequence identity to the amino acid sequence of any one of SEQ ID NOs: 63 to 70.
32. The bacterium according to any one of items 22 to 31, wherein the polypeptide having 1-deoxy-D-xylulose-5-phosphate synthase activity is selected from the group consisting of: i) a polypeptide comprising an amino acid sequence of any one of SEQ ID NOs: 63 to 65; and ii) a polypeptide comprising an amino acid sequence, which has at least about 70%, such as at least about 75%, at least about 80%, at least about 85%, at least about 90%, at least about 93%, at least 95%, at least 96%, at least 97%, at least 98%, or at least 99%, sequence identity to the amino acid sequence of any one of SEQ ID NOs: 63 to 65.
33. The bacterium according to any one of items 22 to 32, wherein the polypeptide having 1-deoxy-D-xylulose-5-phosphate synthase activity is selected from the group consisting of: i) a polypeptide comprising the amino acid sequence of SEQ ID NO: 63; and ii) a polypeptide comprising an amino acid sequence, which has at least about 70%, such as at least about 75%, at least about 80%, at least about 85%, at least about 90%, at least about 93%, at least 95%, at least 96%, at least 97%, at least 98%, or at least 99%, sequence identity to the amino acid sequence of SEQ ID NO: 63.
34. The bacterium according to any one of items 22 to 32, wherein the polypeptide having 1-deoxy-D-xylulose-5-phosphate synthase activity is selected from the group consisting of: i) a polypeptide comprising the amino acid sequence of SEQ ID NO: 64; and ii) a polypeptide comprising an amino acid sequence, which has at least about 70%, such as at least about 75%, at least about 80%, at least about 85%, at least about 90%, at least about 93%, at least 95%, at least 96%, at least 97%, at least 98%, or at least 99%, sequence identity to the amino acid sequence of SEQ ID NO: 64.
35. The bacterium according to any one of items 22 to 34, wherein the polypeptide having 1-deoxy-D-xylulose-5-phosphate synthase activity is a bacterial polypeptide having 1-deoxy-D-xylulose-5-phosphate synthase activity.
36. The bacterium according to any one of items 1 to 35, which has been further modified to have an increased expression and/or activity of at least one enzyme involved in the purine nucleotide biosynthesis pathway (e.g., at least one enzyme involved in the adenosine monophosphate biosynthesis pathway) compared to an otherwise identical bacterium that does not carry said modification.
37. The bacterium according to item 36, wherein the at least one enzyme involved in the purine nucleotide biosynthesis pathway is selected from the group consisting of: an enzyme having ribose-phosphate diphosphokinase activity, an enzyme having amidophosphoribosyltransferase activity, an enzyme having formyltetrahydrofolate deformylase activity, an enzyme having adenylosuccinate lyase activity, an enzyme having phosphoribosylaminoimidazole-carboxamide formyltransferase activity, an enzyme having adenylosuccinate synthase activity and an enzyme having adenosine kinase activity.
38. The bacterium according to any one of items 1 to 37, which has been further modified to have a decreased expression and/or activity of at least one endogenous enzyme involved in the purine nucleotide degradation pathway compared to an otherwise identical bacterium that does not carry said modification.
39. The bacterium according to item 38, wherein the at least one endogenous enzyme involved in the purine nucleotide degradation pathway is selected from the group consisting of: an enzyme having purine nucleoside phosphorylase activity and an enzyme having adenosine-phosphoribosyltransferase activity.
40. The bacterium according to item 38 or 39, wherein the at least one endogenous enzyme involved in the purine nucleotide degradation pathway is an enzyme having purine nucleoside phosphorylase activity.
41. The bacterium according to any one of items 38 to 40, wherein the at least one endogenous enzyme involved in the purine nucleotide degradation pathway is an enzyme having adenosine-phosphoribosyltransferase activity.
42. The bacterium according to any one of items 1 to 41, which has been further modified to have a decreased expression and/or activity of at least one endogenous enzyme involved in the guanosine monophosphate biosynthesis pathway compared to an otherwise identical bacterium that does not carry said modification.
43. The bacterium according to item 42, wherein the at least one endogenous enzyme involved in the guanosine monophosphate biosynthesis pathway is selected from the group consisting of: an enzyme having IMP dehydrogenase activity and an enzyme having GMP synthetase activity.
44. The bacterium according to item 42 or 43, wherein the at least one endogenous enzyme involved in the guanosine monophosphate biosynthesis pathway is an enzyme having IMP dehydrogenase activity.
45. The bacterium according to any one of items 42 to 44, wherein the at least one endogenous enzyme involved in the guanosine monophosphate biosynthesis pathway is an enzyme having GMP synthetase activity.
46. The bacterium according to any one of items 1 to 45, which has been further modified to have an increased protein expression of a polypeptide having cytochrome P450 monooxygenase (CYP450) activity compared to an otherwise identical bacterium that does not carry said modification.
47. The bacterium according to item 46, wherein the increase in protein expression of the polypeptide having cytochrome P450 monooxygenase (CYP450) activity is achieved by increasing the number of copies of a gene encoding said polypeptide.
48. The bacterium according to item 47, wherein the increase in the number of copies of the gene is achieved by introducing into the bacterium one or more exogenous nucleic acid molecules (such as one or more vectors) comprising the gene operably linked to a promoter that is functional in the bacterium to cause the production of an mRNA molecule.
49. The bacterium according to any one of items 1 to 48, wherein the bacterium comprises an exogenous nucleic acid molecule (such as a vector) comprising a nucleotide sequence encoding the polypeptide having cytochrome P450 monooxygenase (CYP450) activity.
50. The bacterium according to item 49, wherein the exogenous nucleic acid molecule further comprises a promoter that is functional in the bacterium to cause the production of an mRNA molecule and that is operably linked to the nucleotide sequence encoding the polypeptide.
51. The bacterium according to any one of items 48 to 50, wherein the exogenous nucleic acid molecule is a vector.
52. The bacterium according to any one of items 48 to 50, wherein the exogenous nucleic acid molecule is stably integrated into the genome of the bacterium.
53. The bacterium according to any one of items 46 to 52, wherein the polypeptide having cytochrome P450 monooxygenase (CYP450) activity is selected from the group consisting of:
   i) a polypeptide comprising an amino acid sequence of any one of SEQ ID NOs: 93 to 95 and
   ii) a polypeptide comprising an amino acid sequence, which has at least about 70%, such as at least about 75%, at least about 80%, at least about 85%, at least about 90%, at least about 93%, at least 95%, at least 96%, at least 97%, at least 98%, or at least 99%, sequence identity to the amino acid sequence of any one of SEQ ID NOs: 93 to 95.
54. The bacterium according to any one of items 1 to 53, wherein the bacterium is of the family selected from the group consisting of Enterobacteriaceae, Bacillaceae, Lactobacillaceae and Corynebacteriaceae.
55. The bacterium according to any one of items 1 to 53, wherein the bacterium is of the family selected from the group consisting of Bacillaceae and Corynebacteriaceae.
56. The bacterium according to any one of items 1 to 53, wherein the bacterium is of the family Bacillaceae.
57. The bacterium according to any one of items 1 to 53, wherein the bacterium is of the family Corynebacteriaceae.
58. The bacterium according to any one of items 1 to 53, wherein the bacterium is of the genus Bacillus, Lactococcus, Lactobacillus, Clostridium, Corynebacterium, Geobacillus, Thermoanaerobacterium, Streptococcus, Pseudomonas, Streptomyces, Escherichia, Shigella, Acinetobacter, Citrobacter, Salmonella, Klebsiella, Enterobacter, Erwinia, Kluyvera, Serratia, Cedecea, Morganella, Hafnia, Edwardsiella, Providencia, Proteus or Yersinia.
59. The bacterium according to any one of items 1 to 53, wherein the bacterium is of the genus Bacillus or Corynebacterium.
60. The bacterium according to any one of items 1 to 53, wherein the bacterium is of the genus Bacillus.
61. The bacterium according to any one of items 1 to 53, wherein the bacterium is of the genus Corynebacterium.
62. The bacterium according to any one of items 1 to 53, wherein the bacterium is Bacillus subtilis.
63. The bacterium according to any one of items 1 to 53, wherein the bacterium is Corynebacterium stationis.
64. Method for producing an isoprenoid cytokinin or riboside derivative thereof, comprising cultivating a bacterium according to any one of items 1 to 63 under suitable culture conditions in a suitable culture medium.
65. The method according to item 64, wherein the isoprenoid cytokinin or riboside derivative thereof is selected from the group consisting of trans-zeatin (tZ), trans-zeatin riboside (tZR), N⁶-(D2-isopentenyl)adenine (iP), N(6)-(dimethylallyl)adenosine (iPR), dihydrozeatin (DZ), ribosyl dihydrozeatin (DZR), and combinations thereof.
66. The method according to item 65, wherein the isoprenoid cytokinin or riboside derivative thereof is trans-zeatin (tZ) and trans-zeatin riboside (tZR), respectively.
67. The method according to item 64, wherein the method is for producing trans-zeatin (tZ).

### BRIEF DESCRIPTION OF THE FIGURES

FIG. 1: Isoprenoid cytokinins: A) Ribosides: N6-(D2-isopentenyl)adenine riboside (iPR), trans-zeatin riboside (tZR), cis-zeatin riboside (cZR) and dihydrozeatin riboside (DZR). B) Free bases: N⁶-(D2-isopentenyl)adenine (iP), trans-zeatin (tZ), cis-zeatin (cZ) and dihydrozeatin (DZ).

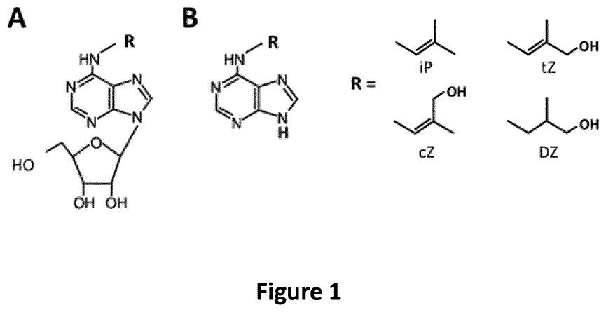

FIG. 2: Schematic representation of the MEP metabolic pathway.

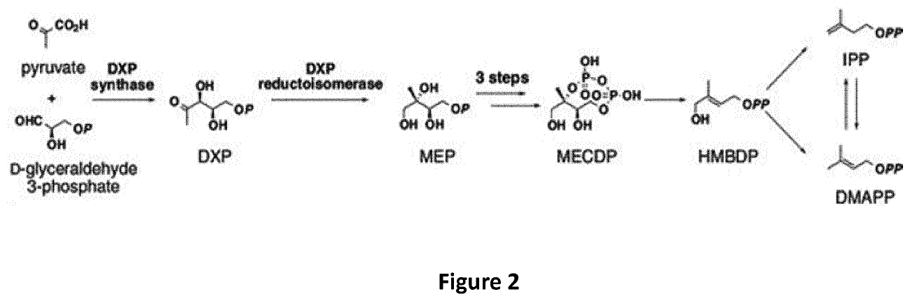

FIG. 3: Isoprenoid cytokinin biosynthesis pathway with heterologous expression of IPT (EC 2.5.1.27) and LOG (EC 3.2.2.n1).

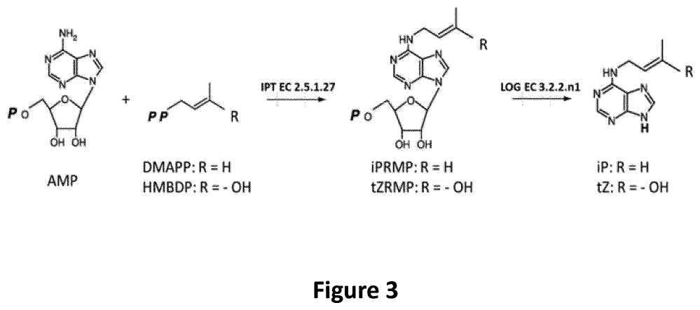

FIG. 4: Production of trans-zeatin and related isoprenoid cytokinins of Bacillus subtilis strains in experiments in shaker-scale experiments. VKPM B2116—parent strain, TZAB14, TZAB15—IPT-LOG.

FIG. 5: Production of trans-zeatin and related isoprenoid cytokinins of Bacillus subtilis strains in experiments in shaker-scale experiments with adenine sulfate. VKPM B2116—parent strain, TZAB14—IPT-LOG.

FIG. 6: Production of trans-zeatin and related isoprenoid cytokinins by Bacillus subtilis strains with IPT and LOG genes and DXS gene in shaker-scale experiments. VKPM B2116—parent strain, TZAB15—IPT-LOG, TZAB43—IPT-LOG-DXS.

FIG. 7: Production of trans-zeatin and related isoprenoid cytokinins by Bacillus subtilis strains expressing IPT (SEQ ID NO: 1) and LOG (SEQ ID NO: 34) or IPT (SEQ ID NO: 2) and LOG (SEQ ID NO: 34) after 28 h in shaker-scale experiments. VKPM B2116—parent strain, TZAB1, TZAB2, TZAB3, TZAB4—strains with IPT (SEQ ID NO: 2) and LOG (SEQ ID NO: 34), TZAB14, TZAB15—strains with IPT (SEQ ID NO: 1) and LOG (SEQ ID NO: 34).

FIG. 8: Production of cytokinins in Bacillus subtilis strain 168 with IPT (SEQ ID NO: 1), LOG (SEQ ID NO: 34) and overexpression of DXS (SEQ ID NO: 63) after 24 h in shaker-scale experiments.

FIG. 9: Production of cytokinins in Bacillus subtilis strain RB50 with IPT (SEQ ID NO: 1), LOG (SEQ ID NO: 34) and overexpression of DXS (SEQ ID NO: 63) after 18 h in shaker-scale experiments.

FIG. 10: Production of cytokinins in Bacillus subtilis strain VKPM B2116 with IPT (SEQ ID NO: 1), LOG (SEQ ID NO: 34) and overexpression of DXS (SEQ ID NO: 63) after 24 h in shaker-scale experiments.

FIG. 11: Production of cytokinins in Escherichia coli strain BL21(DE3) with IPT (SEQ ID NO: 1), LOG (SEQ ID NO: 34) and overexpression of DXS (SEQ ID NO: 63) after 10 h in shaker-scale experiments.

FIG. 12: Production of cytokinins in Corynebacterium stationis with IPT (SEQ ID NO: 1), LOG (SEQ ID NO: 34) and overexpression of DXS (SEQ ID NO: 63) after 48 h in shaker-scale experiments.

FIG. 13: Production of cytokinins in Bacillus subtilis strains expressing LOG 8 (SEQ ID 41) in combination with various IPTs (SEQ ID NO: 1, SEQ ID NO: 6, SEQ ID NO: 7 and SEQ ID NO: 9) after 18 h in shaker-scale experiments.

FIG. 14: Production of cytokinins in Bacillus subtilis strains expressing IPT (SEQ ID NO: 1) in combination with various LOGs (SEQ ID NO: 34-44) after 18 h in shaker-scale experiments.

The present invention is now described in more detail below.

### DETAILED DESCRIPTION OF THE INVENTION

Unless specifically defined herein, all technical and scientific terms used have the same meaning as commonly understood by a skilled artisan in the fields of biochemistry, genetics, and microbiology.

All methods and materials similar or equivalent to those described herein can be used in the practice or testing of the present invention, with suitable methods and materials being described herein. All publications, patent applications, patents, and other references mentioned herein are incorporated by reference in their entirety. In case of conflict, the present specification, including definitions, will prevail. Further, the materials, methods, and examples are illustrative only and are not intended to be limiting, unless otherwise specified.

The practice of the present invention will employ, unless otherwise indicated, conventional techniques of cell biology, cell culture, molecular biology, transgenic biology, microbiology, and recombinant DNA, which are within the skill of the art. Such techniques are explained fully in the literature. See, for example, Current Protocols in Molecular Biology (Frederick M. AUSUBEL, 2000, Wiley and son Inc, Library of Congress, USA); Molecular Cloning: A Laboratory Manual, Third Edition, (Sambrook et al, 2001, Cold Spring Harbor, New York: Cold Spring Harbor Laboratory Press); Oligonucleotide Synthesis (M. J. Gait ed., 1984); Mullis et al. U.S. Pat. No. 4,683,195; Nucleic Acid Hybridization (B. D. Harries & S. J. Higgins eds. 1984); Transcription And Translation (B. D. Hames & S. J. Higgins eds. 1984); Culture Of Animal Cells (R. I. Freshney, Alan R. Liss, Inc., 1987); Immobilized Cells And Enzymes (IRL Press, 1986); B. Perbal, A Practical Guide To Molecular Cloning (1984); the series, Methods In ENZYMOLOGY (J. Abelson and M. Simon, eds.-in-chief, Academic Press, Inc., New York), specifically, Vols. 154 and 155 (Wu et al. eds.) and Vol. 185, “Gene Expression Technology” (D. Goeddel, ed.).

Bacterium of the Invention

As indicated above, the present inventors have engineered bacterial strains, which a) express a heterologous polypeptide having adenylate isopentenyltransferase activity and optionally b) have been modified to have an increased protein expression of a polypeptide having cytokinin riboside 5-monophosphate phosphoribohydrolase activity. As shown in the Examples, such engineered bacterial strains surprisingly show unusually high titers of isoprenoid cytokinins of over 10 mg/L in the supernatant. In consequence, this means that the context of plant cell infection is no longer required and the biosynthetic substrates and cofactors for efficient biosynthesis of isoprenoid cytokinins such as tZ, and iP, and their ribosides tZR and iPR are effectively supplied by the engineered bacterial cell.

The present invention thus provides in a first aspect a bacterium expressing a heterologous polypeptide having adenylate isopentenyltransferase activity. More particularly, the present invention provides a bacterium, which a) expresses a heterologous polypeptide having adenylate isopentenyltransferase activity and optionally b) has been modified to have an increased protein expression of a polypeptide having cytokinin riboside 5-monophosphate phosphoribohydrolase activity compared to an otherwise identical bacterium that does not carry said modification.

Adenylate isopentenyltransferases (IPTs) are a well-defined class of enzymes catalyzing the first step in the de novo cytokinin biosynthesis pathway, the N-prenylation of adenosine 5′-phosphates (AMP, ADP, or ATP) with either dimethylallyl diphosphate (DMAPP) or 1-hydroxy-2-methyl-2-butenyl 4-diphosphate (HMBDP). There are two types of IPTs (EC 2.5.1.27 and EC 2.5.1.112). The IPT enzymes are present in bacteria and plants. IPTs from bacteria, such as from Agrobacterium which contains two IPT homologs, Tzs and Tmr, use AMP as a prenyl acceptor, and HMBDP or DMAPP as the donor (EC 2.5.1.27). The product of the reaction with HMBDP is tZ-nucleotide. Moreover, IPTs from bacteria belong to the Pfam family IPT (PF01745) in the Pfam database of protein families and domains (https://pfam.xfam.org/family/ipt). The IPT Pfam domain genes are phylogenetically scattered and found only in some members of Actinobacteria, Cyanobacteria, α-Proteobacteria, β-Proteobacteria, and γ-Proteobacteria and in the eukaryote Dictyostelium discoideum (Nishii et al., 2018). IPTs from higher plants use predominantly ATP or ADP as a prenyl acceptor and DMAPP as the donor (EC 2.5.1.112) and belong to the Pfam family IPPT (PF01715) (https://pfam.xfam.org/family/ippt). The product of the reaction is iP, which is then hydroxylated at the prenyl side chain to result in tZ-nucleotides.

Tzs of Agrobacterium tumefaciens (IPT (PF01745); EC 2.5.1.27; SEQ ID NO: 1) consists of 2 domains: the N-terminal domain with a five-stranded parallel β-sheet, which is surrounded by 3 α-helices (α1-α3), and the C-terminal domain with 5 α helices (α4-α8). The N-terminal domain contains a nucleotide-binding p loop motif Gly-8-Pro-Thr-Cys-Ser-Gly-Lys-Thr-15, and is structurally related to the p loop-containing nucleoside triphosphate hydrolase (pNTPase) superfamily. The C-terminal side of α8 extends to the N-terminal and is attached to it. The interface between the domains forms a solvent-accessible channel, which binds AMP. The prenylation site of AMP binds to Asp-33 and Ser-45. DMAPP is bound to Asp-173, Tyr-211, and His-214. Thr-10, Asp-33, and Arg-138 are fully conserved among IPTs. In Tzs, the hydrophilic region formed by the side chains contains His-214 and Asp-173, which are two critical amino acid residues within the substrate-binding pocket that distinguish the presence or absence of the hydroxyl group of the prenyl-donor substrate, which allows the use of HMBDP (Sugawara et al. 2008).

Generally, the polypeptide having adenylate isopentenyltransferase activity employed according to the invention will be heterologous to the bacterium, which means that said polypeptide is normally not found in or made (i.e. expressed) by the bacterium, but is derived from a different species. Moreover, the polypeptide having adenylate isopentenyltransferase activity is derived from or corresponds to a member of the Pfam family IPT (PF01745), and preferably is a bacterial polypeptide having adenylate isopentenyltransferase activity. With “bacterial polypeptide having adenylate isopentenyltransferase activity” it is meant that the polypeptide having adenylate isopentenyltransferase activity is derived from a bacterium, such as Agrobacterium tumefaciens.

Polypeptides having adenylate isopentenyltransferase activity most suitable for the biosynthesis of isoprenoid cytokinins including tZ, iP, tZR and iPR, are enzymes capable of using both HMBDP and DMAPP as prenyl donors, such as Tzs or Tmr of Agrobacterium tumefaciens. Moreover, they belong to the Pfam family IPT (PF01745) and contain an Asp residue in a position equivalent to position 173 of SEQ ID NO:1, a Tyr residue in a position equivalent to position 211 of SEQ ID NO:1 and/or a His residue in a position equivalent to position 214 of SEQ ID NO:1.

A polypeptide having adenylate isopentenyltransferase activity for use according to the invention may for instance be a polypeptide having adenylate isopentenyltransferase activity selected from the group consisting of: i) a polypeptide comprising the amino acid sequence of any one of SEQ ID NOs: 1 to 33; and ii) a polypeptide comprising an amino acid sequence, which has at least about 50%, such as at least about 55%, at least about 60%, at least about 65%, at least about 70%, at least about 75%, at least about 80%, at least about 85%, at least about 90%, at least about 93%, at least 95%, at least 96%, at least 97%, at least 98%, or at least 99%, sequence identity to the amino acid sequence of any one of SEQ ID NOs: 1 to 33.

A polypeptide having adenylate isopentenyltransferase activity for use according to the invention may for instance be a polypeptide having adenylate isopentenyltransferase activity selected from the group consisting of: i) a polypeptide comprising the amino acid sequence of any one of SEQ ID NOs: 1 to 33; and ii) a polypeptide comprising an amino acid sequence, which has at least about 70%, such as at least about 75%, at least about 80%, at least about 85%, at least about 90%, at least about 93%, at least 95%, at least 96%, at least 97%, at least 98%, or at least 99%, sequence identity to the amino acid sequence of any one of SEQ ID NOs: 1 to 33.

A polypeptide having adenylate isopentenyltransferase activity for use according to the invention may for instance be a polypeptide having adenylate isopentenyltransferase activity selected from the group consisting of: i) a polypeptide comprising the amino acid sequence of any one of SEQ ID NOs: 1 to 10; and ii) a polypeptide comprising an amino acid sequence, which has at least about 85%, such as at least about 90%, at least about 93%, at least 95%, at least 96%, at least 97%, at least 98%, or at least 99%, sequence identity to the amino acid sequence of any one of SEQ ID NOs: 1 to 10.

A polypeptide having adenylate isopentenyltransferase activity for use according to the invention may for instance be a polypeptide having adenylate isopentenyltransferase activity selected from the group consisting of: i) a polypeptide comprising the amino acid sequence of any one of SEQ ID NOs: 1 to 10; and ii) a polypeptide comprising an amino acid sequence, which has at least about 70%, such as at least about 75%, at least about 80%, at least about 85%, at least about 90%, at least about 93%, at least 95%, at least 96%, at least 97%, at least 98%, or at least 99%, sequence identity to the amino acid sequence of any one of SEQ ID NOs: 1 to 10.

A polypeptide having adenylate isopentenyltransferase activity for use according to the invention may for instance be a polypeptide having adenylate isopentenyltransferase activity selected from the group consisting of: i) a polypeptide comprising the amino acid sequence of any one of SEQ ID NOs: 1 to 10; and ii) a polypeptide comprising an amino acid sequence, which has at least about 85%, such as at least about 90%, at least about 93%, at least 95%, at least 96%, at least 97%, at least 98%, or at least 99%, sequence identity to the amino acid sequence of any one of SEQ ID NOs: 1 to 10.

According to some embodiments, the polypeptide having adenylate isopentenyltransferase activity comprises an amino acid sequence having at least 50%, such as at least 55%, sequence identity with the amino acid sequence SEQ ID NO: 1. According to some embodiments, the polypeptide having adenylate isopentenyltransferase activity comprises an amino acid sequence having at least 60%, such as at least 65%, sequence identity with the amino acid sequence SEQ ID NO: 1. According to some embodiments, the polypeptide having adenylate isopentenyltransferase activity comprises an amino acid sequence having at least 70%, such as at least 75%, sequence identity with the amino acid sequence SEQ ID NO: 1. According to some embodiments, the polypeptide having adenylate isopentenyltransferase activity comprises an amino acid sequence having at least 80%, such as at least 85%, sequence identity with the amino acid sequence SEQ ID NO: 1. According to some embodiments, the polypeptide having adenylate isopentenyltransferase activity comprises an amino acid sequence having at least 90%, such as at least 95%, sequence identity with the amino acid sequence SEQ ID NO: 1. According to some embodiments, the polypeptide having adenylate isopentenyltransferase activity comprises an amino acid sequence having at least 95%, such as at least 97%, sequence identity with the amino acid sequence SEQ ID NO: 1. According to some embodiments, the polypeptide having adenylate isopentenyltransferase activity comprises the amino acid sequence of SEQ ID NO: 1.

According to some embodiments, the polypeptide having adenylate isopentenyltransferase activity comprises an amino acid sequence having at least 50%, such as at least 55%, sequence identity with the amino acid sequence SEQ ID NO: 2. According to some embodiments, the polypeptide having adenylate isopentenyltransferase activity comprises an amino acid sequence having at least 60%, such as at least 65%, sequence identity with the amino acid sequence SEQ ID NO: 2. According to some embodiments, the polypeptide having adenylate isopentenyltransferase activity comprises an amino acid sequence having at least 70%, such as at least 75%, sequence identity with the amino acid sequence SEQ ID NO: 2. According to some embodiments, the polypeptide having adenylate isopentenyltransferase activity comprises an amino acid sequence having at least 80%, such as at least 85%, sequence identity with the amino acid sequence SEQ ID NO: 2. According to some embodiments, the polypeptide having adenylate isopentenyltransferase activity comprises an amino acid sequence having at least 90%, such as at least 95%, sequence identity with the amino acid sequence SEQ ID NO: 2. According to some embodiments, the polypeptide having adenylate isopentenyltransferase activity comprises an amino acid sequence having at least 95%, such as at least 97%, sequence identity with the amino acid sequence SEQ ID NO: 2. According to some embodiments, the polypeptide having adenylate isopentenyltransferase activity comprises the amino acid sequence of SEQ ID NO: 2.

According to some embodiments, the polypeptide having adenylate isopentenyltransferase activity comprises an amino acid sequence having at least 50%, such as at least 55%, sequence identity with the amino acid sequence SEQ ID NO: 3. According to some embodiments, the polypeptide having adenylate isopentenyltransferase activity comprises an amino acid sequence having at least 60%, such as at least 65%, sequence identity with the amino acid sequence SEQ ID NO: 3. According to some embodiments, the polypeptide having adenylate isopentenyltransferase activity comprises an amino acid sequence having at least 70%, such as at least 75%, sequence identity with the amino acid sequence SEQ ID NO: 3. According to some embodiments, the polypeptide having adenylate isopentenyltransferase activity comprises an amino acid sequence having at least 80%, such as at least 85%, sequence identity with the amino acid sequence SEQ ID NO: 3. According to some embodiments, the polypeptide having adenylate isopentenyltransferase activity comprises an amino acid sequence having at least 90%, such as at least 95%, sequence identity with the amino acid sequence SEQ ID NO: 3. According to some embodiments, the polypeptide having adenylate isopentenyltransferase activity comprises an amino acid sequence having at least 95%, such as at least 97%, sequence identity with the amino acid sequence SEQ ID NO: 3. According to some embodiments, the polypeptide having adenylate isopentenyltransferase activity comprises the amino acid sequence of SEQ ID NO: 3.

According to some embodiments, the polypeptide having adenylate isopentenyltransferase activity comprises an amino acid sequence having at least 50%, such as at least 55%, sequence identity with the amino acid sequence SEQ ID NO: 4. According to some embodiments, the polypeptide having adenylate isopentenyltransferase activity comprises an amino acid sequence having at least 60%, such as at least 65%, sequence identity with the amino acid sequence SEQ ID NO: 4. According to some embodiments, the polypeptide having adenylate isopentenyltransferase activity comprises an amino acid sequence having at least 70%, such as at least 75%, sequence identity with the amino acid sequence SEQ ID NO: 4. According to some embodiments, the polypeptide having adenylate isopentenyltransferase activity comprises an amino acid sequence having at least 80%, such as at least 85%, sequence identity with the amino acid sequence SEQ ID NO: 4. According to some embodiments, the polypeptide having adenylate isopentenyltransferase activity comprises an amino acid sequence having at least 90%, such as at least 95%, sequence identity with the amino acid sequence SEQ ID NO: 4. According to some embodiments, the polypeptide having adenylate isopentenyltransferase activity comprises an amino acid sequence having at least 95%, such as at least 97%, sequence identity with the amino acid sequence SEQ ID NO: 4. According to some embodiments, the polypeptide having adenylate isopentenyltransferase activity comprises the amino acid sequence of SEQ ID NO: 4.

According to some embodiments, the polypeptide having adenylate isopentenyltransferase activity comprises an amino acid sequence having at least 50%, such as at least 55%, sequence identity with the amino acid sequence SEQ ID NO: 5. According to some embodiments, the polypeptide having adenylate isopentenyltransferase activity comprises an amino acid sequence having at least 60%, such as at least 65%, sequence identity with the amino acid sequence SEQ ID NO: 5. According to some embodiments, the polypeptide having adenylate isopentenyltransferase activity comprises an amino acid sequence having at least 70%, such as at least 75%, sequence identity with the amino acid sequence SEQ ID NO: 5. According to some embodiments, the polypeptide having adenylate isopentenyltransferase activity comprises an amino acid sequence having at least 80%, such as at least 85%, sequence identity with the amino acid sequence SEQ ID NO: 5. According to some embodiments, the polypeptide having adenylate isopentenyltransferase activity comprises an amino acid sequence having at least 90%, such as at least 95%, sequence identity with the amino acid sequence SEQ ID NO: 5. According to some embodiments, the polypeptide having adenylate isopentenyltransferase activity comprises an amino acid sequence having at least 95%, such as at least 97%, sequence identity with the amino acid sequence SEQ ID NO: 5. According to some embodiments, the polypeptide having adenylate isopentenyltransferase activity comprises the amino acid sequence of SEQ ID NO: 5.

According to some embodiments, the polypeptide having adenylate isopentenyltransferase activity comprises an amino acid sequence having at least 50%, such as at least 55%, sequence identity with the amino acid sequence SEQ ID NO: 6. According to some embodiments, the polypeptide having adenylate isopentenyltransferase activity comprises an amino acid sequence having at least 60%, such as at least 65%, sequence identity with the amino acid sequence SEQ ID NO: 6. According to some embodiments, the polypeptide having adenylate isopentenyltransferase activity comprises an amino acid sequence having at least 70%, such as at least 75%, sequence identity with the amino acid sequence SEQ ID NO: 6. According to some embodiments, the polypeptide having adenylate isopentenyltransferase activity comprises an amino acid sequence having at least 80%, such as at least 85%, sequence identity with the amino acid sequence SEQ ID NO: 6. According to some embodiments, the polypeptide having adenylate isopentenyltransferase activity comprises an amino acid sequence having at least 90%, such as at least 95%, sequence identity with the amino acid sequence SEQ ID NO: 6. According to some embodiments, the polypeptide having adenylate isopentenyltransferase activity comprises an amino acid sequence having at least 95%, such as at least 97%, sequence identity with the amino acid sequence SEQ ID NO: 6. According to some embodiments, the polypeptide having adenylate isopentenyltransferase activity comprises the amino acid sequence of SEQ ID NO: 6.

According to some embodiments, the polypeptide having adenylate isopentenyltransferase activity comprises an amino acid sequence having at least 50%, such as at least 55%, sequence identity with the amino acid sequence SEQ ID NO: 7. According to some embodiments, the polypeptide having adenylate isopentenyltransferase activity comprises an amino acid sequence having at least 60%, such as at least 65%, sequence identity with the amino acid sequence SEQ ID NO: 7. According to some embodiments, the polypeptide having adenylate isopentenyltransferase activity comprises an amino acid sequence having at least 70%, such as at least 75%, sequence identity with the amino acid sequence SEQ ID NO: 7. According to some embodiments, the polypeptide having adenylate isopentenyltransferase activity comprises an amino acid sequence having at least 80%, such as at least 85%, sequence identity with the amino acid sequence SEQ ID NO: 7. According to some embodiments, the polypeptide having adenylate isopentenyltransferase activity comprises an amino acid sequence having at least 90%, such as at least 95%, sequence identity with the amino acid sequence SEQ ID NO: 7. According to some embodiments, the polypeptide having adenylate isopentenyltransferase activity comprises an amino acid sequence having at least 95%, such as at least 97%, sequence identity with the amino acid sequence SEQ ID NO: 7. According to some embodiments, the polypeptide having adenylate isopentenyltransferase activity comprises the amino acid sequence of SEQ ID NO: 7.

According to some embodiments, the polypeptide having adenylate isopentenyltransferase activity comprises an amino acid sequence having at least 50%, such as at least 55%, sequence identity with the amino acid sequence SEQ ID NO: 8. According to some embodiments, the polypeptide having adenylate isopentenyltransferase activity comprises an amino acid sequence having at least 60%, such as at least 65%, sequence identity with the amino acid sequence SEQ ID NO: 8. According to some embodiments, the polypeptide having adenylate isopentenyltransferase activity comprises an amino acid sequence having at least 70%, such as at least 75%, sequence identity with the amino acid sequence SEQ ID NO: 8. According to some embodiments, the polypeptide having adenylate isopentenyltransferase activity comprises an amino acid sequence having at least 80%, such as at least 85%, sequence identity with the amino acid sequence SEQ ID NO: 8. According to some embodiments, the polypeptide having adenylate isopentenyltransferase activity comprises an amino acid sequence having at least 90%, such as at least 95%, sequence identity with the amino acid sequence SEQ ID NO: 8. According to some embodiments, the polypeptide having adenylate isopentenyltransferase activity comprises an amino acid sequence having at least 95%, such as at least 97%, sequence identity with the amino acid sequence SEQ ID NO: 8. According to some embodiments, the polypeptide having adenylate isopentenyltransferase activity comprises the amino acid sequence of SEQ ID NO: 8.

According to some embodiments, the polypeptide having adenylate isopentenyltransferase activity comprises an amino acid sequence having at least 50%, such as at least 55%, sequence identity with the amino acid sequence SEQ ID NO: 9. According to some embodiments, the polypeptide having adenylate isopentenyltransferase activity comprises an amino acid sequence having at least 60%, such as at least 65%, sequence identity with the amino acid sequence SEQ ID NO: 9. According to some embodiments, the polypeptide having adenylate isopentenyltransferase activity comprises an amino acid sequence having at least 70%, such as at least 75%, sequence identity with the amino acid sequence SEQ ID NO: 9. According to some embodiments, the polypeptide having adenylate isopentenyltransferase activity comprises an amino acid sequence having at least 80%, such as at least 85%, sequence identity with the amino acid sequence SEQ ID NO: 9. According to some embodiments, the polypeptide having adenylate isopentenyltransferase activity comprises an amino acid sequence having at least 90%, such as at least 95%, sequence identity with the amino acid sequence SEQ ID NO: 9. According to some embodiments, the polypeptide having adenylate isopentenyltransferase activity comprises an amino acid sequence having at least 95%, such as at least 97%, sequence identity with the amino acid sequence SEQ ID NO: 9. According to some embodiments, the polypeptide having adenylate isopentenyltransferase activity comprises the amino acid sequence of SEQ ID NO: 9.

According to some embodiments, the polypeptide having adenylate isopentenyltransferase activity comprises an amino acid sequence having at least 50%, such as at least 55%, sequence identity with the amino acid sequence SEQ ID NO: 10. According to some embodiments, the polypeptide having adenylate isopentenyltransferase activity comprises an amino acid sequence having at least 60%, such as at least 65%, sequence identity with the amino acid sequence SEQ ID NO: 10. According to some embodiments, the polypeptide having adenylate isopentenyltransferase activity comprises an amino acid sequence having at least 70%, such as at least 75%, sequence identity with the amino acid sequence SEQ ID NO: 10. According to some embodiments, the polypeptide having adenylate isopentenyltransferase activity comprises an amino acid sequence having at least 80%, such as at least 85%, sequence identity with the amino acid sequence SEQ ID NO: 10. According to some embodiments, the polypeptide having adenylate isopentenyltransferase activity comprises an amino acid sequence having at least 90%, such as at least 95%, sequence identity with the amino acid sequence SEQ ID NO: 10. According to some embodiments, the polypeptide having adenylate isopentenyltransferase activity comprises an amino acid sequence having at least 95%, such as at least 97%, sequence identity with the amino acid sequence SEQ ID NO: 10. According to some embodiments, the polypeptide having adenylate isopentenyltransferase activity comprises the amino acid sequence of SEQ ID NO: 10.

Techniques for determining adenylate isopentenyltransferase activity are well known to the skilled person. Exemplary methods have been described, e.g. by Takei, Sakakibara, and Sugiyama (2001) and by Frébortová, Greplová et al, (2015). The adenylate isopentenyltransferase activity may for instance be determined in accordance with any of the following assays:
(1) Enzyme is incubated in a reaction mixture (1M betaine, 20 mM triethanolamine, 50 mM KCl, 10 mM MgCl₂, 1 mM dithiothreitol, 1 mg/ml bovine serum albumin, pH 8.0) with 1 mM AMP and 340 μM DMAPP at 25° C. for 20 min. The reaction is stopped by the addition of a quarter volume of 10% acetate and centrifuged at 18,000×g for 20 min. The resulting supernatant is subjected to cytokinin analysis. One unit of IPT activity is defined as the amount of enzyme that produced 1 μmol of iPMP/min under the condition of the reaction (Takei, Sakakibara, and Sugiyama 2001).
(2) Activity assay is performed at 25° C. overnight in 200 μl of a reaction mixture (100 mM Tris/HCl buffer, pH 7.5, containing 10 mM MgCl₂), with 100 μM AMP and 100 μM DMAPP (Echelon BioSciences, Salt Lake City, UT, USA) as substrates and 100 μl of purified enzyme. To assess substrate preference of IPT, ADP or ATP are used as isoprene chain accepting substrates, whereas isopenthenyl diphosphate or HMBPP (Echelon Bioscences) as isoprene chain donating substrates. The reaction is initiated by adding the isoprenoid substrate and stopped by heating to 95° C. for 5 min to inactivate the enzyme. The IPT activity assay is based on determination of reaction products by HPLC or capillary electrophoresis with UV detection at 268 nm. Cytokinin ribosides and corresponding monophosphates are determined on Symmetry C18 column (2.1×150 mm, 5 μm; Waters, Milford, MA, USA) connected to Alliance 2695 high performance liquid chromatograph (Waters). The column was eluted by a linear gradient of 15 mM ammonium formate, pH 4.0 (A) and methanol (B) using the following solvent mixture: 0-25 min, 5-60% B, 25-26 min, 60-100% B, 26-27 min 100% B. Linear gradient of 15 mM ammonium formate, pH 4.0 (A) and acetonitrile (B) was used for the analysis of oligoribonucleotide hydrolysates (0-30 min, 5-24% B, 30-31 min, 24-100% B). The flow rate is 0.25 ml/min and the column temperature is 30° C. The concentration of the product is determined by a calibration curve method using authentic standard compounds (Olchemim, Olomouc, Czech Republic). Capillary electrophoresis is used for determination of cytokinin di- and triphosphates (Frébortová, Greplová et al, 2015).

Besides expressing a heterologous polypeptide having adenylate isopentenyltransferase activity, the bacterium of the present invention may optionally be (further) modified to have an increased protein expression of a polypeptide having cytokinin riboside 5-monophosphate phosphoribohydrolase activity compared to an otherwise identical bacterium that does not carry said modification.

Thus, according to some embodiments, a bacterium of the present invention expresses a heterologous polypeptide having adenylate isopentenyltransferase activity and has been modified to have an increased protein expression of a polypeptide having cytokinin riboside 5-monophosphate phosphoribohydrolase activity compared to an otherwise identical bacterium that does not carry said modification.

By “increased protein expression” it is meant that the amount of the polypeptide having cytokinin riboside 5-monophosphate phosphoribohydrolase activity produced by the thus modified bacterium is increased compared to an otherwise identical bacterium that does not carry said modification. More particularly, by “increased expression” it is meant that the amount of the polypeptide having cytokinin riboside 5-monophosphate phosphoribohydrolase activity produced by the thus modified bacterium is increased by at least 10%, such as at least 20%, at least 30%, at least 40%, at least 50% at least 60%, at least 70%, at least 80%, at least 90%, at least 100%, at least 150%, at least 200%, at least 300%, at least 400%, at least 500%, at least 600%, at least 700% at least 800%, at least about 900%, at least about 1000%, at least about 2000%, at least about 3000%, at least about 4000%, at least about 5000%, at least about 6000%, at least about 7000%, at least about 8000% at least about 9000% or at least about 10000%, compared to an otherwise identical bacterium that does not carry said modification. The amount of protein in a given cell can be determined by any suitable quantification technique known in the art, such as ELISA, Immunohistochemistry, or Western Blotting.

An increase in protein expression may be achieved by any suitable means well-known to those skilled in the art. For example, an increase in protein expression may be achieved by increasing the number of copies of the gene encoding the polypeptide having cytokinin riboside 5-monophosphate phosphoribohydrolase activity in the bacterium, such as by introducing into the bacterium an exogenous nucleic acid, such as a vector, comprising the gene encoding the polypeptide having cytokinin riboside 5-monophosphate phosphoribohydrolase activity operably linked to a promoter that is functional in the bacterium to cause the production of an mRNA molecule.

An increase in protein expression may also be achieved by the integration of at least a second copy of the gene encoding the polypeptide having cytokinin riboside 5-monophosphate phosphoribohydrolase activity into the genome of the bacterium.

An increase in protein expression may also be achieved by increasing the strength of the promoter operably linked to the gene encoding the polypeptide having cytokinin riboside 5-monophosphate phosphoribohydrolase activity, e.g. by replacing the native promoter with a promoter that enables higher expression and overproduction of polypeptide compared to the native promoter. The promoters that can be used include natural promoters from Bacillus subtilis, Bacillus amyloliquefaciens or similar, such as P43, P15, Pveg, Pylb, PgroES, PsigX, PtrnQ, Ppst, PsodA, PrpsF, PlepA, PliaG, PrpsF, Ppst, PfusA, PsodA, Phag as well as artificial promoters active in Bacillus subtilis or inducible Bacillus subtilis promoters, such as PmtlA, Pspac, PxylA, PsacB, or similar. Further examples include natural promoters from Corynebacterium, such as P CP_2454, Ptuf and Psod, natural promoters from E. coli, such as T7, ParaBAD, Plac, Ptac and Ptrc, and the promoter P F1 derived from the corynephage BFK20.

An increase in protein expression may also be achieved by modifying the ribosome binding site on the mRNA molecule encoding the polypeptide having cytokinin riboside 5-monophosphate phosphoribohydrolase activity. By modifying the sequence of the ribosome binding site, the translation initiation rate may be increased, thus increasing translation efficiency.

According to some embodiments, the increase in the number of copies of the gene is achieved by introducing into the bacterium one or more (such as two or three) exogenous nucleic acid molecules (such as one or more vectors) comprising the gene operably linked to a promoter that is functional in the host cell to cause the production of an mRNA molecule.

According to some embodiments, a bacterium of the invention comprises an exogenous nucleic acid molecule (such as a vector) comprising one or more (such as two, three or four) nucleotide sequences encoding the polypeptide having cytokinin riboside 5-monophosphate phosphoribohydrolase activity. Suitably, the exogenous nucleic acid molecule further comprises a promoter that is functional in the bacterium to cause the production of an mRNA molecule and that is operably linked to the nucleotide sequence encoding said polypeptide having cytokinin riboside 5-monophosphate phosphoribohydrolase activity. According to some embodiments, the exogenous nucleic acid molecule is stably integrated into the genome of the bacterium.

A polypeptide having cytokinin riboside 5-monophosphate phosphoribohydrolase activity is a cytokinin-activating enzyme with the enzymatic function as cytokinin riboside 5′-monophosphate phosphoribohydrolase. Such polypeptides, also referred to as Lonely Guy (LOG) proteins, are encoded in the genomes of a wide range of organisms, and a majority of LOG proteins are prokaryotic. Enzymes from several organisms, such as Oryza sativa, Arabidopsis thaliana, Claviceps purpurea, Mycobacterium tuberculosis and Corynebacterium glutamicum have been characterized as LOGs by biochemical and functional studies. LOG protein was originally characterized as a phytohormone-activating plant enzyme, while bacterial LOG homologs were mistakenly designated as putative lysine decarboxylases (LDCs) without experimental evidence. Their true enzymatic activity was recently confirmed by functional analysis of the Corynebacterium glutamicum homolog (Seo et al. 2016). Two types of LOG proteins were identified in bacteria, dimeric type I LOGs and hexameric type II LOGs. Type II LOG proteins have different oligomeric state and residues at the prenyl-binding site. Type I LOGs can be further divided into 2 subgroups, type Ia and type Ib. Type Ia includes dimeric LOGs from most organisms, whereas type Ib includes dimeric LOGs from Actinomycetales. Type II LOG are divided into type IIa, which include hexameric LOGs from most organisms, and type IIb with LOGs from higher plants (Seo and Kim 2017).

LOG proteins produce active cytokinins via dephosphoribosylation, hydrolysis of the bond between N⁶-substituted bases and ribose 5′-monophosphates in cytokinin precursors such as iPRMP or trans-zeatin riboside 5′-monophosphate (tZRMP). C. glutamicum LOG is composed of two identical monomers with a central β-sheet, formed by seven parallel β-strands, which is surrounded by eight α-helices. LOG proteins contain “PGGXGTXXE” motif that contributes to the formation of an active site. The active site is formed in the pocket of the dimer, and the conserved “PGGXGTXXE” motif is on the surface of the pocket. The “PGGXGTXXE” motif is a nucleotide-binding site, and the conserved residues stabilize bound AMP (Seo et al. 2016). The motif is highly conserved in all LOG enzymes (Seo and Kim 2017).

The polypeptide having cytokinin riboside 5-monophosphate phosphoribohydrolase activity may be derived from the same species as the bacterium in which it is expressed or may be derived from a species different to the one in which it is expressed (i.e. it is heterologous). According to some embodiments, the polypeptide having cytokinin riboside 5-monophosphate phosphoribohydrolase activity is derived from the same species as the bacterium in which it is expressed. According to some embodiments, the polypeptide having cytokinin riboside 5-monophosphate phosphoribohydrolase activity is derived from a species different from the one in which it is expressed (i.e. it is heterologous).

According to some embodiments, the polypeptide having cytokinin riboside 5-monophosphate phosphoribohydrolase activity is a bacterial polypeptide having cytokinin riboside 5-monophosphate phosphoribohydrolase activity. With “bacterial polypeptide having cytokinin riboside 5-monophosphate phosphoribohydrolase activity” it is meant that the polypeptide having cytokinin riboside 5-monophosphate phosphoribohydrolase activity is naturally derived from a bacterium, such as Corynebacterium glutamicum.

A polypeptide having cytokinin riboside 5′-monophosphate phosphoribohydrolase activity for use according to the invention may for instance be a polypeptide having cytokinin riboside 5′-monophosphate phosphoribohydrolase activity selected from the group consisting of: i) a polypeptide comprising an amino acid sequence of any one of SEQ ID NOs: 34 to 62; and ii) a polypeptide comprising an amino acid sequence, which has at least about 70%, such as at least about 75%, at least about 80%, at least about 85%, at least about 90%, at least about 93%, at least 95%, at least 96%, at least 97%, at least 98%, or at least 99%, sequence identity to the amino acid sequence of any one of SEQ ID NOs: 34 to 62.

A polypeptide having cytokinin riboside 5′-monophosphate phosphoribohydrolase activity for use according to the invention may for instance be a polypeptide having cytokinin riboside 5′-monophosphate phosphoribohydrolase activity selected from the group consisting of: i) a polypeptide comprising an amino acid sequence of any one of SEQ ID NOs: 34 to 44; and ii) a polypeptide comprising an amino acid sequence, which has at least about 70%, such as at least about 75%, at least about 80%, at least about 85%, at least about 90%, at least about 93%, at least 95%, at least 96%, at least 97%, at least 98%, or at least 99%, sequence identity to the amino acid sequence of any one of SEQ ID NOs: 34 to 44.

According to some embodiments, the polypeptide having cytokinin riboside 5′-monophosphate phosphoribohydrolase activity comprises the amino acid sequence of SEQ ID NO: 34 or an amino acid sequence having at least 70%, such as at least 75%, sequence identity with SEQ ID NO: 34. According to some embodiments, the polypeptide having cytokinin riboside 5′-monophosphate phosphoribohydrolase activity comprises the amino acid sequence of SEQ ID NO: 34 or an amino acid sequence having at least 80%, such as at least 85%, sequence identity with SEQ ID NO: 34. According to some embodiments, the polypeptide having cytokinin riboside 5′-monophosphate phosphoribohydrolase activity comprises the amino acid sequence of SEQ ID NO: 34 or an amino acid sequence having at least 90%, such as at least 95%, sequence identity with SEQ ID NO: 34. According to some embodiments, the polypeptide having cytokinin riboside 5′-monophosphate phosphoribohydrolase activity comprises the amino acid sequence of SEQ ID NO: 34.

According to some embodiments, the polypeptide having cytokinin riboside 5′-monophosphate phosphoribohydrolase activity comprises the amino acid sequence of SEQ ID NO: 35 or an amino acid sequence having at least 70%, such as at least 75%, sequence identity with SEQ ID NO: 35.

According to some embodiments, the polypeptide having cytokinin riboside 5-monophosphate phosphoribohydrolase activity comprises the amino acid sequence of SEQ ID NO: 35 or an amino acid sequence having at least 80%, such as at least 85%, sequence identity with SEQ ID NO: 35. According to some embodiments, the polypeptide having cytokinin riboside 5-monophosphate phosphoribohydrolase activity comprises the amino acid sequence of SEQ ID NO: 35 or an amino acid sequence having at least 90%, such as at least 95%, sequence identity with SEQ ID NO: 35. According to some embodiments, the polypeptide having cytokinin riboside 5-monophosphate phosphoribohydrolase activity comprises the amino acid sequence of SEQ ID NO: 35.

According to some embodiments, the polypeptide having cytokinin riboside 5-monophosphate phosphoribohydrolase activity comprises the amino acid sequence of SEQ ID NO: 36 or an amino acid sequence having at least 70%, such as at least 75%, sequence identity with SEQ ID NO: 36. According to some embodiments, the polypeptide having cytokinin riboside 5-monophosphate phosphoribohydrolase activity comprises the amino acid sequence of SEQ ID NO: 36 or an amino acid sequence having at least 80%, such as at least 85%, sequence identity with SEQ ID NO: 36. According to some embodiments, the polypeptide having cytokinin riboside 5-monophosphate phosphoribohydrolase activity comprises the amino acid sequence of SEQ ID NO: 36 or an amino acid sequence having at least 90%, such as at least 95%, sequence identity with SEQ ID NO: 36. According to some embodiments, the polypeptide having cytokinin riboside 5-monophosphate phosphoribohydrolase activity comprises the amino acid sequence of SEQ ID NO: 36.

According to some embodiments, the polypeptide having cytokinin riboside 5-monophosphate phosphoribohydrolase activity comprises the amino acid sequence of SEQ ID NO: 37 or an amino acid sequence having at least 70%, such as at least 75%, sequence identity with SEQ ID NO: 37. According to some embodiments, the polypeptide having cytokinin riboside 5-monophosphate phosphoribohydrolase activity comprises the amino acid sequence of SEQ ID NO: 37 or an amino acid sequence having at least 80%, such as at least 85%, sequence identity with SEQ ID NO: 37. According to some embodiments, the polypeptide having cytokinin riboside 5-monophosphate phosphoribohydrolase activity comprises the amino acid sequence of SEQ ID NO: 37 or an amino acid sequence having at least 90%, such as at least 95%, sequence identity with SEQ ID NO: 37. According to some embodiments, the polypeptide having cytokinin riboside 5-monophosphate phosphoribohydrolase activity comprises the amino acid sequence of SEQ ID NO: 37.

According to some embodiments, the polypeptide having cytokinin riboside 5-monophosphate phosphoribohydrolase activity comprises the amino acid sequence of SEQ ID NO: 38 or an amino acid sequence having at least 70%, such as at least 75%, sequence identity with SEQ ID NO: 38. According to some embodiments, the polypeptide having cytokinin riboside 5-monophosphate phosphoribohydrolase activity comprises the amino acid sequence of SEQ ID NO: 38 or an amino acid sequence having at least 80%, such as at least 85%, sequence identity with SEQ ID NO: 38. According to some embodiments, the polypeptide having cytokinin riboside 5-monophosphate phosphoribohydrolase activity comprises the amino acid sequence of SEQ ID NO: 38 or an amino acid sequence having at least 90%, such as at least 95%, sequence identity with SEQ ID NO: 38. According to some embodiments, the polypeptide having cytokinin riboside 5-monophosphate phosphoribohydrolase activity comprises the amino acid sequence of SEQ ID NO: 38.

According to some embodiments, the polypeptide having cytokinin riboside 5-monophosphate phosphoribohydrolase activity comprises the amino acid sequence of SEQ ID NO: 39 or an amino acid sequence having at least 70%, such as at least 75%, sequence identity with SEQ ID NO: 39. According to some embodiments, the polypeptide having cytokinin riboside 5-monophosphate phosphoribohydrolase activity comprises the amino acid sequence of SEQ ID NO: 39 or an amino acid sequence having at least 80%, such as at least 85%, sequence identity with SEQ ID NO: 39. According to some embodiments, the polypeptide having cytokinin riboside 5-monophosphate phosphoribohydrolase activity comprises the amino acid sequence of SEQ ID NO: 39 or an amino acid sequence having at least 90%, such as at least 95%, sequence identity with SEQ ID NO: 39. According to some embodiments, the polypeptide having cytokinin riboside 5-monophosphate phosphoribohydrolase activity comprises the amino acid sequence of SEQ ID NO: 39.

According to some embodiments, the polypeptide having cytokinin riboside 5-monophosphate phosphoribohydrolase activity comprises the amino acid sequence of SEQ ID NO: 40 or an amino acid sequence having at least 70%, such as at least 75%, sequence identity with SEQ ID NO: 40. According to some embodiments, the polypeptide having cytokinin riboside 5-monophosphate phosphoribohydrolase activity comprises the amino acid sequence of SEQ ID NO: 40 or an amino acid sequence having at least 80%, such as at least 85%, sequence identity with SEQ ID NO: 40. According to some embodiments, the polypeptide having cytokinin riboside 5-monophosphate phosphoribohydrolase activity comprises the amino acid sequence of SEQ ID NO: 40 or an amino acid sequence having at least 90%, such as at least 95%, sequence identity with SEQ ID NO: 40.

According to some embodiments, the polypeptide having cytokinin riboside 5-monophosphate phosphoribohydrolase activity comprises the amino acid sequence of SEQ ID NO: 40.

According to some embodiments, the polypeptide having cytokinin riboside 5-monophosphate phosphoribohydrolase activity comprises the amino acid sequence of SEQ ID NO: 41 or an amino acid sequence having at least 70%, such as at least 75%, sequence identity with SEQ ID NO: 41. According to some embodiments, the polypeptide having cytokinin riboside 5-monophosphate phosphoribohydrolase activity comprises the amino acid sequence of SEQ ID NO: 41 or an amino acid sequence having at least 80%, such as at least 85%, sequence identity with SEQ ID NO: 41. According to some embodiments, the polypeptide having cytokinin riboside 5-monophosphate phosphoribohydrolase activity comprises the amino acid sequence of SEQ ID NO: 41 or an amino acid sequence having at least 90%, such as at least 95%, sequence identity with SEQ ID NO: 41. According to some embodiments, the polypeptide having cytokinin riboside 5-monophosphate phosphoribohydrolase activity comprises the amino acid sequence of SEQ ID NO: 41.

According to some embodiments, the polypeptide having cytokinin riboside 5-monophosphate phosphoribohydrolase activity comprises the amino acid sequence of SEQ ID NO: 42 or an amino acid sequence having at least 70%, such as at least 75%, sequence identity with SEQ ID NO: 42. According to some embodiments, the polypeptide having cytokinin riboside 5-monophosphate phosphoribohydrolase activity comprises the amino acid sequence of SEQ ID NO: 42 or an amino acid sequence having at least 80%, such as at least 85%, sequence identity with SEQ ID NO: 42. According to some embodiments, the polypeptide having cytokinin riboside 5-monophosphate phosphoribohydrolase activity comprises the amino acid sequence of SEQ ID NO: 42 or an amino acid sequence having at least 90%, such as at least 95%, sequence identity with SEQ ID NO: 42. According to some embodiments, the polypeptide having cytokinin riboside 5-monophosphate phosphoribohydrolase activity comprises the amino acid sequence of SEQ ID NO: 42.

According to some embodiments, the polypeptide having cytokinin riboside 5-monophosphate phosphoribohydrolase activity comprises the amino acid sequence of SEQ ID NO: 43 or an amino acid sequence having at least 70%, such as at least 75%, sequence identity with SEQ ID NO: 43. According to some embodiments, the polypeptide having cytokinin riboside 5-monophosphate phosphoribohydrolase activity comprises the amino acid sequence of SEQ ID NO: 43 or an amino acid sequence having at least 80%, such as at least 85%, sequence identity with SEQ ID NO: 43.

According to some embodiments, the polypeptide having cytokinin riboside 5′-monophosphate phosphoribohydrolase activity comprises the amino acid sequence of SEQ ID NO: 43 or an amino acid sequence having at least 90%, such as at least 95%, sequence identity with SEQ ID NO: 43. According to some embodiments, the polypeptide having cytokinin riboside 5′-monophosphate phosphoribohydrolase activity comprises the amino acid sequence of SEQ ID NO: 43.

According to some embodiments, the polypeptide having cytokinin riboside 5′-monophosphate phosphoribohydrolase activity comprises the amino acid sequence of SEQ ID NO: 44 or an amino acid sequence having at least 70%, such as at least 75%, sequence identity with SEQ ID NO: 44. According to some embodiments, the polypeptide having cytokinin riboside 5′-monophosphate phosphoribohydrolase activity comprises the amino acid sequence of SEQ ID NO: 44 or an amino acid sequence having at least 80%, such as at least 85%, sequence identity with SEQ ID NO: 44. According to some embodiments, the polypeptide having cytokinin riboside 5′-monophosphate phosphoribohydrolase activity comprises the amino acid sequence of SEQ ID NO: 44 or an amino acid sequence having at least 90%, such as at least 95%, sequence identity with SEQ ID NO: 44. According to some embodiments, the polypeptide having cytokinin riboside 5′-monophosphate phosphoribohydrolase activity comprises the amino acid sequence of SEQ ID NO: 44.

Techniques for determining cytokinin riboside 5′-monophosphate phosphoribohydrolase activity are well known to the skilled person. Exemplary methods have been described, e.g. by Seo et al. (2016). The cytokinin riboside 5′-monophosphate phosphoribohydrolase activity may for instance be determined in accordance with the following method:

The phosphoribohydrolase activity is determined by detecting adenine ring compounds separated by thin-layer chromatography (TLC) method. The enzyme reaction is carried out in a mixture of 20 mM AMP, 36 mM Tris-HCl, pH 8.0, and 23 μM purified enzyme at 30° C., and then the reaction is stopped by heating the mixture at 95° C. for 1.5 min. The reaction mixture is then dotted on a PEI-cellulose-F plastic TLC sheet (Merck Millipore). The mobile phase is 1 M sodium chloride. After development in the TLC chamber, the sheet is dried completely. Adenine ring-including compounds are detected by UV lamp (290 nm) (Seo et al. 2016).

In order to increase the supply of the isopentenyl side chain precursor of isoprenoid cytokinins, metabolic flux through the methylerythritol 4-phosphate (MEP) pathway may be increased. This is achieved primarily through overexpression of 1-Deoxy-D-xylulose 5-phosphate (DXP) synthase (DXS).

Hence, according to some embodiments, the bacterium of the present invention is characterized in that it has been (further) modified to have an increased protein expression of a polypeptide having 1-deoxy-D-xylulose-5-phosphate synthase activity compared to an otherwise identical bacterium that does not carry said modification.

By “increased protein expression” it is meant that the amount of the polypeptide having 1-deoxy-D-xylulose-5-phosphate synthase activity produced by the thus modified bacterium is increased compared to an otherwise identical bacterium that does not carry said modification. More particularly, by “increased expression” it is meant that the amount of the polypeptide having 1-deoxy-D-xylulose-5-phosphate synthase activity produced by the thus modified bacterium is increased by at least 10%, such as at least 20%, at least 30%, at least 40%, at least 50% at least 60%, at least 70%, at least 80%, at least 90%, at least 100%, at least 150%, at least 200%, at least 300%, at least 400%, at least 500%, at least 600%, at least 700% at least 800%, at least about 900%, at least about 1000%, at least about 2000%, at least about 3000%, at least about 4000%, at least about 5000%, at least about 6000%, at least about 7000%, at least about 8000% at least about 9000% or at least about 10000%, compared an otherwise identical bacterium that does not carry said modification. The amount of protein in a given cell can be determined by any suitable quantification technique known in the art, such as ELISA, Immunohistochemistry, or Western Blotting.

An increase in protein expression may be achieved by any suitable means well-known to those skilled in the art. For example, an increase in protein expression may be achieved by increasing the number of copies of the gene or genes encoding the polypeptide having 1-deoxy-D-xylulose-5-phosphate synthase activity in the bacterium, such as by introducing into the bacterium an exogenous nucleic acid, such as a vector, comprising the gene or genes encoding the polypeptide having 1-deoxy-D-xylulose-5-phosphate synthase activity operably linked to a promoter that is functional in the bacterium to cause the production of an mRNA molecule.

An increase in protein expression may also be achieved by the integration of at least a second copy of the gene encoding the polypeptide having 1-deoxy-D-xylulose-5-phosphate synthase activity into the genome of the bacterium.

An increase in protein expression may also be achieved by increasing the strength of the promoter operably linked to the gene encoding the polypeptide having 1-deoxy-D-xylulose-5-phosphate synthase activity, e.g. by replacing the native promoter with a promoter that enables higher expression and overproduction of polypeptide compared to the native promoter. The promoters that can be used include natural promoters from Bacillus subtilis, Bacillus amyloliquefaciens or similar, such as P43, P15, Pveg, Pylb, PgroES, PsigX, PtrnQ, Ppst, PsodA, PrpsF, PlepA, PliaG, PrpsF, Ppst, PfusA, PsodA, Phag as well as artificial promoters active in Bacillus subtilis or inducible Bacillus subtilis promoters, such as PmtlA, Pspac, PxylA, PsacB, or similar. Further examples include natural promoters from Corynebacterium, such as P CP_2454, Ptuf and Psod, natural promoters from E. coli, such as T7, ParaBAD, Plac, Ptac and Ptrc, and the promoter P F1 derived from the corynephage BFK20.

An increase in protein expression may also be achieved by modifying the ribosome binding site on the mRNA molecule encoding the polypeptide having 1-deoxy-D-xylulose-5-phosphate synthase activity. By modifying the sequence of the ribosome binding site, the translation initiation rate may be increased, thus increasing translation efficiency.

According to some embodiments, the increase in the number of copies of the gene is achieved by introducing into the bacterium one or more (such as two or three) exogenous nucleic acid molecules (such as one or more vectors) comprising the gene operably linked to a promoter that is functional in the bacterium to cause the production of an mRNA molecule.

According to some embodiments, a bacterium of the invention comprises an exogenous nucleic acid molecule (such as a vector) comprising one or more (such as two, three, or four) nucleotide sequences encoding the polypeptide having 1-deoxy-D-xylulose-5-phosphate synthase activity. Suitably, the exogenous nucleic acid molecule further comprises a promoter that is functional in the bacterium to cause the production of an mRNA molecule and that is operably linked to the nucleotide sequence encoding said polypeptide having 1-deoxy-D-xylulose-5-phosphate synthase activity. According to some embodiments, the exogenous nucleic acid molecule is stably integrated into the genome of the bacterium.

A polypeptide having 1-deoxy-D-xylulose-5-phosphate synthase activity is an enzyme that catalyzes the condensation between D-glyceraldehyde 3-phosphate and pyruvate to produce 1-deoxy-D-xylulose 5-phosphate (DXP) (DXS; E.C. 2.2.1.7). DXS catalyzes the first enzymatic step of the isoprenoid biosynthesis of HMBDP and DMAPP in the methylerythritol phosphate (MEP) pathway, whose kcat/Km value is substantially lower than for other enzymes in this pathway (Kuzuyama et al. 2000). DXS is present in a single copy in eubacteria, whereas green algae and higher plants have two or more genes encoding DXS, which form three distinguishable groups. In higher plants, the expression of the distinct DXS isoenzymes depends on the type of tissue and developmental stage.

DXS is highly conserved in bacteria and plants. Its protein sequence has about 20% identity with transketolase and pyruvate dehydrogenase E1 subunit. All three enzymes catalyze similar reactions and require coenzyme thiamine pyrophosphate (TPP). DXS from E. coli contains three domains (1, 11, and 111), which are homologous to the equivalent domains in transketolase and the E1 subunit of pyruvate dehydrogenase. Two DXS monomers are arranged side-by-side in a dimer. Domain I (residues 1-319) is above domains II (residues 320-495) and III (residues 496-629) of the same monomer. All three domains have the α/β fold with a central, mostly parallel β-sheet between a helices. Domain I contains a five-stranded parallel β-sheet, domain II contains a six-stranded parallel β-sheet, and domain II contains a five-stranded β-sheet with the first strand anti-parallel to the other four strands. Domain I has several extended surface segments, at the N-terminus (residues 1-49), after the first strand (residues 81-122), and in the connection between the fourth and fifth strands (residues 184-250).

The active site of DXS is located at the interface of domains I and II in the same monomer. The C-terminal ends of the central parallel β-sheets of the two domains are pointed towards each other, and the TPP coenzyme is located at the bottom of a pocket in this interface. The amino-pyrimidine ring of TPP interacts with domain II, while the pyrophosphate group interacts with domain I. The C2 atom of the thiazolium ring is exposed to the substrate-binding site. The C2 atom participates in the reaction. The pyrophosphate group of TPP has numerous polar interactions with the enzyme. The active site is composed of a magnesium ion bound between the two phosphate groups, and the side chains of Asp154, Asn183 and Met185. The Gly153-Asp-Gly155-Asn183 sequence in DXS is consistent with the TPP binding motif «GDG-X(25-30)-N». The C2 constitutes pyruvate-binding site. GAP is located in the pocket (Xiang et al., 2007).

Increasing DXS activity is recognized as the most effective strategy for increased terpenoid biosynthesis in many species, also in B. subtilis (Yang et al. 2019), as several studies indicate that the formation of DXP is the limiting step of the MEP pathway. A single amino acid mutation in Dxs of E. coli and Deinococcus radiodurans increased their catalytic activities. The mutation Y392F of E. coli Dxs increased the relative catalytic activity by more than 2.5-fold compared to the wild type (Xiang et al. 2012). DXS is regulated by a negative feedback mechanism by the end products of the MEP pathway, IPP, and DMAPP (Banerjee et al. 2013). DXS has been a subject of site-directed mutagenesis for alleviating the negative feedback inhibition of IPP and DMAPP. Mutation at A147G/A352G of Populus trichocarpa DXS reduced its IPP binding affinity slightly, but it increased Km for TPP and pyruvate, and it decreased the catalytic efficiency of the enzyme about 15-fold (Banerjee et al., 2016). Overexpression of several other enzymes of the pathway besides DXS has also been tested with mixed results in different bacteria. Also, the overexpression of the entire MEP pathway as artificial operons (operon dxs-ispD-ispF-ispH and ispC/dxr-ispE-ispG-ispA) has been tested in B. subtilis (Xue et al. 2015).

The polypeptide having 1-deoxy-D-xylulose-5-phosphate synthase activity may be derived from the same species as the bacterium in which it is expressed or may be derived from a species different from the one in which it is expressed (i.e. it is heterologous). According to some embodiments, the polypeptide having 1-deoxy-D-xylulose-5-phosphate synthase activity is derived from the same species as the bacterium in which it is expressed. According to some embodiments, the polypeptide having 1-deoxy-D-xylulose-5-phosphate synthase activity is derived from a species different from the one in which it is expressed (i.e. it is heterologous).

Preferably, the polypeptide having 1-deoxy-D-xylulose-5-phosphate synthase activity is a bacterial polypeptide having 1-deoxy-D-xylulose-5-phosphate synthase activity. With “bacterial polypeptide having 1-deoxy-D-xylulose-5-phosphate synthase activity” it is meant that the polypeptide having 1-deoxy-D-xylulose-5-phosphate synthase activity is naturally derived from a bacterium, such as Bacillus subtilis.

A polypeptide having 1-deoxy-D-xylulose-5-phosphate synthase activity for use according to the invention may for instance be a polypeptide having 1-deoxy-D-xylulose-5-phosphate synthase activity selected from the group consisting of: i) a polypeptide comprising an amino acid sequence of any one of SEQ ID NOs: 63 to 70; and ii) a polypeptide comprising an amino acid sequence, which has at least about 70%, such as at least about 75%, at least about 80%, at least about 85%, at least about 90%, at least about 93%, at least 95%, at least 96%, at least 97%, at least 98%, or at least 99%, sequence identity to the amino acid sequence of any one of SEQ ID NOs: 63 to 70.

According to some embodiments, the polypeptide having 1-deoxy-D-xylulose-5-phosphate synthase activity comprises the amino acid sequence of SEQ ID NO: 63 or an amino acid sequence having at least 70%, such as at least 75%, sequence identity with SEQ ID NO: 63. According to some embodiments, the polypeptide having 1-deoxy-D-xylulose-5-phosphate synthase activity comprises the amino acid sequence of SEQ ID NO: 63 or an amino acid sequence having at least 80%, such as at least 85%, sequence identity with SEQ ID NO: 63. According to some embodiments, the polypeptide 1-deoxy-D-xylulose-5-phosphate synthase activity comprises the amino acid sequence of SEQ ID NO: 63 or an amino acid sequence having at least 90%, such as at least 95%, sequence identity with SEQ ID NO: 63. According to some embodiments, the polypeptide having 1-deoxy-D-xylulose-5-phosphate synthase activity comprises the amino acid sequence of SEQ ID NO: 63.

According to some embodiments, the polypeptide having 1-deoxy-D-xylulose-5-phosphate synthase activity comprises the amino acid sequence of SEQ ID NO: 64 or an amino acid sequence having at least 70%, such as at least 75%, sequence identity with SEQ ID NO: 64. According to some embodiments, the polypeptide having 1-deoxy-D-xylulose-5-phosphate synthase activity comprises the amino acid sequence of SEQ ID NO: 64 or an amino acid sequence having at least 80%, such as at least 85%, sequence identity with SEQ ID NO: 64. According to some embodiments, the polypeptide 1-deoxy-D-xylulose-5-phosphate synthase activity comprises the amino acid sequence of SEQ ID NO: 64 or an amino acid sequence having at least 90%, such as at least 95%, sequence identity with SEQ ID NO: 64. According to some embodiments, the polypeptide having 1-deoxy-D-xylulose-5-phosphate synthase activity comprises the amino acid sequence of SEQ ID NO: 64.

According to some embodiments, the polypeptide having 1-deoxy-D-xylulose-5-phosphate synthase activity comprises the amino acid sequence of SEQ ID NO: 65 or an amino acid sequence having at least 70%, such as at least 75%, sequence identity with SEQ ID NO: 65. According to some embodiments, the polypeptide having 1-deoxy-D-xylulose-5-phosphate synthase activity comprises the amino acid sequence of SEQ ID NO: 65 or an amino acid sequence having at least 80%, such as at least 85%, sequence identity with SEQ ID NO: 65. According to some embodiments, the polypeptide 1-deoxy-D-xylulose-5-phosphate synthase activity comprises the amino acid sequence of SEQ ID NO: 65 or an amino acid sequence having at least 90%, such as at least 95%, sequence identity with SEQ ID NO: 65. According to some embodiments, the polypeptide having 1-deoxy-D-xylulose-5-phosphate synthase activity comprises the amino acid sequence of SEQ ID NO: 65.

According to some embodiments, the polypeptide having 1-deoxy-D-xylulose-5-phosphate synthase activity comprises the amino acid sequence of SEQ ID NO: 66 or an amino acid sequence having at least 70%, such as at least 75%, sequence identity with SEQ ID NO: 66. According to some embodiments, the polypeptide having 1-deoxy-D-xylulose-5-phosphate synthase activity comprises the amino acid sequence of SEQ ID NO: 66 or an amino acid sequence having at least 80%, such as at least 85%, sequence identity with SEQ ID NO: 66. According to some embodiments, the polypeptide 1-deoxy-D-xylulose-5-phosphate synthase activity comprises the amino acid sequence of SEQ ID NO: 66 or an amino acid sequence having at least 90%, such as at least 95%, sequence identity with SEQ ID NO: 66. According to some embodiments, the polypeptide having 1-deoxy-D-xylulose-5-phosphate synthase activity comprises the amino acid sequence of SEQ ID NO: 66.

According to some embodiments, the polypeptide having 1-deoxy-D-xylulose-5-phosphate synthase activity comprises the amino acid sequence of SEQ ID NO: 67 or an amino acid sequence having at least 70%, such as at least 75%, sequence identity with SEQ ID NO: 67. According to some embodiments, the polypeptide having 1-deoxy-D-xylulose-5-phosphate synthase activity comprises the amino acid sequence of SEQ ID NO: 67 or an amino acid sequence having at least 80%, such as at least 85%, sequence identity with SEQ ID NO: 67. According to some embodiments, the polypeptide 1-deoxy-D-xylulose-5-phosphate synthase activity comprises the amino acid sequence of SEQ ID NO: 67 or an amino acid sequence having at least 90%, such as at least 95%, sequence identity with SEQ ID NO: 67. According to some embodiments, the polypeptide having 1-deoxy-D-xylulose-5-phosphate synthase activity comprises the amino acid sequence of SEQ ID NO: 67.

According to some embodiments, the polypeptide having 1-deoxy-D-xylulose-5-phosphate synthase activity comprises the amino acid sequence of SEQ ID NO: 68 or an amino acid sequence having at least 70%, such as at least 75%, sequence identity with SEQ ID NO: 68. According to some embodiments, the polypeptide having 1-deoxy-D-xylulose-5-phosphate synthase activity comprises the amino acid sequence of SEQ ID NO: 68 or an amino acid sequence having at least 80%, such as at least 85%, sequence identity with SEQ ID NO: 68. According to some embodiments, the polypeptide 1-deoxy-D-xylulose-5-phosphate synthase activity comprises the amino acid sequence of SEQ ID NO: 68 or an amino acid sequence having at least 90%, such as at least 95%, sequence identity with SEQ ID NO: 68. According to some embodiments, the polypeptide having 1-deoxy-D-xylulose-5-phosphate synthase activity comprises the amino acid sequence of SEQ ID NO: 68.

As noted above, modification of the wild type DXS sequence can increase the catalytic activities. The present invention thus particularly contemplates the use of mutant DXS having increased activity or an inactivated negative feedback mechanism compared to the wild type DXS enzyme from which it is derived. Non-limiting examples of such mutant DXS enzymes are those set out in SEQ ID NOs. 69 and 70.

According to some embodiments, the polypeptide having 1-deoxy-D-xylulose-5-phosphate synthase activity comprises the amino acid sequence of SEQ ID NO: 69 or an amino acid sequence having at least 70%, such as at least 75%, sequence identity with SEQ ID NO: 69, with the proviso that the amino acid at position 392 is not Y, preferably with the proviso that the amino acid at position 392 is F. According to some embodiments, the polypeptide having 1-deoxy-D-xylulose-5-phosphate synthase activity comprises the amino acid sequence of SEQ ID NO: 69 or an amino acid sequence having at least 80%, such as at least 85%, sequence identity with SEQ ID NO: 69, with the proviso that the amino acid at position 392 is not Y, preferably with the proviso that the amino acid at position 392 is F. According to some embodiments, the polypeptide 1-deoxy-D-xylulose-5-phosphate synthase activity comprises the amino acid sequence of SEQ ID NO: 69 or an amino acid sequence having at least 90%, such as at least 95%, sequence identity with SEQ ID NO: 69, with the proviso that the amino acid at position 392 is not Y, preferably with the proviso that the amino acid at position 392 is F. According to some embodiments, the polypeptide having 1-deoxy-D-xylulose-5-phosphate synthase activity comprises the amino acid sequence of SEQ ID NO: 69.

According to some embodiments, the polypeptide having 1-deoxy-D-xylulose-5-phosphate synthase activity comprises the amino acid sequence of SEQ ID NO: 70 or an amino acid sequence having at least 70%, such as at least 75%, sequence identity with SEQ ID NO: 70, with the proviso that the amino acid at position 389 is not Y, preferably with the proviso that the amino acid at position 389 is F. According to some embodiments, the polypeptide having 1-deoxy-D-xylulose-5-phosphate synthase activity comprises the amino acid sequence of SEQ ID NO: 70 or an amino acid sequence having at least 80%, such as at least 85%, sequence identity with SEQ ID NO: 70, with the proviso that the amino acid at position 389 is not Y, preferably with the proviso that the amino acid at position 389 is F. According to some embodiments, the polypeptide 1-deoxy-D-xylulose-5-phosphate synthase activity comprises the amino acid sequence of SEQ ID NO: 70 or an amino acid sequence having at least 90%, such as at least 95%, sequence identity with SEQ ID NO: 70, with the proviso that the amino acid at position 389 is not Y, preferably with the proviso that the amino acid at position 389 is F. According to some embodiments, the polypeptide having 1-deoxy-D-xylulose-5-phosphate synthase activity comprises the amino acid sequence of SEQ ID NO: 70.

Techniques for determining 1-deoxy-D-xylulose-5-phosphate synthase activity are well known to the skilled person. Exemplary methods have been described, e.g. by Kuzuyama et al (2000) and by Kudoh et al (2017). The 1-deoxy-D-xylulose-5-phosphate synthase activity may for instance be determined in accordance with the following methods:
(1) The 1-deoxy-D-xylulose-5-phosphate synthase activity is determined by DXS assay (Kuzuyama et al., 2000): The standard assay system consists of 100 mM Tris-HCl (pH 8.0) containing 1 mM MgCl₂, 2 mM dl-dithiothreitol, 1 mM sodium pyruvate, 2 mM dl-glyceraldehyde 3-phosphate, and 150 μM thiamine diphosphate in a final volume of 0.5 ml. The reaction is initiated by adding the enzyme solution to the complete assay mixture at 37° C., and after a 10 min-incubation, the reaction is halted by incubation at 100° C. for 1 min. Next, the reaction mixture is treated with alkaline phosphatase at 56° C. for 60 min to dephosphorylate completely the reaction product, DXP. Production of the resulting dephosphorylated compound, 1-deoxyxylulose (DX), is monitored by a refractive index spectrometer (model RI-71; Showa Denko, Tokyo, Japan) with a Shodex KS-801 (8-by 300-mm) column (Showa Denko), eluted with H₂O at a flow rate of 1 ml/min at 80° C. DX is eluted at 8.6 min under this condition. The amount of DX production is precisely estimated by using chemically synthesized DX as the standard. One unit of DXS activity is defined as the amount of the enzyme that caused the production of 1 μmol of DXP per min at 37° C. Production of DXP by the DXS is monitored at 195 nm by high-performance liquid chromatography with a Senshu Pak NH2-1251-N (4.6-by 250-mm) column (Senshu Science, Tokyo, Japan) eluted with 100 mM KH₂PO₄ (pH 3.5) at a flow rate of 1 ml/min. DXP is eluted at 8.1 min under this condition.
(2) A coupled enzyme assay of DXS (Kudoh et al., 2017): DXS activity is measured using a coupled enzyme assay with DXR from E. coli as the coupling enzyme. In this assay, DXP generated by the DXS activity is further converted to MEP. As this step consumes NADPH, the overall reaction can be measured spectrophotometrically at 340 nm. Assay mixtures contains 100 mM Tris/HCl (pH 7.8), 10 mM MgCl₂, 0.3 mM thiamine pyrophosphate (TPP), 1 mM dithiothreitol (DTT), 0.3 mM nicotinamide adenine dinucleotide phosphate (NADPH), various concentrations of sodium pyruvate (0.05-5 mM) and D,L-GAP (0.2-2.0 mM), and DXR (100 or 50 mg/ml). The mixtures are incubated at 30° C. for 2 min inside a temperature-controlled spectrophotometer (model UV-1800, Shimadzu, Kyoto, Japan) and added to the DXS sample (final concentration of 50 or 25 mg/ml) to start the reaction. The reaction is traced by monitoring the absorption at 340 nm at 30° C.

Purine nucleotides are structural components of DNA and RNA, energy carriers (i.e. ATP and GTP), enzyme cofactors (i.e. NAD⁺ and NADP⁺), and as such are essential metabolites for cellular physiology. The synthesis of purine nucleotides starts with the synthesis of 5′-phosphoribosylpyrophosphate (PRPP) from D-ribose 5-phosphate and ATP. The enzyme PRPP synthase (Ribose-phosphate diphosphokinase; EC 2.7.6.1) catalyzes the transfer of the diphosphoryl group of ATP to the D-ribose 5-phosphate, with the simultaneous formation of AMP. PRPP synthase is ubiquitous among free-living organisms. Most bacteria have one gene encoding PRPP synthase, whereas more than one gene is present in eukaryotes. The next step is the synthesis of 5-phospho-β-D-ribosylamine from PRPP and glutamine catalyzed by glutamine 5-phosphoribosyl-1-pyrophosphate (PRPP) amidotransferase (amidophosphoribosyltransferase; EC 2.4.2.14), which is encoded by purF in B. subtilis. This is the rate-limiting reaction of purine de novo synthesis. The further 10 steps leading from 5-phospho-β-D-ribosylamine to the synthesis of inosine-5-phosphate (IMP) are catalyzed by the enzymes encoded in the pur operon. The pur operon is negatively regulated by PurR repressor at transcriptional level encoded by purR, and by PurBoxes, specific DNA sequences in the upstream control regions of affected genes. IMP is the branching point for the synthesis of AMP and GMP. AMP is synthesized from IMP in two enzymatic steps, catalyzed by PurA and PurB, whereas GMP is synthesized from IMP by GuaB and GuaA. The expression of genes or operons encoding enzymes of the purine synthesis is regulated by purine bases and nucleosides in the growth medium. The enzymes PRPP synthetase, PRPP amidotransferase, adenylosuccinate synthetase and IMP dehydrogenase are regulated by the feedback inhibition of the end products of the pathway. PurR repressor inhibits the initiation of transcription. The salvage pathways also participate to generate the corresponding mononucleotides AMP and GMP by utilization of hypoxanthine, guanine, and adenine.

The purine nucleotide biosynthesis pathway is well-studied because of the role of purine nucleotides in the primary metabolism. It includes both the de novo synthesis pathway and the salvage pathway. Deregulation of the purine nucleotide biosynthesis pathway at transcription and metabolic levels enhanced the metabolic flow through the purine nucleotide biosynthesis pathway and consequently increased the yield of products directly stemming from the purine nucleotide biosynthesis pathway: inosine, guanosine, adenosine, and riboflavin. Various modifications including gene overexpression, gene deletions, and enzyme deregulation by mutations had been used successfully for increasing the yield of purine nucleotide biosynthesis pathway products.

Exemplary modifications in B. subtilis enzymes with a positive effect on the increase in purine flux are listed in Table 1 below.

TABLE 1
Modifications in B. subtilis enzymes with a positive effect on the increase in purine flux.

| | Modifications | | |
|---|---|---|---|
| Enzyme | and mutations | Effect of mutation | Reference |
| PRPP synthetase (prs) | N120S and L1351, | Deregulated enzyme: | (Zakataeva et al., |
| | overexpression | release of negative | 2012) |
| | | feedback inhibition from | |
| | | ADP and GDP, significant | |
| | | increase of sensitivity to | |
| | | inorganic phosphate (Pi) | |
| | | activation, increased | |
| | | purine production | |
| PRPP amidotransferase) | S283A, K305Q, | Deregulated enzyme: | (Chen et al., |
| (purF) | R307Q and | release of the negative | 1997) |
| | S347A, | feedback inhibition, | |
| | overexpression | increased purine | |
| | | production | |
| PRPP amidotransferase) | D293V, K316Q, | Deregulated enzyme: | (Shi et al., 2009) |
| (purF) | S400W, | release of the negative | |
| | overexpression | feedback inhibition, | |
| | | increased purine | |
| | | production | |
| 5′-UTR of pur operon | Disruption of | Upregulation of pur | (Shi et al., 2014) |
| | guanine | operon | |
| | riboswitch | | |
| Promoter Ppur | Deletion of | Increased expression of | (Asahara et al., |
| | attenuator | pur operon genes (genes | 2010) |
| | region, changes | of purine biosynthesis | |
| | in −10 sequence | pathway except prs and | |
| | | purF) | |
| PurR | Gene inactivation | Deletion of the | (Shi et al., 2009) |
| | (deletion) | repressor of pur operon- | |
| | | increased expression | |
| | | of pur operon genes | |
| | | (genes of purine | |
| | | biosynthesis pathway | |
| | | except prs and purF) | |
| Adenylosuccinate | P242N | Significantly reduced | (Wang et al., |
| synthetase (purA) | | enzyme activity | 2016) |
| | | (decreased flux to AMP, | |
| | | increased flux to GMP) | |
| Adenylosuccinate | Overexpression | Increased flux to AMP | (Li et al. 2019) |
| synthetase (purA) | | | |
| IMP dehydrogenase | A226V, | Significantly reduced | (Asahara et al., |
| (guaB) | resistance to | enzyme activity | 2010) |
| | mycophenolic | (decreased flux to GMP) | |
| | acid (MPA) | | |
| IMP dehydrogenase | deletion | Auxotrophy for | Patent |
| (guaB) | | guanosine, increased | U.S. Pat. No. |
| | | production of | 3,730,836A |
| | | AMP/adenosine | |
| GMP synthetase (guaA) | deletion | Auxotrophy for | Patent |
| | | guanosine, increased | U.S. Pat. No. |
| | | production of | 3,730,836A |
| | | AMP/adenosine | |
| YvrH, two-component | R222Q | Deregulation of purine | (Wang et al., |
| response regulator | | biosynthesis , increase in | 2011) |
| | | purine metabolites | |
| Purine nucleoside | Deletion, | Deletion of degradation | (Asahara et al., |
| phosphorylase (deoD) | inactivation | of adenosine to adenine | 2010) |
| Purine nucleoside | Deletion, | Prevention of adenosine | (Wang et al., |
| phosphorylase (pupG) | inactivation | and inosine degradation, | 2016) |
| | | increased accumulation | |
| | | of inosine | |
| hypoxanthine-guanine- | deletion | Inactivation of purine- | (Peifer et al., |
| phosphoribosyltransferase | | depleting reactions | 2012) |
| (HGPRTase; gene hpt) | | | |
| adenosine- | Resistance to 2- | Decreased degradation | (Christiansen, |
| phosphoribosyltransferase | fluoroadenine | of IMP, AMP synthesis | Schou and |
| (APRTase; gene apt) | | via the salvage pathway | Nygaard, 1997) |
| | | is enhanced | |
| hypoxanthine-guanine- | Resistance to 8- | Increased xanthosine | (Konishi and Shiro, |
| phosphoribosyltransferase | azaguanine | and guanosine | 1968) |
| (HGPRTase; gene hpt) | | production (shift from | |
| | | inosine production) | |
| hypoxanthine-guanine- | Resistance to 8- | Changed purine flow | (Christiansen, |
| phosphoribosyltransferase | thioguanine | | Schou and |
| (HGPRTase; gene hpt) | | | Nygaard, 1997) |
| xanthine | Resistance to 8- | Changed purine flow | (Christiansen, |
| phosphoribosyltransferase | azaxanthine | | Schou and |
| (XPRTase; gene xpt) | | | Nygaard, 1997) |

Hence, according to some embodiments, the bacterium of the present invention is characterized in that it has been (further) modified to have an increased expression and/or activity of at least one enzyme involved in the purine nucleotide biosynthesis pathway compared to an otherwise identical bacterium that does not carry said modification.

According to some embodiments, the bacterium of the present invention is characterized in that it has been (further) modified to have an increased expression and/or activity of at least one enzyme involved in the adenosine monophosphate biosynthesis pathway compared to an otherwise identical bacterium that does not carry said modification.

The at least one enzyme involved in the purine nucleotide biosynthesis pathway (e.g., the at least enzyme involved in the adenosine monophosphate biosynthesis pathway) may be derived from the same species as the bacterium in which it is expressed or may be derived from a species different to the one in which it is expressed (i.e. it is heterologous). According to some embodiments, at least one enzyme involved in the purine nucleotide biosynthesis pathway (e.g., the at least enzyme involved in the adenosine monophosphate biosynthesis pathway) is derived from the same species as the bacterium in which it is expressed. According to some embodiments, the at least one enzyme involved in the purine nucleotide biosynthesis pathway (e.g., the at least enzyme involved in the adenosine monophosphate biosynthesis pathway) is derived from a species different from the one in which it is expressed (i.e. it is heterologous).

By “increased protein expression” it is meant that the amount of the enzyme involved in the purine nucleotide biosynthesis pathway (e.g., the enzyme involved in the adenosine monophosphate biosynthesis pathway) produced by the thus modified bacterium is increased compared to an otherwise identical bacterium that does not carry said modification. More particularly, by “increased expression” it is meant that the amount of the enzyme involved in the purine nucleotide biosynthesis pathway (e.g., the enzyme involved in the adenosine monophosphate biosynthesis pathway) produced by the thus modified bacterium is increased by at least 10%, such as at least 20%, at least 30%, at least 40%, at least 50% at least 60%, at least 70%, at least 80%, at least 90%, at least 100%, at least 150%, at least 200%, at least 300%, at least 400%, at least 500%, at least 600%, at least 700% at least 800%, at least about 900%, at least about 1000%, at least about 2000%, at least about 3000%, at least about 4000%, at least about 5000%, at least about 6000%, at least about 7000%, at least about 8000% at least about 9000% or at least about 10000%, compared to an otherwise identical bacterium that does not carry said modification. The amount of protein in a given cell can be determined by any suitable quantification technique known in the art, such as ELISA, Immunohistochemistry, or Western Blotting.

An increase in protein expression may be achieved by any suitable means well-known to those skilled in the art. For example, an increase in protein expression may be achieved by increasing the number of copies of the gene encoding the enzyme involved in the purine nucleotide biosynthesis pathway (e.g., the enzyme involved in the adenosine monophosphate biosynthesis pathway) in the bacterium, such as by introducing into the bacterium an exogenous nucleic acid, such as a vector, comprising the gene encoding the enzyme involved in the purine nucleotide biosynthesis pathway (e.g., the enzyme involved in the adenosine biosynthesis pathway) operably linked to a promoter that is functional in the bacterium to cause the production of an mRNA molecule.

An increase in protein expression may also be achieved by the integration of at least a second copy of the gene encoding the enzyme involved in the purine nucleotide biosynthesis pathway (e.g., the enzyme involved in the adenosine monophosphate biosynthesis pathway) into the genome of the bacterium.

An increase in protein expression may also be achieved by increasing the strength of the promoter operably linked to the gene encoding the enzyme involved in the purine nucleotide biosynthesis pathway (e.g., the enzyme involved in the adenosine monophosphate biosynthesis pathway), e.g. by replacing the native promoter with a promoter that enables higher expression and overproduction of the enzyme compared to the native promoter. The promoters that can be used include natural promoters from Bacillus subtilis, Bacillus amyloliquefaciens or similar, such as P43, P15, Pveg, Pylb, PgroES, PsigX, PtrnQ, Ppst, PsodA, PrpsF, PlepA, PliaG, PrpsF, Ppst, PfusA, PsodA, Phag as well as artificial promoters active in Bacillus subtilis or inducible Bacillus subtilis promoters, such as PmtlA, Pspac, PxylA, PsacB, or similar. Further examples include natural promoters from Corynebacterium, such as P CP_2454, Ptuf and Psod, natural promoters from E. coli, such as T7, ParaBAD, Plac, Ptac and Ptrc, and the promoter P F1 derived from the corynephage BFK20.

An increase in protein expression may also be achieved by modifying the ribosome binding site on the mRNA molecule encoding the enzyme involved in the purine nucleotide biosynthesis pathway. By modifying the sequence of the ribosome binding site, the translation initiation rate may be increased, thus increasing translation efficiency.

According to some embodiments, the increase in the number of copies of the gene is achieved by introducing into the bacterium one or more (such as two or three) exogenous nucleic acid molecules (such as one or more vectors) comprising the gene operably linked to a promoter that is functional in the host cell to cause the production of an mRNA molecule.

According to some embodiments, a bacterium of the invention comprises an exogenous nucleic acid molecule (such as a vector) comprising one or more (such as two, three, or four) nucleotide sequences encoding the enzyme involved in the purine nucleotide biosynthesis pathway (e.g., the enzyme involved in the adenosine monophosphate biosynthesis pathway). Suitably, the exogenous nucleic acid molecule further comprises a promoter that is functional in the bacterium to cause the production of an mRNA molecule and that is operably linked to the nucleotide sequence encoding said the enzyme involved in the purine nucleotide biosynthesis pathway (e.g., said enzyme involved in the adenosine biosynthesis monophosphate pathway). According to some embodiments, the exogenous nucleic acid molecule is stably integrated into the genome of the bacterium.

The at least one enzyme involved in the purine nucleotide biosynthesis pathway may be an enzyme selected from the group consisting of: an enzyme having ribose-phosphate diphosphokinase activity, an enzyme having amidophosphoribosyltransferase activity, an enzyme having formyltetrahydrofolate deformylase activity, an enzyme having phosphoribosylamine-glycine ligase activity, an enzyme having phosphoribosylglycineamide formyltransferase activity, an enzyme having phosphoribosylformylglycinamidine synthase activity, an enzyme having phosphoribosylformylglycineamidine cyclo-ligase activity, an enzyme having N5-carboxyaminoimidazole ribonucleotide synthetase activity, an enzyme having N5-carboxyaminoimidazole ribonucleotide mutase activity, an enzyme having phosphoribosylaminoimidazolesuccinocarboxamide synthase activity, an enzyme having adenylosuccinate lyase activity, an enzyme having phosphoribosylaminoimidazole-carboxamide formyltransferase activity, an enzyme having IMP cyclohydolase activity, an enzyme having adenylosuccinate synthase activity, an enzyme having adenylate kinase activity, an enzyme having ATP synthase activity, an enzyme having adenosine kinase activity, an enzyme having IMP dehydrogenase activity and an enzyme having GMP synthase activity.

The at least one enzyme involved in the adenosine monophosphate biosynthesis pathway may be an enzyme selected from the group consisting of: an enzyme having ribose-phosphate diphosphokinase activity, an enzyme having amidophosphoribosyltransferase activity, an enzyme having formyltetrahydrofolate deformylase activity, an enzyme having phosphoribosylamine-glycine ligase activity, an enzyme having phosphoribosylglycineamide formyltransferase activity, an enzyme having phosphoribosylformylglycinamidine synthase activity, an enzyme having phosphoribosylformylglycineamidine cyclo-ligase activity, an enzyme having N5-carboxyaminoimidazole ribonucleotide synthetase activity, an enzyme having N5-carboxyaminoimidazole ribonucleotide mutase activity, an enzyme having phosphoribosylaminoimidazolesuccinocarboxamide synthase activity, an enzyme having adenylosuccinate lyase activity, an enzyme having phosphoribosylaminoimidazole-carboxamide formyltransferase activity, an enzyme having IMP cyclohydolase activity, an enzyme having adenylosuccinate synthase activity, an enzyme having adenylate kinase activity, an enzyme having ATP synthase activity and an enzyme having adenosine kinase activity.

According to some embodiments, the at least one enzyme involved in the purine nucleotide biosynthesis pathway (such as in the adenosine monophosphate biosynthesis pathway) is selected from the group consisting of: an enzyme having ribose-phosphate diphosphokinase activity, an enzyme having amidophosphoribosyltransferase activity, an enzyme having formyltetrahydrofolate deformylase activity, an enzyme having adenylosuccinate lyase activity, an enzyme having phosphoribosylaminoimidazole-carboxamide formyltransferase activity, an enzyme having adenylosuccinate synthase activity and an enzyme having adenosine kinase activity.

According to some embodiments, the bacterium of the present invention has been (further) modified to have any one of the modifications as disclosed in Table 1 in relation to one or more of its endogenous enzymes involved in the purine nucleotide biosynthesis pathway. Particularly, the bacterium may be the result of random mutagenesis making it resistant to an inhibitor of the enzyme in question.

According to some embodiments, the bacterium of the present invention is characterized in that it has been (further) modified to have a decreased expression and/or activity of at least one endogenous enzyme involved in the purine nucleotide degradation pathway compared to an otherwise identical bacterium that does not carry said modification.

According to some embodiments, the bacterium of the invention may be modified to have a decreased expression of at least one endogenous enzyme involved in the purine nucleotide degradation pathway compared to an otherwise identical bacterium that does not carry said modification.

According to some embodiments, the bacterium of the invention may be modified to have a decreased expression level of the endogenous gene encoding said at least one endogenous enzyme involved in the purine nucleotide degradation pathway compared to an otherwise identical bacterium that does not carry said modification. The expression level of the endogenous gene may, for example, be decreased by at least 50%, such as by at least 60%, at least 65%, at least 70%, at least 75%, at least 80%, at least 85%, at least 90%, at least 95% or at least 100% compared to the otherwise identical bacterium.

According to some embodiments, the endogenous gene encoding said enzyme has been inactivated, such as by deletion of part of or the entire gene sequence.

According to some embodiments, the endogenous gene encoding said enzyme has been inactivated by introducing or expressing in the microorganism a rare-cutting endonuclease able to selectively inactivate by DNA cleavage, preferably by a double-strand break, the endogenous gene encoding said enzyme. A rare-cutting endonuclease to be used in accordance with the present invention to inactivate the endogenous gene may, for instance, be a transcription activator-like effector (TALE) nuclease, meganuclease, zinc-finger nuclease (ZFN), or RNA-guided endonuclease.

One way to inactivate the endogenous gene encoding said enzyme is to use the CRISPRi system. The CRISPRi system was developed as a tool for targeted repression of gene expression or for blocking targeted locations on the genome. The CRISPRi system consists of the catalytically inactive, “dead” Cas9 protein (dCas9) and a guide RNA that defines the binding site for the dCas9 to DNA.

Thus, according to some embodiments, the endogenous gene encoding said enzyme is inactivated by introducing or expressing in the bacterium an RNA-guided endonuclease, such as a catalytically inactive Cas9 protein, and a single guide RNA (sgRNA) specifically hybridizing (e.g. binding) under cellular conditions with the genomic DNA encoding a said enzyme.

According to some embodiments, the expression of said at least one endogenous enzyme involved in the purine nucleotide degradation pathway is decreased by way of inhibition.

Inhibition of the expression of the said endogenous enzyme may be achieved by any suitable means known in the art. For example, the expression may be inhibited by gene silencing techniques involving the use of inhibitory nucleic acid molecules, such as antisense oligonucleotides, ribozymes, or interfering RNA (RNAi) molecules, such as microRNA (miRNA), small interfering RNA (siRNA), or short hairpin RNA (shRNA).

According to some embodiments, the expression of said at least one endogenous enzyme involved in the purine nucleotide degradation pathway is decreased (e.g., inhibited) by transcriptional and/or translational repression of the endogenous gene encoding said polypeptide.

According to some embodiments, the expression of said at least one endogenous enzyme involved in the purine nucleotide degradation pathway is inhibited by introducing or expressing in the bacterium an inhibitory nucleic acid molecule. For example, the inhibitory nucleic acid molecule may be introduced by way of an exogenous nucleic acid molecule comprising a nucleotide sequence encoding said inhibitory nucleic acid molecule operably linked to a promoter, such as an inducible promoter, that is functional in the bacterium to cause the production of said inhibitory nucleic acid molecule. Suitably, the inhibitory nucleic acid molecule is one that specifically hybridizes (e.g. binds) under cellular conditions with cellular mRNA and/or genomic DNA encoding the endogenous enzyme. Depending on the target, transcription of the encoding genomic DNA and/or translation of the encoding mRNA is/are inhibited.

According to some embodiments, the inhibitory nucleic acid molecule is an antisense oligonucleotide, ribozyme, or interfering RNA (RNAi) molecule. Preferably, such nucleic acid molecule comprises at least 10 consecutive nucleotides of the complement of the cellular mRNA and/or genomic DNA encoding the polypeptide or enzyme of interest (e.g., the cellular mRNA and/or genomic DNA encoding the polypeptide.

According to some embodiments, the inhibitory nucleic acid is an antisense oligonucleotide. Such antisense oligonucleotide is a nucleic acid molecule (either DNA or RNA), which specifically hybridizes (e.g. binds) under cellular conditions with the cellular mRNA and/or genomic DNA encoding the polypeptide.

According to some embodiments, the inhibitory nucleic acid molecule is a ribozyme, such as a hammerhead ribozyme. A ribozyme molecule is designed to catalytically cleave the mRNA transcript to prevent translation of the polypeptide.

According to some embodiments, the inhibitory nucleic acid molecule is an interfering RNA (RNAi) molecule. RNA interference is a biological process in which RNA molecules inhibit expression, typically destroying specific mRNA. Exemplary types of RNAi molecules include microRNA (miRNA), small interfering RNA (siRNA), and short hairpin RNA (shRNA). According to some embodiments, the RNAi molecule is a miRNA. According to some embodiments, the RNAi molecule is a siRNA. According to some embodiments, the RNAi molecule is an shRNA.

According to some embodiments, the bacterium of the invention has been modified to have a decreased activity of at least one endogenous enzyme involved in the purine nucleotide degradation pathway compared to an otherwise identical bacterium that does not carry said modification.

A decrease of the activity of the endogenous enzyme involved in the purine nucleotide degradation pathway may be achieved by any suitable means known in the art. For example, the activity may be decreased by introducing one or more mutations in the active site of the enzyme resulting in the reduction or loss of activity. Thus, according to some embodiments, the activity of the endogenous enzyme involved in the purine nucleotide degradation pathway is decreased by at least one active-site mutation resulting in the reduction or loss of activity. At least one active-site mutation may, for example, be at least one non-conservative amino acid substitution.

According to some embodiments, the at least one enzyme involved in the purine nucleotide degradation pathway is selected from the group consisting of: a purine nucleoside phosphorylase and adenosine-phosphoribosyltransferase. According to some embodiments, the at least one endogenous enzyme involved in the purine nucleotide degradation pathway is a purine nucleoside phosphorylase. According to some embodiments, the at least one endogenous enzyme involved in the purine nucleotide degradation pathway is adenosine-phosphoribosyltransferase.

According to some embodiments, the bacterium of the invention has been (further) modified to have a decreased expression and/or activity of at least one endogenous enzyme involved in the guanosine monophosphate biosynthesis pathway compared to an otherwise identical bacterium that does not carry said modification.

According to some embodiments, the bacterium of the invention may be modified to have a decreased expression level of the endogenous gene encoding said at least one endogenous enzyme involved in the guanosine monophosphate biosynthesis pathway compared to an otherwise identical bacterium that does not carry said modification. The expression level of the endogenous gene may, for example, be decreased by at least 50%, such as by at least 60%, at least 65%, at least 70%, at least 75%, at least 80%, at least 85%, at least 90%, at least 95% or at least 100% compared to the otherwise identical bacterium.

According to some embodiments, the endogenous gene encoding said enzyme has been inactivated, such as by deletion of part of or the entire gene sequence.

According to some embodiments, the endogenous gene encoding said enzyme has been inactivated by introducing or expressing in the microorganism a rare-cutting endonuclease able to selectively inactivate by DNA cleavage, preferably by a double-strand break, the endogenous gene encoding said enzyme. A rare-cutting endonuclease to be used in accordance with the present invention to inactivate the endogenous gene may, for instance, be a transcription activator-like effector (TALE) nuclease, meganuclease, zinc-finger nuclease (ZFN), or RNA-guided endonuclease.

One way to inactivate the endogenous gene encoding said enzyme is to use the CRISPRi system. The CRISPRi system was developed as a tool for targeted repression of gene expression or for blocking targeted locations on the genome. The CRISPRi system consists of the catalytically inactive, “dead” Cas9 protein (dCas9) and a guide RNA that defines the binding site for the dCas9 to DNA.

Thus, according to some embodiments, the endogenous gene encoding said enzyme is inactivated by introducing or expressing in the bacterium an RNA-guided endonuclease, such as a catalytically inactive Cas9 protein, and a single guide RNA (sgRNA) specifically hybridizing (e.g. binding) under cellular conditions with the genomic DNA encoding a said enzyme.

According to some embodiments, the expression of said at least one endogenous enzyme involved in the guanosine monophosphate biosynthesis pathway is decreased by way of inhibition.

Inhibition of the expression of the said endogenous enzyme may be achieved by any suitable means known in the art. For example, the expression may be inhibited by gene silencing techniques involving the use of inhibitory nucleic acid molecules, such as antisense oligonucleotides, ribozymes, or interfering RNA (RNAi) molecules, such as microRNA (miRNA), small interfering RNA (siRNA), or short hairpin RNA (shRNA).

According to some embodiments, the expression of said at least one endogenous enzyme involved in the guanosine monophosphate biosynthesis pathway is decreased (e.g., inhibited) by transcriptional and/or translational repression of the endogenous gene encoding said polypeptide.

According to some embodiments, the expression of said at least one endogenous enzyme involved in the guanosine monophosphate biosynthesis pathway is inhibited by introducing or expressing in the bacterium an inhibitory nucleic acid molecule. For example, the inhibitory nucleic acid molecule may be introduced by way of an exogenous nucleic acid molecule comprising a nucleotide sequence encoding said inhibitory nucleic acid molecule operably linked to a promoter, such as an inducible promoter, that is functional in the bacterium to cause the production of said inhibitory nucleic acid molecule. Suitably, the inhibitory nucleic acid molecule is one that specifically hybridizes (e.g. binds) under cellular conditions with cellular mRNA and/or genomic DNA encoding the endogenous enzyme. Depending on the target, transcription of the encoding genomic DNA and/or translation of the encoding mRNA is/are inhibited.

According to some embodiments, the inhibitory nucleic acid molecule is an antisense oligonucleotide, ribozyme, or interfering RNA (RNAi) molecule. Preferably, such nucleic acid molecule comprises at least 10 consecutive nucleotides of the complement of the cellular mRNA and/or genomic DNA encoding the polypeptide or enzyme of interest (e.g., the cellular mRNA and/or genomic DNA encoding the polypeptide.

According to some embodiments, the inhibitory nucleic acid is an antisense oligonucleotide. Such antisense oligonucleotide is a nucleic acid molecule (either DNA or RNA), which specifically hybridizes (e.g. binds) under cellular conditions with the cellular mRNA and/or genomic DNA encoding the polypeptide.

According to some embodiments, the inhibitory nucleic acid molecule is a ribozyme, such as a hammerhead ribozyme. A ribozyme molecule is designed to catalytically cleave the mRNA transcript to prevent translation of the polypeptide.

According to some embodiments, the inhibitory nucleic acid molecule is an interfering RNA (RNAi) molecule. RNA interference is a biological process in which RNA molecules inhibit expression, typically destroying specific mRNA. Exemplary types of RNAi molecules include microRNA (miRNA), small interfering RNA (siRNA), and short hairpin RNA (shRNA). According to some embodiments, the RNAi molecule is a miRNA. According to some embodiments, the RNAi molecule is a siRNA. According to some embodiments, the RNAi molecule is an shRNA.

According to some embodiments, the bacterium of the invention has been modified to have a decreased activity of at least one endogenous enzyme involved in the guanosine monophosphate biosynthesis pathway compared to an otherwise identical bacterium that does not carry said modification.

A decrease of the activity of the endogenous enzyme involved in the guanosine monophosphate biosynthesis pathway may be achieved by any suitable means known in the art. For example, the activity may be decreased by introducing one or more mutations in the active site of the enzyme resulting in the reduction or loss of activity. Thus, according to some embodiments, the activity of the endogenous enzyme involved in the guanosine monophosphate biosynthesis pathway is decreased by at least one active-site mutation resulting in the reduction or loss of activity. At least one active-site mutation may, for example, be at least one non-conservative amino acid substitution.

According to some embodiments, the at least one enzyme involved in the guanosine monophosphate biosynthesis pathway is selected from the group consisting of: IMP dehydrogenase and GMP synthetase.

According to some embodiments, the at least one enzyme involved in the guanosine monophosphate biosynthesis pathway is IMP dehydrogenase.

According to some embodiments, the at least one enzyme involved in the guanosine monophosphate biosynthesis pathway is GMP synthetase.

CYP450s are a diverse group of heme-containing enzymes, which catalyze a wide range of oxidative reactions. Cytochrome P450 monooxygenases (CYP450) also catalyze the hydroxylation of isopentenyladenine-type cytokinins. In Arabidopsis, 2 cytochrome P450 monooxygenases for hydroxylation of isopentenyladenine-type cytokinins are present, CYP735A1 and CYP735A2. The CYP735As catalyze a stereo-specific reaction of hydroxylation of iP-nucleotides, and with lower affinity the hydroxylation of the iP-nucleoside or iP to synthesize tZ (Takei et al., 2004b). Most CYP450s of plant origin are membrane-anchored to the endoplasmic reticulum (ER). Cytochrome P450 monooxygenases are also present in bacteria, such as Rhodococcus fascians. The cytochromes P450 generally fall into two broad classes, depending on the nature of the auxiliary protein(s). Class I P450s are found on the membranes of mitochondria and in bacteria, and class II cytochromes P450 are typified by the liver microsomal enzymes in mammalian cells. Class I P450s are three-component systems comprising a flavin adenine dinucleotide (FAD)-containing reductase, an iron-sulfur protein (ferredoxin), and the P450. Class 11 cytochromes P450 are composed of an FAD-containing, flavin mononucleotide (FMN)-containing NADPH-dependent cytochrome P450 reductase and a P450. Class III and class IV CYP450s have also been described in bacteria, but class I are the most common CYP450s in bacteria. The best-characterized bacterial cytochrome P450 monooxygenase system from Pseudomonas putida P450cam consists of three soluble proteins: putidaredoxin reductase; putidaredoxin, an intermediary iron-sulfur protein; and the cytochrome P450cam. The CYP450 Fas1 is encoded in the fas region of the linear plasmid and is thought to hydroxylate cytokinins produced by R. fascians (Frebort et al., 2011).

Cytochrome P450 monooxygenase (CYP450) can hydroxylate iP-nucleotides to produce trans-zeatin in three enzymatic steps from AMP and DMAPP. The enzyme can stereo-specifically hydroxylate the prenyl side chain of iPRMP and can thus make a bypass to tZRMP, which can be further activated to produce trans-zeatin.

Hence, according to some embodiments, the bacterium of the present invention is characterized in that it has been (further) modified to have increased protein expression of a polypeptide having cytochrome P450 monooxygenase (CYP450) activity compared to an otherwise identical bacterium that does not carry said modification.

By “increased protein expression” it is meant that the amount of the polypeptide having cytochrome P450 monooxygenase (CYP450) activity produced by the thus modified bacterium is increased compared to an otherwise identical bacterium that does not carry said modification. More particularly, by “increased expression” it is meant that the amount of the polypeptide having cytochrome P450 monooxygenase (CYP450) activity produced by the thus modified bacterium is increased by at least 10%, such as at least 20%, at least 30%, at least 40%, at least 50% at least 60%, at least 70%, at least 80%, at least 90%, at least 100%, at least 150%, at least 200%, at least 300%, at least 400%, at least 500%, at least 600%, at least 700% at least 800%, at least about 900%, at least about 1000%, at least about 2000%, at least about 3000%, at least about 4000%, at least about 5000%, at least about 6000%, at least about 7000%, at least about 8000% at least about 9000% or at least about 10000%, compared to an otherwise identical bacterium that does not carry said modification. The amount of protein in a given cell can be determined by any suitable quantification technique known in the art, such as ELISA, Immunohistochemistry, or Western Blotting.

An increase in protein expression may be achieved by any suitable means well-known to those skilled in the art. For example, an increase in protein expression may be achieved by increasing the number of copies of the gene encoding the polypeptide having cytochrome P450 monooxygenase (CYP450) activity in the bacterium, such as by introducing into the bacterium an exogenous nucleic acid, such as a vector, comprising the gene encoding the polypeptide having cytochrome P450 monooxygenase (CYP450) activity operably linked to a promoter that is functional in the bacterium to cause the production of an mRNA molecule.

An increase in protein expression may also be achieved by the integration of at least a second copy of the gene encoding the polypeptide having cytochrome P450 monooxygenase (CYP450) activity into the genome of the bacterium.

An increase in protein expression may also be achieved by increasing the strength of the promoter operably linked to the gene encoding the polypeptide having cytochrome P450 monooxygenase (CYP450) activity. The promoters that can be used include natural promoters from Bacillus subtilis, Bacillus amyloliquefaciens or similar, such as P43, P15, Pveg, Pylb, PgroES, PsigX, PtrnQ, Ppst, PsodA, PrpsF, PlepA, PliaG, PrpsF, Ppst, PfusA, PsodA, Phag as well as artificial promoters active in Bacillus subtilis or inducible Bacillus subtilis promoters, such as PmtlA, Pspac, PxylA, PsacB, or similar. Further examples include natural promoters from Corynebacterium, such as P CP_2454, Ptuf and Psod, natural promoters from E. coli, such as T7, ParaBAD, Plac, Ptac and Ptrc, and the promoter P F1 derived from the corynephage BFK20.

An increase in protein expression may also be achieved by modifying the ribosome binding site on the mRNA molecule encoding the polypeptide having cytochrome P450 monooxygenase (CYP450) activity. By modifying the sequence of the ribosome binding site the translation initiation rate may be increased, thus increasing translation efficiency.

According to some embodiments, the increase in the number of copies of the gene is achieved by introducing into the bacterium one or more (such as two or three) exogenous nucleic acid molecules (such as one or more vectors) comprising the gene operably linked to a promoter that is functional in the host cell to cause the production of an mRNA molecule.

According to some embodiments, a bacterium of the invention comprises an exogenous nucleic acid molecule (such as a vector) comprising one or more (such as two, three, or four) nucleotide sequences encoding the polypeptide having cytochrome P450 monooxygenase (CYP450) activity. Suitably, the exogenous nucleic acid molecule further comprises a promoter that is functional in the bacterium to cause the production of an mRNA molecule and that is operably linked to the nucleotide sequence encoding said polypeptide having cytochrome P450 monooxygenase (CYP450) activity. According to some embodiments, the exogenous nucleic acid molecule is stably integrated into the genome of the bacterium.

The polypeptide having cytochrome P450 monooxygenase (CYP450) activity may be derived from the same species as the bacterium in which it is expressed or may be derived from a species different to the one in which it is expressed (i.e. it is heterologous). According to some embodiments, the polypeptide having cytochrome P450 monooxygenase (CYP450) activity is derived from the same species as the bacterium in which it is expressed. According to some embodiments, the polypeptide having cytochrome P450 monooxygenase (CYP450) activity is derived from a species different from the one in which it is expressed (i.e. it is heterologous). By way of example, in case that a bacterium does not have an endogenous gene encoding a polypeptide having cytochrome P450 monooxygenase (CYP450) activity, then the polypeptide having cytochrome P450 monooxygenase (CYP450) activity expressed in said bacterium will be heterologous to said bacterium.

According to some embodiments, the polypeptide having cytochrome P450 monooxygenase (CYP450) activity is a bacterial polypeptide having cytochrome P450 monooxygenase (CYP450) activity. With “bacterial polypeptide having cytochrome P450 monooxygenase (CYP450) activity” it is meant that the polypeptide having cytochrome P450 monooxygenase (CYP450) activity is naturally derived from a bacterium, such as Rhodococcus fascians. A non-limiting example of a bacterial polypeptide having cytochrome P450 monooxygenase (CYP450) activity is set out in SEQ ID NO: 93.

According to some embodiments, the polypeptide having cytochrome P450 monooxygenase (CYP450) activity is a plant polypeptide having cytochrome P450 monooxygenase (CYP450) activity. With “plant polypeptide having cytochrome P450 monooxygenase (CYP450) activity” it is meant that the polypeptide having cytochrome P450 monooxygenase (CYP450) activity is naturally derived from a plant, such as Arabidopsis thaliana. Non-limiting examples of a plant polypeptide having cytochrome P450 monooxygenase (CYP450) activity are set out in SEQ ID NOs: 94 and 95.

A polypeptide having cytochrome P450 monooxygenase (CYP450) activity for use according to the invention may for instance be a polypeptide having cytochrome P450 monooxygenase (CYP450) activity selected from the group consisting of: i) a polypeptide comprising an amino acid sequence of any one of SEQ ID NOs: 93 to 95; and ii) a polypeptide comprising an amino acid sequence, which has at least about 70%, such as at least about 75%, at least about 80%, at least about 85%, at least about 90%, at least about 93%, at least 95%, at least 96%, at least 97%, at least 98%, or at least 99%, sequence identity to the amino acid sequence of any one of SEQ ID NOs: 93 to 95.

According to some embodiments, the polypeptide having cytochrome P450 monooxygenase (CYP450) activity comprises the amino acid sequence of SEQ ID NO: 93 or an amino acid sequence having at least 70%, such as at least 75%, sequence identity with SEQ ID NO: 93. According to some embodiments, the polypeptide having cytochrome P450 monooxygenase (CYP450) activity comprises the amino acid sequence of SEQ ID NO: 93 or an amino acid sequence having at least 80%, such as at least 85%, sequence identity with SEQ ID NO: 93. According to some embodiments, the polypeptide having cytochrome P450 monooxygenase (CYP450) activity comprises the amino acid sequence of SEQ ID NO: 93 or an amino acid sequence having at least 90%, such as at least 95%, sequence identity with SEQ ID NO: 93. According to some embodiments, the polypeptide having cytochrome P450 monooxygenase (CYP450) activity comprises the amino acid sequence of SEQ ID NO: 93.

According to some embodiments, the polypeptide having cytochrome P450 monooxygenase (CYP450) activity comprises the amino acid sequence of SEQ ID NO: 94 or an amino acid sequence having at least 70%, such as at least 75%, sequence identity with SEQ ID NO: 94. According to some embodiments, the polypeptide having cytochrome P450 monooxygenase (CYP450) activity comprises the amino acid sequence of SEQ ID NO: 94 or an amino acid sequence having at least 80%, such as at least 85%, sequence identity with SEQ ID NO: 94. According to some embodiments, the polypeptide having cytochrome P450 monooxygenase (CYP450) activity comprises the amino acid sequence of SEQ ID NO: 94 or an amino acid sequence having at least 90%, such as at least 95%, sequence identity with SEQ ID NO: 94. According to some embodiments, the polypeptide having cytochrome P450 monooxygenase (CYP450) activity comprises the amino acid sequence of SEQ ID NO: 94.

According to some embodiments, the polypeptide having cytochrome P450 monooxygenase (CYP450) activity comprises the amino acid sequence of SEQ ID NO: 95 or an amino acid sequence having at least 70%, such as at least 75%, sequence identity with SEQ ID NO: 95. According to some embodiments, the polypeptide having cytochrome P450 monooxygenase (CYP450) activity comprises the amino acid sequence of SEQ ID NO: 95 or an amino acid sequence having at least 80%, such as at least 85%, sequence identity with SEQ ID NO: 95. According to some embodiments, the polypeptide having cytochrome P450 monooxygenase (CYP450) activity comprises the amino acid sequence of SEQ ID NO: 95 or an amino acid sequence having at least 90%, such as at least 95%, sequence identity with SEQ ID NO: 95. According to some embodiments, the polypeptide having cytochrome P450 monooxygenase (CYP450) activity comprises the amino acid sequence of SEQ ID NO: 95.

A bacterium according to the present invention can be produced from any suitable bacterium. The bacterium may be Gram-positive or Gram-negative. Non-limiting examples for Gram-negative bacterial host cells include species from the genera Escherichia, Erwinia, Klebsiella, and Citrobacter.

Non-limiting examples of Gram-positive bacterial host cells include species from the genera Bacillus, Lactococcus, Lactobacillus, Clostridium, Corynebacterium, Streptomyces, Streptococcus, and Cellulomonas. According to some embodiments, the bacterium of the present invention is Gram-positive. According to some embodiments, the bacterium of the present invention is Gram-negative.

According to some embodiments, the bacterium of the present invention is a bacterium of the family selected from the group consisting of Enterobacteriaceae, Bacillaceae, Lactobacillaceae, and Corynebacteriaceae. According to some embodiments, the recombinant host cell is a bacterium of the family Enterobacteriaceae. According to some embodiments, the recombinant host cell is a bacterium of the family Bacillaceae. According to some embodiments, the recombinant host cell is a bacterium of the family Corynebacteriaceae.

According to some embodiments, the bacterium of the present invention is a bacterium, which may be a bacterium of the genus Bacillus, Lactococcus, Lactobacillus, Clostridium, Corynebacterium, Geobacillus, Thermoanaerobacterium, Streptococcus, Pseudomonas, Streptomyces, Escherichia, Shigella, Acinetobacter, Citrobacter, Salmonella, Klebsiella, Enterobacter, Erwinia, Kluyvera, Serratia, Cedecea, Morganella, Hafnia, Edwardsiella, Providencia, Proteus, or Yersinia.

According to some embodiments, the bacterium of the present invention is a bacterium of the genus Bacillus. Non-limiting examples of a bacterium of the genus Bacillus are Bacillus subtilis, Bacillus amyloliquefaciens, Bacillus licheniformis, and Bacillus mojavensis. According to some embodiments, the bacterium of the present invention is Bacillus subtilis.

According to some embodiments, the bacterium of the present invention is a bacterium of the genus Corynebacterium. Non-limiting examples of a bacterium of the genus Corynebacterium are Corynebacterium glutamicum and Corynebacterium stationis. According to some, the bacterium of the present invention is Corynebacterium glutamicum. According to some, the bacterium of the present invention is Corynebacterium stationis. Within the context of the present invention, Corynebacterium stationis and Corynebacterium ammoniagenes refer to the same species and may be used interchangeably.

According to some embodiments, the bacterium of the present invention is a bacterium of the genus Escherichia. A non-limiting example of a bacterium of the genus Escherichia is Escherichia coli. According to some, the bacterium of the present invention is Escherichia coli.

As noted above, a bacterium of the invention is modified to express one or more polypeptides as detailed herein, which may mean that an exogenous nucleic acid molecule, such as a DNA molecule, which comprises a nucleotide sequence encoding said polypeptide has been introduced in the bacterium. Thus, a bacterium of the present invention may comprise an exogenous nucleic acid molecule, such as a DNA molecule, which comprises a nucleotide sequence encoding the polypeptide in question. Techniques for introducing an exogenous nucleic acid molecule, such as a DNA molecule, into a bacterial cell are well-known to those of skill in the art and include transformation (e.g., heat shock or natural transformation) among others.

In order to facilitate (over)expression of a polypeptide in the bacterium, the exogenous nucleic acid molecule may comprise suitable regulatory elements such as a promoter that is functional in the bacterial cell to cause the production of an mRNA molecule and that is operably linked to the nucleotide sequence encoding said polypeptide.

Promoters useful in accordance with the invention are any known promoters that are functional in a given host cell to cause the production of an mRNA molecule. Many such promoters are known to the skilled person. Such promoters include promoters normally associated with other genes, and/or promoters isolated from any bacteria. The use of promoters for protein expression is generally known to those skilled in the art of molecular biology, for example, see Sambrook et al., Molecular cloning: A Laboratory Manual, Cold Spring Harbor Laboratory, Cold Spring Harbor, N.Y., 1989. The promoter employed may be inducible, such as a temperature-inducible promoter (e.g., a pL or pR phage lambda promoters, each of which can be controlled by the temperature-sensitive lambda repressor c1857). The term “inducible” used in the context of a promoter means that the promoter only directs transcription of an operably linked nucleotide sequence if a stimulus is present, such as a change in temperature or the presence of a chemical substance (“chemical inducer”). As used herein, “chemical induction” according to the present invention refers to the physical application of an exogenous or endogenous substance (incl. macromolecules, e.g., proteins or nucleic acids) to a host cell. This has the effect of causing the target promoter present in the host cell to increase the rate of transcription. Alternatively, the promoter employed may be constitutive. The term “constitutive” used in the context of a promoter means that the promoter is capable of directing transcription of an operably linked nucleotide sequence in the absence of a stimulus (such as heat shock, chemicals, etc.).

Temperature induction systems work, for example, by employing promoters that are repressed by thermolabile repressors. These repressors are active at lower temperatures for example at 30° C., while unable to fold correctly at 37° C. and are therefore inactive. Such circuits therefore can be used to directly regulate the genes of interest also by genome integration of the genes along with the repressors. Examples of such a temperature-inducible expression system are based on the pL and/or pR X phage promoters, which are regulated by the thermolabile c1857 repressor. Similar to the genome integrated DE3 system, the expression of the T7 RNA polymerase gene may also be controlled using a temperature-controlled promoter system, while the expression of the genes of interest can be controlled using a T7 promoter.

Non-limiting examples of promoters functional in bacteria include both constitutive and inducible promoters such as T7 promoter, the beta-lactamase and lactose promoter systems; alkaline phosphatase (phoA) promoter, a tryptophan (trp) promoter system, tetracycline promoter, lambda-phage promoter, ribosomal protein promoters; and hybrid promoters such as the tac promoter. Other bacterial and synthetic promoters are also suitable.

Besides a promoter, the exogenous nucleic acid molecule may further comprise at least one regulatory element selected from a 5′ untranslated region (5′UTR) and 3′ untranslated region (3′ UTR). Many such 5′ UTRs and 3′ UTRs derived from prokaryotes and eukaryotes are well known to the skilled person. Such regulatory elements include 5′ UTRs and 3′ UTRs normally associated with other genes, and/or 5′ UTRs and 3′ UTRs isolated from any bacteria.

Usually, the 5′ UTR contains a ribosome binding site (RBS), also known as the Shine-Dalgarno sequence, which is usually 3-10 base pairs upstream from the initiation codon.

The exogenous nucleic acid molecule may be a vector or part of a vector, such as an expression vector. Normally, such a vector remains extrachromosomal within the bacterial cell, which means that it is found outside of the nucleus or nucleoid region of the bacterium.

It is also contemplated by the present invention that the exogenous nucleic acid molecule is stably integrated into the genome of the bacterium. Means for stable integration into the genome of a host cell, e.g., by homologous recombination, are well known to the skilled person.

Method of the Invention

The present invention also provides methods for producing an isoprenoid cytokinin or riboside derivative thereof, comprising cultivating a bacterium according to the present invention under suitable culture conditions in a suitable culture medium.

The method may further comprise collecting the isoprenoid cytokinin or riboside derivative thereof from the culture medium.

According to some embodiments, the isoprenoid cytokinin or riboside derivative thereof is selected from the group consisting of trans-zeatin (tZ), trans-zeatin riboside (tZR), N⁶-(D2-isopentenyl)adenine (iP), N⁶-(dimethylallyl)adenosine (iPR), dihydrozeatin (DZ), ribosyl dihydrozeatin (DZR), and combinations thereof.

According to some embodiments, the isoprenoid cytokinin or riboside derivative thereof is trans-zeatin (tZ) and trans-zeatin riboside (tZR), respectively.

The culture medium employed may be any conventional medium suitable for culturing a bacterium cell in question, and may be composed according to the principles of the prior art. The medium will usually contain all nutrients necessary for the growth and survival of the respective bacterium, such as carbon and nitrogen sources and other inorganic salts. Suitable media, e.g. minimal or complex media, are available from commercial suppliers or may be prepared according to published receipts, e.g. the American Type Culture Collection (ATCC) Catalogue of strains. Non-limiting standard media well known to the skilled person include Luria Bertani (LB) broth, Sabouraud Dextrose (SD) broth, MS broth, Yeast Peptone Dextrose, BMMY, GMMY, or Yeast Malt Extract (YM) broth, which are all commercially available. A non-limiting example of suitable media for culturing bacterial cells, such as B. subtilis, L. lactis or E. coli cells, including minimal media and rich media such as Luria Broth (LB), M9 media, M17 media, SA media, MOPS media, Terrific Broth, YT and others.

The carbon source may be any suitable carbon substrate known in the art, and particularly any carbon substrate commonly used in the cultivation of microorganisms and/or fermentation. Non-limiting examples of suitable fermentable carbon substrates include carbohydrates (e.g., C5 sugars such as arabinose or xylose, or C6 sugars such as glucose), glycerol, glycerine, acetate, dihydroxyacetone, one-carbon source, methanol, methane, oils, animal fats, animal oils, plant oils, fatty acids, lipids, phospholipids, glycerolipids, monoglycerides, diglycerides, triglycerides, renewable carbon sources, polypeptides (e.g., a microbial or plant protein or peptide), yeast extract, component from a yeast extract, peptone, casaminoacids or any combination of two or more of the foregoing.

As the nitrogen source, various ammonium salts such as ammonia and ammonium sulfate, other nitrogen compounds such as amines, a natural nitrogen source such as peptone, soybean-hydrolysate, and digested fermentative microorganism can be used. As minerals, potassium monophosphate, magnesium sulfate, sodium chloride, ferrous sulfate, manganese sulfate, calcium chloride, and the like can be used.

In order to further improve the production of the isoprenoid cytokinin or riboside derivative thereof, such as is trans-zeatin (tZ) and trans-zeatin riboside (tZR), respectively, the culture medium may be supplemented with a source of adenine or adenosine, such as adenine sulfate. Thus, according to some embodiments, the culture medium comprises adenine sulfate. The concentration of adenine sulfate in the culture medium may generally be in the range from about 0.1 g/L to about 4 g/L, such as from about 1 g/L to about 3.5 g/L. According to some embodiments, the concentration of adenine sulfate in the culture medium is in the range from about 2.5 g/L to about 3.5 g/L. However, other sources of adenine or adenosine, such as yeast extract, are also contemplated for use in accordance with the invention.

The cultivation can be preferably performed under aerobic conditions, such as by a shaking culture, and by a stirring culture with aeration, at a temperature of about 20 to about 45° C., such as about 30 to 38° C., such as at about 37° C. According to some embodiments, the cultivation is performed at a temperature of about 30 to 38° C., such as at about 37° C. The pH of the culture is usually above 5, such as in a range from about 6 to about 8, preferably from about 6.5 to about 7.5, more preferably from about 6.8 to about 7.2. According to some embodiments, the cultivation is performed at a pH from about 6 to about 8. The pH of the culture can be adjusted with ammonia, calcium carbonate, various acids, various bases, and buffers. The cultivation may be carried out for a period in the range from 10 to 70 h, such as in the range of 10 to 24 h or 10 to 48 h.

After cultivation, solids such as cells can be removed from the culture medium by centrifugation or membrane filtration. The isoprenoid cytokinin or riboside derivative thereof can be collected by a conventional method for the isolation and purification of chemical compounds from a medium.

Well-known purification procedures include, but are not limited to, centrifugation or filtration, precipitation, ion exchange, chromatographic methods such as e.g. ion-exchange chromatography or gel filtration chromatography, and crystallization methods.

The present invention thus provides an isoprenoid cytokinin or riboside derivative thereof obtainable by a method as detailed herein.

Abbreviations
iP—N⁶-(D2-isopentenyl)adenine
iPR—N⁶-(D2-isopentenyl)adenine riboside alias N⁶-(D2-isopentenyl)adenosine
tZ—trans-zeatin
tZR—trans-ribosylzeatin alias trans-zeatin riboside
DZ—dihydrozeatin
DZR—ribosyl dihydrozeatin alias dihydrozeatin riboside
cZ—cis-zeatin
MVA pathway—mevalonate biosynthesis pathway
MEP pathway—methylerythritol phosphate biosynthesis pathway
DMAPP—dimethylallyl diphosphate
HMBDP—1-hydroxy-2-methyl-2-butenyl 4-diphosphate
DXP—1-deoxy-D-xylulose-5-phosphate
DXS—1-deoxy-D-xylulose-5-phosphate synthase; DXP-synthase (EC 2.2.1.7)
tZRMP—trans-zeatin riboside 5′-monophosphate
iPRMP—N⁶-(D2-isopentenyl)adenine riboside 5′-monophosphate
DZRMP—dihydrozeatin riboside 5′-monophosphate
cZRMP—cis-zeatin riboside 5′-monophosphate
IPT—Adenylate isopentenyltransferase (EC 2.5.1.27)
LOG—cytokinin riboside 5-monophosphate phosphoribohydrolase ‘Lonely guy’ (EC 3.2.2.n1)
CYP450—cytochrome P450 monooxygenase

### Certain Other Definitions

As used herein, a “polypeptide having adenylate isopentenyltransferase activity” or a “polypeptide, which has adenylate isopentenyltransferase activity” means a polypeptide that catalyzes the reactions: Dimethylallyl diphosphate+AMP<=>diphosphate+N(6)-(dimethylallyl)adenosine 5-phosphate (EC 2.5.1.27) and optionally 1-hydroxy-2-methyl-2-butenyl 4-diphosphate+AMP<=>diphosphate+trans-zeatin riboside 5-phosphate. Non-limiting examples of such polypeptides are provided in SEQ ID NOs: 1 to 33.

As used herein, a “polypeptide having cytokinin riboside 5-monophosphate phosphoribohydrolase activity” or a “polypeptide which has cytokinin riboside 5-monophosphate phosphoribohydrolase activity” means a polypeptide that catalyzes the reaction: N(6)-(Delta(2)-isopentenyl)-adenosine 5-phosphate+H(2)O<=>N(6)-(dimethylallyl)adenine+D-ribose 5-phosphate (EC 3.2.2.n1). Non-limiting examples of such polypeptides are provided in SEQ ID NOs: 34 to 62.

As used herein, a “polypeptide having 1-deoxy-D-xylulose-5-phosphate synthase activity” or a “polypeptide, which has 1-deoxy-D-xylulose-5-phosphate synthase activity” means a polypeptide that catalyzes the reaction: Pyruvate+D-glyceraldehyde 3-phosphate<=>1-deoxy-D-xylulose 5-phosphate+CO(2) (EC 2.2.1.7). Non-limiting examples of such polypeptides are provided in SEQ ID NOs: 63 to 70.

As used herein, an “enzyme having ribose-phosphate diphosphokinase activity” or an “enzyme, which has ribose-phosphate diphosphokinase activity” means an enzyme that catalyzes the reaction: ATP+D-ribose 5-phosphate<=>AMP+5-phospho-alpha-D-ribose 1-diphosphate (EC 2.7.6.1). An enzyme having ribose-phosphate diphosphokinase activity is encoded by the bacterial gene prs or an ortholog thereof.

As used herein, an “enzyme having amidophosphoribosyltransferase activity” or an “enzyme, which has amidophosphoribosyltransferase activity” means an enzyme that catalyzes the reaction: 5-phospho-beta-D-ribosylamine+diphosphate+L-glutamate<=>L-glutamine+5-phospho-alpha-D-ribose 1-diphosphate+H₂O (EC 2.4.2.14). An enzyme having amidophosphoribosyltransferase activity is encoded by the bacterial gene purF or an ortholog thereof.

As used herein, an “enzyme having formyltetrahydrofolate deformylase activity” or an “enzyme, which has formyltetrahydrofolate deformylase activity” means an enzyme that catalyzes the reaction: 10-formyltetrahydrofolate+H₂O<=>formate+tetrahydrofolate (EC 3.5.1.10). An enzyme having amidophosphoribosyltransferase activity is encoded by the bacterial gene purU or an ortholog thereof.

As used herein, an “enzyme having phosphoribosylamine-glycine ligase activity” or an “enzyme, which has phosphoribosylamine-glycine ligase activity” means an enzyme that catalyzes the reaction: ATP+5-phospho-beta-D-ribosylamine+glycine<=>ADP+phosphate+N¹-(5-phospho-beta-D-ribosyl)glycinamide (EC 6.3.4.13). An enzyme having phosphoribosylamine-glycine ligase activity is encoded by the bacterial gene purD or an ortholog thereof.

As used herein, an “enzyme having phosphoribosylglycineamide formyltransferase activity” or an “enzyme, which has phosphoribosylglycineamide formyltransferase activity” means an enzyme that catalyzes the reaction: 10-formyltetrahydrofolate+N¹-(5-phospho-beta-D-ribosyl)glycinamide<=>tetrahydrofolate+N²-formyl-N¹-(5-phospho-beta-D-ribosyl)glycinamide (EC 2.1.2.2). An enzyme having phosphoribosylglycineamide formyltransferase activity is encoded by the bacterial gene purT or an ortholog thereof.

As used herein, an “enzyme having phosphoribosylformylglycinamidine synthase activity” or an “enzyme, which has phosphoribosylformylglycinamidine synthase activity” means an enzyme that catalyzes the reaction: ATP+N²-formyl-N¹-(5-phospho-beta-D-ribosyl)glycinamide+L-glutamine+H₂O<=>ADP+phosphate+2-(formamido)-N¹-(5-phospho-beta-D-ribosyl)acetamidine+L-glutamate (EC 6.3.5.3). An enzyme having phosphoribosylformylglycinamidine synthase activity is encoded by the bacterial gene purL or an ortholog thereof.

As used herein, an “enzyme having phosphoribosylformylglycineamidine cyclo-ligase activity” or an “enzyme, which has phosphoribosylformylglycineamidine cyclo-ligase activity” means an enzyme that catalyzes the reaction: ATP+2-(formamido)-N¹-(5-phospho-beta-D-ribosyl)acetamidine<=>ADP+phosphate+5-amino-1-(5-phospho-beta-D-ribosyl)imidazole (EC 6.3.3.1). An enzyme having phosphoribosylformylglycineamidine cyclo-ligase activity is encoded by the bacterial gene purM or an ortholog thereof.

As used herein, an “enzyme having N5-carboxyaminoimidazole ribonucleotide synthetase activity” or an “enzyme, which has N5-carboxyaminoimidazole ribonucleotide synthetase activity” means an enzyme that catalyzes the reaction: ATP+5-amino-1-(5-phospho-D-ribosyl)imidazole+HCO₃⁻<=>ADP+phosphate+5-carboxyamino-1-(5-phospho-D-ribosyl)imidazole (EC 6.3.4.18). An enzyme having N5-carboxyaminoimidazole ribonucleotide synthetase activity is encoded by the bacterial gene purK or an ortholog thereof.

As used herein, an “enzyme having N5-carboxyaminoimidazole ribonucleotide mutase activity” or an “enzyme, which has N5-carboxyaminoimidazole ribonucleotide mutase activity” means an enzyme that catalyzes the reaction: 5-carboxyamino-1-(5-phospho-D-ribosyl)imidazole<=>5-amino-1-(5-phospho-D-ribosyl)imidazole-4-carboxylate (EC 5.4.99.18). An enzyme having N5-carboxyaminoimidazole ribonucleotide mutase activity is encoded by the bacterial gene purE or an ortholog thereof.

As used herein, an “enzyme having phosphoribosylaminoimidazolesuccinocarboxamide synthase activity” or an “enzyme, which has phosphoribosylaminoimidazolesuccinocarboxamide synthase activity” means an enzyme that catalyzes the reaction: ATP+5-amino-1-(5-phospho-D-ribosyl)imidazole-4-carboxylate+L-aspartate<=>ADP+phosphate+(S)-2-(5-amino-1-(5-phospho-D-ribosyl)imidazole-4-carboxamido)succinate (EC 6.3.2.6). An enzyme having phosphoribosylaminoimidazolesuccinocarboxamide synthase activity is encoded by the bacterial gene purC or an ortholog thereof.

As used herein, an “enzyme having adenylosuccinate lyase activity” or an “enzyme, which has adenylosuccinate lyase activity” means an enzyme that catalyzes the reaction: (S)-2-(5-amino-1-(5-phospho-D-ribosyl)imidazole-4-carboxamido)succinate<=>fumarate+5-amino-1-(5-phospho-D-ribosyl)imidazole-4-carboxamide (EC 4.3.2.2). An enzyme having adenylosuccinate lyase activity is encoded by the bacterial gene purB or an ortholog thereof.

As used herein, an “enzyme having phosphoribosylaminoimidazole-carboxamide formyltransferase activity” or an “enzyme, which has phosphoribosylaminoimidazole-carboxamide formyltransferase activity” means an enzyme that catalyzes the reaction: 10-formyltetrahydrofolate+5-amino-1-(5-phospho-D-ribosyl)imidazole-4-carboxamide<=>tetrahydrofolate+5-formamido-1-(5-phospho-D-ribosyl)imidazole-4-carboxamide (EC 2.1.2.3). An enzyme having phosphoribosylaminoimidazole-carboxamide formyltransferase activity is encoded by the bacterial gene purH or an ortholog thereof.

As used herein, an “enzyme having IMP cyclohydrolase activity” or an “enzyme, which has IMP cyclohydrolase activity” means an enzyme that catalyzes the reaction: IMP+H₂O<=>5-formamido-1-(5-phospho-D-ribosyl)imidazole-4-carboxamide (EC 3.5.4.10). An enzyme having IMP cyclohydrolase activity is, for example, encoded by the bacterial gene purH or an ortholog thereof.

As used herein, an “enzyme having adenylosuccinate synthase activity” or an “enzyme, which has adenylosuccinate synthase activity” means an enzyme that catalyzes the reaction: GTP+IMP+L-aspartate<=>GDP+phosphate+N⁶-(1,2-dicarboxyethyl)-AMP (EC 6.3.4.4). An enzyme having adenylosuccinate synthase activity is encoded by the bacterial gene purA or an ortholog thereof.

As used herein, an “enzyme having adenylate kinase activity” or an “enzyme, which has adenylate kinase activity” means an enzyme that catalyzes the reaction: ATP+AMP<=>2 ADP (EC 2.7.4.3). An enzyme having adenylate kinase activity is encoded by the bacterial gene adk or an ortholog thereof.

As used herein, an “enzyme having ATP synthase activity” or an “enzyme, which has ATP synthase activity” means an enzyme that catalyzes the reaction: ATP+H₂O+H⁺ (cytosol)<=>ADP+phosphate+H⁺ (periplasm) (EC 3.6.3.14). An enzyme having ATP synthase activity is, for example, the ATP synthase F₀ or F₁ complex encoded by the bacterial atp operon (including the genes atpB, atpF, atpE, atpD, atpG, atpA, atpH and atpC) or orthologs thereof.

As used herein, an “enzyme having adenosine kinase activity” or an “enzyme, which has adenosine kinase activity” means an enzyme that catalyzes the reaction: ATP+adenosine<=>ADP+AMP (EC 2.7.1.20). An enzyme having adenosine kinase activity is encoded by the bacterial gene adk or orthologs thereof.

As used herein, an “enzyme having IMP dehydrogenase activity” or an “enzyme, which has IMP dehydrogenase activity” means an enzyme that catalyzes the reaction: Inosine 5-phosphate+NAD⁺+H₂O<=>xanthosine 5-phosphate+NADH (EC 1.1.1.205). An enzyme having IMP dehydrogenase activity is encoded by the bacterial gene guaB or an ortholog thereof.

As used herein, an “enzyme having GMP synthase activity” or an “enzyme, which has GMP synthase activity” means an enzyme that catalyzes the reaction: ATP+XMP+L-glutamine+H₂O<=>AMP+diphosphate+GMP+L-glutamate (EC 6.3.5.2). An enzyme having GMP synthase activity is encoded by the bacterial gene guaA or an ortholog thereof.

As used herein, an “enzyme having purine nucleoside phosphorylase activity” or an “enzyme, which has purine nucleoside phosphorylase activity” means an enzyme that catalyzes the reaction: Purine nucleoside+phosphate<=>purine+alpha-D-ribose 1-phosphate (EC 2.4.2.1). An enzyme having purine nucleoside phosphorylase activity is, for example, encoded by the bacterial gene deoD or an ortholog thereof.

As used herein, an “enzyme having adenosine phosphoribosyltransferase activity” or an “enzyme, which has adenosine phosphoribosyltransferase activity” means an enzyme that catalyzes the reaction: AMP+diphosphate<=>adenine+5-phospho-alpha-D-ribose 1-diphosphate (EC 2.4.2.7). An enzyme having adenosine phosphoribosyltransferase activity is, for example, encoded by the bacterial gene apt or an ortholog thereof.

As used herein, “purine nucleotide biosynthesis pathway” is understood to include both the de novo biosynthesis pathway and the salvage pathway by which nucleotides are synthesized.

As used herein, “adenosine monophosphate biosynthesis pathway” is understood to include both the de novo biosynthesis pathway and the salvage pathway by which adenosine monophosphate is synthesized.

As used herein, “guanosine monophosphate biosynthesis pathway” is understood to include both the de novo biosynthesis pathway and the salvage pathway by which guanosine monophosphate is synthesized.

As used herein, a “polypeptide having cytochrome P450 monooxygenase (CYP450) activity” or a “polypeptide, which has cytochrome P450 monooxygenase (CYP450) activity” means a polypeptide that catalyzes the trans-hydroxylation reactions: 1) N6-(Δ2-isopentenyl)-adenosine 5-monophosphate+a reduced [NADPH-hemoprotein reductase]+oxygen→trans-zeatin riboside monophosphate+an oxidized [NADPH-hemoprotein reductase]+H₂O; 2) N6-(Δ2-isopentenyl)-adenosine 5-triphosphate+a reduced [NADPH-hemoprotein reductase]+oxygen→trans-zeatin riboside triphosphate+an oxidized [NADPH-hemoprotein reductase]+H₂O; 3) N6-(Δ2-isopentenyl)-adenosine 5-diphosphate+a reduced [NADPH-hemoprotein reductase]+oxygen→trans-zeatin riboside diphosphate+an oxidized [NADPH-hemoprotein reductase]+H₂O (EC 1.14.14.-). A polypeptide having cytochrome P450 monooxygenase (CYP450) activity is, for example, encoded by the bacterial gene FAS1, or Arabidopsis thaliana (plant) genes CYP735A1 and CYP735A2, or an ortholog thereof. Non-limiting examples include SEQ ID 93 to 95.

“Polypeptide” and “protein” are used interchangeably herein to denote a polymer of at least two amino acids covalently linked by an amide bond, regardless of length or post-translational modification (e.g., glycosylation, phosphorylation, lipidation, myristoylation, ubiquitination, etc.). Included within this definition are D- and L-amino acids, and mixtures of D- and L-amino acids.

“Nucleic acid” or “polynucleotide” are used interchangeably herein to denote a polymer of at least two nucleic acid monomer units or bases (e.g., adenine, cytosine, guanine, thymine) covalently linked by a phosphodiester bond, regardless of length or base modification.

“Recombinant” or “non-naturally occurring” when used with reference to, e.g., a bacterium, nucleic acid, or polypeptide, refers to a material, or a material corresponding to the natural or native form of the material, that has been modified in a manner that would not otherwise exist in nature, or is identical thereto but produced or derived from synthetic materials and/or by manipulation using recombinant techniques. Non-limiting examples include, among others, recombinant bacterial cells expressing genes that are not found within the native (non-recombinant) form of the cell or express native genes that are otherwise expressed at a different level.

“Heterologous” or “exogenous” as used herein in the context of a gene or nucleic acid molecule refer to a gene or nucleic acid molecule (i.e. DNA or RNA molecule) that does not occur naturally as part of the genome of the bacterium in which it is present or which is found in a location or locations in the genome that differ from that in which it occurs in nature. Thus, a “heterologous” or “exogenous” gene or nucleic acid molecule is not endogenous to the bacterium and has been exogenously introduced into the microorganism. A “heterologous” gene or nucleic acid molecule DNA molecule may be from a different organism, a different species, a different genus, or a different kingdom, as the host DNA.

“Heterologous” as used herein in the context of a polypeptide means that a polypeptide is normally not found in or made (i.e. expressed) by the host microorganism, but derived from a different organism, a different species, a different genus, or a different kingdom.

As used herein, the term “ortholog” or “orthologs” refers to genes, nucleic acid molecules encoded thereby, i.e., mRNA, or proteins encoded thereby that are derived from a common ancestor gene but are present in different species.

By “decreased expression level” of a gene it is meant that the amount of the transcription product, respectively the amount of the polypeptide encoded by said gene produced by the modified bacterium is decreased compared to an otherwise identical bacterium that does not carry said modification. More particularly, by “decreased expression level” of a gene it is meant that the amount of the transcription product, respectively the amount of the polypeptide encoded by said gene produced by the modified bacterium is decreased by at least 10%, such as at least 20%, at least 30%, at least 40%, at least 50% at least 60%, at least 70%, at least 80%, at least 90% or at least 100%, compared to an otherwise identical bacterium that does not carry said modification. The level of expression of a gene can be determined by well-known methods, including PCR, Southern blotting, and the like. In addition, the level of gene expression can be estimated by measuring the amount of mRNA transcribed from the gene using various well-known methods, including Northern blotting, quantitative RT-PCR, and the like. The amount of the polypeptide encoded by the gene can be measured by well-known methods, including ELISA, Immunohistochemistry, or Western Blotting, and the like.

Expression of a gene can be decreased by introducing a mutation into the gene in the genome of the modified bacterium so that the intracellular activity of the polypeptide encoded by the gene is decreased as compared to an otherwise identical bacterium that does not carry the said mutation. Mutations, which result in a decreased expression of the gene include the replacement of one nucleotide or more to cause an amino acid substitution in the polypeptide encoded by the gene (missense mutation), the introduction of a stop codon (nonsense mutation), deletion, or insertion of nucleotides to cause a frameshift, insertion of a drug-resistance gene, or deletion of a part of the gene or the entire gene (Qiu and Goodman, 1997; Kwon et al., 2000). The expression can also be decreased by modifying an expression regulating sequence such as the promoter, the Shine-Dalgarno (SD) sequence, etc. Expression of the gene can also be decreased by gene replacement (Datsenko and Wanner, 2000), such as the “lambda-red mediated gene replacement”. The lambda-red mediated gene replacement is a particularly suitable method to inactive one or more genes as described herein.

“Inactivating”, “inactivation” and “inactivated”, when used in the context of a gene, means that the gene in question no longer expresses a functional protein. It is possible that the modified DNA region is unable to naturally express the gene due to the deletion of a part of or the entire gene sequence, the shifting of the reading frame of the gene, the introduction of missense/nonsense mutation(s), or the modification of an adjacent region of the gene, including sequences controlling gene expression, such as a promoter, enhancer, attenuator, ribosome-binding site, etc. Preferably, a gene of interest is inactivated by the deletion of a part of or the entire gene sequence, such as by gene replacement. Inactivation may also be accomplished by introducing or expressing a rare-cutting endonuclease able to selectively inactivate by DNA cleavage, preferably by the double-strand break, the gene of interest. A “rare-cutting endonuclease” within the context of the present invention includes transcription activator-like effector (TALE) nucleases, meganucleases, zinc-finger nucleases (ZFN), and RNA-guided endonucleases.

The presence or absence of a gene in the genome of a bacterium can be detected by well-known methods, including PCR, Southern blotting, and the like. In addition, the level of gene expression can be estimated by measuring the amount of mRNA transcribed from the gene using various well-known methods, including Northern blotting, quantitative RT-PCR, and the like. The amount of the protein encoded by the gene can be measured by well-known methods, including SDS-PAGE followed by an immunoblotting assay (Western blotting analysis), and the like.

By “increased expression level” of a gene it is meant that the amount of the transcription product, respectively the amount of the polypeptide encoded by said gene produced by the modified bacterium is increased compared to an otherwise identical bacterium that does not carry said modification. More particularly, by “increased expression level” of a gene it is meant that the amount of the transcription product, respectively the amount of the polypeptide encoded by said gene produced by the modified bacterium is increased by at least 10%, such as at least 20%, at least 30%, at least 40%, at least 50% at least 60%, at least 70%, at least 80%, at least 90%, at least 100%, at least 150%, at least 200%, at least 300%, at least 400%, at least 500%, at least 600%, at least 700% at least 800%, at least about 900%, at least about 1000%, at least about 2000%, at least about 3000%, at least about 4000%, at least about 5000%, at least about 6000%, at least about 7000%, at least about 8000% at least about 9000% or at least about 10000%, compared to an otherwise identical bacterium that does not carry said modification. The level of expression of a gene can be determined by well-known methods, including PCR, Southern blotting, and the like. In addition, the level of gene expression can be estimated by measuring the amount of mRNA transcribed from the gene using various well-known methods, including Northern blotting, quantitative RT-PCR, and the like. The amount of the polypeptide encoded by the gene can be measured by well-known methods, including ELISA, Immunohistochemistry, or Western Blotting, and the like.

By “increased expression level” of a polypeptide it is meant that the amount of the polypeptide in question produced by the modified microorganism is increased compared to an otherwise identical bacterium that does not carry said modification. More particularly, by “increased expression level” of a polypeptide it is meant that the amount of the polypeptide in question produced by the modified bacterium is increased by at least 10%, such as at least 20%, at least 30%, at least 40%, at least 50% at least 60%, at least 70%, at least 80%, at least 90%, at least 100%, at least 150%, at least 200%, at least 300%, at least 400%, at least 500%, at least 600%, at least 700% at least 800%, at least about 900%, at least about 1000%, at least about 2000%, at least about 3000%, at least about 4000%, at least about 5000%, at least about 6000%, at least about 7000%, at least about 8000% at least about 9000% or at least about 10000%, compared an otherwise identical bacterium that does not carry said modification. The amount of a polypeptide produced in a given cell can be determined by any suitable quantification technique known in the art, such as ELISA, Immunohistochemistry, or Western Blotting.

An increase in polypeptide expression may be achieved by any suitable means well-known to those skilled in the art. For example, an increase in polypeptide expression may be achieved by increasing the number of copies of the gene or genes encoding the polypeptide in the microorganism, such as by introducing into the microorganism an exogenous nucleic acid, such as a vector, comprising the gene or genes encoding the polypeptide operably linked to a promoter that is functional in the microorganism to cause the production of an mRNA molecule. An increase in polypeptide expression may also be achieved by the integration of at least a second copy of the gene or genes encoding the polypeptide into the genome of the microorganism. An increase in polypeptide expression may also be achieved by increasing the strength of the promoter(s) operably linked to the gene or genes encoding the polypeptide. An increase in polypeptide expression may also be achieved by modifying the ribosome binding site on the mRNA molecule encoding the polypeptide. By modifying the sequence of the ribosome binding site, the translation initiation rate may be increased, thus increasing translation efficiency.

As used herein, “decreased”, “decreasing” or “decrease of” expression of a polypeptide (e.g. an enzyme involved in the purine nucleotide degradation pathway) means that the expression of said polypeptide in a modified bacterium is reduced compared to the expression of said polypeptide in an otherwise identical bacterium that does not carry said modification (control). The expression of a polypeptide in a modified bacterium may be reduced by at least about 10%, and preferably by at least about 20%, at least about 25%, at least about 30%, at least about 35%, at least about 40%, at least about 45%, at least about 50%, at least about 55%, at least about 60%, at least about 65%, at least about 70%, at least about 75%, at least about 80%, at least about 85%, at least about 90%, at least about 95%, at least about 99% or 100%, or any percentage, in whole integers between 10% and 100% (e.g., 6%, 7%, 8%, etc.), compared to the expression of said polypeptide in an otherwise identical bacterium that does not carry said modification (control). More particularly, “decreased”, “decreasing” or “decrease of” expression of a polypeptide means that the amount of the polypeptide in the modified bacterium is reduced by at least about 10%, and preferably by at least about 20%, at least about 30%, at least about 35%, at least about 40%, at least about 45%, at least about 50%, at least about 55%, at least about 60%, at least about 65%, at least about 70%, at least about 75%, at least about 80%, at least about 85%, at least about 90%, at least about 95%, at least about 99% or 100%, or any percentage, in whole integers between 10% and 100% (e.g., 6%, 7%, 8%, etc.), compared to the amount of said polypeptide in an otherwise identical bacterium that does not carry said modification (control). The expression or amount of a polypeptide in a microorganism can be determined by any suitable means known in the art, including techniques such as ELISA, Immunohistochemistry, Western Blotting, or Flow Cytometry.

As used herein, “decreased”, “decreasing” or “decrease of” activity of a polypeptide (e.g. an enzyme involved in the purine nucleotide degradation pathway) means that the catalytic activity of said polypeptide in a modified bacterium is reduced compared to the catalytic activity of said polypeptide in an otherwise identical bacterium that does not carry said modification (control). The activity of a polypeptide in a modified bacterium may be reduced by at least about 10%, and preferably by at least about 20%, at least about 25%, at least about 30%, at least about 35%, at least about 40%, at least about 45%, at least about 50%, at least about 55%, at least about 60%, at least about 65%, at least about 70%, at least about 75%, at least about 80%, at least about 85%, at least about 90%, at least about 95%, at least about 99% or 100%, or any percentage, in whole integers between 10% and 100% (e.g., 6%, 7%, 8%, etc.), compared to the expression of said polypeptide in an otherwise identical bacterium that does not carry said modification (control). The activity of a polypeptide in a microorganism can be determined by any suitable protein and enzyme activity assay.

As used herein, “inhibitor of the enzyme” refers to any chemical compound, natural or synthetic, that inhibits the catalytic activity of the enzyme. An inhibitor of the enzyme does not necessarily need to achieve 100% or complete inhibition. In this regard, an inhibitor of the enzyme can induce any level of inhibition.

“Substitution” or “substituted” refers to the modification of the polypeptide by replacing one amino acid residue with another, for instance, the replacement of a Serine residue with a Glycine or Alanine residue in a polypeptide sequence is an amino acid substitution. When used with reference to a polynucleotide, “substitution” or “substituted” refers to a modification of the polynucleotide by replacing one nucleotide with another, for instance, the replacement of cytosine with thymine in a polynucleotide sequence is a nucleotide substitution.

“Conservative substitution”, when used with reference to a polypeptide, refers to a substitution of an amino acid residue with a different residue having a similar side chain, and thus typically involves the substitution of the amino acid in the polypeptide with amino acids within the same or similar class of amino acids. By way of example and not limitation, an amino acid with an aliphatic side chain may be substituted with another aliphatic amino acid, e.g., alanine, valine, leucine, and isoleucine; an amino acid with a hydroxyl side chain is substituted with another amino acid with a hydroxyl side chain, e.g., serine and threonine; an amino acid having an aromatic side chain is substituted with another amino acid having an aromatic side chain, e.g., phenylalanine, tyrosine, tryptophan, and histidine; an amino acid with a basic side chain is substituted with another amino acid with a basic side chain, e.g., lysine and arginine; an amino acid with an acidic side chain is substituted with another amino acid with an acidic side chain, e.g., aspartic acid or glutamic acid; and a hydrophobic or hydrophilic amino acid is replaced with another hydrophobic or hydrophilic amino acid, respectively.

“Non-conservative substitution”, when used with reference to a polypeptide, refers to a substitution of an amino acid in a polypeptide with an amino acid with significantly differing side chain properties. Non-conservative substitutions may use amino acids between, rather than within, the defined groups and affects (a) the structure of the peptide backbone in the area of the substitution (e.g., serine for glycine), (b) the charge or hydrophobicity, or (c) the bulk of the side chain. By way of example and not limitation, an exemplary non-conservative substitution can be an acidic amino acid substituted with a basic or aliphatic amino acid; an aromatic amino acid substituted with a small amino acid, and a hydrophilic amino acid substituted with a hydrophobic amino acid.

“Expression” includes any step involved in the production of a polypeptide (e.g., encoded enzyme) including, but not limited to, transcription, post-transcriptional modification, translation, post-translational modification, and secretion.

As used herein, the “regulatory region” of a gene refers to a nucleic acid sequence that affects the expression of a coding sequence. Regulatory regions are known in the art and include, but are not limited to, promoters, enhancers, transcription terminators, polyadenylation sites, matrix attachment regions, and/or other elements that regulate the expression of a coding sequence.

As used herein, “vector” refers to a nucleic acid molecule capable of transporting another nucleic acid molecule to which it has been linked. One type of vector is a “plasmid”, which refers to a circular double-stranded nucleic acid loop into which additional nucleic acid segments can be ligated. Certain vectors are capable of directing the expression of genes to which they are operatively linked. Such vectors are referred to herein as “expression vectors”. Certain other vectors are capable of facilitating the insertion of an exogenous nucleic acid molecule into a genome of a bacterium. Such vectors are referred to herein as “transformation vectors”. In general, vectors of utility in recombinant nucleic acid techniques are often in the form of plasmids. In the present specification, “plasmid” and “vector” can be used interchangeably as the plasmid is the most commonly used form of a vector. Large numbers of suitable vectors are known to those of skill in the art and commercially available.

As used herein, “promoter” refers to a sequence of DNA, usually upstream (5) of the coding region of a structural gene, which controls the expression of the coding region by providing recognition and binding sites for RNA polymerase and other factors, which may be required for initiation of transcription. The selection of the promoter will depend upon the nucleic acid sequence of interest. A suitable “promoter” is generally one, which is capable of supporting the initiation of transcription in a bacterium of the invention, causing the production of an mRNA molecule. “Strong” promoters include, for example, natural promoters from Bacillus subtilis, Bacillus amyloliquefaciens or similar, such as P43, P15, Pveg, Pylb, PgroES, PsigX, PtrnQ, Ppst, PsodA, PrpsF, PlepA, PliaG, PrpsF, Ppst, PfusA, PsodA, Phag as well as artificial promoters active in Bacillus subtilis or inducible Bacillus subtilis promoters, such as PmtlA, Pspac, PxylA, PsacB, or similar, that enable efficient expression and overproduction of proteins. Further examples of “strong” promoters include natural promoters from Corynebacterium, such as P CP_2454, Ptuf and Psod, natural promoters from E. coli, such as T7, and the promoter P F1 derived from the corynephage BFK20.

As used herein, “operably linked” refers to a juxtaposition wherein the components described are in a relationship permitting them to function in their intended manner. A control sequence “operably linked” to a coding sequence is ligated in such a way that expression of the coding sequence is achieved under conditions compatible with the control sequence. A promoter sequence is “operably-linked” to a gene when it is in sufficient proximity to the transcription start site of a gene to regulate transcription of the gene.

“Percentage of sequence identity,” “% sequence identity” and “percent identity” are used herein to refer to comparisons between an amino acid sequence and a reference amino acid sequence. The “% sequence identity”, as used herein, is calculated from the two amino acid sequences as follows: The sequences are aligned using Version 9 of the Genetic Computing Group's GAP (global alignment program), using the default BLOSUM62 matrix with a gap open penalty of −12 (for the first null of a gap) and a gap extension penalty of −4 (for each additional null in the gap). After alignment, percentage identity is calculated by expressing the number of matches as a percentage of the number of amino acids in the reference amino acid sequence.

“Reference sequence” or “reference amino acid sequence” refers to a defined sequence to which another sequence is compared. In the context of the present invention, a reference amino acid sequence may, for example, be an amino acid sequence set forth in SEQ ID NO: 1.

As used herein, the term “about” means plus or minus 10% of the numerical value of the number with which it is being used.

Where a numerical limit or range is stated herein, the endpoints are included. Also, all values and sub-ranges within a numerical limit or range are specifically included as if explicitly written out.

As used herein, the indefinite articles “a” and “an” mean “at least one” or “one or more” unless the context clearly dictates otherwise.

As used herein, the terms “comprising”, “including”, “having” and grammatical variants thereof are to be taken as specifying the stated features, steps, or components but do not preclude the addition of one or more additional features, steps, components or groups thereof.

Having generally described this invention, a further understanding can be obtained by reference to certain specific examples, which are provided herein for purposes of illustration only, and are not intended to be limiting unless otherwise specified.

### EXAMPLES

### Example 1: Selection of a Starting Strain

Different Bacillus strains can be used as starting strains for the engineering of isoprenoid cytokinin production (Table 2). Bacillus spp. strains can be isolated from nature or obtained from culture collections. Among others, starting strains for isoprenoid cytokinin production can be selected among Bacillus subtilis strains that have already been subjected to classical methods of mutagenesis and selection to overproduce metabolites related to the purine nucleotide biosynthetic pathway. For example, strains overproducing riboflavin, inosine, guanosine or adenosine may be selected. Strains subjected to random mutagenesis and toxic metabolic inhibitors from purine nucleotide and riboflavin pathway are preferred and are included in Table 3.

TABLE 2
Potential non-GMO starting strains that could be used for the development of
isoprenoid-cytokinins-producing strain.

| | | Alternative strain | | | |
|---|---|---|---|---|---|
| Species | Strain | name | Product | Availability | Remarks |
| B. subtilis | 168 | ATCC6051 | none | yes | Type strain |
| B. subtilis subsp. | W23 | ATCC 23059/ | none | yes | Type strain |
| spizizenii | | NRRL B-14472 | | | |
| B. subtilis | RB50 | NRRL B18502 | riboflavin | yes | Developed by |
| | | | | | Roche/DSM |
| B. subtilis | RB58 | ATCC55053 | riboflavin | yes | Containing |
| | | | | | additional copy of |
| | | | | | rib operon |
| B. subtilis | VNII Genetika | VKPM B2116, | riboflavin | yes | Developed by VNII |
| | 304 | VBB38 | | | Genetika |
| B. subtilis | FERM-P 1657 | | riboflavin | no | Ajinomoto |
| B. subtilis | FERM-P 2292 | | riboflavin | no | Ajinomoto |
| B. subtilis | AJ12644 | FERM BP-3855 | riboflavin | no | Ajinomoto |
| B. subtilis | AJ12643 | FERM BP-3856 | riboflavin | no | Ajinomoto |
| B. subtilis | ATCC13952 | | inosine | yes | |
| B. subtilis | ATCC19221 | IFO 14123 | guanosine | yes | |
| B. subtilis | ATCC13956 | IFO 14124 | inosine | yes | |
| Bacillus sp. | ATCC21615 | ASB-5741 | none | yes | Asahi Chemical |
| | | | | | Industry |
| Bacillus sp. | ATCC21616 | ASB-5741-2059A | adenosine | yes | Asahi Chemical |
| | | | | | Industry |
| Corynebacterium stationis | ATCC6872 | DSM 20305, IAM | adenosine | yes | |
| (ammoniagenes) | | 1645, NCTC 2399 | | | |

TABLE 3
Metabolic inhibitors interfering with the purine
nucleotide or the riboflavin biosynthesis pathway.

| Metabolic inhibitor | Type of inhibitor |
|---|---|
| 8-azaguanine | purine analogue |
| thioguanine | purine analogue |
| 8-azaxanthine | purine analogue |
| decoyinine | purine analogue |
| roseoflavin | structural analogue of |
| | riboflavin |

Bacillus subtilis VKPM B2116 is a hybrid strain of B. subtilis 168 (most common B. subtilis host strain with approx. 4 Mbp genome) with a 6.4 kbp island of DNA from the strain B. subtilis W23. Such architecture is common for most B. subtilis industrial strains and was obtained by transforming B. subtilis 168 (tryptophan auxotroph trpC) with W23 (prototrophic TrpC+) DNA. The 6.4 kbp W23 island in the genome of VKPM B21216 is the same as in B. subtilis SMY, which is one of the B. subtilis legacy strains with genome publicly available (Zeigler et al. 2008). B. subtilis VKPM B2116 is a direct descendant strain of B. subtilis SMY, obtained by classical mutagenesis and selection. Another name for this strain is B. subtilis VNII Genetika 304. The description of the construction of the strain is in Soviet Union patent SU908092, filed in 1980. The mutations were obtained by subsequent mutagenesis and selection of metabolic inhibitors. The strain VKPM B2116 is resistant to roseoflavin, a toxic analogue of vitamin B2, due to a mutation in the ribC gene, encoding a flavin kinase. This strain is also resistant to 8-azaguanine, a toxic purine analogue.

### Example 2: Synthesis of Synthetic Genes IPT SEQ ID 1 and LOG SEQ ID 34 for Isoprenoid Cytokinin Biosynthesis, Optimized for Bacillus subtilis

The amino acid sequences of adenylate dimethylallyltransferase (IPT) Tzs from Agrobacterium tumefaciens (syn. Agrobacterium fabrum (strain C58/ATCC 33970), gene tzs, E.C.2.5.1.27, UniProt: P58758) (SEQ ID NO: 1) and of cytokinin riboside 5′-monophosphate phosphoribohydrolase (LOG) from Corynebacterium glutamicum (strain ATCC 13032/DSM 20300/NCIMB 10025, gene Cg/2379, E.C. 3.2.2.n1, UniProt: Q8NN34) (SEQ ID NO: 34) were used for generating codon-optimized nucleotide sequences for gene expression in B. subtilis by using GENEius (Eurofins). The synthetic DNA fragment IPT-LOG (SEQ ID NO: 71) was designed with the addition of an RBS sequence, and with short adapter sequences at both ends of the synthetic fragment for further assembly of the synthetic isoprenoid cytokinin operon expression cassette.

### Example 3: Synthesis of Synthetic Genes IPT SEQ ID 2 and LOG SEQ ID 34 for Isoprenoid Cytokinin Biosynthesis, Optimized for Bacillus subtilis

The amino acid sequences of adenylate dimethylallyltransferase (IPT) Tmr from Agrobacterium tumefaciens (syn. Agrobacterium fabrum (strain C58/ATCC 33970), gene izt, E.C.2.5.1.27, Uniprot: P0A3L5) (SEQ ID NO: 2) and of cytokinin riboside 5′-monophosphate phosphoribohydrolase (LOG) from Corynebacterium glutamicum (gene Cg/2379, E.C. 3.2.2.n1, UniProt: Q8NN34) (SEQ ID NO: 34) were used for generating codon-optimized nucleotide sequences for gene expression in B. subtilis using GENEius (Eurofins). The synthetic DNA fragment IPT-LOG (SEQ ID NO: 72) was designed with the addition of an RBS sequence, and with short adapter sequences at both ends of the synthetic fragment for further assembly of the synthetic isoprenoid cytokinin operon expression cassette.

### Example 4: Assembly of Synthetic Isoprenoid Cytokinin Operons Containing IPT and LOG, and Transformation into B. subtilis

The synthetic fragments containing synthetic genes IPT-LOG (SEQ ID NO: 71 and SEQ ID NO: 72) for isoprenoid cytokinin biosynthesis were assembled in artificial isoprenoid cytokinin operons. The initial and end fragments containing gene integration homology, the promoter sequence, and the erythromycin selectable marker (SEQ ID NO: 73) were designed and synthesized for stable genome integration into the amyE locus in the genome of B. subtilis.

The first step of the artificial operon assembly was PCR amplification of separate DNA fragments performed using primer pair SEQ ID NO: 74 and SEQ ID 75 for the initial fragment SEQ ID NO: 75, and for synthetic fragments IPT-LOG SEQ ID NO: 71 and SEQ ID NO: 72 containing genes for isoprenoid-cytokinin biosynthesis. The primer set SEQ ID NO: 77 and SEQ ID NO: 78 was used for amplification of the end fragment SEQ ID NO: 79.

The fragments were amplified using Eppendorf cycler and Phusion polymerase (Thermo Fisher) in the buffer provided by the manufacturer and with the addition of 200 μM dNTPs, 0.5 μM of each primer, and approximately 10 ng of template in a final volume of 50 μl for 30 cycles using the PCR cycling conditions: 98° C. 30 s, 30 cycles of (98° C. 30 s, 68.5° C. 25 s, 72° C. 23/25 s), 72° C. 5 min, 10° C. hold.

PCR reaction products of each fragment were run on 0.8% agarose gel, excised from the gel, and extracted from the gel by GeneJET Gel Extraction Kit (Thermo Fisher) according to the protocol provided by the manufacturer. The fragments were assembled into an artificial operon by repetitive steps of restriction and ligation. A combination of SpeI (BcuI) and XbaI restriction sites was used to provide compatible restriction ends for a successful ligation. After each step of ligation, the combined fragments were used as a new template for the next PCR amplification. The restriction was done in 50 μl volume with the addition of 5 μl FD green buffer (Thermo Fisher Scientific), 2-3 μl of the selected enzyme (SpeI (BcuI), and XbaI, Thermo Fisher), and up to app. 1500 ng of PCR fragment. After restriction digest, the digested DNA fragments were cleaned with Wizard SV Gel and PCR Clean-up system according to the protocol provided by the manufacturer. The first two fragments were used in the ligation reaction with 2.5 U of T4 DNA ligase (Thermo Fisher) in the buffer provided by the manufacturer and the addition of 5% PEG 4000 and both fragments in a 1:1 molar ratio to the final volume of 15 μl. In the next step, 1 μl of inactivated ligation was used as a template in a new 50 μL PCR with a primer set SEQ ID NO: 80 and SEQ ID NO: 81, and the same PCR mix and PCR cycling conditions as previously but with longer elongation time. The restriction digest, cleaning, and ligation steps were repeated for ligation of the end fragment. PCR was run on 0.8% agarose gel, the fragment was excised from the gel, digested and cleaned as before, and ligated as before. The final operon containing the amy E homology, promoter with RBS sequence, IPT and LOG genes, and erythromycin resistance cassette was amplified using the primer pair SEQ ID NO: 76 and SEQ ID NO: 77, cleaned and ligated as described before. The constructed synthetic trans-zeatin operons containing IPT-LOG SEQ ID NO: 82 and SEQ ID NO: 83 were used for the transformation of Bacillus subtilis VKPM B2116. The transformation with IPT-LOG operon SEQ ID NO: 83 resulted in the transformed strains TZAB1, TZAB2, TZAB3, and TZAB4. The transformation with IPT-LOG operon SEQ ID NO: 82 resulted in the transformed strains TZAB14 and TZAB15. The correct integration of the artificial operons at amyE integration site was confirmed by cPCR.

TABLE 4
Bacillus subtilis strains obtained
by transformation of IPT-LOG operons.

| | IPT-LOG SEQ ID 83 | IPT-LOG SEQ ID 82 |
|---|---|---|
| VKPM B2116 | / | / |
| TZAB1 | + | / |
| TZAB2 | + | / |
| TZAB3 | + | / |
| TZAB4 | + | / |
| TZAB14 | / | + |
| TZAB15 | / | + |

### Example 5: Increased Isoprenoid Cytokinin Biosynthesis by Increasing of the Isoprenoid Precursor Supply with the Overexpression of the Dxs

The isoprenoid side chain for the biosynthesis of isoprenoid cytokinins is synthesized by the MEP pathway in B. subtilis. The protein sequence of 1-deoxyxylulose-5-phosphate synthase (DXS, E.C. 2.2.1.7) from B. subtilis was used for generation of the synthetic nucleotide sequence of dxs by using a codon-optimizing feature GENEius (Eurofins) for gene expression in B. subtilis. The synthetic DNA fragments of dxs SEQ ID NO: 84 and SEQ ID NO: 85 were designed in two overlapping parts, which were joined by overlap PCR. The entire joined synthetic gene dxs SEQ ID NO: 86 was assembled in an artificial operon. The initial and end fragments containing lacA homology, the promoter sequence, and the spectinomycin selectable marker SEQ ID NO: 87 were designed and synthesized for stable genome integration into the genome of B. subtilis. The fragments were assembled as described in Example 4.

The first step of the artificial operon assembly was PCR amplification of separate DNA fragments performed with a set of primers SEQ ID NO: 74 and SEQ ID NO: 75 for the initial fragment SEQ ID NO: 88, the end fragment SEQ ID 89, and the joined synthetic gene dxs SEQ ID NO: 86.

The fragments were amplified using Eppendorf cycler and Phusion polymerase (Thermo Fisher) in the buffer provided by the manufacturer and with the addition of 200 μM dNTPs, 0.5 μM of each primer, and approximately 10 ng of template in a final volume of 50 μl for 30 cycles. The PCR cycling conditions used were 98° C. 30 s, 30 cycles of (98° C. 30 s, 71° C. 25 s, 72° C. 23/25 s), 72° C. 5 min, 10° C. hold.

PCR reaction products of each fragment were run on 0.8% agarose gel, excised from the gel, and extracted from the gel by GeneJET Gel Extraction Kit (Thermo Fisher) according to the protocol provided by the manufacturer. The fragments were assembled into an artificial operon by repetitive steps of restriction and ligation. A combination of SpeI (BcuI) and XbaI restriction sites was used to provide compatible restriction ends for a successful ligation. After each step of ligation, the combined fragments were used as a new template for the next PCR amplification. The restriction was done in 50 μl volume with the addition of 5 μl FD green buffer (Thermo Fisher Scientific), 2-3 μl of the selected enzyme (SpeI (BcuI), and XbaI, Thermo Fisher), and up to app. 1500 ng of PCR fragment. After restriction digest, the digested DNA fragments were cleaned with Wizard SV Gel and PCR Clean-up system according to the protocol provided by the manufacturer. The first two fragments were used in the ligation reaction with 2.5 U of T4 DNA ligase (Thermo Fisher) in the buffer provided by the manufacturer and both fragments in a 1:1 molar ratio to the final volume of 15 μl. In the next step, 1 μl of inactivated ligation was used as a template in a new 50 μL PCR with a primer set SEQ ID NO: 74 and SEQ ID NO: 75, and the same PCR mix and PCR cycling conditions as previously but with longer elongation time. The restriction digest, cleaning and ligation steps were repeated for ligation of the end fragment. In the last step, 1 μl of inactivated ligation was used as a template for complete synthetic dxs operon SEQ ID NO: 90 in a new 50 μL PCR using the primers SEQ ID NO: 91 and SEQ ID NO: 92, and the same PCR mix and PCR cycling conditions as previously but with longer elongation time. PCR was run on 0.8% agarose gel, the fragment was excised from the gel, cleaned as before, and ligated as before. The constructed synthetic dxs operon was used for the transformation of Bacillus subtilis TZAB15 with IPT-LOG operon at amyE (SEQ ID NO: 80). The transformation with the artificial DXS operon (SEQ ID NO: 90) resulted in the transformed strain TZAB43. The correct integration of the artificial operon at lacA integration site was confirmed by cPCR.

TABLE 5
Bacillus subtilis strains obtained by transformation
of the strain B. subtilis TZAB15 with DXS operon.

| | IPT-LOG SEQ ID 81 | DXS SEQ ID 88 |
|---|---|---|
| VKPM B2116 | / | / |
| TZAB15 | + | / |
| TZAB43 | + | + |

### Example 6: Cultivation of Bacillus subtilis Strains for Production of Isoprenoid Cytokinins

All of the constructed strains and control strain have been cultivated following this procedure. The frozen stocks of the strains VKPM B2116, TZAB1, TZAB2, TZAB3, TZAB4, TZAB14, TZAB15 and TZAB43, preserved in 20% glycerol at −80° C. were spread onto solid seed medium containing erythromycin and lincomycin in the appropriate concentration and incubated for approximately 1 day at 37° C. For further testing, a vegetative-stage medium was inoculated with 1 to 5 plugs of the culture on solid seed plates per baffled 250-mL Erlenmeyer flask containing 25 mL of vegetative medium and appropriate amounts of antibiotics. The cultures were incubated at 37° C. for 18-20 h at 220 RPM. The culture in the vegetative medium was used as seed culture, and it was inoculated into the production medium. A 10-% inoculum was used (2.5 mL per 25 mL of production medium in 250-mL Erlenmeyer flask). The production medium was used as described or it was amended with adenine sulfate (end concentration 3 g/L). The cultures were incubated at 30° C. or 37° C. for up to 48 h at 220 RPM. The fermented cultures were sampled and analyzed as described in the Example 7. The titer of trans-zeatin, trans-zeatin riboside, and isopentenyl adenine was measured using the LC/MS as described in the Example 7.

TABLE 6
Composition of the solid seed medium

| Compound | Per 1 L |
|---|---|
| Tryptone | 10 g |
| Yeast extract | 5 g |
| NaCl | 5 g |
| Maltose | 20 g |
| Agar | 20 g |
| pH 7.2-7.4 | |

TABLE 7
Composition of the vegetative medium

| Compound | Per 1 L | |
|---|---|---|
| Molasses | 20 | g |
| CSL | 20 | g |
| Yeast extract | 5 | g |
| MgSO₄*7H₂O | 0.5 | g |
| NH₄)₂SO₄ | 5 | g |

The ingredients of the vegetative medium (Table 7) were mixed and the pH was set to 7.2-7.4. KH₂PO₄—K₂HPO₄ solution was then added in the final concentration for KH₂PO₄ 1.5 g/L and K₂HPO₄ 3.5 g/L. The medium was distributed into Erlenmeyer flasks (25 ml/250 ml-baffled Erlenmeyer flask) and autoclaved 30 min, 121° C. Sterile glucose was added after autoclaving in the final concentration of 7.5 g/L. Antibiotics were added before inoculation.

TABLE 8
Composition of the production medium

| Compound | Per 1 L | |
|---|---|---|
| Yeast | 20 | g |
| CSL | 5 | g |
| MgSO₄*7H₂O | 0.5 | g |

The ingredients of the production medium (Table 8) were mixed and the pH was set to 7.2-7.4. KH₂PO₄—K₂HPO₄ solution was then added in the final concentration for KH₂PO₄ 1.5 g/L and K₂HPO₄ 3.5 g/L. The medium was autoclaved at 121° C. for 30 min. Sterile urea solution (20 ml of stock solution, final concentration is 6 g/L), sterile glucose solution (500 ml of stock solution, final concentration is 100 g/L glucose) were added after autoclaving to obtain 1 L of production medium.

Appropriate antibiotics were added before inoculation. The medium was then distributed into sterile Erlenmeyer flasks (25 ml/250 ml-baffled Erlenmeyer flask).

### Example 7: Analysis of Isoprenoid Cytokinins

All of the strains that had been cultivated following the procedure described in the Example 6 were analysed according to this procedure. The fermented production medium was sampled and immediately frozen at −20° C. For extraction of the metabolites, the fermented production medium was diluted 1:1 with the extraction buffer composed of a 1:1 mixture of methanol and 100 mM ammonium acetate pH 4. The samples were extracted for 1 h at room temperature with constant agitation, centrifuged at 4000-4500 RPM for 15 min, and filtered (0.22 um). The samples were immediately analyzed by LC/MS or stored at −20° C. until analysis.

The samples were analyzed on Thermo Accela 1250 HPLC instrument coupled to Thermo TSQ Quantum Access MAX, MS/MS capable mass spectrometer. The method is based on Thermo Accucore C30, 150×4.6 mm, 2.6 um particle size column, kept at 60° C., with mobile phase A—0.1% formic acid in water and mobile phase B—methanol, in gradient program, with starting conditions: 95% A, linear gradient increase in B % to 50% at 10 min, and 5 min stabilization to initial conditions, at 1 ml/min flow rate. Mass spectrometer was equipped with hESI ion source, operated in positive (+) mode, with spray voltage set at 4600 V, vaporizer temperature at 350° C., collision pressure at 1.0 torr and 10 V collision energy. Trans-zeatin was observed in MRM mode with transitions from parent 219.9 m/z to daughter ions: 185.2, 148.0 and 136.0.

Trans-zeatin riboside (tZR) was observed in MRM mode with transitions from parent 352.5 m/z to daughter ions: 220.1, 202.1, 148.0 and 136.1. Isopentenyl adenine (iP) was observed in MRM mode with transitions from parent 204.4 m/z to daughter ions: 148.4, 136.3, and 119.2. Isopentenyl adenine riboside (iPR) was observed in MRM mode with transitions from parent 336.5 m/z to daughter ions: 204.1, 148.0, and 113.1.

### Example 8: Production of Trans-Zeatin and Related Isoprenoid Cytokinins by Bacillus subtilis Strains with Heterologous Expression of IPT and LOG

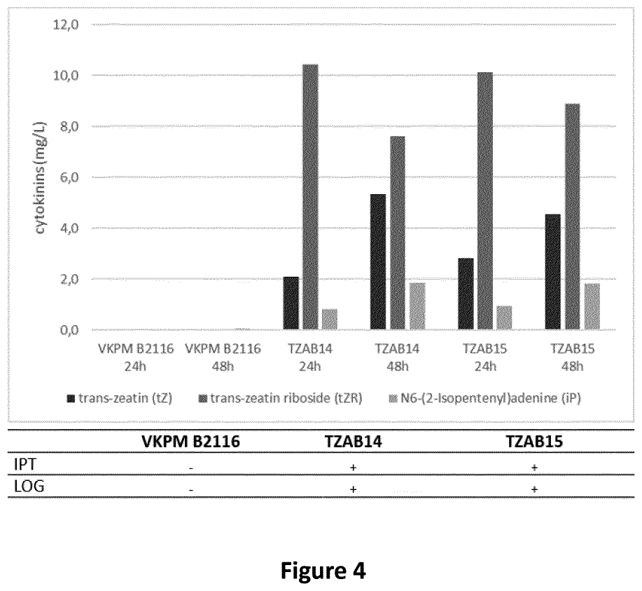

The cultivation was performed as described in the Example 6. The extraction and analysis were performed as described in Example 7. The yields of isoprenoid cytokinins detected are shown in FIG. 4. The strains expressing IPT-LOG SEQ ID NO: 82 produce isoprenoid cytokinins in the amounts up to 10 mg/L (see FIG. 4).

### Example 9: Production of Trans-Zeatin and Related Isoprenoid Cytokinins by Bacillus subtilis Strains with Heterologous Expression of IPT and LOG, and with Adenine Sulfate in the Growth Medium

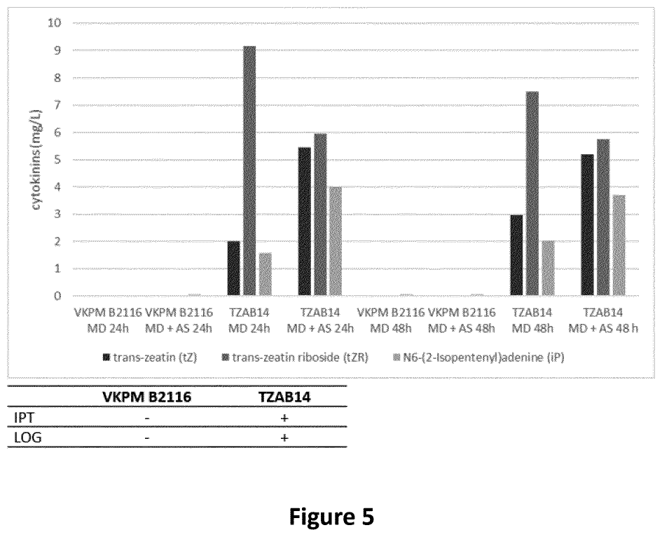

The cultivation was performed as described in the Example 6. The production medium was composed as described in the Example 6, or it was amended with adenine sulfate in the final concentration 3 g/L. The fermentation broth was extracted and analyzed as described in the Example 7. The results are shown in FIG. 5. The production of isoprenoid cytokinins in the strain TZAB14 with IPT-LOG SEQ ID NO: 82 is increased in the medium containing adenine sulfate compared to the medium without adenine sulfate.

### Example 10: Production of Trans-Zeatin and Related Isoprenoid Cytokinins by Bacillus subtilis Strains with Heterologous Expression of IPT-LOG SEQ ID 82 and Overexpression of DXS

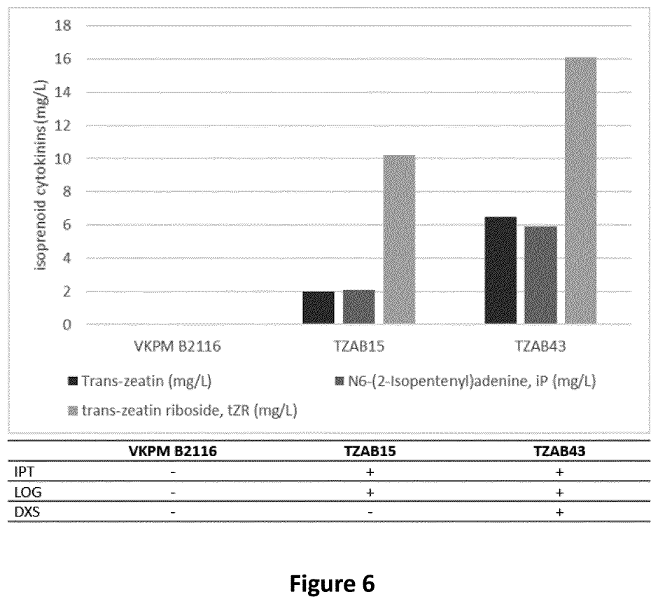

The cultivation was performed as described in the Example 6. The extraction and analysis were performed as described in the Example 7. The yields are shown in FIG. 6. The production of isoprenoid cytokinins is increased in the strains with IPT-LOG operon SEQ ID NO: 82 and the DXS operon SEQ ID NO: 90.

### Example 11: Analysis of Trans-Zeatin and Related Isoprenoid Cytokinins Production by Bacillus subtilis Strains with IPT-LOG (SEQ ID NO: 83)

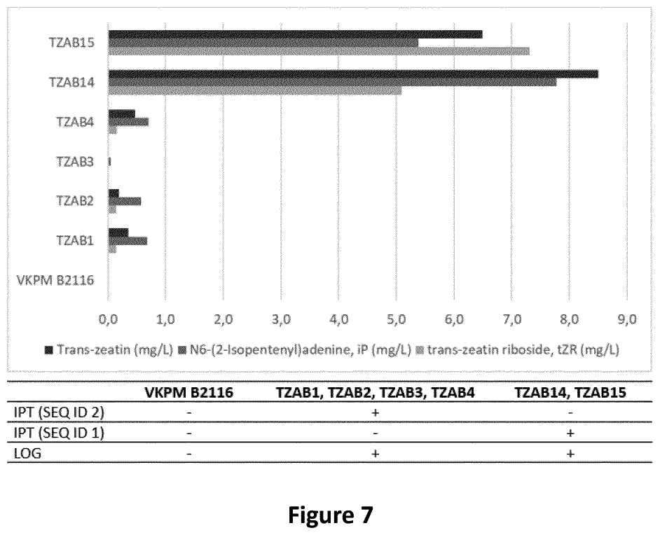

The cultivation was performed as described in the Example 6. The extraction and analysis were performed as described in the Example 7. The strains TZAB1, TZAB2, TZAB3 and TZAB4 with IPT-LOG SEQ ID NO: 83 and the strains TZAB14 and TZAB 15 with IPT-LOG SEQ ID NO: 82 produced isoprenoid cytokinins. The results are shown in FIG. 7.

### Example 12: Production of Trans-Zeatin and Related Isoprenoid Cytokinins with Heterologous Expression of IPT-LOG Operon, IPT and DXS in Bacillus subtilis Strains

The synthetic DNA fragments containing genes IPT1-LOG 1 (SEQ ID NO: 71) for isoprenoid cytokinin biosynthesis was assembled in artificial isoprenoid cytokinin operons as described in Example 4.

The IPT1 expression cassette containing only IPT1 (SEQ ID NO: 1) was assembled by PCR amplification of first part of fragments IPT1-LOG 1 (SEQ ID NO: 82), using primers SEQ ID NO: 123 and SEQ ID NO: 183 and separately final part of IPT1-LOG 1 (SEQ ID NO: 82) using primers SEQ ID NO: 123 and SEQ ID NO: 128, with Eppendorf cycler and Phusion polymerase (Thermo Fisher) in the buffer provided by the manufacturer and with the addition of 200 μM dNTPs, 0.5 μM of each primer, and approximately 10 ng of template in a final volume of 50 μl for 30 cycles using the PCR cycling conditions: 98° C. 30 s, 30 cycles of (98° C. 30 s, 68.5° C. 25 s, 72° C., 25 s), 72° C. 5 min, 10° C. hold. The PCR reaction products was run on 0.8% agarose gel, excised from the gel, and extracted from the gel by GeneJET Gel Extraction Kit (Thermo Fisher) according to the protocol provided by the manufacturer. The fragments were assembled into an artificial IPT1 expression cassette by restriction and ligation. A XbaI restriction sites was used to provide compatible restriction ends for a successful ligation. After ligation, the combined fragments were used as a new template for the next PCR amplification. The restriction was done in 50 μl volume with the addition of 5 μl FD green buffer (Thermo Fisher Scientific), 2-3 μl of the selected XbaI (Thermo Fisher) restriction enzyme, and up to app. 1500 ng of PCR fragment. After restriction digest, the digested DNA fragments were cleaned with Wizard SV Gel and PCR Clean-up system according to the protocol provided by the manufacturer. The first two fragments were used in the ligation reaction with 2.5 U of T4 DNA ligase (Thermo Fisher) in the buffer provided by the manufacturer and the addition of 5% PEG 4000 and both fragments in a 1:1 molar ratio to the final volume of 15 μl. In the next step, 1 μl of inactivated ligation was used as a template in a new 50 μL PCR with a primer set SEQ ID NO: 123 and SEQ ID NO: 128, and the same PCR mix and PCR cycling conditions as previously but with longer elongation time. Generating the final expression cassette with IPT fragment (SEQ ID NO: 184).

The final expression cassettes constructed for trans-zeatin operon containing IPT1-LOG 1 (SEQ ID NO: 82) or IPT fragment (SEQ ID NO: 184) were then used for the transformation of Bacillus subtilis VKPM B2116, Bacillus subtilis 168 and Bacillus subtilis RB50 strains and resulted in the development of new transformed strains presented in Table 9. The correct integration of the artificial operons at amyE integration site was confirmed by cPCR.

The synthetic fragments containing dxs gene (SEQ ID NO: 86) were assembled in expression cassette as described in Example 5. The constructed DXS expression cassette (SEQ ID NO: 90) was used for the transformation of Bacillus subtilis VKPM B2116, Bacillus subtilis 168 and Bacillus subtilis RB50 derived strains and resulted in the development of new transformed strains presented in Table 9.

The correct integration of the artificial operon at lacA integration site was confirmed by cPCR.

TABLE 9
Bacillus subtilis strains obtained by transformation
of DXS, IPT and IPT-LOG operons.

| | | IPT1-LOG1 | IPT1 | DXS |
|---|---|---|---|---|
| | | SEQ ID | SEQ ID | SEQ ID |
| Strain | Parent strain | 82 | 179 | 90 |
| VKPM B2116 | / | / | / | / |
| TZ2917 | VKPM B2116 | / | + | / |
| TZ2927 | VKPM B2116 | + | / | / |
| TZ2980 | VKPM B2116 | / | + | + |
| TZ2998 | VKPM B2116 | + | / | + |
| 168 | / | / | / | / |
| TZ2720 | 168 | / | + | / |
| TZ2722 | 168 | + | / | / |
| TZ2764 | 168 | + | + | + |
| TZ2782 | 168 | + | / | + |
| RB50 | / | / | / | / |
| TZ2835 | RB50 | / | + | / |
| TZ2851 | RB50 | + | / | / |
| TZ2870 | RB50 | / | + | + |
| TZ2904 | RB50 | + | / | + |

All of the constructed strains and control strain have been cultivated following the procedure described int the Example 6. The frozen stocks of the strains preserved in 20% glycerol at −80° C. were spread onto solid seed medium containing erythromycin and lincomycin in the appropriate concentration and incubated for approximately 1 day at 37° C. For further testing, a vegetative-stage medium was inoculated with 1 to 5 plugs of the culture on solid seed plates per baffled 250-mL Erlenmeyer flask containing 25 mL of vegetative medium and appropriate amounts of antibiotics.

The cultures were incubated at 37° C. for 8-20 h at 220 RPM. The culture in the vegetative medium was used as seed culture, and it was inoculated into the production medium. A 10-% inoculum was used (2.5 mL per 25 mL of production medium in 250-mL Erlenmeyer flask). The production medium was amended with tryptophan (final concentration 50 mg/L) for evaluation of Bacillus subtilis 168 derived strains. The cultures were incubated at 30° C., 34° C. or 37° C. for up to 48 h at 220 RPM. The fermented cultures were sampled and analyzed as described in the Example 7. The titters of trans-zeatin (tZ), trans-zeatin riboside (tZR), isopentenyl adenine riboside (iPR) and isopentenyl adenine (iP) were measured using the LC/MS as described in the Example 7.

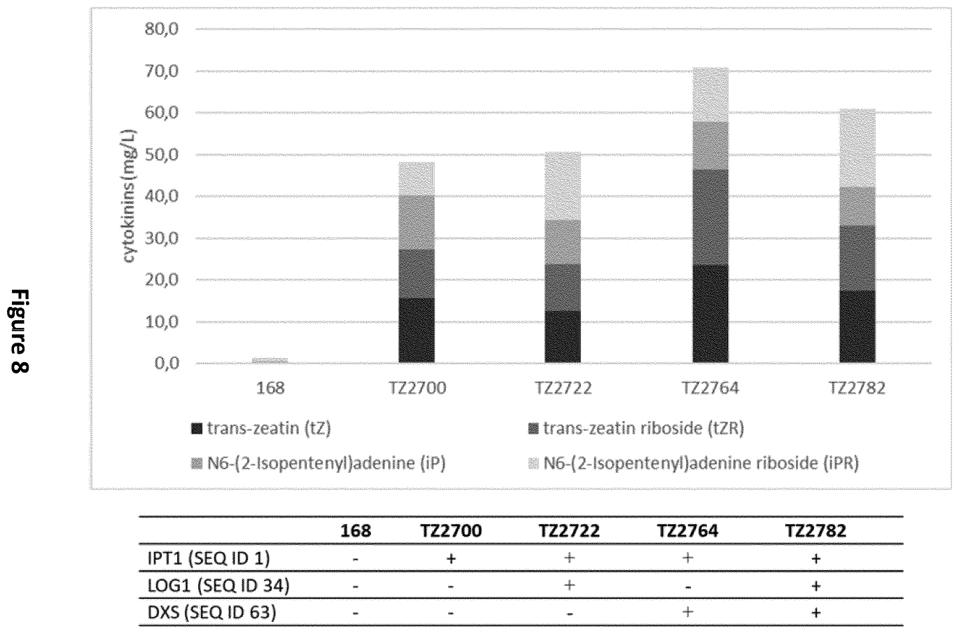

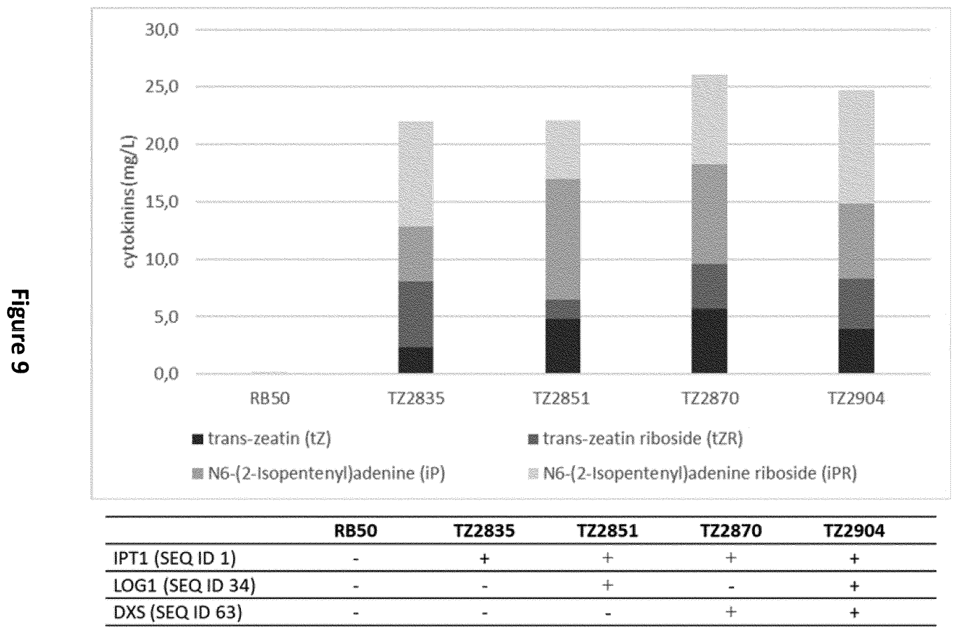

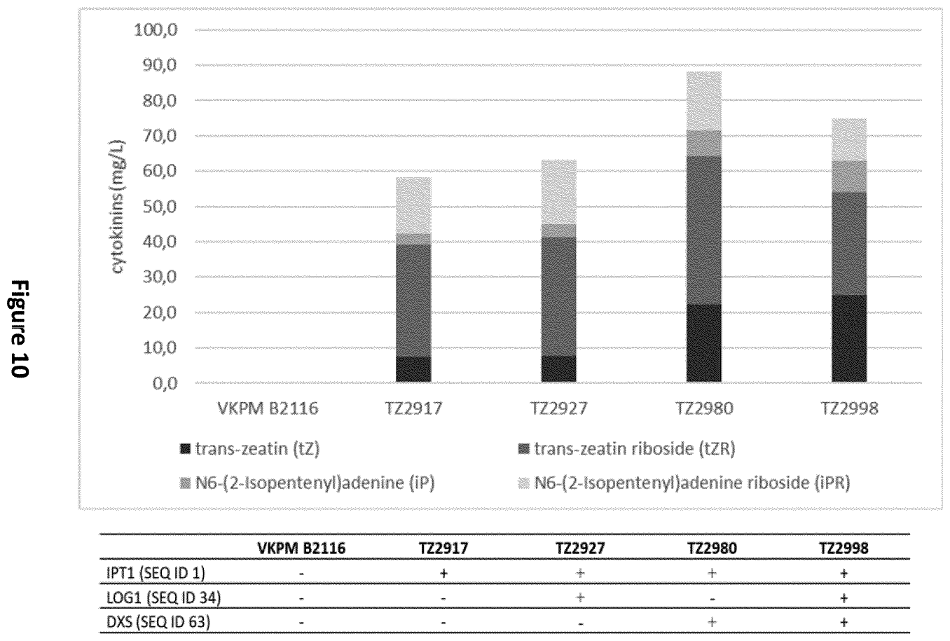

The extraction and analysis were performed as described in Example 7. The yields of isoprenoid cytokinins detected are shown in Table 17, FIG. 8, FIG. 9 and FIG. 10. The strains expressing IPT-LOG SEQ ID NO: 82 produce isoprenoid cytokinins in total amounts up to 60 mg/L (see FIG. 10) and the production of isoprenoid cytokinins is further overall increased in the strains with IPT-LOG operon (SEQ ID NO: 82) and the DXS operon (SEQ ID NO: 90).

### Example 13: Production of Trans-Zeatin and Related Isoprenoid Cytokinins with Heterologous Expression of IPT, IPT1-LOG 1 Operon and DXS in Escherichia coli BL21(DE3) Strains

To evaluate Escherichia coli as a possible isoprenoid cytokinins producing strain, two sets of expression plasmids were constructed.

First set was the plasmid for heterologous cytokinin gene/operon expression of synthetic gene IPT (SEQ ID NO: 179) and IPT-LOG operon (SEQ ID NO: 82). IPT gene or IPT-LOG operon were assembled into pBBR1 plasmid vector. pBBR1 vector has heterologous expression system based on promoter inducible with isopropyl-β-D-1-thiogalactopyranoside (IPTG): Lacl^{Q}/P_{lacuvs}-T7 and contains chloramphenicol resistance cassette as selection marker.

The second plasmid was cloned for heterologous expression of DXS (SEQ ID NO: 90) to increase the isoprenoid precursor supply. DXS gene was assembled into p15A plasmid vector. p15A vector has expression system which is based on the XyIS/Pm: where XyIS in the positive regulator of m-toluate inducible Pm promoter and contains kanamycin resistance cassette as selection marker.

The first step of the expression plasmids assembly was PCR amplification of insert fragments: IPT-LOG, DXS and vector backbones: pBBR1 and p15A. IPT DNA fragment was amplified with primer set SEQ ID NO: 163 and SEQ ID NO: 182, while IPT-LOG DNA fragment was amplified with primer set SEQ ID NO: 163 and SEQ ID NO: 164 for IPT and SEQ ID NO: 165 and SEQ ID NO: 166 for LOG amplification. The primer set SEQ ID NO: 167 and SEQ ID NO: 168 was used for amplification the pBBR1 vector backbone. DXS fragment was amplified with primer set: SEQ ID NO: 169 and SEQ ID NO: 170; and the primer set SEQ ID NO: 171 and SEQ ID NO: 172 was used for amplification the p15A vector backbone. The fragments were amplified using Eppendorf cycler and Phusion polymerase (Thermo Fisher) in the buffer provided by the manufacturer and with the addition of 200 μM dNTPs, 0.5 μM of each primer, and approximately 10 ng of template in a final volume of 50 μl for 30 cycles. PCR reaction products of each fragment were run on 0.8% agarose gel, excised from the gel, and extracted from the gel by GeneJET Gel Extraction Kit (Thermo Fisher) according to the protocol provided by the manufacturer. The mass of each fragment was measured using the NanoDrop instrument, absorbance at 260 nm. The fragments were assembled into final expression construct by 10 μl HIFI assembly reaction (NEBuilder HiFi DNA Assembly Master Mix). The reaction was set up on ice: to 5 μl of HiFi DNA Assembly Master Mix fragments were added in DNA molar ratio vector: insert=1:2 (total amount of fragments was up to 0.2 pmols). The reaction was filled to the final volume of 10 μl with nuclease free water. Samples were incubated in a thermocycler at 50° C. for 60 minutes. In the next step, 1 μl of the chilled assembled product was used for transformation of competent E. coli BL21(DE3) cells. Plasmid DNA was isolated from the obtained strains and correct assembly was confirmed by sequencing.

The transformation of the p15A plasmid for expression of DXS gene resulted in the transformed strain TZ3077. The transformation of the pBBR1 plasmid for expression of IPT gene resulted in the transformed strain TZAB3079. The transformation of pBBR1 plasmid for expression of IPT-LOG operon resulted in the transformed strains TZ3082. Additionally, IPT gene and IPT-LOG operon expression plasmid was transformed into TZ3077 (E. coli BL21(DE3) strain with DXS expression plasmid). Obtained strain with two plasmids: one for DXS gene and the other for IPT gene expression was saved as TZAB3087. Obtained strain with two plasmids: one for DXS gene and the other for IPT-LOG operon expression was saved as TZ3091. All transformants were confirmed by cPCR and are listed in the Table 10 below.

TABLE 10
Escherichia coli BL21(DE3) strains obtained by transformation
of DXS and IPT-LOG expression plasmids.

| | | DXS | IPT | IPT-LOG |
|---|---|---|---|---|
| | Parent | SEQ | SEQ | SEQ |
| | strain | ID 90 | ID 179 | ID 82 |
| Escherichia coli | / | / | / | / |
| BL21(DE3) | | | | |
| TZ3077 | BL21(DE3) | + | / | / |
| TZ3079 | BL21(DE3) | / | + | / |
| TZ3082 | BL21(DE3) | / | / | + |
| TZ3087 | BL21(DE3) | + | + | / |
| TZ3091 | BL21(DE3) | + | / | + |

Constructed strains (TZ3077 to TZ3091) and control strain Escherichia coli BL21(DE3) are preserved in 20% glycerol at −80° C. Strains were always cultivated in the presence of appropriate concentration of selection antibiotic (kanamycin/chloramphenicol). First, they were spread onto solid seed medium—2YT agar plates (Table 11) and incubated for approximately 17 h at 37° C. For further testing, a vegetative-stage medium—2YT was inoculated with one single colony of the culture on solid seed plates per 250-mL Erlenmeyer flask containing 50 mL of vegetative medium and appropriate amounts of antibiotics. The cultures were incubated at 37° C. for 17 h at 220 RPM. The culture in the vegetative medium (Table 12) was used as seed culture: 2.5% inoculum was used for inoculation of 50 mL of production medium in 250-mL Erlenmeyer flask. As production media (Table 13), 2YT supplemented with glucose was used (sterile glucose was added after autoclaving in the final concentration of 25 g/L). The cultures were incubated at 37° C. for 10 h at 220 RPM. After 2 h of fermentation, strains with expression plasmids were induced. Strains with IPT/IPT-LOG expression plasmid were induced with IPTG in the final concentration 150 μM. Strains with DXS expression plasmid were induced with m-toluate in the final concentration 1 mM. Strains with both expression systems (DXS and IPT/IPT-LOG) were induced with both inductors (150 μM IPTG and 1 mM m-toluate). After 10 hours of fermentation, cultures were sampled for analysis of trans-zeatin and related isoprenoid cytokinins. Extraction protocol used was as described in the Example 7. The titers of trans-zeatin, trans-zeatin riboside, isopentenyl adenine and isopentenyl adenine riboside were measured using the LC/MS as described in the Example 7.

TABLE 11
Composition of the solid seed medium: 2YT plates

| Compound | Per 1 L |
|---|---|
| Tryptone | 16 g |
| Yeast extract | 10 g |
| NaCl | 5 g |
| Agar | 15 g |
| pH 7.0 | |

TABLE 12
Composition of the vegetative medium, 2YT

| Compound | Per 1 L |
|---|---|
| Tryptone | 16 g |
| Yeast extract | 10 g |
| NaCl | 5 g |
| pH 7.0 | |

TABLE 13
Composition of the production medium, 2YT + glucose

| Compound | Per 1 L |
|---|---|
| Tryptone | 16 g |
| Yeast extract | 10 g |
| NaCl | 5 g |
| Glucose (added after autoclaving) | 25 g |
| pH 7.0 | |

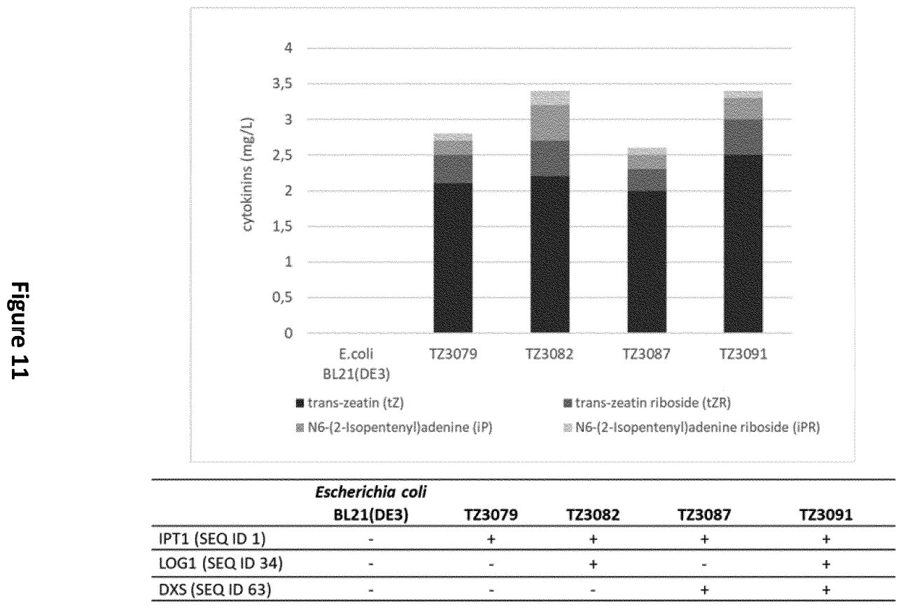

The yields of isoprenoid cytokinins detected are shown in FIG. 11. Since strain growth was different in the presence of one or two selection antibiotics and different inductors used, cytokinin measurements were normalized by dividing by the optical density of each culture at 600 nm (OD600).

The strains expressing IPT produce isoprenoid cytokinins in the amounts up to 2.8 mg/L (see FIG. 11 and Table 17). The strains expressing IPT-LOG produce isoprenoid cytokinins in the amounts up to 3.4 mg/L. The production of isoprenoid cytokinins is increased in the strains with expression of IPT-LOG and additional expression of DXS for isoprenoid precursor supply to 3.4 mg/L.

### Example 14: Assembly of IPT1-LOG 1 Operon, IPT1 and DXS Genes into Cloning Vectors, and Transformation of Plasmids into Bacterium Corynebacterium stationis

The synthetic fragments containing genes IPT1-LOG 1 (SEQ ID NO: 178) for isoprenoid cytokinin biosynthesis were amplified from previously constructed synthetic trans-zeatin operon SEQ ID NO: 82 using primer set SEQ ID NO: 173 and SEQ ID NO: 174 for fragment SEQ ID NO: 178 and for expressing gene IPT1 (SEQ ID NO: 180) primer set SEQ IN NO: 173 and SEQ ID NO: 177 were used for construction of fragment SEQ ID NO: 180. Fragments were amplified using Eppendorf cycler and Phusion polymerase (Thermo Fisher) in the buffer provided by the manufacturer and with the addition of 200 μM dNTPs, 0.5 μM of each primer, and approximately 10 ng of template in a final volume of 50 μl for 30 cycles using the PCR cycling conditions: 98° C. 30 s, 30 cycles of (98° C. 30 s, 63.3° C. 25 s, 72° C. 45 s), 72° C. 5 min, 10° C. hold.

The synthetic dxs gene (SEQ ID NO: 181) was amplified from previously constructed synthetic dxs operon SEQ ID NO: 90 using primer set SEQ ID NO: 175 and SEQ ID NO: 176 using Eppendorf cycler and Phusion polymerase (Thermo Fisher) in the buffer provided by the manufacturer and with the addition of 200 μM dNTPs, 0.5 μM of each primer, and approximately 10 ng of template in a final volume of 50 μl for 30 cycles using the PCR cycling conditions: 98° C. 30 s, 30 cycles of (98° C. 30 s, 67.6° C. 25 s, 72° C. 60 s), 72° C. 5 min, 10° C. hold. PCR reaction products of each fragment were run on 0.8% agarose gel, excised from the gel, and extracted from the gel by GeneJET Gel Extraction Kit (Thermo Fisher) according to the protocol provided by the manufacturer.

The IPT1 gene and IPT1-LOG 1 isoprenoid cytokinin operons were further cloned into the pVWEx6 plasmid (Henke et al. 2021) and dxs gene expression cassette into the pECXT99APsyn plasmid by HiFi assembly reaction using NEBuilder HiFi DNA Assembly Master Mix according to the protocol provided by the manufacturer. Both plasmids were previously linearized by digestion with BamH/FD restriction enzyme. HiFi reaction mixture was further used for electroporation of electrocompetent DH10β E. coli cells. Kanamycin selection marker enabled selection of pVWEx6+IPT1-LOG 1 and pVWEx6+IPT1 transformants while tetracycline was used for selection of pECXT99APsyn+dxs transformants. Transformants were confirmed by colony PCR. For plasmid isolation colonies were inoculated into 2TY medium with appropriate antibiotics and incubated at 37° C. overnight. Plasmids were isolated from overnight cultures by GeneJET Plasmid Miniprep Kit (Thermo Fisher) plasmid extraction kit according to the protocol provided by the manufacturer. Isolated plasmids were further analyzed by digestion with KpnI and XbaI FD restriction enzymes.

For transformation of generated plasmids into the Corynebacterium stationis DSM 20305 electrocompetent cells were prepared according to Yili et al. 2015. Transformation with isolated plasmids was performed by electroporator BioRad using 2 mm cuvettes. Approximately 250 ng of plasmid was introduced to 50 μL aliquot of previously prepared electrocompetent cells, transferred into 2 mm cuvette and exposed to electrical pulse. Cells were immediately transferred into 2 mL Eppendorf tube containing 1 mL of regeneration medium. After 3 hours of incubation at shaker set to 30° C. and 200 rpm, transformants were plated onto recovery agar plates LBHIS with selection markers in appropriate concentrations. Obtained transformants (presented in Table 14) were verified by colony PCR.

For transformants with both, pVWEx6+ipt1-log 1 and pECXT99APsyn+dxs as well as pVWEx6+ipt1 and pECXT99APsyn+dxs constructs, confirmed transformants were used for preparation of new electrocompetent cells for second generation of strains. Electrocompetent cells were transformed with additional plasmid and selected on plates with both kanamycin and tetracycline. Colony PCR verification of transformants was made for both plasmids.

TABLE 14
Corynebacterium stationis strains obtained
by transformation with pWVEx6 + IPT1-LOG1
or/and pECXT99APsyn + DXS plasmids.

| | pVWEx6 + | pVWEx6 + | pECXT99APsyn + |
|---|---|---|---|
| | IPT1-LOG1 | IPT1 | DXS |
| Corynebacterium | / | / | / |
| stationis | | | |
| DSM 20305 | | | |
| TZ3138 | / | + | / |
| TZ3139 | + | / | / |
| TZ3136 | / | / | + |
| TZ3142 | / | + | + |
| TZ3146 | + | / | + |

### Example 15: Production of Trans-Zeatin and Related Isoprenoid Cytokinins by Corynebacterium stationis Heterologous Expression

All of the constructed strains and control strain have been cultivated following this procedure. The frozen stocks of the strains Corynebacterium stationis DSM 20305, TZ3136, TZ3138, TZ3139, TZ3142 and TZ3146, preserved in 20% glycerol and stored at −80° C., were spread onto solid seed medium containing tetracycline or/and kanamycin in the appropriate concentration and incubated for approximately 1 day at 30° C. For further testing, a vegetative-stage medium was inoculated with 5 plugs of the culture on solid seed plates per baffled 250-mL Erlenmeyer flask containing 25 mL of vegetative medium and appropriate amounts of antibiotics. The cultures were incubated at 30° C. for 18 h at 200 RPM. The culture in the vegetative medium was used as seed culture, and it was inoculated into the production medium. A 10-% inoculum was used (2.5 mL per 25 mL of production medium in baffled 250-mL Erlenmeyer flask). The cultures were incubated at 30° C. for up to 48 h at 200 RPM. The fermented cultures were sampled and analyzed as described in the Example 7. Titres of trans-zeatin, trans-zeatin riboside, isopentenyl adenine and isopentenyl adenine riboside were measured using the LC/MS as described in the Example 7.

TABLE 15
Composition of the solid seed medium
for Corynebacterium stationis

| Compound | Per 1 L |
|---|---|
| Tryptone | 10 g |
| NaCl | 5 g |
| Yeast extract | 5 g |
| Agar | 20 g |

TABLE 16
Composition of the production medium
for Corynebacterium stationis

| Compound | Per 1 L |
|---|---|
| Tryptone | 10 g |
| NaCl | 5 g |
| Yeast extract | 5 g |
| Glucose | 20 g |

### Example 16: Analysis of Isoprenoid Cytokinins by Corynebacterium stationis Heterologous Expression

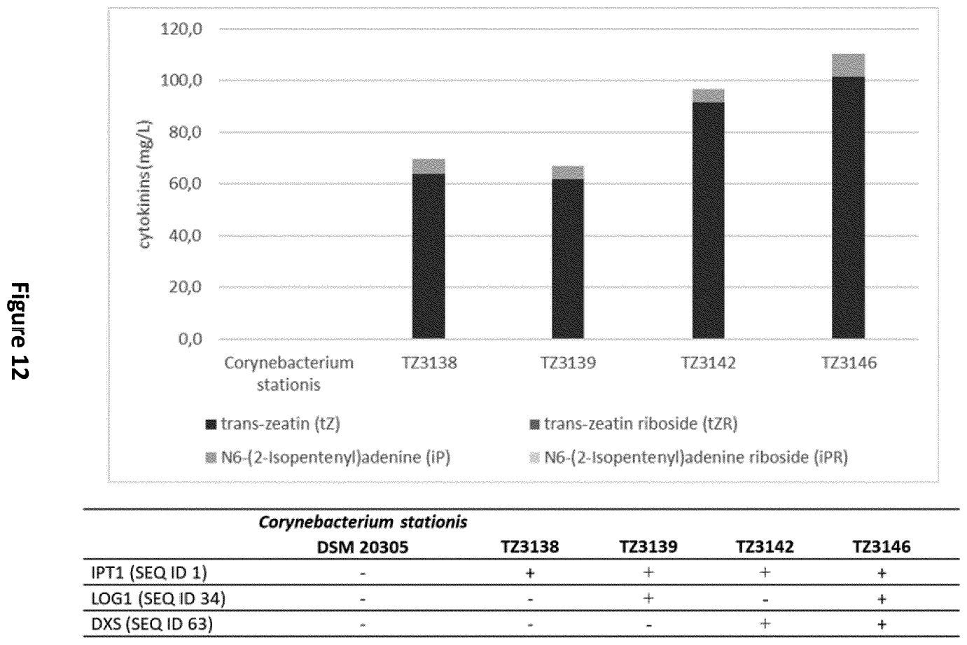

Isoprenoid cytokinin production of Corynebacterium stationis transformants was tested in fermentation process. The cultivation was performed as described in the Example 15. The extraction and analysis were performed according to procedure described in the Example 7. The results are shown in FIG. 12 and Table 17.

TABLE 17
Comparison of total cytokinins and trans-zeatin produced in strains of E. coli BL21 (DE3),
Bacillus subtilis 168 and Corynebacterium stationis DSM 20305 with genetic modification IPT1,
IPT1 + DXS, IPT1-LOG1 and IPT1-LOG1 + DXS in the early stationary phase of growth.

| | Products | No | | IPT1 + | IPT1- | IPT1-LOG1 + |
|---|---|---|---|---|---|---|
| Strain | (mg/L) | modification | IPT1 | DXS | LOG1 | DXS |
| E. coli BL21 (DE3) | total | Not detected | 2.8 | 2.6 | 3.4 | 3.4 |
| | cytokinins | | | | | |
| | trans-zeatin | Not detected | 2.1 | 2.0 | 2.2 | 2.5 |
| Bacillus subtilis 168 | total | Not detected | 48.2 | 70.8 | 50.7 | 60.9 |
| | cytokinins | | | | | |
| | trans-zeatin | Not detected | 15.7 | 23.6 | 12.5 | 17.4 |
| Corynebacterium | total | Not detected | 69.7 | 96.7 | 66.8 | 110.4 |
| stationis | cytokinins | | | | | |
| DSM 20305 | trans-zeatin | Not detected | 63.8 | 91.4 | 61.7 | 101.3 |

### Example 17: Enhancement of Purine Nucleotide Biosynthesis Pathway in Bacillus subtilis by Overexpression of the purA Gene

The synthetic fragment containing purA gene (SEQ ID NO: 98) used for enhancement of purine nucleotide biosynthesis pathway was assembled in the artificial gene expression cassette. The initial (SEQ ID NO: 96) and end fragment (SEQ ID NO: 97) containing gene integration homology, the promoter sequence, and the zeocin selectable marker were designed and synthesized for stable genome integration into the yybN locus in the genome of B. subtilis.

The first step of the artificial operon assembly was PCR amplification of separate DNA fragments performed using primer pair SEQ ID NO: 99 and SEQ ID NO: 100 for the initial fragment SEQ ID NO: 96, and primer set SEQ ID NO: 101 and SEQ ID NO: 102 for end fragment SEQ ID NO: 97. The primer set SEQ ID NO: 103 and SEQ ID NO: 104 was used for amplification of fragment containing genes for purA overexpression SEQ ID NO: 98. The fragments were amplified using Eppendorf cycler and Phusion polymerase (Thermo Fisher) in the buffer provided by the manufacturer and with the addition of 200 μM dNTPs, 0.5 μM of each primer, and approximately 10 ng of template in a final volume of 50 μl for 30 cycles using the PCR cycling conditions: 98° C. 30 s, 30 cycles of (98° C. 30 s, 65° C. 25 s, 72° C. 23/25 s), 72° C. 5 min, 10° C. hold. PCR reaction products of each fragment were run on 0.8% agarose gel, excised from the gel, and extracted from the gel by GeneJET Gel Extraction Kit (Thermo Fisher) according to the protocol provided by the manufacturer. The fragments were assembled into an artificial operon by repetitive steps of restriction and ligation. A combination of SpeI (BcuI) and XbaI restriction sites was used to provide compatible restriction ends for a successful ligation. After each step of ligation, the combined fragments were used as a new template for the next PCR amplification. The restriction was done in 50 μl volume with the addition of 5 μl FD green buffer (Thermo Fisher Scientific), 2-3 μl of the selected enzyme (SpeI (BcuI), and XbaI, Thermo Fisher), and up to app. 1500 ng of PCR fragment. After restriction digest, the digested DNA fragments were cleaned with Wizard SV Gel and PCR Clean-up system according to the protocol provided by the manufacturer. The first two fragments were used in the ligation reaction with 2.5 U of T4 DNA ligase (Thermo Fisher) in the buffer provided by the manufacturer and the addition of 5% PEG 4000 and both fragments in a 1:1 molar ratio to the final volume of 15 μl. In the next step, 1 μl of inactivated ligation was used as a template in a new 50 μL PCR with a primer set SEQ ID NO: 102 and SEQ ID NO: 105, and the same PCR mix and PCR cycling conditions as previously but with longer elongation time. The restriction digest, cleaning, and ligation steps were repeated for ligation of the end fragment. PCR was run on 0.8% agarose gel, the fragment was excised from the gel, digested and cleaned as before, and ligated as before. The final operon containing the yybN homology, promoter with RBS sequence, purA genes, and zeocin resistance cassette was amplified using the primer pair SEQ ID NO: 106 and SEQ ID NO: 107, cleaned and ligated as described before. The constructed synthetic operon was used for the transformation of Bacillus subtilis VKPM B2116. The transformation with purA operon SEQ ID NO: 108 resulted in the transformed strain BS19. The correct integration of the artificial operons at yybN integration site was confirmed by cPCR.

### Example 18: Assembly of Synthetic Isoprenoid Cytokinin Operons Containing Various Homologues of the IPT Gene in Combination with the LOG 8 Gene, and Transformation into B. subtilis

The synthetic fragments containing synthetic LOG gene (LOG 8—SEQ ID NO: 116) and various IPT genes (SEQ ID NOs: 129, 154-156) for isoprenoid cytokinin biosynthesis were assembled in artificial isoprenoid cytokinin operons. The initial and end fragments containing gene integration homology, the promoter sequence, and the erythromycin selectable marker (SEQ ID NO: 73) were designed and synthesized for stable genome integration into the amyE locus in the genome of B. subtilis.

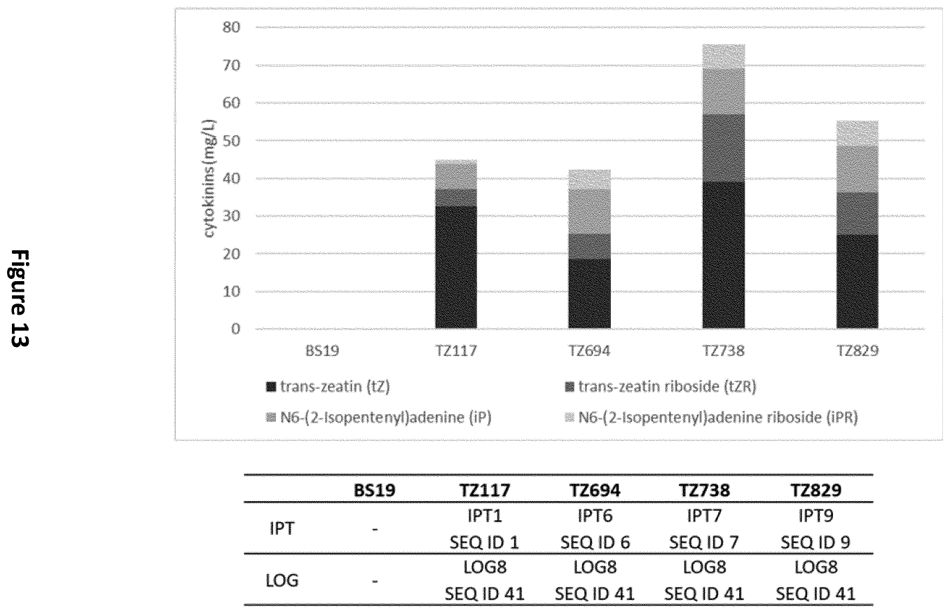

The first step of the artificial operon assembly was PCR amplification of separate DNA fragments performed using primer pair SEQ ID NO: 74 and SEQ ID: 75 for the initial fragment SEQ ID NO: 76, and for synthetic fragment LOG 8 (SEQ ID NO: 116) and fragments IPT1, IPT6, IPT7 and IPT9 (SEQ ID NOs: 179, 151, 152 and 153, respectively) containing genes for isoprenoid-cytokinin biosynthesis. The primer set SEQ ID NO: 77 and SEQ ID NO: 78 was used for amplification of the end fragment SEQ ID NO: 79. The fragments were amplified using Eppendorf cycler and Phusion polymerase (Thermo Fisher) in the buffer provided by the manufacturer and with the addition of 200 μM dNTPs, 0.5 μM of each primer, and approximately 10 ng of template in a final volume of 50 μl for 30 cycles using the PCR cycling conditions: 98° C. 30 s, 30 cycles of (98° C. 30 s, 65° C. 25 s, 72° C. 30 s), 72° C. 5 min, 10° C. hold. PCR reaction products of each fragment were run on 0.8% agarose gel, excised from the gel, and extracted from the gel by GeneJET Gel Extraction Kit (Thermo Fisher) according to the protocol provided by the manufacturer. The fragments were assembled into an artificial operon by repetitive steps of restriction and ligation. A combination of SpeI (BcuI), VspI, NdeI and SnaBI restriction sites was used to provide compatible restriction ends for a successful ligation. After each step of ligation, the combined fragments were used as a new template for the next PCR amplification. The restriction was done in 50 μl volume with the addition of 5 μl FD green buffer (Thermo Fisher Scientific), 2-3 μl of the selected enzyme (SpeI (BcuI), VspI, NdeI and SnaBI, Thermo Fisher), and up to app. 1500 ng of PCR fragment. After restriction digest, the digested DNA fragments were cleaned with Wizard SV Gel and PCR Clean-up system according to the protocol provided by the manufacturer. Initial fragment and various IPT fragments (SEQ ID NO: 179, 151, 152 and 153) were used in ligation reactions with 2.5 U of T4 DNA ligase (Thermo Fisher) in the buffer provided by the manufacturer and the addition of 5% PEG 4000 and both fragments in a 1:1 molar ratio to the final volume of 15 μl. In the next step, 1 μl of each inactivated ligation was used as a template in a new 50 μL PCR with a primer set SEQ ID NO: 120 and SEQ ID NO: 121, and the same PCR mix and PCR cycling conditions as previously but with longer elongation time. DNA fragments were extracted from agarose gel as described before. The restriction digest (VspI and NdeI), cleaning, and ligation steps were repeated for ligation of AmyE+IPT1 operon, AmyE+IPT6 operon, AmyE+IPT7 operon and AmyE+IPT9 operon (SEQ ID NO: 129, 154, 155 and 156, respectively) and fragment containing LOG 8 gene (SEQ ID NO: 116). 1 μl of inactivated ligation was further used as a template in a new 50 μL PCR with a primer set SEQ ID NO: 122 and SEQ ID NO: 123, and the same PCR mix and PCR cycling conditions as previously but with longer elongation time. DNA fragments were extracted from agarose gel as described before. XbaI enzymatic digestion was performed to ligate operons AmyE 0+IPT1/6/7/9+LOG 8 (SEQ ID NO: 136, 157, 158 and 159, respectively) and operon EryR+AmyE END (SEQ ID NO: 140), previously ligated by SnaBI restriction site and PCR amplified using primer set SEQ ID NO: 126 and SEQ ID NO: 127. The final operons containing the amy E homology, promoter with RBS sequence, various IPT genes and gene LOG 8, and erythromycin resistance cassette were amplified using the primer pair SEQ ID NO: 128 and SEQ ID NO: 123, cleaned and ligated as described before. The constructed synthetic trans-zeatin operons containing IPT1-LOG 8, IPT6-LOG 8, IPT7-LOG 8 and IPT9-LOG 8 (SEQ ID NOs: 147, 160, 161 and 162, respectively) were used for the transformation of Bacillus subtilis BS19 (described in Example 17). All transformants were confirmed by cPCR and are listed in the Table 18 below. All of the constructed strains have been cultivated as described in the Example 6. The extraction and analysis were performed as described in the Example 7 and the yields of cytokinins are shown in FIG. 13.

TABLE 18
Bacillus subtilis strains obtained
by transformation of IPT-LOG8 operons.

| Strain | IPT-LOG operon | Expressed IPT |
|---|---|---|
| TZ117 | IPT1-LOG8 (SEQ ID NO: 147) | IPT1 (SEQ ID NO: 1 |
| TZ694 | IPT6-LOG8 (SEQ ID NO: 160) | IPT6 (SEQ ID NO: 6) |
| TZ738 | IPT7-LOG8 (SEQ ID NO: 161) | IPT7 (SEQ ID NO: 7) |
| TZ829 | IPT9-LOG8 (SEQ ID NO: 162) | IPT9 (SEQ ID NO: 9) |

### Example 19: Assembly of Synthetic Isoprenoid Cytokinin Operons Containing IPT1 and Various LOGs, and Transformation into B. subtilis

The synthetic fragments containing synthetic gene IPT1 (SEQ ID NO: 71) and various LOG genes (SEQ ID NO: 110-119) for isoprenoid cytokinin biosynthesis were assembled in artificial isoprenoid cytokinin operons. The initial and end fragments containing gene integration homology, the promoter sequence, and the erythromycin selectable marker were designed and synthesized for stable genome integration into the amyE locus in the genome of B. subtilis.

The first step of the artificial operon assembly was PCR amplification of separate DNA fragments performed using primer pair SEQ ID NO: 74 and SEQ ID NO: 75 for the initial fragment SEQ ID NO: 76, and for synthetic fragment IPT1 SEQ ID NO: 71 and fragments LOG 2-LOG 11 (SEQ ID NO: 110-119) containing genes for isoprenoid-cytokinin biosynthesis. The primer set SEQ ID NO: 126 and SEQ ID NO: 127 was used for amplification of the end fragment containing erythromycin selectable marker (SEQ ID NO: 140). The fragments were amplified using Eppendorf cycler and Phusion polymerase (Thermo Fisher) in the buffer provided by the manufacturer and with the addition of 200 μM dNTPs, 0.5 μM of each primer, and approximately 10 ng of template in a final volume of 50 μl for 30 cycles using the PCR cycling conditions: 98° C. 30 s, 30 cycles of (98° C. 30 s, 65° C. 25 s, 72° C. 30 s), 72° C. 5 min, 10° C. hold. PCR reaction products of each fragment were run on 0.8% agarose gel, excised from the gel, and extracted from the gel by GeneJET Gel Extraction Kit (Thermo Fisher) according to the protocol provided by the manufacturer. The fragments were assembled into an artificial operon by repetitive steps of restriction and ligation. A combination of SpeI (BcuI), VspI, NdeI XbaI and SnaBI restriction sites was used to provide compatible restriction ends for a successful ligation. After each step of ligation, the combined fragments were used as a new template for the next PCR amplification. The restriction was done in 50 μl volume with the addition of 5 μl FD green buffer (Thermo Fisher Scientific), 2-3 μl of the selected enzyme (SpeI (BcuI), VspI, NdeI, XbaI and SnaBI, Thermo Fisher), and up to app. 1500 ng of PCR fragment. After restriction digest, the digested DNA fragments were cleaned with Wizard SV Gel and PCR Clean-up system according to the protocol provided by the manufacturer.

The first two fragments were used in the ligation reaction with 2.5 U of T4 DNA ligase (Thermo Fisher) in the buffer provided by the manufacturer and the addition of 5% PEG 4000 and both fragments in a 1:1 molar ratio to the final volume of 15 μl. In the next step, 1 μl of inactivated ligation was used as a template in a new 50 μL PCR with a primer set SEQ ID NO: 120 and SEQ ID NO: 121, and the same PCR mix and PCR cycling conditions as previously but with longer elongation time. DNA fragments were extracted from agarose gel as described before. The restriction digest (VspI and NdeI), cleaning, and ligation steps were repeated for ligation of fragments containing various LOG genes (SEQ ID NO: 110-119). 1 μl of inactivated ligation was further used as a template in a new 50 μL PCR with a primer set SEQ ID NO: 122 and SEQ ID NO: 123, and the same PCR mix and PCR cycling conditions as previously but with longer elongation time. DNA fragments were extracted from agarose gel as described before. XbaI enzymatic digestion was performed to ligate operons AmyE 0+IPT1+LOG 2-11 (SEQ ID NO: 130-139) and operon EryR+AmyE END (SEQ ID NO: 140), previously ligated by SnaBI restriction site and PCR amplified using primer set SEQ ID NO: 126 and SEQ ID NO: 127.

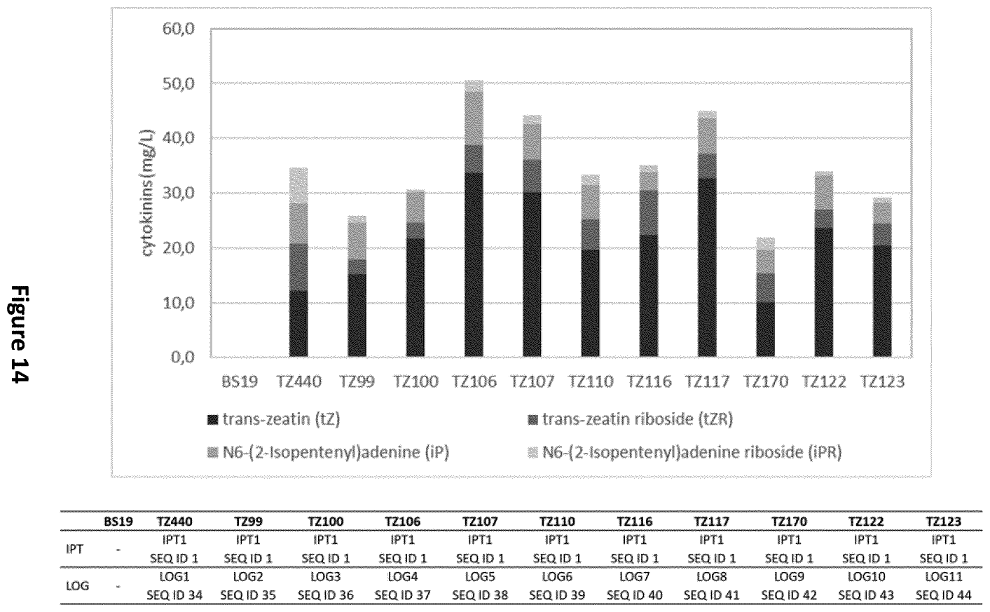

The final operons containing the amyE homology, promoter with RBS sequence, IPT1 and various LOG genes, and erythromycin resistance cassette were amplified using the primer pair SEQ ID NO: 128 and SEQ ID NO: 123, cleaned and ligated as described before. The constructed synthetic trans-zeatin operons containing IPT1-LOG 2-11 (SEQ ID NO: 141-150) were used for the transformation of Bacillus subtilis BS19 (described in Example 17). All transformants were confirmed by cPCR and are listed in the Table 19 below. All of the constructed strains have been cultivated as described in the Example 6. The extraction and analysis were performed as described in the Example 7 and the yields of cytokinins are shown in FIG. 14.

TABLE 19
Bacillus subtilis strains obtained
by transformation of IPT-LOG operons.

| Strain | IPT-LOG operon | Expressed LOG |
|---|---|---|
| TZ440 | IPT1-LOG1 (SEQ ID NO: 82) | LOG1 (SEQ ID NO: 34) |
| TZ99 | IPT1-LOG2 (SEQ ID NO: 141) | LOG2 (SEQ ID NO: 35) |
| TZ100 | IPT1-LOG3 (SEQ ID NO: 142) | LOG3 (SEQ ID NO: 36) |
| TZ106 | IPT1-LOG4 (SEQ ID NO: 143) | LOG4 (SEQ ID NO: 37) |
| TZ107 | IPT1-LOG5 (SEQ ID NO: 144) | LOG5 (SEQ ID NO: 38) |
| TZ110 | IPT1-LOG6 SEQ ID NO: 145) | LOG6 (SEQ ID NO: 39) |
| TZ116 | IPT1-LOG7 SEQ ID NO: 146) | LOG7 (SEQ ID NO: 40) |
| TZ117 | IPT1-LOG8 SEQ ID NO: 147) | LOG8 (SEQ ID NO: 41) |
| TZ170 | IPT1-LOG9 SEQ ID NO: 148) | LOG9 (SEQ ID NO: 42) |
| TZ122 | IPT1-LOG10 SEQ ID NO: 149) | LOG10 (SEQ ID NO: 43) |
| TZ123 | IPT1-LOG11 SEQ ID NO: 150) | LOG11 (SEQ ID NO: 44) |

### LIST OF REFERENCES CITED IN THE DESCRIPTION

Akiyoshi, D. E., D. A. Regier, G. Jen, and M. P. Gordon. 1985. “Cloning and Nucleotide Sequence of the Tzs Gene from Agrobacterium Tumefaciens Strain T37.” Nucleic Acids Research 13 (8): 2773-88. https://doi.org/10.1093/nar/13.8.2773.
Akiyoshi, Donna E., Dean A. Regier, and Milton P. Gordon. 1987. “Cytokinin Production by Agrobacterium and Pseudomonas Spp.” Journal of Bacteriology 169 (9): 4242-48. https://doi.org/10.1128/jb.169.9.4242-4248.1987.
Arkhipova, T. N., S. U. Veselov, A. I. Melentiev, E. V. Martynenko, and G. R. Kudoyarova. 2005. “Ability of Bacterium Bacillus Subtilis to Produce Cytokinins and to Influence the Growth and Endogenous Hormone Content of Lettuce Plants.” Plant and Soil 272 (1-2): 201-9. https://doi.org/10.1007/s11104-004-5047-x.
Asahara, T. et al. (2010) ‘Accumulation of gene-targeted Bacillus subtilis mutations that enhance fermentative inosine production’, Applied Microbiology and Biotechnology. Springer-Verlag, 87(6), pp. 2195-2207. doi: 10.1007/s00253-010-2646-8.
Banerjee, A. et al. (2013) ‘Feedback inhibition of deoxy-D-xylulose-5-phosphate synthase regulates the methylerythritol 4-phosphate pathway’, Journal of Biological Chemistry. doi: 10.1074/jbc.M113.464636.
Chen, S. et al. (1997) ‘Mechanism of the synergistic end-product regulation of Bacillus subtilis glutamine phosphoribosylpyrophosphate amidotransferase by nucleotides’, Biochemistry, 36(35), pp. 10718-10726. doi: 10.1021/bi9711893.
Christiansen, L. C., Schou, S. and Nygaard, P. E. R. (1997) ‘Xanthine Metabolism in Bacillus subtilis Characterization of the xpt-pbuX Operon and Evidence for Purine- and Nitrogen-Controlled Expression of Genes Involved in Xanthine Salvage and Catabolism’, 179(8), pp. 2540-2550.
Datsenko K A, Wanner B L: One-step inactivation of chromosomal genes in Escherichia coli K-12 using PCR products. Proc Natl Acad Sci USA 2000, 97:6640-6645.
Frebort, I., M. Kowalska, T. Hluska, J. Frebortova, P. Galuszka, Marta Kowalska1 Ivo Fre\acute{ } bort1,*, Toma\ddot{ } s\hat{ } Hluska1, et al. 2011. “Evolution of Cytokinin Biosynthesis and Degradation.” Journal of Experimental Botany 62 (8): 2431-52. https://doi.org/10.1093/jxb/err004.
Frebortová, Jitka, Marta Greplova, Michael F. Seidl, Alexander Heyl, and Ivo Frebort. 2015. “Biochemical Characterization of Putative Adenylate Dimethylallyltransferase and Cytokinin Dehydrogenase from Nostoc Sp. PCC 7120.” PLoS ONE 10 (9). https://doi.org/10.1371/journal.pone.0138468.
Henke, Nadja A., Irene Krahn, and Volker F. Wendisch (2021). “Improved Plasmid-Based Inducible and Constitutive Gene Expression in Corynebacterium glutamicum”. Microorganisms, 9(1), p. 204. https://doi.org/10.3390/microorganisms9010204
Julsing, Mattijs K., Michael Rijpkema, Herman J. Woerdenbag, Wim J. Quax, and Oliver Kayser. 2007. “Functional Analysis of Genes Involved in the Biosynthesis of Isoprene in Bacillus Subtilis.” Applied Microbiology and Biotechnology 75 (6): 1377-84. https://doi.org/10.1007/s00253-007-0953-5.
Kakimoto, T. 2001. “Identification of Plant Cytokinin Biosynthetic Enzymes as Dimethylallyl Diphosphate:ATP/ADP Isopentenyltransferases.” Plant and Cell Physiology. https://doi.org/10.1093/pcp/pce112.
Kamada-Nobusada, Tomoe, and Hitoshi Sakakibara. 2009. “Molecular Basis for Cytokinin Biosynthesis.” Phytochemistry 70 (4): 444-49. https://doi.org/10.1016/j.phytochem.2009.02.007.
Konishi, S. and Shiro, T. (1968) ‘Fermentative Production of Guanosine by 8-Azaguanine Resistant of Bacillus subtilis’, Agricultural and Biological Chemistry, 32(3), pp. 396-398. doi: 10.1080/00021369.1968.10859067.
Kurakawa, Takashi, Nanae Ueda, Masahiko Maekawa, Kaoru Kobayashi, Mikiko Kojima, Yasuo Nagato, Hitoshi Sakakibara, and Junko Kyozuka. 2007. “Direct Control of Shoot Meristem Activity by a Cytokinin-Activating Enzyme.” Nature 445 (7128): 652-55. https://doi.org/10.1038/nature05504.
Kuzuyama, T. et al. (2000) ‘Cloning and characterization of 1-deoxy-D-xylulose 5-phosphate synthase from Streptomyces sp. Strain CL190, which uses both the mevalonate and nonmevalonate pathways for isopentenyl diphosphate biosynthesis.’, Journal of bacteriology. American Society for Microbiology (ASM), 182(4), pp. 891-7. doi: 10.1128/jb.182.4.891-897.2000.
Kwon D H, Pena J A, Osato M S, Fox J G, Graham D Y, Versalovic J: Frameshift mutations in rdxA and metronidazole resistance in North American Helicobacter pylori isolates. J Antimicrob Chemother 2000, 46(5): 793-796.
Li, Biao, Zhi-Ying Ying Yan, Xiao-Na Na Liu, Jun Zhou, Xia-Yuan Yuan Wu, Ping Wei, Hong-Hua Hua Jia, and Xiao-Yu Yu Yong. 2019. “Increased Fermentative Adenosine Production by Gene-Targeted Bacillus subtilis Mutation.” Journal of Biotechnology 298 (June): 1-4. https://doi.org/10.1016/j.jbiotec.2019.04.007.
Mok, Machteld C., Ruth C. Martin, and David W. S. Mok. 2000. “Cytokinins: Biosynthesis Metabolism and Perception.” In Vitro Cellular & Developmental Biology—Plant 36 (2): 102-7. https://doi.org/10.1007/s11627-000-0021-7.
Nishii K, Wright F, Chen Y-Y, Müller M (2018) Tangled history of a multigene family: The evolution of ISOPENTENYLTRANSFERASE genes. PLoS ONE 13(8): e0201198. https://doi.org/10.1371/journal.pone.0201198
Patel, Pooja P, Purvi M Rakhashiya, Kiran S Chudasama, and Vrinda S Thaker. 2012. “Isolation, Purification and Estimation of Zeatin from Corynebacterium aurimucosum.” European Journal of Experimental Biology 2 (1): 1-8.
Peifer, S. et al. (2012) ‘Metabolic engineering of the purine biosynthetic pathway in Corynebacterium glutamicum results in increased intracellular pool sizes of IMP and hypoxanthine’, Microbial Cell Factories. BioMed Central, 11(1), p. 138. doi: 10.1186/1475-2859-11-138.
Powell, G. K., and R. O. Morris. 1986. “Nucleotide Sequence and Expression of a Pseudomonas savastanoi Cytokinin Biosynthetic Gene: Homology with Agrobacterium tumefaciens Tmr and Tzs Loci.” Nucleic Acids Research 14 (6): 2555-65. https://doi.org/10.1093/nar/14.6.2555.
Qui Z and Goodman M F: The Escherichia coli polB locus is identical to dinA, the structural gene for DNA polymerase II. Characterization of Pol II purified from a polB mutant. J Biol Chem. 1997, 272(13): 8611-8617.
Regier, Dean A., and Roy O. Morris. 1982. “Secretion of Trans-Zeatin by Agrobacterium tumefaciens: A Function Determined by the Nopaline Ti Plasmid.” Biochemical and Biophysical Research Communications 104 (4): 1560-66. https://doi.org/10.1016/0006-291X(82)91429-2.
Sakakibara, Hitoshi. 2005. “Cytokinin Biosynthesis and Regulation.” Vitamins and Hormones. Vitam Horm. https://doi.org/10.1016/S0083-6729(05)72008-2.
Sakakibara, Hitoshi. 2006. “Cytokinins: Activity, Biosynthesis, and Translocation.” Annual Review of Plant Biology 57 (1): 431-49. https://doi.org/10.1146/annurev.arplant.57.032905.105231.
Sakakibara, Hitoshi, Hiroyuki Kasahara, Nanae Ueda, Mikiko Kojima, Kentaro Takei, Shojiro Hishiyama, Tadao Asami, et al. 2005. “Agrobacterium tumefaciens Increases Cytokinin Production in Plastids by Modifying the Biosynthetic Pathway in the Host Plant.” Proceedings of the National Academy of Sciences of the United States of America 102 (28): 9972-77. https://doi.org/10.1073/pnas.0500793102.
Scarbrough, E., D. J. Armstrong, and F. Skoog. 1973. “Isolation of Cis Zeatin from Corynebacterium fascians Cultures.” Proceedings of the National Academy of Sciences of the United States of America 70 (12 II): 3825-29. https://doi.org/10.1073/pnas.70.12.3825.
Schäfer, Martin, Christoph Brütting, Ivan David Meza-Canales, Dominik K. Großkinsky, Radomira Vankova, Ian T. Baldwin, and Stefan Meldau. 2015. “The Role of Cis-Zeatin-Type Cytokinins in Plant Growth Regulation and Mediating Responses to Environmental Interactions.” Journal of Experimental Botany 66 (16): 4873-84. https://doi.org/10.1093/jxb/erv214.
Seo, Hogyun, and Kyung-Jin Jn Kim. 2017. “Structural Basis for a Novel Type of Cytokinin-Activating Protein.” Scientific Reports 7 (1): 45985. https://doi.org/10.1038/srep45985.
Seo, Hogyun, Sangwoo Kim, Hye-Young Sagong, Hyeoncheol Francis Son, Kyeong Sik Jin, II-Kwon Kim, and Kyung-Jin Kim. 2016. “Structural Basis for Cytokinin Production by LOG from Corynebacterium glutamicum” 6 (1): 31390. https://doi.org/10.1038/srep31390.
Shi, S. et al. (2009) ‘Transcriptome analysis guided metabolic engineering of Bacillus subtilis for riboflavin production’, Metabolic Engineering. Academic Press, 11(4-5), pp. 243-252. doi: 10.1016/j.ymben.2009.05.002.
Shi, T. et al. (2014) ‘Deregulation of purine pathway in Bacillus subtilis and its use in riboflavin biosynthesis’, Microbial Cell Factories, 13(1), pp. 1-16. doi: 10.1186/s12934-014-0101-8.
Stirk, Wendy A., and J. van Staden. 2010. “Flow of Cytokinins through the Environment.” Plant Growth Regulation 62 (November 2010): 101-16. https://doi.org/10.1007/s10725-010-9481-x.
Streletskii, Rostislav A, Aleksey V Kachalkin, Anna M Glushakova, Andrey M Yurkov, and Vladimir V Demin. 2019. “Yeasts Producing Zeatin.” PeerJ 7: e6474. https://doi.org/10.7717/peerj.6474.
Sugawara, Hajime, Nanae Ueda, Mikiko Kojima, Nobue Makita, Tomoyuki Yamaya, and Hitoshi Sakakibara. 2008. “Structural Insight into the Reaction Mechanism and Evolution of Cytokinin Biosynthesis.” Proceedings of the National Academy of Sciences 105 (7): 2734-39. https://doi.org/10.1073/pnas.0707374105.
Takei, Kentaro, Hitoshi Sakakibara, and Tatsuo Sugiyama. 2001. “Identification of Genes Encoding Adenylate Isopentenyltransferase, a Cytokinin Biosynthesis Enzyme, in Arabidopsis thaliana.” Journal of Biological Chemistry 276 (28): 26405-10. https://doi.org/10.1074/jbc.M102130200.
Takei, Kentaro, Tomoyuki Yamaya, and Hitoshi Sakakibara. 2004. “Arabidopsis CYP735A1 and CYP735A2 Encode Cytokinin Hydroxylases That Catalyse the Biosynthesis of Trans-Zeatin.” Journal of Biological Chemistry 279 (40): 41866-72. https://doi.org/10.1074/jbc.M406337200.
Wang, X. et al. (2016) ‘Directed evolution of adenylosuccinate synthetase from Bacillus subtilis and its application in metabolic engineering’, Journal of Biotechnology. Elsevier B.V., 231 (May), pp. 115-121. doi: 10.1016/j.jbiotec.2016.05.032.
Wang, Z. et al. (2011) ‘Enhancement of riboflavin production with Bacillus subtilis by expression and site-directed mutagenesis of zwf and gnd gene from Corynebacterium glutamicum.’, Bioresource technology, 102(4), pp. 3934-40. doi: 10.1016/j.biortech.2010.11.120.
Xiang, S. et al. (2007) ‘Crystal Structure of 1-Deoxy-d-xylulose 5-Phosphate Synthase, a Crucial Enzyme for Isoprenoids Biosynthesis’, Journal of Biological Chemistry, 282(4), pp. 2676-2682. doi: 10.1074/jbc.M610235200.
Xiang, S. et al. (2012) ‘1-Deoxy-d-Xylulose 5-Phosphate Synthase (DXS), a Crucial Enzyme for Isoprenoids Biosynthesis’, in Isoprenoid Synthesis in Plants and Microorganisms. New York, NY: Springer New York, pp. 17-28. doi: 10.1007/978-1-4614-4063-5_2.
Xue, D. et al. (2015) ‘Enhanced C30 carotenoid production in Bacillus subtilis by systematic overexpression of MEP pathwaygenes’, Applied Microbiology and Biotechnology, 99(14), pp. 5907-5915. doi: 10.1007/s00253-015-6531-3.
Yang, S. et al. (2019) ‘Modular Pathway Engineering of Bacillus subtilis to promote de novo biosynthesis of menaquinone-7’, ACS Synthetic Biology. American Chemical Society, 8(1), pp. 70-81. doi: 10.1021/acssynbio.8b00258.
Yili Ruan, Linjiang Zhu, and Qi Li. (2015) “Improving the electro-transformation efficiency of Corynebacterium glutamicum by weakening its cell wall and increasing the cytoplasmic membrane fluidity.” Biotechnology Letters, 37, pp. 2445-2452 doi: 10.1007/s10529-015-1934-x
Zakataeva, N. P. et al. (2012) ‘Wild-type and feedback-resistant phosphoribosyl pyrophosphate synthetases from Bacillus amyloliquefaciens: Purification, characterization, and application to increase purine nucleoside production’, Applied Microbiology and Biotechnology, 93(5), pp. 2023-2033. doi: 10.1007/s00253-011-3687-3.
Zeigler, Daniel R., Zoltan Pragai, Sabrina Rodriguez, Bastien Chevreux, Andrea Muffler, Thomas Albert, Renyuan Bai, Markus Wyss, and John B. Perkins. 2008. “The Origins of 168, W23, and Other Bacillus Subtilis Legacy Strains.” Journal of Bacteriology 190 (21): 6983-95. https://doi.org/10.1128/JB.00722-08.

1. A Gram-positive bacterium, which expresses a heterologous polypeptide, wherein the heterologous polypeptide has adenylate isopentenyltransferase activity.

2-33. (canceled)

34. The Gram-positive bacterium according to claim 1, wherein the bacterium is of the family Bacillaceae or Corynebacteriaceae.

35. The Gram-positive bacterium according to claim 1, wherein the bacterium is of the genus Bacillus or Corynebacterium.

36. The Gram-positive bacterium according to claim 1, wherein the bacterium is Bacillus subtilis or Corynebacterium stationis.

37. The Gram-positive bacterium according to claim 1, wherein the heterologous polypeptide having adenylate isopentenyltransferase activity is selected from the group consisting of:
i) a polypeptide comprising an amino acid sequence of any one of SEQ ID NOs: 1 to 33; and
ii) a polypeptide comprising an amino acid sequence, which has at least 75% sequence identity to the amino acid sequence of any one of SEQ ID NOs: 1 to 33.

38. The Gram-positive bacterium according to claim 1, wherein the bacterium has been modified to have an increased protein expression of a polypeptide having cytokinin riboside 5′-monophosphate phosphoribohydrolase activity compared to an otherwise identical bacterium that does not carry said modification.

39. The Gram-positive bacterium according to claim 38, wherein the polypeptide having cytokinin riboside 5′-monophosphate phosphoribohydrolase activity is selected from the group consisting of:
i) a polypeptide comprising an amino acid sequence of any one of SEQ ID NOs: 34 to 62 and
ii) a polypeptide comprising an amino acid sequence, which has at least 75% sequence identity to the amino acid sequence of any one of SEQ ID NOs: 34 to 62.

40. The Gram-positive bacterium according to claim 1, wherein the bacterium has been modified to have an increased protein expression of a polypeptide having 1-deoxy-D-xylulose-5-phosphate synthase activity compared to an otherwise identical bacterium that does not carry said modification.

41. The Gram-positive bacterium according to claim 40, wherein the polypeptide having 1-deoxy-D-xylulose-5-phosphate synthase activity is selected from the group consisting of:
i) a polypeptide comprising an amino acid sequence of any one of SEQ ID NOs: 63 to 70; and
ii) a polypeptide comprising an amino acid sequence, which has at least 75% sequence identity to the amino acid sequence of any one of SEQ ID NOs: 63 to 70.

42. The Gram-positive bacterium according to claim 1, wherein the bacterium has been modified to have an increased expression and/or activity of at least one enzyme involved in the purine nucleotide biosynthesis pathway compared to an otherwise identical bacterium that does not carry said modification.

43. The Gram-positive bacterium according to claim 42, wherein the at least one enzyme involved in the purine nucleotide biosynthesis pathway is selected from the group consisting of: an enzyme having ribose-phosphate diphosphokinase activity, an enzyme having amidophosphoribosyltransferase activity, an enzyme having formyltetrahydrofolate deformylase activity, an enzyme having adenylosuccinate lyase activity, an enzyme having phosphoribosylaminoimidazole-carboxamide formyltransferase activity, an enzyme having adenylosuccinate synthase activity and an enzyme having adenosine kinase activity.

44. The Gram-positive bacterium according to claim 1, wherein said bacterium has been modified to have a decreased expression and/or activity of at least one endogenous enzyme involved in the purine nucleotide degradation pathway compared to an otherwise identical bacterium that does not carry said modification.

45. The Gram-positive bacterium according to claim 44, wherein the at least one endogenous enzyme involved in the purine nucleotide degradation pathway is selected from the group consisting of: an enzyme having purine nucleoside phosphorylase activity and an enzyme having adenosine-phosphoribosyltransferase activity.

46. The Gram-positive bacterium according to claim 1, wherein the bacterium has been modified to have a decreased expression and/or activity of at least one endogenous enzyme involved in the guanosine monophosphate biosynthesis pathway compared to an otherwise identical bacterium that does not carry said modification.

47. The Gram-positive bacterium according to claim 46, wherein the at least one endogenous enzyme involved in the guanosine monophosphate biosynthesis pathway is selected from the group consisting of: an enzyme having IMP dehydrogenase activity and an enzyme having GMP synthetase activity.

48. The Gram-positive bacterium according to claim 1, wherein the bacterium has been modified to have an increased protein expression of a polypeptide having cytochrome P450 monooxygenase (CYP450) activity compared to an otherwise identical bacterium that does not carry said modification.

49. The Gram-positive bacterium according to claim 48, wherein the polypeptide having cytochrome P450 monooxygenase (CYP450) activity is selected from the group consisting of:
i) a polypeptide comprising an amino acid sequence of any one of SEQ ID NOs: 93 to 95 and
ii) a polypeptide comprising an amino acid sequence, which has at least 75% sequence identity to the amino acid sequence of any one of SEQ ID NOs: 93 to 95.

50. A method for producing an isoprenoid cytokinin or riboside derivative thereof, comprising cultivating a bacterium according to claim 1 under suitable culture conditions in a suitable culture medium.

51. The method according to claim 50, wherein the isoprenoid cytokinin or riboside derivative thereof is selected from the group consisting of trans-zeatin (tZ), trans-zeatin riboside (tZR), N⁶-(D2-isopentenyl)adenine (iP), N(6)-(dimethylallyl)adenosine (iPR), dihydrozeatin (DZ), ribosyl dihydrozeatin (DZR), and combinations thereof.

52. The method according to claim 50, wherein the isoprenoid cytokinin or riboside derivative thereof is trans-zeatin (tZ) and trans-zeatin riboside (tZR), respectively.


SEQUENCE LISTING (184 sequences)

SEQ ID NO: 1 - protein, 243 aa, Agrobacterium tumefaciens
MLLHLIYGPTCSGKTDMAIQIAQETGWPVVALDRVQCCPQIATGSGRPLESELQSTRRIYLDSRPLTEGILDAESAHRRLIFEVDWRKSEEGLILEGGSISLLNCMAKSPFWRSGFQWHVKRLRLGDSDAFLTRAKQRVAEMFAIREDRPSLLEELAELWNYPAARPILEDIDGYRCAIRFARKHDLAISQLPNIDAGRHVELIEAIANEYLEHALSQERDFPQWPEDGAGQPVCPVTLTRIR

SEQ ID NO: 2 - protein, 240 aa, Agrobacterium tumefaciens
MDLRLIFGPTCTGKTSTAVALAQQTGLPVLSLDRVQCCPQLSTGSGRPTVEELKGTSRLYLDDRPLVKGIIAAKQAHERLMGEVYNYEAHGGLILEGGSISLLKCMAQSSYWSADFRWHIIRHELADEETFMNVAKARVKQMLRPAAGLSIIQELVDLWKEPRLRPILKEIDGYRYAMLFASQNQITSDMLLQLDADMEDKLIHGIAQEYLIHARRQEQKFPRVNAAAYDGFEGHPFGMY

SEQ ID NO: 3 - protein, 234 aa, Pseudomonas savastanoi
MKIYLIWGATCTGKTEHSIKLSKSTGWPVIVLDRVQCCFDIATGSGRPHPEELQSTRRIYLDNRRISEGVISAEEANDRLKLEVNKHIDSGGVILEGGSISLLKLISKDPYWCDRFIWSQHRMRLQDTDVFMDKAKARVRRMLVGSTETTGLLDELVAAQSDLNAKLAIQDIDGYRYIMNYAQARRLSITQLLNVMTGDMKEELINGIALEYYEHAKWQERDFPAEWLAERSTR

SEQ ID NO: 4 - protein, 236 aa, Erwinia herbicola
MRGNVYLLWGATTTGKTAFSVSVAKERGWSVIALDRFQGYHEISTGSGAPTNNELEKTERIYITPQRGLADGIITAKDYNYWLKSRVLSLPEGKSDFIIEGGSVSLLKEMVSDEFWADFLWEIKIFRAPSKDVFIKRAKRRITDMLHPEDGRPSIMDEVSSFFMNHKTIHPLEDIDGYRTIIDYCRCKKIGFDKLKIMTTDQEKIIISGIAEEYYAHALWQEQETPCFPHSWSRRW

SEQ ID NO: 5 - protein, 255 aa, Rhodococcus fascians
MKESTMAQTQARFDRVRWEPGVYAIVGATGIGKSAEASKLALSHSAPIVVADRIQCYSDLLVTSGRAFDAKVEGLNRVWLDNRTIHQGNFDPDEAFDRLIKVLTSYVDRGEAVVMEGGSISLILRFAQTISNLPFPAVVNVMPIPDRQHYFAQQCARARQMLRGDSTGRNLLTELAEAWVLGDQHNFIASVAGLDCVLDWCATHSVTPEELANRDLTTEVLDELAASMGGRYVEHGVLQQEIFLRTFGAPGVTAR

SEQ ID NO: 6 - protein, 243 aa, Agrobacterium tumefaciens
MILHLIYGPTCSGKTDMAIQIAQETGWPVVALDRVQCCPQIATGSGRPLESELQSTRRIYLDSRPLTEGILDAESAHRRLIFEVDWRKSEDGLILEGGSISLLNCMAKSPFWRSGFQWHVKRLRLGDSDAFLTRAKQRVAEMFAIREDRPSLLEELAELWNYPAARPILEDIDGYRCAIRFARKHDLAISQLPNIDAGRHVELIEAIANEYLEHALSQERDFPQWPEDGAGQPVCPVTLTRIR

SEQ ID NO: 7 - protein, 238 aa, Ralstonia solanacearum
MPVRLYLIWGATTTGKTAQSVALARSAGAPVISLDRVQCCHELAVGSGRPSPSELLGTRREYLCEREVSRGVVSAAEANQLLLDKVARYATQERALILEGGSVSLINAMIRDARWSERGEWILRRIPVPGRAAFMAAARKRVREMLDPPPGQAGILDELQGLWGYPRNHAVLEDIDGYRQIIRYANALQVPICRITSIDPNAKALLIERIAQEYWEHALWQEQEFLGIPASWMRADDA

SEQ ID NO: 8 - protein, 249 aa, Xanthomonas oryzae
MSCILHLIWGPTCSGKTEVAVALAEQTGWPVIALDRVQCCPEMSTCSGRPLPAELRATRRIYLASRAVREGIIAADEAHALLVDLVDRRSAAGGVILEGGSISLINRMILDPYWANDWQWRSHRLLLGECADFLDRALRRVEKMLRTGMGRPSLLEELVALWPDPALRPTIEDVDGYRYSIRFARQWNVPVGQLLSIGDEMKQRLIHGIAEEYLGHAQWQERDLLTLPANWQAQDVSMGGSKSGLHHRL

SEQ ID NO: 9 - protein, 243 aa, Neorhizobium sp. NCHU2750; identical to SEQ ID NO: 1

SEQ ID NO: 10 - protein, 237 aa, Streptomyces hainanensis
MHIIAGPTGIGKSAAATELARATGAPIVVADRIQCFPELATTSARAGGEAADVERIWLAERAVADGDFAVADALEALIGTLRGLRERHPFVIVEGGSISLLWDFAELLPELGFRATVRLMHIADRREYVAGLTRRARGMLVPEGAATSAIEELAIAWKSVEQRSFVASINGFKGILEWCATHSFAVESLASAALTEEQVGELSRMVALRHAEHGVLQDRIFSMVFDGWCGEPDAVTD

SEQ ID NO: 11 - protein, 256 aa, Streptomyces ipomoeae
MADTGTHVETSPLVHLIAGPTGVGKSAAATELARETGAPVVVADRLQCFTDLATASARAGAEVPDVQRYWLGDRTVADGDLSASAATDALVALVERLAIRHPLVIVEGGSISLLRELSERRAALSWRLTVRLMPLPDRSDYVAALTRRARTMLAPPPPERGLLDELATLWRDPHRRYFAASVNGFETVLECCAKYSLDVKTLHKRNLPEHILARMAALIAERHAEHGILQHQVFTEIFADRISGSRCDLGALERVA

SEQ ID NO: 12 - protein, 257 aa, Streptomyces turgidiscabies
MEKVLKMTARIRTARGRGTIGVRALVGPTGVGKSAAATALARDTGAPVLVADRIQCFTDLVVTSGRAADAHVLEVDRIWLSDRTVSDGDYALGEASEDLLSRLGSLVTDRGLVVLEGGSISLLLRFAELMENLPYSVSVDLLPIFDKSYYSARQIARARKMLLPDSSGRSLLTEFAAAWRTPGQRRFAASVSGLDSICEWCAKHSVEPEEVADVDLSEKMLGELSTMLGERYAAHGFTQERLFLKTFDHHVRNSPDA

SEQ ID NO: 13 - protein, 256 aa, Streptomyces turgidiscabies
MGEIPADCTVRPYVHLIAGPTGAGKSAVANDLARVTGAPVVVADRLQCFTDLATTSARAGAQAPGVRRYWLADRTVADGDLDAAAAADSLFGLVERLGSRHPFVIVEGGSISLLKVLAARCAALPWQLSVRVLPLPAYGDYIEALTCRARTMLAPPTPERSLLEELAVLWADPRHRRFAASVNGFEAVLECCAKYSLNVGTIHKQAPPDPIRSQMASLIALRHAEHGILQSRVFTETFGSRLPEQILDPSVLGRAA

SEQ ID NO: 14 - protein, 243 aa, Agrobacterium rhizogenes
MLLYLIYGPTCSGKTDIAIQIAQKTGWPVVALDRVQCCPQIATGSGRPLPSELQSTRRIYLDSRRLTKGIIDAEGAHRRLILEVDWQESEEGLILEGGSVSLLNCMAKSPYWKSGFQWHVKRLRLGDSDAFLARAKQRVTEMFAIREDRPSLLEELAELWNYPATRPILEDIDGYRCAIRFARKHDLAINQLPDIDAERQQDLIEAIAKEYLEHAIMQERDFPQWPEDGARQPVGPATLMRIQ

SEQ ID NO: 15 - protein, 240 aa, Agrobacterium tumefaciens
MDLHLIFGPTCTGKTTTAIALAQQTGLPVLSLDRVQCCPQLSTGSGRPTVEELKGTTRLYLDDRPLVEGIIAAKQAHHRLIEEVYNHEANGGLILEGGSTSLLNCMARNSYWSADFRWHIIRHKLPDQETFMKAAKARVKQMLHPAAGHSIIQELVYLWNEPRLRPILKEIDGYRYAMLFASQNQITADMLLQLDANMEGKLINGIAQEYFIHARQQEQKFPQVNAAAFDGFEGHPFGMY

SEQ ID NO: 16 - protein, 240 aa, Agrobacterium tumefaciens; identical to SEQ ID NO: 2

SEQ ID NO: 17 - protein, 240 aa, Agrobacterium vitis
MDLRLIFGPTCTGKTSTAIALAQQTGLPVLSLDRVQCCPQLSTGSGRPTVEELKGTTRLYLDDRPLVKGIIAAEQAHERLIAEVYNYEAHGGLILEGGSISLLKCMAQSGYWSADFRWHIIRHKLADEETFMKAAKARVKQMLCPAIGPSLIQELVYLWNEPRLRPILKEIDGYRYAMLFASQNRITPDMLLQLDADMEGKLIHGIAQEYLIHARRQEHEFPPVSAAAFEGFEGPPFGAY

SEQ ID NO: 18 - protein, 236 aa, Agrobacterium vitis
MEAHLIFGPTSTGKTSVAIALAKRTGFPVIVLDRIQCYSQLSVGGGRPSAAEFQGTRRIYLIEGSLDEGVISAERAHECLVAAVEAHKPEGGVILEGGSISLFKRMAQSSYWNCGFTWHVTRLHLGGEEIFLAAAKKRINQMMQPDEQGNSFLGELVSVWKTTALRATLEGICGYRYAIEFAGKQGLEMDALTSLNRRQLEQLVHGMAHEYLCYARQQEQELPLPSLAGGEGPPFQ

SEQ ID NO: 19 - protein, 240 aa, Halomonas anticariensis
MRRHYILGTTASGKTSASISLAEMVNAPVVVLDRIQCFQELWTVSSRPQDSELGATKRFYLDDREISHGNLGVEDAYTKLVNIVDKLSNYHSFVILEGGSVSLCRLIVERAELCRGSTVEIRWNNPDDPAYVQKIRERVVQMMEPRVDRRSIVDELKYAWDYKEQRDFITSICGFDGILSWCDDNNFHIEDVDFECLPLSAIGEITDRVAESHRKYARTQFEMLNIMEQAMIATGVKVVR

SEQ ID NO: 20 - protein, 233 aa, Lonsdalea quercina
MDTTLHLIWGPTSTGKTARSVAIAEKTGAPVLVLDRIQCCPEVTMGSGRPTEQELRGTERVYLSSRPIEQSIISAPQAVERLHQLVDHFAKIHPLIILEGGSVSILQEMVKHQTWGQQYAWTFEPIPMPSAEHFLNRARKRIWEMFYPGGGLKSLLDEMVQIWAHPEYRDTLADIDGYRCIISYLRQRGRRVDEIPRMSLHEHKQMAEDIAQEYWQHAVWQTDNFPEIPPCWR

SEQ ID NO: 21 - protein, 238 aa, Ralstonia solanacearum
MPIRLYLIWGATTTGKTAQSVALARATGAPVISLDRVQCCHELAVGSGRPLPSELLGTRREYLCDRSVSSGVIPAADANQLLFDKVVRYSAHERALILEGGSVSLINAMIRDARWSALGEWALRRIVLPGRAVFIAQAIKRVRDMLEPPPGHASILDELEGLWAHPGNHAVLEDVDGYRQIIRYARASQVPIDRITSIDRSTMATLVERIAQEYWEHALWQEQEFLGIPATWRRADDA

SEQ ID NO: 22 - protein, 239 aa, Rhizobium radiobacter
MDLRLIFGPTCTGKTSTAIALAQQTGLPVLSLDRVQCCPQLSTGSGRPTVEELKGTTRLYLDDRPLVKGIITAKQAHERLIAEVHNHEAKGGLILEGGSISLLRCMAQSRYWNADFRWHIIRNELADEESFMSVAKTRVKQMLRPSAGLSIIQELVQLWREPRLRPILEGIDGYRYALLFATQNQITPDMLLQLDADMENKLIHGIAQEFLIHARRQEQKFPLVGATAVEAFEGPPFRM

SEQ ID NO: 23 - protein, 252 aa, Rhizobium tumorigenes
MLIYLIYGPTCSGKTDLAIQIARETGWPVIALDRVQCCPQIATGSGRPLESELQSTQRIYLDSRPLTEGVMEAEAAHLQLISAIEDRKSSPGLILEGGSISLLNCMAKDSYWNAGFRWHVRRLRLPTPDVFLPRARLRVTEMFAIKDERPSLLEELADLWNQEVLRPVLEDIDGYRCAIQFARENNIEIGELLHLDPMRREALIAAIAEEYLEHARWQEREFPDWQEGGNVGLTPLPASHDTGSPRDLRSLG

SEQ ID NO: 24 - protein, 236 aa, Streptomyces bingchenggensis
MHVIAGPTGVGKSAAATALARITGAPIVVADRIQCFTDLATTSARAGEETVEVERSWLGDRTVADGDYPPEEAADALVRRLAELGERHRFVIVEGGSISLLRDFAERLPDLPYRLTVRLLHIPDREGYVVRLARRARRMLAPGGSQTSMLEELAALWESPDHRFFAASVNGLEAILEWCATYSVRVESLASYELSEAQLEKMAWMVAERHAEHGILQERIFSKVFDGIPSAVYGPA

SEQ ID NO: 25 - protein, 246 aa, Streptomyces glauciniger
MISLHLILGPTGVGKTSRSVALALRHETPVLVLDRVQCHPQLAIGSGRPTQVELQDTVRYYLTERPLHHGVITAADAVDRLIHHRKMIHDSGVDRLILEGGSISILAELLTRTDWYEDATMAVECWLEASASAYEARVTERVEHMLGYSDHSGRTMLDELAELWLHPEARPRLHSIAGYREVIDLCAAEGLDPMYLRGLAARLRRTQCATAITSAHLDYARRQRLALATALPELVQRGFEVTLCEI

SEQ ID NO: 26 - protein, 294 aa, Streptomyces hainanensis
MARSGRRSLARSERSRTSLNVSTHRPIGRPRSRAPIATREEPTVRHIAAEVPLTVRLIVGPTGVGKSAAASDVARRTGAPVVVADRIQCFTDLATTSARAGADEPDVTRAWLGDRTVADGDYPAPEAAAALVRAVRRVARPGRPVVVEGGSISLLAYLVDHHLPVLPWRFTVTSLTRPAPSRYLAALTARAHAMLRPEPPLAGLLDELAALWRVPGHRWFAASVNGFEAALEWCAKYGLDPTEPTLVGLPPRLVDELALLIAERHAEHGADQERAFAALFAPAARAGVRLGDAA

SEQ ID NO: 27 - protein, 256 aa, Streptomyces sp. NRRL WC-3618
MAEISNDRTARPFVHLIAGPTGAGKSAAANELARVTGAPVVVADRLQCFTDLVTTSARAGAEARGVRRYWLADRTVADGDLGAVAAADSLVGLVERLGRQHRFVIVEGGSISLLKALAERRATLPWQLSVRVLPLPAYGDYVEALTRRAQTMIAPPTPDPSLLEELATLWADPRLRRFAASVNGFEAVLEICAKYSLDVETIHKQVPPAPVLSQMASLIALRHAEHGILQSYVFTEIFEEQIPVQVSDPGVLVRAA

SEQ ID NO: 28 - protein, 239 aa, Trinickia caryophylli
MTIRLYMIWGPTTTGKTALSVALARMVGVPVLSLDRVQCCHEIAVGSGRPSPEELEGTAREYLCHRRVADGVVSAAEANRLLKAKVLDYSRHLPALILEGGSVSLFNEMTADDSWAHIAEWVLQRVPLPAQDVFMKRARGRARAMLDAPAGERSMLDELVDLWRDPRTHAVLEDIDGYRQLIGYARAAGIPVVELSNAAPEIFDDWVELIAREYWEHACWQEQRFASPPTCWRHVAHAW

SEQ ID NO: 29 - protein, 238 aa, Xanthomonas albilineans
MNGALYLIWGPTCSGKTALAVALAEQSGWPVIALDRIQCYPELATGSGRPLSSELRGTRRIYLASRAVREGIIAADEAHALLKDMVGRHSAAGGVILEGGSISLINRMIADPYWASGWQWHSRRLRLGDPAAFLDRALRRVEQMLRTHQEHPSLLEELVALWPDPALRPILEDVDGYRYAIRFARHWSLPVADLLSMRDEMKQRLIRGIAEEYLEHAQWQERDLLALPASWQAQELST

SEQ ID NO: 30 - protein, 238 aa, Chondromyces crocatus
MKAHCIVGATGTGKTARATSLARQTGAPVLVLDRFQVFEDLASGTGRPLSREVAGTTRLYLGHRYMMDGELTRQDALLDFLRTVGKLAVDHDFIILEGGSISLNEALIEESLLGTTGVTVERMRMTSVKAYEQGLRRRARMMLTPSAERPSMIEELARGWDNPRLRAFLSSITGYDAIVDHCEQAGIEPAELAAGPVDPVLIDEVVLSHMKYAAKQTAAFNQLFPEQSSFPGCQPCTA

SEQ ID NO: 31 - protein, 239 aa, Dickeya dianthicola
MSTILYLLWGPTSTGKTSHSVILAKKYGFPVIALDRFQGYQEIKTGSGAPDQAELQGTERIYITPSHHLTESVVSSEEAHNILKKKISILLNRNSSVILEGGSVSLLTKIVMDDYWSQFSWRIRRFHMISKKDFIEKAQKRVTSMIYPQNGGSSILKETSLFFRKNLTIEPLEDIDGYRKIIQYCKEKNIDFDSLDKLNSNEKDNLIDIISNEYYEHALWQEKNFPGFPASWSWETLKN

SEQ ID NO: 32 - protein, 244 aa, Nostoc sp.
MRLHIILGPTSVGKTDRSVVLAKQTKAPVIVLDRIQIYQEIATGSGRPLIDELEGTTRIYLEERQLADGNLNTLESLSLALQHIDRLSSQHKLLILEGGSISLCTALWKSRILENYQTTIEYVKVENEELYQSRLWRRMQNALISNPRRPSLIEELSRVWQDPHKLALVRTVVGYDVLIHWCQKYGLSPDQMWKSFQDNSFYINLMQEMFLAYMQYSQNQRQAFDQLAVEYKQQQALLASTVTL

SEQ ID NO: 33 - protein, 283 aa, Dictyostelium discoideum
MKILLIIGSTGVGKTDLSINYSKKYNAPVVVLDRIQCFPELSITSGRPDESEYFGSKRIYLTDLLVEPGNENIKKTFYVNKLINILNEIKNNYDTQNLPNEKGYGCIFEGGSISLLKELLTKINKLPYKITCVIYIRPSDSIDNHKLYKAKIFKRVSQMLFPTEEGNDSQILEVKRILNKGKTVNAQGEINLEYYEKIKQVLISLVGLVGIEDVIHFLDKQYDQKNITSKLDPNYLNEIQSQLIETITLAHYNYALSQIELIDSLIKQLPKSIEYCLKNIEIN

SEQ ID NO: 34 - protein, 195 aa, Corynebacterium glutamicum
MTSLFDAPTLQRVTVFTGSALGSSSLYTQAAQTLAKTAVDRGIDLVYGGGKVGLMGIVADAFLESGGEAFGVITESLMKGELGHEKLTELEIVPDMHIRKRRMAELGDGFIAMPGGAGTLEELFEVWTWQQLGIHQKPVALYDVDGFWQPLLEMLEQMTQRGFIKRDFFECLIVESDPHALLKAMQTWTPPAPKW

SEQ ID NO: 35 - protein, 191 aa, Bacillus subtilis
MKTICVFAGSNPGGNEAYKRKAAELGVYMAEQGIGLVYGGSRVGLMGTIADAIMENGGTAIGVMPSGLFSGEVVHQNLTELIEVNGMHERKAKMSELADGFISMPGGFGTYEELFEVLCWAQIGIHQKPIGLYNVNGYFEPMMKMVKYSIQEGFSNESHLKLIHSSSRPDELIEQMQNYSYPILEKKWTEI

SEQ ID NO: 36 - protein, 256 aa, Corynebacterium glutamicum
MAPKQTPSPEKNRNLVGPVLQRRQTEGTFDQRLLEMRADHNWKHADPWRVLRIQSEFVAGFDALHEMPKAVTVFGSARIKEDHPYYKAGVELGEKLVAADYAVVTGGGPGLMEAPNKGASEANGLSVGLGIELPHEQHLNPYVDLGLNFRYFFARKTMFLKYSQAFVCLPGGFGTLDELFEVLCMVQTGKVTNFPIVLIGTEFWAGLVDWIRHRLVEEGMIDEKDVDRMLVTDDLDQAVKFIVDAHAGLDVARRHN

SEQ ID NO: 37 - protein, 195 aa, Pseudomonas aeruginosa
MTLRSVCVFCGASPGASPVYQEAAVALGRHLAERGLTLVYGGGAVGLMGTVADAALAAGGEVIGIIPQSLQEAEIGHKGLTRLEVVDGMHARKARMAELADAFIALPGGLGTLEELFEVWTWGQLGYHAKPLGLLEVNGFYDPLLTFLDHLVDERFVRAEHRGMLQRGASPEALLDALAAWTPSVAPKWVDRTPQ

SEQ ID NO: 38 - protein, 256 aa, Rhodococcus fascians
MNSVDDPGSYEQIGSEKPAKHKGSVVLRRGRNTETSTSDQRLLDERGSGNWIHSDTWRVLRIQSEFVEGFGALAEVPRAVTVFGSARTPVDHPEYERARELGAALAREGFAVITGGGPGVMEGANRGASEADGYSIGLGIELPFEQGLNDWVDLGVNFRYFFVRKTMFVKYSQAFVCLPGGFGTLDELFEALTLVQTAKVTRFPIVLLGKDFWSGLLDWIKTTLVKEGKVSEKDLDLIHCTDSVDEAVRIVREAQQ

SEQ ID NO: 39 - protein, 198 aa, Rhodococcus fascians
MNLRPMPATTVSAQARPTPKSVTVFCGAMPGRGTKYGQLAEGMGRAIARSKLRLVYGGARVGLMGTLANAALDSGGTVVGVIPESFTAIPEAAHHGLTELHVVHDMHQRKALMAELGDAFIALPGGVGTAEEFFEVLTWSHLGLHNKPCVLLNDNEYYRPLLSYIEHAAVEGFITPATRSRVIVCKDIEGAIAAIRSP

SEQ ID NO: 40 - protein, 187 aa, Corynebacterium stationis
MKSVAIYCGSSMGSSPRYAEVAKEMGEELARRNLRVVYGGGNVGLMGTVADAALAAGGEVVGVIPRQLINREMAHAGLTDLEVVDTMAQRKTRMEELADAFVCLPGGVGTLEEVVEVLTMQQLGHVQGPVGLVNVDGFWEPFVRTLESMVACGFVQSRYIDAIALSSDPAEILDEFAGWSPIGAKWS

SEQ ID NO: 41 - protein, 275 aa, Corynebacterium stationis
MAPLRSNKDGKPRVPRTTESAETNRMLRGPMLLRTDGNQTSTYDQRLLESGADHAWKHADPWRVLRIQGEFVAGFDALSDLPKAVTVYGSARTKEDTPEYAQGIDLGAKLVEHDYAVVTGGGPGIMEAANRGAYEAGGLSVGLGIELPFEQGLNKYVDLGINFRYFFARKTMFLKYSQAFVCLPGGMGTMDELFEMLCMVQTGKVTNYPIVLIGTEYWSGLVEWMANTLVAEGYINPQDMDLFLLTDSVDEAVEHIVAAHKEMTDERVSRLNHDA

SEQ ID NO: 42 - protein, 252 aa, Streptomyces coelicolor
MATGNPEGKKRPPEEQRLGPVLRRRGQVQESTTDQRLLDERAPTDWVHTDPWRVLRIQSEFIEGFGTLAELPPAISVFGSARTPADSPEYDAGVRLGRGLVEAGFAVITGGGPGAMEAANKGALEAKGTSVGLGIELPFEQGLNPYVDIGLNFRYFFVRKMMFVKYAQGFVVLPGGLGTLDELFEALTLVQTQKVTRFPIVLFGSEYWGGLVDWLRGTLVAQGKAAEKDLMLFHVTDDVDEAVALVSKEAGR

SEQ ID NO: 43 - protein, 215 aa, Arabidopsis thaliana
MEIKGESMQKSKFRRICVFCGSSQGKKSSYQDAAVDLGNELVSRNIDLVYGGGSIGLMGLVSQAVHDGGRHVIGIIPKTLMPRELTGETVGEVRAVADMHQRKAEMAKHSDAFIALPGGYGTLEELLEVITWAQLGIHDKPVGLLNVDGYYNSLLSFIDKAVEEGFISPTAREIIVSAPTAKELVKKLEEYAPCHERVATKLCWEMERIGYSSEE

SEQ ID NO: 44 - protein, 306 aa, Arabidopsis thaliana
MGVSMLDSSWSFRLSSKMSPSPTQKLASSLTERDPISLNLRFDHSIRAYSARALKCRSVKSEDFVERSSPFEVKKELEICFDLVHRLGRGIVYLGSARIPPNHSHYLQAQELSREAATLLDCTTWSGAGPGLMDAVTEGALEAEKPVGGIKIEKEAGEWTASKFHPYLPPQNYHTCRFFSARKHGLVDAVIRNNVSEKTAIIALPGGIGTLDEMFEILALIQLERIGSALPVPFIVMNYDAFYSKLLEFIETCENLGTVSKGEVSALWKVCNNNFEALTYLAEFYDLPDGSAKLETELRKTTPTRG

SEQ ID NO: 45 - protein, 194 aa, Burkholderia sp. TNe-862
MKAVCVYCGSAEGANPIYAEAAQAFGRALVAANLALVYGGGKVGLMGVIADTVMAAGGRAIGVIPELLVDKEVGHTGLTELHVVPDMHHRKKMMADFSDAFVAMPGGAGTLEELFEVYTWAQLGYHRKPVGVLNIGGFYDPLVALLDHTVREGFMRQTYREMLQIDGDPAALIARLKRYHAPAKDKWADLRENV

SEQ ID NO: 46 - protein, 194 aa, Paraburkholderia sp. SOS3
MKAVCVYCGSAKGANPIYAEAAHAFGRALVDANLALVYGGGKVGLMGMIADSVMAAGGRAIGVIPELLVDKEIGHHGLTELHVVSDMHERKKLMADLSDAFVALPGGAGTLEELFEVYTWAQLGYHRKPVGILNIAGFYDPLIAMLEHTVREGFMQQTYFETLQIDNEAAALLGKLQHYRPLAQDKWSQHRDKV

SEQ ID NO: 47 - protein, 195 aa, Pseudomonas sp. HAR-UPW-AIA-41
MPLQSICVFCGASVGHDPAYQQAAEALGRTLAERGLRLIYGGGEVGLMGKLADAVLAAGGEVTGIIPQSLQRSEIGHKGLTRLEVVDGMHARKARMAELSDAFIALPGGLGTLEELFEVWTWAQLGYHTKPIGLLDVAGFYRGLSAFLDQTVSAGFIRPPHRGLLHQAEDPAQLLDLLQQQQGSVTPKWDARQPI

SEQ ID NO: 48 - protein, 195 aa, Pseudomonas putida
MPVRSVCVFCGASMGANPAYREAAVALGQAIARRGLTLVYGGGAVGLMGVVADAAMAAGGEVVGIIPQSLLDAEVGHKGLTRLEVVDGMHARKARMAELSDAFIALPGGLGTLEELFEVWTWGQLGYHAKPLGLLDVNGFYDKLGGFLDHIVEEGFVRPQHRAMLLLGQQPDELLEGMDSFVAPVAPKWVDKQPD

SEQ ID NO: 49 - protein, 203 aa, Bradyrhizobium sp. Leo170
MQNQNAKALKSVCIFCGSSPGRDPIYLQTAVATGQALAKAGVTIVYGGGKVGLMGAVADAALAAGGRVVGVIPRALVEREIGHTGLSQLHIVETMHERKTMMAQLSDGFIALPGGAGTLEELFEEWTWAQLGIHQKPCGLLNVKDYFAPLIAMVERMVAEGFTIAAYAEMLIIETKVDALLSRFGTYQPPPRKWLRTEAAAKP

SEQ ID NO: 50 - protein, 200 aa, Rhodococcus hoagii
MRITVFMGSATGHDPAHLQTATQFGRDLAAAGIGLVYGGGRVGLMGAVADAVVAGGGETVGVIPRHLADKEIAHPGLSTLEVVESMHARKQRMAELADAFVVLPGGAGTLDEFFEIWTWQQLGLHDKPVFLFDADGFWQPLVGLLDHLVDAGFVAAYQREALLVAADLDDVRTAMTTWAPPAPKWSGTAEPANDTDLERA

SEQ ID NO: 51 - protein, 185 aa, Streptomyces gilvigriseus
MRVAVFTGSSFGSSPAFARAAEELGRGLAEAGMGIVYGGGHVGLMGAVADAALAAGGEVSGVIPRALAERELQHEGLTRLRIVETMHERKAAMAAEADAFVALPGGAGTLEELFEAWTWQHLGYHAKPVVLYDVDGFWQPLRAMVSGMVESGFIRAEFRDTLPTAGSVAELLGLLKDWRAPGGKW

SEQ ID NO: 52 - protein, 195 aa, Pseudomonas fluorescens
MSIASVCVFCGASTGTDPAYREAAQALGRALAERKLTLVYGGGAVGLMGIVADAALAAGGEVIGIIPQSLKDKEIGHSGLTRLEVVDGMHARKARMAELSDAFIALPGGLGTLEELFEVWTWGQLGYHGKPLGLLEVNGFYSKLTAFLDHIVGEGFVRAPHRDMLQVSESAQDLLDALDEWQPSVQPKWAEQKPS

SEQ ID NO: 53 - protein, 195 aa, Pseudomonas oryzihabitans
MSLKAVCVFCGAKSGASPVFEQAAAQLGRALAERGLTLVYGGGKVGLMGVVADAALAAGGEVIGIIPQSLKTAELGHTGCTRLEVVDGMHARKARMAELADAFIALPGGLGTLEELFEVWTWGQLGYHAKPMGLLEVDGFYAQLEGFLDHLVAQGFVAAPHRAMLQVEDSPIALLDALAAWQPTAPPRWAEPAPR

SEQ ID NO: 54 - protein, 189 aa, Amycolatopsis rhizosphaerae
MRRICVFCGSSSGNDAVYAEQAAALGKLLAERGIGLVYGGASVGIMGVVADAALAAGGEVIGVIPKHLMRVEIGHADLTELHVTAGMHERKAKMAELSDGFLALPGGAGTLEELAEVWTWAQLGLHHKPIGLVDVAGYYRPLREFADHMVAEGFLRPQHRDLIFIDPDPVVLLDAFESYEPPTVAKWIG

SEQ ID NO: 55 - protein, 193 aa, Corynebacterium provencense
MTAAAPVRKVALYLGSATGDSPAYRETTVRTVEVLADAGCELVYGGGSVGLMGVAADTALAAGMPVHGVITRHLMDGETGHPGLTDLEVVDTMTRRKERMAEIADAFIVLPGGIGTLEEFFEVWVAQVLGIHTRPVALVDVDNYWQPLTDMIDHMVSRGFLSAVHRDHLIRVNDPEMIPTALDVWTPAAAKWS

SEQ ID NO: 56 - protein, 195 aa, Erwinia typographi
MNTVGIFCGSSEGHSTAYIDAARHVGKYLAEQGISIVYGGGRVGLMGAVADAALAYGGHVTGVIPKSLLEREIAHTGLSELLVVDNMHQRKNMMAELSSGFIALPGGAGTAEEIFEQWTWAQLGIHDKPLAFLNVNHFYDPLTVMVQRMVSDGFMKQDYANMLRFSENLHDIIDYFKTYTPPVSKWTTRNENHHV

SEQ ID NO: 57 - protein, 194 aa, Paraburkholderia sp. BL10I2N1
MKSVCVYCGSSDGVKPLYAEAARAFGRAIVEARLALVYGGGKVGLMGVIADEVMAAGGRAIGVIPELLVNKEVGHEGLTELHVVPDMHQRKKMMADLSDAFIAMPGGAGTLEELFEVYTWAQLGYHQKPVAVLNIDGYYDPLISMLQHTVQEGFMRQTYLDILQIDSDPVSLISKLQRYLPPARDKWADNREAV

SEQ ID NO: 58 - protein, 195 aa, Pseudomonas putida
MSLRSVCVFCGASTGASPVYREAAIALGQAIAGRGLTLVYGGGAVGLMGIVADAAMAAGGEVIGIIPVSLQRAEIAHPGLTRLEVVDGMHARKARMAELSDAFIALPGGLGTFEELFEVWTWGQLGYHGKPLGLLEVNGFYEKLSGFLDHVVEQGFVRPQHRSMLQMNEAPDALLDALDAWEPVVQPKWVDQPPS

SEQ ID NO: 59 - protein, 199 aa, Acinetobacter baylyi
MVKKFMNSVCIFCGSAVGTDPVFIQAAQSTGKRIAEQGKTLVYGGGRSGLMGVVADSALQAGGHVIGVIPTALVDRELAHPHLTELHVVKNMHERKTKMAELSDGFIALPGGAGTLEEIFEQWTWAQLGIHQKPCGFLNIKGFYDDLLKMIQGTVDYGFTQSRFAEKLIASDDIEIILNQFEHYLAPTPKWGDHAKVHP

SEQ ID NO: 60 - protein, 185 aa, Arcobacter mytili
MKIAVFCGSSMGNNIKYLNATKALGKFFAQNNIDVVYGGGKVGLMGAIADSVMENGGKVYGVIPEKLKSKELAHTGITELHVVSTMHERKAMMAELADAFVTLPGGAGTLEEIFEAWTWAQLGYHNKPCAFYNVDGFYDKLFELINNMCECEFLKPEYSNMLIKTDNKEELLEAIKNYKAPLNKW

SEQ ID NO: 61 - protein, 189 aa, Blastococcus endophyticus
MRIAVFTGSQSGPESHRTAAAALAGDLARAGVGIVYGGGHVGLMGVVADAALAAGGEVVGVIPQHLVDDELAHPRLPRLEIVSSMHERKARMAELSDAFVALPGAAGTLEELFEAWTWGMLGLHAKPTVLVDVDGFWQPLLAQLRRMADDGYLDRRRLDALGVVTDAAGLLAFVEGYEHPPRKWTPTPA

SEQ ID NO: 62 - protein, 194 aa, Burkholderia sp. OLGA172
MKSVCVYCGSSDGVRPLYAEAARAFGRALVQADLALVYGGGKVGLMGVIADTVMAEGGRAVGVIPELLVNKEVGHNGLTELHVVPDMHHRKKMMADLSDAFVAMPGGAGTLEELFEVYTWAQLGYHQKPVALLNIDGFYDPLIALLKHTVDEGFMRQTYFDILQRDADPVALIDKLQRYKPPASDKWAIKRDAV

SEQ ID NO: 63 - protein, 633 aa, Bacillus subtilis
MDLLSIQDPSFLKNMSIDELEKLSDEIRQFLITSLSASGGHIGPNLGVVELTVALHKEFNSPKDKFLWDVGHQSYVHKLLTGRGKEFATLRQYKGLCGFPKRSESEHDVWETGHSSTSLSGAMGMAAARDIKGTDEYIIPIIGDGALTGGMALEALNHIGDEKKDMIVILNDNEMSIAPNVGAIHSMLGRLRTAGKYQWVKDELEYLFKKIPAVGGKLAATAERVKDSLKYMLVSGMFFEELGFTYLGPVDGHSYHELIENLQYAKKTKGPVLLHVITKKGKGYKPAETDTIGTWHGTGPYKINTGDFVKPKAAAPSWSGLVSGTVQRMAREDGRIVAITPAMPVGSKLEGFAKEFPDRMFDVGIAEQHAATMAAAMAMQGMKPFLAIYSTFLQRAYDQVVHDICRQNANVFIGIDRAGLVGADGETHQGVFDIAFMRHIPNMVLMMPKDENEGQHMVHTALSYDEGPIAMRFPRGNGLGVKMDEQLKTIPIGTWEVLRPGNDAVILTFGTTIEMAIEAAEELQKEGLSVRVVNARFIKPIDEKMMKSILKEGLPILTIEEAVLEGGFGSSILEFAHDQGEYHTPIDRMGIPDRFIEHGSVTALLEEIGLTKQQVANRIRLLMPPKTHKGIGS

SEQ ID NO: 64 - protein, 620 aa, Escherichia coli
MSFDIAKYPTLALVDSTQELRLLPKESLPKLCDELRRYLLDSVSRSSGHFASGLGTVELTVALHYVYNTPFDQLIWDVGHQAYPHKILTGRRDKIGTIRQKGGLHPFPWRGESEYDVLSVGHSSTSISAGIGIAVAAEKEGKNRRTVCVIGDGAITAGMAFEAMNHAGDIRPDMLVILNDNEMSISENVGALNNHLAQLLSGKLYSSLREGGKKVFSGVPPIKELLKRTEEHIKGMVVPGTLFEELGFNYIGPVDGHDVLGLITTLKNMRDLKGPQFLHIMTKKGRGYEPAEKDPITFHAVPKFDPSSGCLPKSSGGLPSYSKIFGDWLCETAAKDNKLMAITPAMREGSGMVEFSRKFPDRYFDVAIAEQHAVTFAAGLAIGGYKPIVAIYSTFLQRAYDQVLHDVAIQKLPVLFAIDRAGIVGADGQTHQGAFDLSYLRCIPEMVIMTPSDENECRQMLYTGYHYNDGPSAVRYPRGNAVGVELTPLEKLPIGKGIVKRRGEKLAILNFGTLMPEAAKVAESLNATLVDMRFVKPLDEALILEMAASHEALVTVEENAIMGGAGSGVNEVLMAHRKPVPVLNIGLPDFFIPQGTQEEMRAELGLDAAGMEAKIKAWLA

SEQ ID NO: 65 - protein, 633 aa, Corynebacterium stationis
MGILNKVKSPQDLKKLSDDKVEELAAEIRQFLIDKVSVTGGHLGPNLGVVELTIALHRVFDSPRDPIVFDTSHQSYVHKILTGRADQFDTLRQKDGLSGYTDRGESEHDWTESSHASAAISVIDGLSKAFCIKGESRRNAVAVVGDGALTGGMCWEALNNISANKERNAVIVVNDNGRSYSPTIGGLSENLSRIRTQHGYDELMEHGKKTLKSLGWVGNRTFDALRAVKEGVKSSILPTEMFPELGMKYIGPINGHDLDALDHALSYARDYEGPIIVHVVTEKGHGFAPAVNEPQDQMHSTGAIDPVTGVPKGKSQPGWTAVFSEELIEAAKKRDDIVAITAAMAGPTGLIPFAEQFPNRFFDVGIAEQHAMASASGLALGGLHPVVAIYSTFLNRAFDQLLMDIALLKQPVTIVLDRAGVTGSDGASHNGVWDMALTTIIPGIHVAAPRDGQRLRELFQESLEIKSGPSVVRFPKGNLLEDMDAVATTEDGVDIIFESEQERFADAEAKKVLVISIGAMAARSLGAAQILEDKGMAVTVVDPRWLCPVAPSLIEMADEHDIVVVAEDGMMRAGVGSLFDEAFSAAEVDTPLRRVAFPSIFPKHGSRGEVLEEVGMDADGIAAAVTEWVDDLH

SEQ ID NO: 66 - protein, 642 aa, Streptomyces avermitilis
MPLLTRIRGPRDLDRLSLEQLDQLAEEIRTFLVDAVSKTGGHLGPNLGVVELTIALHRVFDSPKDRVLFDTGHQSYVHKLLTGRQDFSKLKMKGGLSGYPSQAESEHDVIENSHASTVLGWADGLAKANQVLERDDHVVAVIGDGALTGGMAWEALNNIAAAKDRPLVIVVNDNERSYAPTIGGLANHLATLRTTDGYERFLARGKDLLERTPVVGRPLYETLHGAKKGLKDFIAPQGMFEDLGLKYVGPIDGHDIEALESALARAKRFGGPVIVHCLTEKGRGYQPALQDEADRFHAVGKIHPDTGLPIASSGADWTSVFGEEMVKLGQERKDIVAITAAMLQPVGLDKFAKVFPKRVYDVGIAEQHAAVSAAGLATGGLHPVFAVYATFLNRAFDQVLMDVALHKCGVTFVLDRAGVTGTDGASHNGMWDMSILQVVPGLRLAAPRDADQVRAQLREAVDVDDAPTVVRFSKGAVGPAVPAVGRIGGMDVLREPGTDAPDVLLVSVGALAPMCLEIAGLLDKQGISATVVDPRWVKPVDEAMAPLAERHRVVVTVEDNSRVGGVGSAIAQALRDAGVDVPLRDFGIPPRFLDHASRGEVMAEIGLTAPDIARQVTGLVSKLDGRFERPTAEIDSVEPARD

SEQ ID NO: 67 - protein, 656 aa, Streptomyces coelicolor
MTILENIRGPRDLKALSEAELGELSDEIRDFLVHAVARTGGHLGPNLGVVELTVALHRVFESPDDRILWDTGHQSYVHKLLTGRQDFSKLRSKGGLSGYPSREESAHDVIENSHASTVLGWADGLAKARQVQGERSHVVAVIGDGALTGGMAWEALNNIAAARDRPLIIVVNDNERSYAPTIGGLANHLATLRTTDGYERALAWGKDVLQRTPVVGHTVYEALHGAKKGFKDAFAPQGLFEDLGLKYLGPIDGHDIGAVESALRRAKRFHGPVLVHCLTEKGRGYEPALRDEEDHFHTVGVMDPLTCEPLGPAGGPSWTSVFGEEIVRIGEERADVVAITAAMLHPVGLAPFAERFPDRVWDVGIAEQHAAVSAAGLATGGLHPVVAVYATFLNRAFDQLLMDVALHRCGVTFVLDRAGVTGVDGASHNGMWDMSVLQVVPGLRIAAPRDADQLRTQLREAVAVDDAPTLLRFPKESVGPAVPAVDRIGGLDVLHTADRSEVLLVAVGVMAPVCLGAAELLEARGIGCTVVDPRWVKPVDPALAPLAARHRLVAVVEDNSRAAGVGSAVALALGDAEVDVPVRRFGIPEQFLAHAKRAEVLADIGLTPVDVAGRIAASLALAEPAGDRAGGPAVEQPGDGRMSGDGRIVMPAQGEN

SEQ ID NO: 68 - protein, 629 aa, Deinococcus radiodurans
MNELPGTSDTPLLDQIHGPKDLKRLSREQLPALTEELRGEIVRVCSRGGLHLASSLGAVDIITALHYVLDSPRDRILFDVGHQAYAHKILTGRRDQMADIKKEGGISGFTKVSESEHDAITVGHASTSLANALGMALARDAQGKDFHVAAVIGDGSLTGGMALAALNTIGDMGRKMLIVLNDNEMSISENVGAMNKFMRGLQVQKWFQEGEGAGKKAVEAVSKPLADFMSRAKNSTRHFFDPASVNPFAAMGVRYVGPVDGHNVQELVWLLERLVDLDGPTILHIVTTKGKGLSYAEADPIYWHGPAKFDPATGEYVPSSAYSWSAAFGEAVTEWAKTDPRTFVVTPAMREGSGLVEFSRVHPHRYLDVGIAEEVAVTTAAGMALQGMRPVVAIYSTFLQRAYDQVLHDVAIEHLNVTFCIDRAGIVGADGATHNGVFDLSFLRSIPGVRIGLPKDAAELRGMLKYAQTHDGPFAIRYPRGNTAQVPAGTWPDLKWGEWERLKGGDDVVILAGGKALDYALKAAEDLPGVGVVNARFVKPLDEEMLREVGGRARALITVEDNTVVGGFGGAVLEALNSMNLHPTVRVLGIPDEFQEHATAESVHARAGIDAPAIRTVLAELGVDVPIEV

SEQ ID NO: 69 - protein, 620 aa, Artificial Sequence
  Mutant Escherichia coli enzyme DXS (Y392F)
MSFDIAKYPTLALVDSTQELRLLPKESLPKLCDELRRYLLDSVSRSSGHFASGLGTVELTVALHYVYNTPFDQLIWDVGHQAYPHKILTGRRDKIGTIRQKGGLHPFPWRGESEYDVLSVGHSSTSISAGIGIAVAAEKEGKNRRTVCVIGDGAITAGMAFEAMNHAGDIRPDMLVILNDNEMSISENVGALNNHLAQLLSGKLYSSLREGGKKVFSGVPPIKELLKRTEEHIKGMVVPGTLFEELGFNYIGPVDGHDVLGLITTLKNMRDLKGPQFLHIMTKKGRGYEPAEKDPITFHAVPKFDPSSGCLPKSSGGLPSYSKIFGDWLCETAAKDNKLMAITPAMREGSGMVEFSRKFPDRYFDVAIAEQHAVTFAAGLAIGGYKPIVAIFSTFLQRAYDQVLHDVAIQKLPVLFAIDRAGIVGADGQTHQGAFDLSYLRCIPEMVIMTPSDENECRQMLYTGYHYNDGPSAVRYPRGNAVGVELTPLEKLPIGKGIVKRRGEKLAILNFGTLMPEAAKVAESLNATLVDMRFVKPLDEALILEMAASHEALVTVEENAIMGGAGSGVNEVLMAHRKPVPVLNIGLPDFFIPQGTQEEMRAELGLDAAGMEAKIKAWLA

SEQ ID NO: 70 - protein, 633 aa, Artificial Sequence
  Mutant Bacillus subtilis enzyme DXS (Y389F)
MDLLSIQDPSFLKNMSIDELEKLSDEIRQFLITSLSASGGHIGPNLGVVELTVALHKEFNSPKDKFLWDVGHQSYVHKLLTGRGKEFATLRQYKGLCGFPKRSESEHDVWETGHSSTSLSGAMGMAAARDIKGTDEYIIPIIGDGALTGGMALEALNHIGDEKKDMIVILNDNEMSIAPNVGAIHSMLGRLRTAGKYQWVKDELEYLFKKIPAVGGKLAATAERVKDSLKYMLVSGMFFEELGFTYLGPVDGHSYHELIENLQYAKKTKGPVLLHVITKKGKGYKPAETDTIGTWHGTGPYKINTGDFVKPKAAAPSWSGLVSGTVQRMAREDGRIVAITPAMPVGSKLEGFAKEFPDRMFDVGIAEQHAATMAAAMAMQGMKPFLAIFSTFLQRAYDQVVHDICRQNANVFIGIDRAGLVGADGETHQGVFDIAFMRHIPNMVLMMPKDENEGQHMVHTALSYDEGPIAMRFPRGNGLGVKMDEQLKTIPIGTWEVLRPGNDAVILTFGTTIEMAIEAAEELQKEGLSVRVVNARFIKPIDEKMMKSILKEGLPILTIEEAVLEGGFGSSILEFAHDQGEYHTPIDRMGIPDRFIEHGSVTALLEEIGLTKQQVANRIRLLMPPKTHKGIGS

SEQ ID NO: 71 - DNA, 1451 bp, Artificial Sequence
  Synthetic DNA fragment tzs-LOG (IPT-LOG)
cgcagcatacgcagcgaaatcagcatcaccggagaatcccaacgaagccaactagtatgttattacacctgatctacggtcctacatgtagtggtaaaactgatatggctatacaaatcgcgcaagaaacaggttggcccgtcgttgctcttgatcgtgtgcagtgctgtccacagattgcgacaggatccggcagacctctggaaagcgagttacaatctacccggcggatttatttagactcacgccctttgacagaaggcattcttgacgcggaatccgctcatcgccgtcttatattcgaggtagattggaggaaatcggaagaaggcttaatcctcgaaggagggtccatctcgcttctaaattgtatggctaagtctccattttggagatctggattccagtggcacgtcaagcgcctgagacttggggattcagatgcgtttctgacgcgagcaaaacagcgtgtggctgagatgtttgctattagagaagatcggccaagtttattggaggagctcgccgagctgtggaattacccggcagctcgcccgattttggaagacattgacggctatcgatgtgccattagatttgcaaggaaacacgatctcgccatatcacagctgccgaacatcgatgccggccggcatgttgaactgatcgaagccattgcaaacgaatatcttgaacatgcgctaagccaagaacgcgactttcctcaatggccggaggacggcgcaggacaaccagtatgcccggttaccttaacgagaatccgttaaaaaagaggggagggaaacattatgatgacatcgctttttgacgcgcctacgcttcaacgcgtaacggttttcactgggagcgccttgggctcctctagtctttatactcaggctgctcaaaccctggctaaaaccgcggtagaccgagggattgatctggtttatggcggtggaaaagtcggcctcatgggtattgttgctgatgcattccttgaaagcgggggtgaggcattcggcgtgataacagagtcactgatgaaaggagaattaggccatgagaaactgacggagttggaaatcgtgcccgacatgcatattcgtaaaaggcggatggcggaacttggggatggattcattgcgatgcctggaggagccggcaccctggaagaactgtttgaagtctggacatggcagcagttaggaattcatcaaaagccggttgcactctatgacgtcgatggcttctggcaaccgttattggaaatgttggaacaaatgacacagagaggatttatcaagagagatttttttgaatgcctcatcgtcgaatcagatccgcacgcattattaaaggccatgcagacatggacacctccagccccaaaatggtaaattaattctagagtcgatccccgggttcgccagcaatgactaccggcagcc

SEQ ID NO: 72 - DNA, 1442 bp, Artificial Sequence
  Synthetic DNA fragment tmr-LOG (IPT-LOG)
cgcagcatacgcagcgaaatcagcatcaccggagaatcccaacgaagccaactagtatggatttaagactgattttcggtccaacctgtacgggaaagacttccaccgcagttgcgttagctcaacagacaggcctcccggttttatcattggatcgggttcagtgttgcccgcaactgtccacgggctctggccgacccacagtggaggaactaaaaggaacatcgcggttatatttggatgaccgaccattggtgaaaggtataatagctgcgaaacaggcacacgagagattgatgggcgaagtgtacaactatgaagcgcatggggggctgattttggagggaggaagtatctcactgttaaaatgcatggctcaatcaagctattggagcgctgatttccgctggcatattataagacacgaattggcagatgaagaaacatttatgaatgtcgcgaaagcacgggtaaagcagatgttacgtcctgccgcaggccttagcatcattcaagagttagtggatctttggaaggagccgcgccttagacctatcctgaaagaaatagatggctatcgttacgctatgctctttgcatctcagaatcagattacttctgatatgctgctgcaacttgacgcggacatggaagacaaactcattcatggtattgcccaagaataccttattcatgcgaggcgccaagaacaaaagtttcccagagtaaacgccgccgcctatgacgggtttgaaggccatccgtttggaatgtattaaaaaagaggggagggaaacattatgatgacatcgctttttgacgcgcctacgcttcaacgcgtaacggttttcactgggagcgccttgggctcctctagtctttatactcaggctgctcaaaccctggctaaaaccgcggtagaccgagggattgatctggtttatggcggtggaaaagtcggcctcatgggtattgttgctgatgcattccttgaaagcgggggtgaggcattcggcgtgataacagagtcactgatgaaaggagaattaggccatgagaaactgacggagttggaaatcgtgcccgacatgcatattcgtaaaaggcggatggcggaacttggggatggattcattgcgatgcctggaggagccggcaccctggaagaactgtttgaagtctggacatggcagcagttaggaattcatcaaaagccggttgcactctatgacgtcgatggcttctggcaaccgttattggaaatgttggaacaaatgacacagagaggatttatcaagagagatttttttgaatgcctcatcgtcgaatcagatccgcacgcattattaaaggccatgcagacatggacacctccagccccaaaatggtaaattaattctagagtcgatccccgggttcgccagcaatgactaccggcagcc

SEQ ID NO: 73 - DNA, 958 bp, Artificial Sequence
  Erythromycin selectable marker
gagggattcgtcatgttggtattccaaatgcgttatgtagataaaacatctactgttttgaaacagactaaaaacagtgattacgcagataaataaatacgttagattaattcctaccagtgactaatcttatgactttttaaacagataactaaaattacaaacaaatcgtttaacttctgtatttgtttatagatgtaatcacttcaggagtgattacatgaacaaaaatataaaatattctcaaaactttttaacgagtgaaaaagtactcaaccaaataataaaacaattgaatttaaaagaaaccgataccgtttacgaaattggaacaggtaaagggcatttaacgacgaaactggctaaaataagtaaacaggtaacgtctattgaattagacagtcatctattcaacttatcgtcagaaaaattaaaactgaacattcgtgtcactttaattcaccaagatattctacagtttcaattccctaacaaacagaggtataaaattgttgggaatattccttaccatttaagcacacaaattattaaaaaagtggtttttgaaagccatgcgtctgacatctatctgattgttgaagaaggattctacaagcgtaccttggatattcaccgaacactagggttgctcttgcacactcaagtctcgattcagcaattgcttaagctgccagcggaatgctttcatcctaaaccaaaagtaaacagtgtcttaataaaacttacccgccataccacagatgttccagataaatattggaagctatatacgtactttgtttcaaaatgggtcaatcgagaatatcgtcaactgtttactaaaaatcagtttcatcaagcaatgaaacacgccaaagtaaacaatttaagtaccgttacttatgagcaagtattgtctatttttaatagttatctattatttaacgggaggaaataa

SEQ ID NO: 74 - DNA, 22 bp, Artificial Sequence
  Primer TwistBio For
gaagtgccattccgcctgacct

SEQ ID NO: 75 - DNA, 21 bp, Artificial Sequence
  Primer TwistBio Rev
cactgagcctccacctagcct

SEQ ID NO: 76 - DNA, 1680 bp, Artificial Sequence
  Initial fragment amyE
gaagtgccattccgcctgaccttcgcctttgcggtagtggtgcttacgatgtacgacagggggattccccatacattcttcgcttggctgaaaatgattcttctttttatcgtctgcggcggcgttctgtttctgcttcggtatgtgattgtgaagctggcttacagaagagcggtaaaagaagaaataaaaaagaaatcatcttttttgtttggaaagcgagggaagcgttcacagtttcgggcagctttttttataggaacattgatttgtattcactctgccaagttgttttgatagagtgattgtgataattttaaatgtaagcgttaacaaaattctccagtcttcacatcggtttgaaaggaggaagcggaagaatgaagtaagagggatttttgactccgaagtaagtcttcaaaaaatcaaataaggagtgtcaagaatgtttgcaaaacgattcaaaacctctttactgccgttattcgctggatttttattgctgtttcatttggttctggcaggaccggcggctgcgagtgctgaaacggcgaacaaatcgaatgagcttacagcaccgtcgatcaaaagcggaaccattcttcatgcatggaattggtcgttcaatacgttaaaacacaatatgaaggatattcatgatgcaggatatacagccattcagacatctccgattaaccaagtaaaggaagggaatcaaggagataaaagcatgtcgaactggtactggctgtatcagccgacatcgtatcaaattggcaaccgttacttaggtactgaacaagaatttaaagaaatgtgtgcagccgctgaagaatatggcataaaggtcattgttgacgcggtcatcaatcataccaccagtgattatgccgcgatttccaatgaggttaagagtattccaaactggacacatggaaacacacaaattaaaaactggtctgatcgatgggatgtcacgcagaattcattgctcgggctgtatgactggaatacacaaaatacacaagtacagtcctatctgaaacggttcttagacagggcattgaatgacggggcagacggttttcgatttgatgccgccaaacatatagagcttccagatgatggcagttacggcagtcaattttggccgaatatcacaaatacatctgcagagttccaatacggagaaatcctgcaggatagtgcctccagagatgctgcttatgcgaattatatggatgtgacagcgtctaactatgggcattccataaggtccgctttaaagaatcgtaatctgggcgtgtcgaatatctcccactatgcatctgatgtgtctgcggacaagctagtgacatgggtagagtcgcatgatacacgggtgcgcatgatcgtatggttcactgtccaccaaccaaaactgtgctcagtaccgccaatatttctcccttggggggtacaaagaggtgtccctagaagagatccacgctgtgtaaaaattttacaaaaaggtattgactttccctacagggtgtgtaataatttaattacaggcgggggcaaccccgctcagtacctagagcgtaaaagaggggagggaaacactagttggcttcgttgggattctccggtgatgctgatttcgctgcgtatgctgcgaggctaggtggaggctcagtg

SEQ ID NO: 77 - DNA, 32 bp, Artificial Sequence
  Primer For for end fragment with erythromycin selectable marker
aaatctagaaattaagaaggagggattcgtca

SEQ ID NO: 78 - DNA, 44 bp, Artificial Sequence
  Primer Rev for end fragment with erythromycin selectable marker
cactgagcctccacctagcctaatactcatggcttgtgaccgct

SEQ ID NO: 79 - DNA, 1911 bp, Artificial Sequence
  End fragment containing erythromycin selectable marker
gaagtgccattccgcctgacctcacggtagtcattgctggcgaacgcgtctagaaattaagaaggagggattcgtcatgttggtattccaaatgcgttatgtagataaaacatctactgttttgaaacagactaaaaacagtgattacgcagataaataaatacgttagattaattcctaccagtgactaatcttatgactttttaaacagataactaaaattacaaacaaatcgtttaacttctgtatttgtttatagatgtaatcacttcaggagtgattacatgaacaaaaatataaaatattctcaaaactttttaacgagtgaaaaagtactcaaccaaataataaaacaattgaatttaaaagaaaccgataccgtttacgaaattggaacaggtaaagggcatttaacgacgaaactggctaaaataagtaaacaggtaacgtctattgaattagacagtcatctattcaacttatcgtcagaaaaattaaaactgaacattcgtgtcactttaattcaccaagatattctacagtttcaattccctaacaaacagaggtataaaattgttgggaatattccttaccatttaagcacacaaattattaaaaaagtggtttttgaaagccatgcgtctgacatctatctgattgttgaagaaggattctacaagcgtaccttggatattcaccgaacactagggttgctcttgcacactcaagtctcgattcagcaattgcttaagctgccagcggaatgctttcatcctaaaccaaaagtaaacagtgtcttaataaaacttacccgccataccacagatgttccagataaatattggaagctatatacgtactttgtttcaaaatgggtcaatcgagaatatcgtcaactgtttactaaaaatcagtttcatcaagcaatgaaacacgccaaagtaaacaatttaagtaccgttacttatgagcaagtattgtctatttttaatagttatctattatttaacgggaggaaataattctatgagtcgcttttgtaaatttggaaagttacacgttactaaagggaatgtagatggatccttttaatggagtgtacgccaagcgcggtcaattggcataacgtcaacaaggcaaagcgcccgggcatgaatctgctgtcatccatgcaaatgattgcccacggctcggacagcgttctctatttccaataccgcaaatcacgggggtcatcagaaaaattacacggagcggttgtggatcatgacaatagcccgaagaaccgcgtctttcaagaagtggccaaggtaggcgagacattggaacggctgtccgaagttgtcggaacgaagaggccggctcaaaccgcgattttatatgactgggaaaatcattgggcgctcgaggatgctcaggggtttgcgaaggcgacaaaacgttatccgcaaacgcttcagcagcattaccgcacattctgggaacacgatatccctgtcgacgtcatcacgaaagaacaagacttttcaccatataaactgctgatcgtcccgatgctgtatttaatcagcgaggacaccgtttcccgtttaaaagcgtttacggctgacggcggcaccttagtcatgacgtatatcagcggggttgtgaatgagcatgacttaacatacacaggcggatggcatccggatcttcaagctatatttggagttgagcctcttgaaacggacaccctgtatccgaaggatcgaaacgctgtcagctaccgcagccaaatatatgaaatgaaggattatgcaaccgtgattgatgtaaagacagcttcagtggaagcggtgtatcaagaagatttttatgcgcgcacgccagcggtcacaagccatgagtattaggctaggtggaggctcagtg

SEQ ID NO: 80 - DNA, 20 bp, Artificial Sequence
  Primer For for IPT-LOG operon
gcggtagtggtgcttacgat

SEQ ID NO: 81 - DNA, 25 bp, Artificial Sequence
  Primer Rev for IPT-LOG operon
aggatccatctacattccctttagt

SEQ ID NO: 82 - DNA, 3968 bp, Artificial Sequence
  Complete synthetic trans-zeatin operon containi ng IPT-LOG (with TZS)
gcggtagtggtgcttacgatgtacgacagggggattccccatacattcttcgcttggctgaaaatgattcttctttttatcgtctgcggcggcgttctgtttctgcttcggtatgtgattgtgaagctggcttacagaagagcggtaaaagaagaaataaaaaagaaatcatcttttttgtttggaaagcgagggaagcgttcacagtttcgggcagctttttttataggaacattgatttgtattcactctgccaagttgttttgatagagtgattgtgataattttaaatgtaagcgttaacaaaattctccagtcttcacatcggtttgaaaggaggaagcggaagaatgaagtaagagggatttttgactccgaagtaagtcttcaaaaaatcaaataaggagtgtcaagaatgtttgcaaaacgattcaaaacctctttactgccgttattcgctggatttttattgctgtttcatttggttctggcaggaccggcggctgcgagtgctgaaacggcgaacaaatcgaatgagcttacagcaccgtcgatcaaaagcggaaccattcttcatgcatggaattggtcgttcaatacgttaaaacacaatatgaaggatattcatgatgcaggatatacagccattcagacatctccgattaaccaagtaaaggaagggaatcaaggagataaaagcatgtcgaactggtactggctgtatcagccgacatcgtatcaaattggcaaccgttacttaggtactgaacaagaatttaaagaaatgtgtgcagccgctgaagaatatggcataaaggtcattgttgacgcggtcatcaatcataccaccagtgattatgccgcgatttccaatgaggttaagagtattccaaactggacacatggaaacacacaaattaaaaactggtctgatcgatgggatgtcacgcagaattcattgctcgggctgtatgactggaatacacaaaatacacaagtacagtcctatctgaaacggttcttagacagggcattgaatgacggggcagacggttttcgatttgatgccgccaaacatatagagcttccagatgatggcagttacggcagtcaattttggccgaatatcacaaatacatctgcagagttccaatacggagaaatcctgcaggatagtgcctccagagatgctgcttatgcgaattatatggatgtgacagcgtctaactatgggcattccataaggtccgctttaaagaatcgtaatctgggcgtgtcgaatatctcccactatgcatctgatgtgtctgcggacaagctagtgacatgggtagagtcgcatgatacacgggtgcgcatgatcgtatggttcactgtccaccaaccaaaactgtgctcagtaccgccaatatttctcccttggggggtacaaagaggtgtccctagaagagatccacgctgtgtaaaaattttacaaaaaggtattgactttccctacagggtgtgtaataatttaattacaggcgggggcaaccccgctcagtacctagagcgtaaaagaggggagggaaacactagtatgttattacacctgatctacggtcctacatgtagtggtaaaactgatatggctatacaaatcgcgcaagaaacaggttggcccgtcgttgctcttgatcgtgtgcagtgctgtccacagattgcgacaggatccggcagacctctggaaagcgagttacaatctacccggcggatttatttagactcacgccctttgacagaaggcattcttgacgcggaatccgctcatcgccgtcttatattcgaggtagattggaggaaatcggaagaaggcttaatcctcgaaggagggtccatctcgcttctaaattgtatggctaagtctccattttggagatctggattccagtggcacgtcaagcgcctgagacttggggattcagatgcgtttctgacgcgagcaaaacagcgtgtggctgagatgtttgctattagagaagatcggccaagtttattggaggagctcgccgagctgtggaattacccggcagctcgcccgattttggaagacattgacggctatcgatgtgccattagatttgcaaggaaacacgatctcgccatatcacagctgccgaacatcgatgccggccggcatgttgaactgatcgaagccattgcaaacgaatatcttgaacatgcgctaagccaagaacgcgactttcctcaatggccggaggacggcgcaggacaaccagtatgcccggttaccttaacgagaatccgttaaaaaagaggggagggaaacattatgatgacatcgctttttgacgcgcctacgcttcaacgcgtaacggttttcactgggagcgccttgggctcctctagtctttatactcaggctgctcaaaccctggctaaaaccgcggtagaccgagggattgatctggtttatggcggtggaaaagtcggcctcatgggtattgttgctgatgcattccttgaaagcgggggtgaggcattcggcgtgataacagagtcactgatgaaaggagaattaggccatgagaaactgacggagttggaaatcgtgcccgacatgcatattcgtaaaaggcggatggcggaacttggggatggattcattgcgatgcctggaggagccggcaccctggaagaactgtttgaagtctggacatggcagcagttaggaattcatcaaaagccggttgcactctatgacgtcgatggcttctggcaaccgttattggaaatgttggaacaaatgacacagagaggatttatcaagagagatttttttgaatgcctcatcgtcgaatcagatccgcacgcattattaaaggccatgcagacatggacacctccagccccaaaatggtaaattaattctagaaattaagaaggagggattcgtcatgttggtattccaaatgcgttatgtagataaaacatctactgttttgaaacagactaaaaacagtgattacgcagataaataaatacgttagattaattcctaccagtgactaatcttatgactttttaaacagataactaaaattacaaacaaatcgtttaacttctgtatttgtttatagatgtaatcacttcaggagtgattacatgaacaaaaatataaaatattctcaaaactttttaacgagtgaaaaagtactcaaccaaataataaaacaattgaatttaaaagaaaccgataccgtttacgaaattggaacaggtaaagggcatttaacgacgaaactggctaaaataagtaaacaggtaacgtctattgaattagacagtcatctattcaacttatcgtcagaaaaattaaaactgaacattcgtgtcactttaattcaccaagatattctacagtttcaattccctaacaaacagaggtataaaattgttgggaatattccttaccatttaagcacacaaattattaaaaaagtggtttttgaaagccatgcgtctgacatctatctgattgttgaagaaggattctacaagcgtaccttggatattcaccgaacactagggttgctcttgcacactcaagtctcgattcagcaattgcttaagctgccagcggaatgctttcatcctaaaccaaaagtaaacagtgtcttaataaaacttacccgccataccacagatgttccagataaatattggaagctatatacgtactttgtttcaaaatgggtcaatcgagaatatcgtcaactgtttactaaaaatcagtttcatcaagcaatgaaacacgccaaagtaaacaatttaagtaccgttacttatgagcaagtattgtctatttttaatagttatctattatttaacgggaggaaataattctatgagtcgcttttgtaaatttggaaagttacacgttactaaagggaatgtagatggatcct

SEQ ID NO: 83 - DNA, 3968 bp, Artificial Sequence; identical to SEQ ID NO: 82
  Complete synthetic trans-zeatin operon containi ng IPT-LOG (with TMR)

SEQ ID NO: 84 - DNA, 1080 bp, Artificial Sequence
  synthetic DNA fragment of dxs part1
gaagtgccattccgcctgacctcgcagcatacgcagcgaaatcagcatcaccggagaatcccaacgaagccaactagtatggacttgttgtccattcaagacccgtctttcctaaagaatatgagcattgacgagcttgaaaaattgtccgatgagatccgccaatttctcatcacatctctatcggcctcgggcggacacattggaccgaatctgggggtcgtggaactgacagtagccctccacaaggagttcaactcacccaaagacaaatttctctgggacgtggggcaccaaagctacgttcataaattgcttactggtcgtggcaaagagttcgccacattgcgccagtacaaaggactctgcggattccctaaacgctcggaatccgagcacgatgtgtgggaaacaggtcattcttctacttctctcagcggcgccatgggaatggccgctgcacgagatattaaagggactgatgagtacataatcccaataataggtgacggcgctttaacaggaggaatggccctagaggccttgaaccacataggggatgagaagaaagatatgatagtcattttgaatgataatgagatgtctattgctcccaacgttggagcaatacatagcatgttagggcggttacgtactgcaggaaagtaccagtgggtaaaagatgaacttgaatacctcttcaagaagatacccgctgtaggaggcaagcttgcagccacggccgagagagtgaaggattctcttaaatatatgttggtttcagggatgttctttgaagaattgggattcacgtaccttggtccggttgacggccattcttaccacgagttaattgaaaatttacaatacgcgaaaaaaacgaaggggcctgttctgctgcacgttattaccaaaaaaggaaaaggttataaacctgcggaaaccgatacaattggcacgtggcacggcaccgggccttataaaatcaacacgggggactttgtcaagccgaaagctgccgcgcctagttggtccggcctggttagtgggacggtacagcgaatggcaggctaggtggag

SEQ ID NO: 85 - DNA, 1094 bp, Artificial Sequence
  Synthetic DNA fragment of dxs part2
gaagtgccattccgcctgacctaatcaacacgggggactttgtcaagccgaaagctgccgcgcctagttggtccggcctggttagtgggacggtacagcgaatggcccgggaagatgggaggattgtcgctatcacaccggcaatgccagtaggtagcaagttagaaggttttgcgaaagaatttccggataggatgtttgacgtaggtattgctgagcagcatgcggcgacgatggctgcggcaatggcaatgcagggcatgaagccttttttagctatttattcaacatttctgcaaagagcgtatgaccaagtggtacatgacatttgtcggcaaaatgctaatgtctttattgggattgatcgcgcagggttagtgggtgcagacggggaaacgcatcaaggagtgtttgacatcgcgtttatgcgacatatcccaaatatggtccttatgatgccaaaagacgaaaacgagggtcaacacatggtgcatacagctcttagttatgacgaaggacctatcgcgatgagatttccgagagggaatggattaggcgtcaaaatggatgagcagcttaaaacaattccgattggaacatgggaagtcttacgtccaggaaatgacgcggttatccttacatttggcactaccattgagatggcgattgaggcagcagaagaactccagaaggagggcctgtcagtccgtgttgttaatgcgagatttattaaaccgattgacgaaaagatgatgaaaagtatcctgaaagaaggcctgccgatacttaccatcgaggaagctgttctggaaggaggatttggctcatcaatccttgaatttgcacatgatcagggcgaatatcacacaccgatcgatcgaatgggcatcccggatcgatttatcgagcatggctccgttacagcactgcttgaagagatcggcttaacgaaacagcaggtggcaaacagaatcaggttacttatgccgccgaagacacataagggcatcggctcataaaaaagaggggagggaaacattaatgacgacctggctaacgagtctcgccgatctaggctaggtggaggctcagtg

SEQ ID NO: 86 - DNA, 2040 bp, Artificial Sequence
  Joined synthetic gene dxs complete sequence
gaagtgccattccgcctgacctcgcagcatacgcagcgaaatcagcatcaccggagaatcccaacgaagccaactagtatggacttgttgtccattcaagacccgtctttcctaaagaatatgagcattgacgagcttgaaaaattgtccgatgagatccgccaatttctcatcacatctctatcggcctcgggcggacacattggaccgaatctgggggtcgtggaactgacagtagccctccacaaggagttcaactcacccaaagacaaatttctctgggacgtggggcaccaaagctacgttcataaattgcttactggtcgtggcaaagagttcgccacattgcgccagtacaaaggactctgcggattccctaaacgctcggaatccgagcacgatgtgtgggaaacaggtcattcttctacttctctcagcggcgccatgggaatggccgctgcacgagatattaaagggactgatgagtacataatcccaataataggtgacggcgctttaacaggaggaatggccctagaggccttgaaccacataggggatgagaagaaagatatgatagtcattttgaatgataatgagatgtctattgctcccaacgttggagcaatacatagcatgttagggcggttacgtactgcaggaaagtaccagtgggtaaaagatgaacttgaatacctcttcaagaagatacccgctgtaggaggcaagcttgcagccacggccgagagagtgaaggattctcttaaatatatgttggtttcagggatgttctttgaagaattgggattcacgtaccttggtccggttgacggccattcttaccacgagttaattgaaaatttacaatacgcgaaaaaaacgaaggggcctgttctgctgcacgttattaccaaaaaaggaaaaggttataaacctgcggaaaccgatacaattggcacgtggcacggcaccgggccttataaaatcaacacgggggactttgtcaagccgaaagctgccgcgcctagttggtccggcctggttagtgggacggtacagcgaatggcccgggaagatgggaggattgtcgctatcacaccggcaatgccagtaggtagcaagttagaaggttttgcgaaagaatttccggataggatgtttgacgtaggtattgctgagcagcatgcggcgacgatggctgcggcaatggcaatgcagggcatgaagccttttttagctatttattcaacatttctgcaaagagcgtatgaccaagtggtacatgacatttgtcggcaaaatgctaatgtctttattgggattgatcgcgcagggttagtgggtgcagacggggaaacgcatcaaggagtgtttgacatcgcgtttatgcgacatatcccaaatatggtccttatgatgccaaaagacgaaaacgagggtcaacacatggtgcatacagctcttagttatgacgaaggacctatcgcgatgagatttccgagagggaatggattaggcgtcaaaatggatgagcagcttaaaacaattccgattggaacatgggaagtcttacgtccaggaaatgacgcggttatccttacatttggcactaccattgagatggcgattgaggcagcagaagaactccagaaggagggcctgtcagtccgtgttgttaatgcgagatttattaaaccgattgacgaaaagatgatgaaaagtatcctgaaagaaggcctgccgatacttaccatcgaggaagctgttctggaaggaggatttggctcatcaatccttgaatttgcacatgatcagggcgaatatcacacaccgatcgatcgaatgggcatcccggatcgatttatcgagcatggctccgttacagcactgcttgaagagatcggcttaacgaaacagcaggtggcaaacagaatcaggttacttatgccgccgaagacacataagggcatcggctcataaaaaagaggggagggaaacattaatgacgacctggctaacgagtctcgccgatctaggcta

SEQ ID NO: 87 - DNA, 768 bp, Artificial Sequence
  Spectinomycin selectable marker
gtgaggcgaatatatttgaatacatacgaacaaatcaataaagtgaaaaaaatacttcggaaacatttaaaaaataaccttattggtacttacatgtttggatcaggagttgagagtggactcaaaccaaatagtgatcttgactttttagtcgtcgtatctgaaccattgacagatcaaagtaaagaaatacttatacaaaaaattagacctatttcaaaaaaaataggagataaaagcaacttacgatatattgaattaacaattattattcagcaagaaatggtaccgtggaatcatcctcccaaacaagaatttatttatggagaatggttacaagagctttatgaacaaggatacattcctcagaaggaattaaattcagatttaaccataatgctttaccaagcaaaacgaaaaaataaaagaatatacggaaattatgacttagaggaattactacctgatattccattttctgatgtgagaagagccattatggattcgtcagaggaacttatagataattatcaggatgatgaaaccaactctatattaactttatgccgcatgattttaactatggacacgggtaaaatcataccaaaagatattgcgggaaatgcagtggctgaatcttctccattagaacatagggagagaattttgttagcagttcgtagttatcttggagagaatattgaatggactaatgaaaatgtaaatcttacgataaactatcttaataaccgccttaagaaactttaa

SEQ ID NO: 88 - DNA, 1503 bp, Artificial Sequence
  Initial fragment lacA
gaagtgccattccgcctgacctgccttttaatcccggcaacagcttaatcagtacatccatcattccgaagcatccgacattcgatcattacaaggaattatttgcgggcaaggaaagccttcaatatgtgcagtggtatgtcaactctatgaagatcagcctgtttacaatggcagggtctttgctctgtgtgacgtttacggcctatgcgttttcgcgctttcggtttaaagggaggaaatacgctttaacgctctttttattgctgcagatgattcctcagttttcagctttaattgccttgtttgtgctggcgcaaatcttgggaatgatcaatagccactggctgctaatcttgctttatatcggcggcctgatcccgatgaatacgtatttgatgaaagggtacatggattccattccgatggatttagacgaaagcgccaagattgacggagccagcagcaccagaatcttcttccagatcattctgccattatcaaaaccgatggcggcagtcgtggccatgaacggctttaccggtccgctcggagattttgtgctgtcctcaaccatattgagaacgcctgaatcatatacattgcccgtcggtctattcaatttagtgaatgatgtcatgggggccagctatacgacatttgcggccggagccctgcttatcagcataccggttgccgtcatctttattatgctgcaaaagaattttgtgtccggattaaccgcaggcggaacgaagggctaagagaacaaggaggagaatgtgatgtcaaagcttgaaaaaacgcacgtaacaaaagcaaaatttatgctccatgggggagactacaaccccgatcagtggctggatcggcccgatattttagctgacgatatcaaactgatgaagctttctcatacgaatacgttttctgtcggcatttttgcatggagcgcacttgagccggaggagggcgtatatcaatttgaatggctggatgatatttttgagcggattcacagtataggcggccgggtcatattagcaacgccgagcggagcccgtccggcctggctgtcgcaaacctatccggaagttttgcgcgtcaatgcctcccgcgtcaaacagctgcacggcggaaggcacaaccactgcctcacatctaaagtctaccgagaaaaaacacggcacatcaaccgctacgggtgcgcatgatcgtatggttcactgtccaccaaccaaaactgtgctcagtaccgccaatatttctcccttggggggtacaaagaggtgtccctagaagagatccacgctgtgtaaaaattttacaaaaaggtattgactttccctacagggtgtgtaataatttaattacaggcgggggcaaccccgctcagtacctagagcgtaaaagaggggagggaaacactagttggcttcgttgggattctccggtgatgctgatttcgctgcgtatgctgcgaggctaggtggaggctcagtg

SEQ ID NO: 89 - DNA, 2172 bp, Artificial Sequence
  End fragment with spectinomycin selectable marker
gaagtgccattccgcctgacctcacggtagtcattgctggcgaacgcgtctagagctccggcataaaattaactaaaataattattatgtagataaaaaatttagaagccaatgaaatctataaataaactaaattaagtttatttaattaacaactatggatataaaataggtactaatcaaaatagtgaggcgaatatatttgaatacatacgaacaaatcaataaagtgaaaaaaatacttcggaaacatttaaaaaataaccttattggtacttacatgtttggatcaggagttgagagtggactcaaaccaaatagtgatcttgactttttagtcgtcgtatctgaaccattgacagatcaaagtaaagaaatacttatacaaaaaattagacctatttcaaaaaaaataggagataaaagcaacttacgatatattgaattaacaattattattcagcaagaaatggtaccgtggaatcatcctcccaaacaagaatttatttatggagaatggttacaagagctttatgaacaaggatacattcctcagaaggaattaaattcagatttaaccataatgctttaccaagcaaaacgaaaaaataaaagaatatacggaaattatgacttagaggaattactacctgatattccattttctgatgtgagaagagccattatggattcgtcagaggaacttatagataattatcaggatgatgaaaccaactctatattaactttatgccgcatgattttaactatggacacgggtaaaatcataccaaaagatattgcgggaaatgcagtggctgaatcttctccattagaacatagggagagaattttgttagcagttcgtagttatcttggagagaatattgaatggactaatgaaaatgtaaatcttacgataaactatcttaataaccgccttaagaaactttaacccgggcgcgttgctttcgatgaagttttaccgccatcaccagaccctcaatcgtatgtgtttactgccaatactggagacagcacggtttcggttattgatgcagatttaaacactgttgtaaaaacaatccctttttctgacgtgccaactaatctaggcgttactttcgataaagcattcacgtatgtcctccatggaaataccaaccttgtctcagtgattgataataaaacattaaccatcataaataccattactgttggaggagggccgagaaaaatcgaatttgatccaacagatgagttcgcatacgtgatggccgccggatctatatatgtgattaacacggcctctcaatcggtcattgatgtcattccgatacccggcgctttagactttgctcttgatccgaatggacagtatgtctataccgctaatggaagcagttggtcggttgataaatatgatgtaaatactgggcagttggtagaaagtatcatagatacatttgaatttcccagtttaattacaactccttacgctggaaattttgcgtacgtattgaatggtgagctgtggccgaaaggtgtaacggagattagtttgtcaccattaagcaggggaggggacttcagccgtttatttgaaacgctgaggacgattgtattttctctcgatagtactagagcttattttcaagagccatatagtgaaccgtttctcatcaataacttatacgtagttaatacggccagacagaggatcattgcaaacgtgtctctccccggcgcctttgacctcgccgtaacgccggacaaacagtatatttatgcggcacagcccaacgacaatgctgtgaccgtgtaccgcacaagtgattatacagccgtgacggtgattccggttggcgcgggtccgtctgcgattgcgatgtagctattttatagatcaagagaagcggtgctgtgataagagaatcgtttataaataaatgatccgatttgctgaatggcagccaggataatcagaaacagcagttcacttatagagaaactgagcatccaccaaatcccggctgctgccagtgctcccgcaaacacttttctgaacaggctgccgtgccggaaaagctgccgcaaatacgttcgcatgcaaaacaataaatacaggatcagtagcatcgcaagtcccataggctaggtggaggctcagtg

SEQ ID NO: 90 - DNA, 4245 bp, Artificial Sequence
  Complete synthetic dxs operon
taatcccggcaacagcttaatcagtacatccatcattccgaagcatccgacattcgatcattacaaggaattatttgcgggcaaggaaagccttcaatatgtgcagtggtatgtcaactctatgaagatcagcctgtttacaatggcagggtctttgctctgtgtgacgtttacggcctatgcgttttcgcgctttcggtttaaagggaggaaatacgctttaacgctctttttattgctgcagatgattcctcagttttcagctttaattgccttgtttgtgctggcgcaaatcttgggaatgatcaatagccactggctgctaatcttgctttatatcggcggcctgatcccgatgaatacgtatttgatgaaagggtacatggattccattccgatggatttagacgaaagcgccaagattgacggagccagcagcaccagaatcttcttccagatcattctgccattatcaaaaccgatggcggcagtcgtggccatgaacggctttaccggtccgctcggagattttgtgctgtcctcaaccatattgagaacgcctgaatcatatacattgcccgtcggtctattcaatttagtgaatgatgtcatgggggccagctatacgacatttgcggccggagccctgcttatcagcataccggttgccgtcatctttattatgctgcaaaagaattttgtgtccggattaaccgcaggcggaacgaagggctaagagaacaaggaggagaatgtgatgtcaaagcttgaaaaaacgcacgtaacaaaagcaaaatttatgctccatgggggagactacaaccccgatcagtggctggatcggcccgatattttagctgacgatatcaaactgatgaagctttctcatacgaatacgttttctgtcggcatttttgcatggagcgcacttgagccggaggagggcgtatatcaatttgaatggctggatgatatttttgagcggattcacagtataggcggccgggtcatattagcaacgccgagcggagcccgtccggcctggctgtcgcaaacctatccggaagttttgcgcgtcaatgcctcccgcgtcaaacagctgcacggcggaaggcacaaccactgcctcacatctaaagtctaccgagaaaaaacacggcacatcaaccgctacgggtgcgcatgatcgtatggttcactgtccaccaaccaaaactgtgctcagtaccgccaatatttctcccttggggggtacaaagaggtgtccctagaagagatccacgctgtgtaaaaattttacaaaaaggtattgactttccctacagggtgtgtaataatttaattacaggcgggggcaaccccgctcagtacctagagcgtaaaagaggggagggaaacactagtatggacttgttgtccattcaagacccgtctttcctaaagaatatgagcattgacgagcttgaaaaattgtccgatgagatccgccaatttctcatcacatctctatcggcctcgggcggacacattggaccgaatctgggggtcgtggaactgacagtagccctccacaaggagttcaactcacccaaagacaaatttctctgggacgtggggcaccaaagctacgttcataaattgcttactggtcgtggcaaagagttcgccacattgcgccagtacaaaggactctgcggattccctaaacgctcggaatccgagcacgatgtgtgggaaacaggtcattcttctacttctctcagcggcgccatgggaatggccgctgcacgagatattaaagggactgatgagtacataatcccaataataggtgacggcgctttaacaggaggaatggccctagaggccttgaaccacataggggatgagaagaaagatatgatagtcattttgaatgataatgagatgtctattgctcccaacgttggagcaatacatagcatgttagggcggttacgtactgcaggaaagtaccagtgggtaaaagatgaacttgaatacctcttcaagaagatacccgctgtaggaggcaagcttgcagccacggccgagagagtgaaggattctcttaaatatatgttggtttcagggatgttctttgaagaattgggattcacgtaccttggtccggttgacggccattcttaccacgagttaattgaaaatttacaatacgcgaaaaaaacgaaggggcctgttctgctgcacgttattaccaaaaaaggaaaaggttataaacctgcggaaaccgatacaattggcacgtggcacggcaccgggccttataaaatcaacacgggggactttgtcaagccgaaagctgccgcgcctagttggtccggcctggttagtgggacggtacagcgaatggcccgggaagatgggaggattgtcgctatcacaccggcaatgccagtaggtagcaagttagaaggttttgcgaaagaatttccggataggatgtttgacgtaggtattgctgagcagcatgcggcgacgatggctgcggcaatggcaatgcagggcatgaagccttttttagctatttattcaacatttctgcaaagagcgtatgaccaagtggtacatgacatttgtcggcaaaatgctaatgtctttattgggattgatcgcgcagggttagtgggtgcagacggggaaacgcatcaaggagtgtttgacatcgcgtttatgcgacatatcccaaatatggtccttatgatgccaaaagacgaaaacgagggtcaacacatggtgcatacagctcttagttatgacgaaggacctatcgcgatgagatttccgagagggaatggattaggcgtcaaaatggatgagcagcttaaaacaattccgattggaacatgggaagtcttacgtccaggaaatgacgcggttatccttacatttggcactaccattgagatggcgattgaggcagcagaagaactccagaaggagggcctgtcagtccgtgttgttaatgcgagatttattaaaccgattgacgaaaagatgatgaaaagtatcctgaaagaaggcctgccgatacttaccatcgaggaagctgttctggaaggaggatttggctcatcaatccttgaatttgcacatgatcagggcgaatatcacacaccgatcgatcgaatgggcatcccggatcgatttatcgagcatggctccgttacagcactgcttgaagagatcggcttaacgaaacagcaggtggcaaacagaatcaggttacttatgccgccgaagacacataagggcatcggctcataaattaattctagagctccggcataaaattaactaaaataattattatgtagataaaaaatttagaagccaatgaaatctataaataaactaaattaagtttatttaattaacaactatggatataaaataggtactaatcaaaatagtgaggcgaatatatttgaatacatacgaacaaatcaataaagtgaaaaaaatacttcggaaacatttaaaaaataaccttattggtacttacatgtttggatcaggagttgagagtggactcaaaccaaatagtgatcttgactttttagtcgtcgtatctgaaccattgacagatcaaagtaaagaaatacttatacaaaaaattagacctatttcaaaaaaaataggagataaaagcaacttacgatatattgaattaacaattattattcagcaagaaatggtaccgtggaatcatcctcccaaacaagaatttatttatggagaatggttacaagagctttatgaacaaggatacattcctcagaaggaattaaattcagatttaaccataatgctttaccaagcaaaacgaaaaaataaaagaatatacggaaattatgacttagaggaattactacctgatattccattttctgatgtgagaagagccattatggattcgtcagaggaacttatagataattatcaggatgatgaaaccaactctatattaactttatgccgcatgattttaactatggacacgggtaaaatcataccaaaagatattgcgggaaatgcagtggctgaatcttctccattagaacatagggagagaattttgttagcagttcgtagttatcttggagagaatattgaatggactaatgaaaatgtaaatcttacgataaactatcttaataaccgccttaagaaactttaacccgggcgcgttgctttcgatgaagt

SEQ ID NO: 91 - DNA, 27 bp, Artificial Sequence
  Primer For for dxs operon
taatcccggcaacagcttaatcagtac

SEQ ID NO: 92 - DNA, 20 bp, Artificial Sequence
  Primer Rev for dxs operon
acttcatcgaaagcaacgcg

SEQ ID NO: 93 - protein, 399 aa, Rhodococcus fascians
MAGTADLPLEMRRNGLNPTEELAQVRDRDGVIPVGELYGAPAFLVCRYEDVRRIFADSNRFSNAHTPMFAIPSGGDVIEDELAAMRAGNLIGLDPPDHTRLRHILAAEFSVHRLSRLQPRIAEIVDSALDGLEQAGQPADLMDRYALPVSLLVLCELLGVPYADRDELRDRTARLLDLSASAEQRAVAQREDRRYMATLVTRAQEQPGDDLLGILARKIGDNLSTDELISIISLIMLGGHETTASMIGLSVLALLHHPEQAAMMIEDPNCVNSGIEELLRWLSVAHSQPPRMAVTEVQIAGVTIPAGSFVIPSLLAANRDSNLTDRPDDLDITRGVAGHLAFGHGVHFCLGHSLARMTLRTAVPAVLRRFPDLALSPSHDVRLRSASIVLGLEELQLTW

SEQ ID NO: 94 - protein, 512 aa, Arabidopsis thaliana
MMVTLVLKYVLVIVMTLILRVLYDSICCYFLTPRRIKKFMERQGITGPKPRLLTGNIIDISKMLSHSASNDCSSIHHNIVPRLLPHYVSWSKQYGKRFIMWNGTEPRLCLTETEMIKELLTKHNPVTGKSWLQQQGTKGFIGRGLLMANGEAWHHQRHMAAPAFTRDRLKGYAKHMVECTKMMAERLRKEVGEEVEIGEEMRRLTADIISRTEFGSSCDKGKELFSLLTVLQRLCAQATRHLCFPGSRFLPSKYNREIKSLKTEVERLLMEIIDSRKDSVEIGRSSSYGDDLLGLLLNQMDSNKNNLNVQMIMDECKTFFFTGHETTSLLLTWTLMLLAHNPTWQDNVRDEVRQVCGQDGVPSVEQLSSLTSLNKVINESLRLYPPATLLPRMAFEDIKLGDLIIPKGLSIWIPVLAIHHSNELWGEDANEFNPERFTTRSFASSRHFMPFAAGPRNCIGQTFAMMEAKIILAMLVSKFSFAISENYRHAPIVVLTIKPKYGVQLVLKPLDL

SEQ ID NO: 95 - protein, 518 aa, Arabidopsis thaliana
MLLTILKSLLVIFVTTILRVLYDTISCYWLTPRRIKKIMEQQGVTGPKPRPLTGNILEISAMVSQSASKDCDSIHHDIVGRLLPHYVAWSKQYGKRFIVWNGTDPRLCLTETELIKELLMKHNGVSGRSWLQQQGTKNFIGRGLLMANGQDWHHQRHLAAPAFTGERLKGYARHMVECTSKLVERLRKEVGEGANEVEIGEEMHKLTADIISRTKFGSSFEKGKELFNHLTVLQRRCAQATRHLCFPGSRFLPSKYNREIKSLKKEVERLLIEIIQSRRDCAEMGRSSTHGDDLLGLLLNEMDIDKNNNNNNNNLQLIMDECKTFFFAGHETTALLLTWTTMLLADNPTWQEKVREEVREVFGRNGLPSVDQLSKLTSLSKVINESLRLYPPATLLPRMAFEDLKLGDLTIPKGLSIWIPVLAIHHSEELWGKDANQFNPERFGGRPFASGRHFIPFAAGPRNCIGQQFALMEAKIILATLISKFNFTISKNYRHAPIVVLTIKPKYGVQVILKPLVS

SEQ ID NO: 96 - DNA, 1377 bp, Artificial Sequence
  Fragment 0-yybN-P15
gaaaatgagctcccacataccggctaggtatgtggcgttcttcttcatgttctggcatgcagctgtgaggaggatttgtttgctcaccccctgaacctgcgatagctaagcccgtgcagttgttttgaacctacaaagcttcgttctattttttcttttctttttttagagatttcctgaaatagaaaagcaatcttttctaaccttttctttatggttttgccatacataccgagtaacgactttctggcgtttcgttgattttgtgtaatattctcaaacatatttcaggatttgatttatatgaccgatagtctttgcgatcaattgttgagtacgtaagtgtatgctgattcaacaaatgtatctgtattgttcactttataatgaaatttccattttggaagcaaacccctgactgggtgataacgtctatgtgcgatggcaccaaaaacttggaggtcaaatagtcctttagagatgagagttgttaagtagccggaatgtagagcgacggcttctacttgaaaatcaaatcatgcaatttagtgatccaatcggtcaaggtagggcttggaacagaatcatggacttttctggagttgcaaactatgaatcgtcctcattaaatttataataaaaatattctcaattgagcaaatcgcccaccctttttcctttataacataaaatccaatactatcaataaaattctttacaggttattacagttaagatataatatagttaatcttttacgtaaaaggaggtttttattacttgtgttgaagttttaaataggagtaactccatctccccattacggtcttcatttaatttcatcctgagttttatttagttagatataaaaacgaaaatttcacattatttttaactaaccgatagtataaaatatatattgaaaggattgtaaaattatgaataaattcctgaagagcaatttccgtttccttcttgcggctgctttaggcatatcattacttgctagtagtaattttataaaagccagcaatgataactataaattccatttacttgtaccttatggctatggtaatgcctattcaaatgatgacgggtgcgcatgatcgtatggttcactgtccaccaaccaaaactgtgctcagtaccgccaatatttctcccttggggggtacaaagaggtgtccctagaagagatccacgctgtgtaaaaattttacaaaaaggtattgactttccctacagggtgtgtaataatttaattacaggcgggggcaaccccgctcagtacctagagcgtaaaagaggggagggaaacactagttggcttcgttgggattctccggtgatgctgatttcgctgcgtatgctgcg

SEQ ID NO: 97 - DNA, 1489 bp, Artificial Sequence
  Fragment END-ZeoR-yybN-HOM-R
cggtagtcattgctggcgaacgcgatctagacgaagttatcttgatatggctttttatatgtgttactctacatacagaaaggaggaactaaatatggctaagttgaccagtgcagttcctgttttaacagctagagacgttgctggagcggttgaattctggacggaccgtcttggtttctcaagagacttcgtggaagacgatttcgcaggtgttgtcagagacgatgttacattattcatttcagcagttcaggatcaagttgtacctgacaacacacttgcttgggtttgggtgcgcggcctggacgaactgtacgcagaatggtctgaggtcgtgtcaacgaacttcagagacgcatctggtccagcaatgacagaaattggagagcaaccttggggtagagaatttgcacttagagatcctgcaggaaattgtgttcatttcgtagctgaagagcaagattgaagcttgaattcggatccgtcgactacgtatgccaatgatgatgaagagtcgacatggatgagcgatgatgatatcgcatttagacagaccactcatacagataatccttggaaagtaaatttacagaaatcggcagaaggtaaaggtacaatcatgacattttggttgattaacacagggaatagtaaaattccaaaagcttctaaaatacataacgtaaaacaaggaagcggagctcattattatcatgcaaatagccagggaagccatacatatgtagccttagcagttgaaaacaacaattacagtgcaagttcttatggtatagatggtgtatgggatgaagaaacttggtaaacaagaaccttctaaaaacgttggcaccttttagccacgcgaaaaaatttagaatgataatctttttataagattatcatttttatttattctattaagaattgagtgagaacatggaaagtcatatttatcgcattatcaaaaataaattaactataataatatttactataatcatattgattccttgtgttgatatatcactattgttaatgactaacacagaataccatcctgcatatgcgtttttcctttctggcacttccgtagggcatgcttcacaaatgatcctattatggtttcttccgctttattttttgttactatgtgctgacgattctatacaagattataaaacaggatatcattacattcttataagcaaagttggcagaaaaaaatattgtttggaaaaaatatttacctcttttatcatttcatttttgacaatgtttttgtcactaatattaaactttttattggttcaggttttcttttttaaaggtacatttaaaaatgacttagaccaaattaaattccctgataacagcctttatacttttagtatggcccatccttatatagcaattgtgttattttctattatttgttgtattatgtctggatttgttggagcacttggttcaagcctcagtttacttt

SEQ ID NO: 98 - DNA, 1404 bp, Artificial Sequence
  Fragment 25-solo-BS-purA
cgcagcatacgcagcgaaatcagcatcaccggagaatcccaacgaagccaactagtatgtcaagcgtagtagttgttggcacacagtggggcgacgagggcaagggcaagattacagatttcctgtctgaaaacgcggaggtgatcgcacgctatcagggaggaaacaacgccggccatacaatcaaattcgacggcattacatataaacttcaccttatcccgtctggtatcttttacaaagacaagacatgtgtcatcggaaatggaatggtcgtcgaccctaaagcgctggtcacagaactggcctatcttcacgaacgcaatgttagcacagacaatttacgcattagcaatagagcgcatgtaattctgccttatcatctgaagttagatgaagttgaggaggaacgcaagggtgctaataaaattggtacaacaaagaagggcatcggcccggcttatatggacaaagcggcccgcatcggtatccgcatcgcagatctgcttgacagagatgcgtttgctgagaaactggaaagaaacttagaggagaagaacagactgttagaaaaaatgtatgaaacggaaggatttaaattagaggatattctggacgagtactacgaatacggccagcaaatcaaaaaatatgtatgcgatacatctgtagtactgaacgatgcgctggatgagggaagaagagtattatttgaaggtgcccaaggagttatgctggatatcgatcaaggtacatatccatttgttacaagcagcaatcctgttgctggcggcgtcacaattggatctggcgtaggccctacgaaaattaaacacgtcgttggcgtgtcaaaagcatatacgacaagagttggtgatggaccgttcccgacagaactgaaagatgagattggcgatcaaattagagaggtcggaagagaatatggcacaacaacaggtagaccgagaagagttggttggttcgattcagtcgtggtgagacatgcaagaagagtgtctggcattacggatttatcacttaattcaattgatgttctggctggaatcgaaacgttaaaaatctgtgtggcatatagatataaaggagaaattattgaagaatttccagcctcacttaaagcactggccgaatgcgaacctgtgtacgaagaaatgccaggctggacagaagatattacaggagctaaatcactttcagaactgccggaaaatgcgagacattacctggaaagagtgtcacaactgacaggcattccgctgtcaattttttcagtgggcccggatagatcacaaacaaacgtgctgagatcagtgtacagagcaaattaaattaattctagagtcgatccccgggttcgccagcaatgactaccggcagcccgcc

SEQ ID NO: 99 - DNA, 20 bp, Artificial Sequence
  primer for Fragment 0-yybN-P15 amplification
ggagaatcccaacgaagcca

SEQ ID NO: 100 - DNA, 20 bp, Artificial Sequence
  primer for Fragment 0-yybN-P15 amplification
ggctaggtatgtggcgttct

SEQ ID NO: 101 - DNA, 20 bp, Artificial Sequence
  primer for END-ZeoR-yybN-HOM-R amplification
cggtagtcattgctggcgaa

SEQ ID NO: 102 - DNA, 20 bp, Artificial Sequence
  primer for END-ZeoR-yybN-HOM-R amplification
actgaggcttgaaccaagtg

SEQ ID NO: 103 - DNA, 20 bp, Artificial Sequence
  primer for Fragment 25-solo-BS-purA  amplificat ion
gcagcgaaatcagcatcacc

SEQ ID NO: 104 - DNA, 21 bp, Artificial Sequence
  primer for Fragment 25-solo-BS-purA  amplificat ion
cactgagcctccacctagcct

SEQ ID NO: 105 - DNA, 36 bp, Artificial Sequence
  primer for amplification of purA fragment and end fragment assembly
tttaactagtatgtcttcagtagttgtagtaggtac

SEQ ID NO: 106 - DNA, 20 bp, Artificial Sequence
  primer for final purA operon amplification
aagcccgtgcagttgttttg

SEQ ID NO: 107 - DNA, 24 bp, Artificial Sequence
  primer for final purA operon amplification
cacaattgctatataaggatgggc

SEQ ID NO: 108 - DNA, 4089 bp, Artificial Sequence
  complete purA operon
gaaaatgagctcccacataccggctaggtatgtggcgttcttcttcatgttctggcatgcagctgtgaggaggatttgtttgctcaccccctgaacctgcgatagctaagcccgtgcagttgttttgaacctacaaagcttcgttctattttttcttttctttttttagagatttcctgaaatagaaaagcaatcttttctaaccttttctttatggttttgccatacataccgagtaacgactttctggcgtttcgttgattttgtgtaatattctcaaacatatttcaggatttgatttatatgaccgatagtctttgcgatcaattgttgagtacgtaagtgtatgctgattcaacaaatgtatctgtattgttcactttataatgaaatttccattttggaagcaaacccctgactgggtgataacgtctatgtgcgatggcaccaaaaacttggaggtcaaatagtcctttagagatgagagttgttaagtagccggaatgtagagcgacggcttctacttgaaaatcaaatcatgcaatttagtgatccaatcggtcaaggtagggcttggaacagaatcatggacttttctggagttgcaaactatgaatcgtcctcattaaatttataataaaaatattctcaattgagcaaatcgcccaccctttttcctttataacataaaatccaatactatcaataaaattctttacaggttattacagttaagatataatatagttaatcttttacgtaaaaggaggtttttattacttgtgttgaagttttaaataggagtaactccatctccccattacggtcttcatttaatttcatcctgagttttatttagttagatataaaaacgaaaatttcacattatttttaactaaccgatagtataaaatatatattgaaaggattgtaaaattatgaataaattcctgaagagcaatttccgtttccttcttgcggctgctttaggcatatcattacttgctagtagtaattttataaaagccagcaatgataactataaattccatttacttgtaccttatggctatggtaatgcctattcaaatgatgacgggtgcgcatgatcgtatggttcactgtccaccaaccaaaactgtgctcagtaccgccaatatttctcccttggggggtacaaagaggtgtccctagaagagatccacgctgtgtaaaaattttacaaaaaggtattgactttccctacagggtgtgtaataatttaattacaggcgggggcaaccccgctcagtacctagagcgtaaaagaggggagggaaacactagtatgtcaagcgtagtagttgttggcacacagtggggcgacgagggcaagggcaagattacagatttcctgtctgaaaacgcggaggtgatcgcacgctatcagggaggaaacaacgccggccatacaatcaaattcgacggcattacatataaacttcaccttatcccgtctggtatcttttacaaagacaagacatgtgtcatcggaaatggaatggtcgtcgaccctaaagcgctggtcacagaactggcctatcttcacgaacgcaatgttagcacagacaatttacgcattagcaatagagcgcatgtaattctgccttatcatctgaagttagatgaagttgaggaggaacgcaagggtgctaataaaattggtacaacaaagaagggcatcggcccggcttatatggacaaagcggcccgcatcggtatccgcatcgcagatctgcttgacagagatgcgtttgctgagaaactggaaagaaacttagaggagaagaacagactgttagaaaaaatgtatgaaacggaaggatttaaattagaggatattctggacgagtactacgaatacggccagcaaatcaaaaaatatgtatgcgatacatctgtagtactgaacgatgcgctggatgagggaagaagagtattatttgaaggtgcccaaggagttatgctggatatcgatcaaggtacatatccatttgttacaagcagcaatcctgttgctggcggcgtcacaattggatctggcgtaggccctacgaaaattaaacacgtcgttggcgtgtcaaaagcatatacgacaagagttggtgatggaccgttcccgacagaactgaaagatgagattggcgatcaaattagagaggtcggaagagaatatggcacaacaacaggtagaccgagaagagttggttggttcgattcagtcgtggtgagacatgcaagaagagtgtctggcattacggatttatcacttaattcaattgatgttctggctggaatcgaaacgttaaaaatctgtgtggcatatagatataaaggagaaattattgaagaatttccagcctcacttaaagcactggccgaatgcgaacctgtgtacgaagaaatgccaggctggacagaagatattacaggagctaaatcactttcagaactgccggaaaatgcgagacattacctggaaagagtgtcacaactgacaggcattccgctgtcaattttttcagtgggcccggatagatcacaaacaaacgtgctgagatcagtgtacagagcaaattaaattaattctagacgaagttatcttgatatggctttttatatgtgttactctacatacagaaaggaggaactaaatatggctaagttgaccagtgcagttcctgttttaacagctagagacgttgctggagcggttgaattctggacggaccgtcttggtttctcaagagacttcgtggaagacgatttcgcaggtgttgtcagagacgatgttacattattcatttcagcagttcaggatcaagttgtacctgacaacacacttgcttgggtttgggtgcgcggcctggacgaactgtacgcagaatggtctgaggtcgtgtcaacgaacttcagagacgcatctggtccagcaatgacagaaattggagagcaaccttggggtagagaatttgcacttagagatcctgcaggaaattgtgttcatttcgtagctgaagagcaagattgaagcttgaattcggatccgtcgactacgtatgccaatgatgatgaagagtcgacatggatgagcgatgatgatatccatttagacagaccactcatacagataatccttggaaagtaaatttacagaaatcggcagaaggtaaaggtacaatcatgacattttggttgattaacacagggaatagtaaaattccaaaagcttctaaaatacataacgtaaaacaaggaagcggagctcattattatcatgcaaatagccagggaagccatacatatgtagccttagcagttgaaaacaacaattacagtgcaagttcttatggtatagatggtgtatgggatgaagaaacttggtaaacaagaaccttctaaaaacgttggcaccttttagccacgcgaaaaaatttagaatgataatctttttataagattatcatttttatttattctattaagaattgagtgagaacatggaaagtcatatttatcgcattatcaaaaataaattaactataataatatttactataatcatattgattccttgtgttgatatatcactattgttaatgactaacacagaataccatcctgcatatgcgtttttcctttctggcacttccgtagggcatgcttcacaaatgatcctattatggtttcttccgctttattttttgttactatgtgctgacgattctatacaagattataaaacaggatatcattacattcttataagcaaagttggcagaaaaaaatattgtttggaaaaaatatttacctcttttatcatttcatttttgacaatgtttttgtcactaatattaaactttttattggttcaggttttcttttttaaaggtacatttaaaaatgacttagaccaaattaaattccctgataacagcctttatacttttagtatggcccatccttatatagcaattgtgttattttctattatttgttgtattatgtctggatttgttggagcacttggttcaagcctcagtttacttt

SEQ ID NO: 109 - DNA, 2818 bp, Artificial Sequence
  operon 25-solo-BS-purA fragment + END-ZeoR-yybN -HOM-R fragment
cgcagcatacgcagcgaaatcagcatcaccggagaatcccaacgaagccaactagtatgtcaagcgtagtagttgttggcacacagtggggcgacgagggcaagggcaagattacagatttcctgtctgaaaacgcggaggtgatcgcacgctatcagggaggaaacaacgccggccatacaatcaaattcgacggcattacatataaacttcaccttatcccgtctggtatcttttacaaagacaagacatgtgtcatcggaaatggaatggtcgtcgaccctaaagcgctggtcacagaactggcctatcttcacgaacgcaatgttagcacagacaatttacgcattagcaatagagcgcatgtaattctgccttatcatctgaagttagatgaagttgaggaggaacgcaagggtgctaataaaattggtacaacaaagaagggcatcggcccggcttatatggacaaagcggcccgcatcggtatccgcatcgcagatctgcttgacagagatgcgtttgctgagaaactggaaagaaacttagaggagaagaacagactgttagaaaaaatgtatgaaacggaaggatttaaattagaggatattctggacgagtactacgaatacggccagcaaatcaaaaaatatgtatgcgatacatctgtagtactgaacgatgcgctggatgagggaagaagagtattatttgaaggtgcccaaggagttatgctggatatcgatcaaggtacatatccatttgttacaagcagcaatcctgttgctggcggcgtcacaattggatctggcgtaggccctacgaaaattaaacacgtcgttggcgtgtcaaaagcatatacgacaagagttggtgatggaccgttcccgacagaactgaaagatgagattggcgatcaaattagagaggtcggaagagaatatggcacaacaacaggtagaccgagaagagttggttggttcgattcagtcgtggtgagacatgcaagaagagtgtctggcattacggatttatcacttaattcaattgatgttctggctggaatcgaaacgttaaaaatctgtgtggcatatagatataaaggagaaattattgaagaatttccagcctcacttaaagcactggccgaatgcgaacctgtgtacgaagaaatgccaggctggacagaagatattacaggagctaaatcactttcagaactgccggaaaatgcgagacattacctggaaagagtgtcacaactgacaggcattccgctgtcaattttttcagtgggcccggatagatcacaaacaaacgtgctgagatcagtgtacagagcaaattaaattaattctagacgaagttatcttgatatggctttttatatgtgttactctacatacagaaaggaggaactaaatatggctaagttgaccagtgcagttcctgttttaacagctagagacgttgctggagcggttgaattctggacggaccgtcttggtttctcaagagacttcgtggaagacgatttcgcaggtgttgtcagagacgatgttacattattcatttcagcagttcaggatcaagttgtacctgacaacacacttgcttgggtttgggtgcgcggcctggacgaactgtacgcagaatggtctgaggtcgtgtcaacgaacttcagagacgcatctggtccagcaatgacagaaattggagagcaaccttggggtagagaatttgcacttagagatcctgcaggaaattgtgttcatttcgtagctgaagagcaagattgaagcttgaattcggatccgtcgactacgtatgccaatgatgatgaagagtcgacatggatgagcgatgatgatatccatttagacagaccactcatacagataatccttggaaagtaaatttacagaaatcggcagaaggtaaaggtacaatcatgacattttggttgattaacacagggaatagtaaaattccaaaagcttctaaaatacataacgtaaaacaaggaagcggagctcattattatcatgcaaatagccagggaagccatacatatgtagccttagcagttgaaaacaacaattacagtgcaagttcttatggtatagatggtgtatgggatgaagaaacttggtaaacaagaaccttctaaaaacgttggcaccttttagccacgcgaaaaaatttagaatgataatctttttataagattatcatttttatttattctattaagaattgagtgagaacatggaaagtcatatttatcgcattatcaaaaataaattaactataataatatttactataatcatattgattccttgtgttgatatatcactattgttaatgactaacacagaataccatcctgcatatgcgtttttcctttctggcacttccgtagggcatgcttcacaaatgatcctattatggtttcttccgctttattttttgttactatgtgctgacgattctatacaagattataaaacaggatatcattacattcttataagcaaagttggcagaaaaaaatattgtttggaaaaaatatttacctcttttatcatttcatttttgacaatgtttttgtcactaatattaaactttttattggttcaggttttcttttttaaaggtacatttaaaaatgacttagaccaaattaaattccctgataacagcctttatacttttagtatggcccatccttatatagcaattgtgttattttctattatttgttgtattatgtctggatttgttggagcacttggttcaagcctcagtttacttt

SEQ ID NO: 110 - DNA, 667 bp, Artificial Sequence
  Fragment-LOG2_Bsu
ttctttttgcgccaggtagccatagctggtcatatgatgaaaacaatctgtgtattcgctggcagcaaccctggcggtaatgaagcttacaagagaaaggccgctgaactgggtgtgtatatggcggaacagggaatcggattagtatacggtggctctcgcgtcggactgatgggcacgattgcagacgcaatcatggagaacggcggcacggcaattggagttatgccatctggattattttctggagaagttgtccatcagaatctgacagagctgattgaggtcaatggcatgcacgagagaaaagccaaaatgtcagagctggcggatggtttcatcagcatgcctggaggatttggaacatacgaggagctttttgaagttctttgctgggcgcaaattggcattcatcaaaaaccgattggcctgtataatgtgaacggctattttgaaccgatgatgaagatggtgaaatattcaattcaagaaggctttagcaatgaatcacatcttaaactgatccactcatcatcaagaccggatgaactgatcgaacaaatgcagaattattcatatccgattttagaaaaaaaatggacagaaatttaaattaattctagagtcgatccccgggttcgccagcaatgactaccggcagcccgcc

SEQ ID NO: 111 - DNA, 862 bp, Artificial Sequence
  Fragment-LOG3_Cgl
ttctttttgcgccaggtagccatagctggtcatatgatggcaccgaaacagacaccttctccagaaaaaaatagaaaccttgtgggtccagtgctgcaacgccgccaaacagagggcacgtttgaccagcgcctgcttgaaatgagagcagaccacaattggaagcacgcagacccgtggagagtgttaagaattcagtcagagtttgttgcaggatttgatgctctgcatgagatgccgaaggcggtcacagtgtttggaagcgcgagaattaaagaggatcatccttattataaagcaggtgttgagcttggagaaaaacttgttgccgctgattacgcggttgtaacgggcggaggccctggccttatggaagcaccgaataaaggagcgtcagaagccaatggcttatcagtcggtttaggtattgaacttcctcacgagcagcatttaaacccgtatgtggatctgggattaaattttagatacttttttgctagaaagacgatgtttctgaaatattcacaagcttttgtctgtctgccgggcggcttcggaacactggacgaactgttcgaagtgctgtgcatggtccaaacaggcaaagttacaaacttcccgattgtactgatcggcacagaattctgggctggcctggttgactggatcagacatagactggtcgaagaaggcatgattgatgaaaaagatgtagacagaatgctggtaacagatgatctggatcaagccgttaagttcatcgtcgatgcccatgccggcctggatgtagcgagaagacataactaaattaattctagagtcgatccccgggttcgccagcaatgactaccggcagcccgcc

SEQ ID NO: 112 - DNA, 679 bp, Artificial Sequence
  Fragment-LOG4_Pae
ttctttttgcgccaggtagccatagctggtcatatgatgacgcttagatcagtctgtgtgttctgcggcgcgagcccgggagcttctccggtttatcaagaagctgctgtggcccttggtagacatttagcggaacgcggactgacgttagtatatggcggaggtgccgtaggactgatgggtacagtggccgacgccgctctggcggcgggcggagaagttattggtattatccctcaatcacttcaggaggccgaaattggccataaaggcttaacaagattagaagttgtagatggcatgcatgcacgcaaggctagaatggctgagttagctgatgcctttatcgcgcttccgggcggacttggaacacttgaggaactgtttgaggtctggacatggggccagttaggctaccacgcaaaaccactgggcctgctggaagttaatggcttctatgatcctctgctgacatttctggaccacctggtcgatgaaagatttgtgagagcggaacatagaggcatgctgcagagaggagcgtcacctgaagcactgctggacgcactggcagcatggacaccgtcagtcgcaccgaaatgggttgatagaacaccacaataaattaattctagagtcgatccccgggttcgccagcaatgactaccggcagcccgcc

SEQ ID NO: 113 - DNA, 862 bp, Artificial Sequence
  Fragment-LOG5_Rfa
ttctttttgcgccaggtagccatagctggtcatatgatgaacagcgttgacgaccctggttcatacgaacagattggcagcgagaagccggcaaagcataaaggctctgttgtactgcgcagaggcagaaatacggaaacatctacgtcagaccaacgcttattagatgaaagaggttcaggtaattggatccattctgatacgtggcgcgtcttacgcattcagagcgaatttgtggaaggctttggtgcgctggccgaagttcctagagctgtcacagtattcggcagcgccagaacgccagtcgatcacccggaatacgagagagctagagagttaggagccgcgcttgctagagaaggatttgcggtaattacaggcggtggaccaggcgtcatggagggagctaacagaggagcgtcagaggcggatggctattcaattggccttggcatcgagctgccgttcgaacaaggcttaaacgactgggtagatcttggagtgaatttcagatatttttttgtaagaaaaacaatgttcgtgaaatattcacaggcctttgtgtgccttccgggcggctttggaacactggacgaactgtttgaagcactgacacttgttcaaacagcaaaagtcacaagatttccgattgttctgctgggaaaggacttctggtcaggactgctggattggattaaaacaacactggttaaagagggcaaagtctcagaaaaagatctggatctgatccattgtacagattcagtggatgaagcagtgagaatcgttagagaagcacaacagtaaattaattctagagtcgatccccgggttcgccagcaatgactaccggcagcccgcc

SEQ ID NO: 114 - DNA, 688 bp, Artificial Sequence
  Fragment-LOG6_Rfa
ttctttttgcgccaggtagccatagctggtcatatgatgaatttacgcccgatgcctgccacgacggttagcgcccaagcaagaccaacgccaaagtcagttacggtgttctgcggagctatgccgggtagaggaacaaaatacggtcaacttgctgagggcatgggtagagctattgctagaagcaaattaagacttgtatatggaggagcgagagtcggccttatgggaacactggcaaacgcagccttagactctggcggtacagtcgttggagtcattcctgaatcatttacagctatccctgaagccgcccaccacggactgacagaactgcatgtagtgcacgatatgcatcaaagaaaagccttaatggctgagctgggcgatgcattcattgcacttcctggcggcgtcggcacagcggaggagttttttgaggtgctgacatggagccatctgggcctgcataacaaaccgtgcgttctgctgaatgataatgaatactatagaccgctgctgtcatatattgaacatgcagcggtcgaaggctttatcacacctgcgacaagatcaagagtaatcgtgtgtaaagacattgaaggcgcgattgcggcgattagatcaccgtaaattaattctagagtcgatccccgggttcgccagcaatgactaccggcagcccgcc

SEQ ID NO: 115 - DNA, 655 bp, Artificial Sequence
  Fragment-LOG7_Cst
ttctttttgcgccaggtagccatagctggtcatatgatgaaatcagtagcgatttattgcggatcatcaatgggatctagccctagatatgcagaagtagctaaagagatgggagaggaacttgcaagacgcaatctgagagtagtgtatggaggaggaaacgtcggactgatgggtacagtggccgacgcagcgttagcggccggtggcgaggtggtaggcgtaattccaagacagttaattaaccgcgagatggcacatgccggtctgacagatttagaagtggtggacacaatggcgcagagaaaaacgagaatggaggaacttgccgatgcttttgtgtgtctgcctggcggtgttggcacgctggaagaagtcgttgaagttcttacaatgcaacaacttggccacgtccaaggcccggtcggcctggttaatgttgacggcttttgggaaccgtttgtcagaacactggaatcaatggtcgcttgcggcttcgttcagtcaagatacatcgatgctatcgcactgtcttcagatccggctgaaattctggatgaattcgcaggctggagcccgatcggcgcgaagtggagctaaattaattctagagtcgatccccgggttcgccagcaatgactaccggcagcccgcc

SEQ ID NO: 116 - DNA, 919 bp, Artificial Sequence
  Fragment-LOG8_Cst
ttctttttgcgccaggtagccatagctggtcatatgatggcccctcttcgctcaaacaaagatggcaaacctagagttcctagaacgacggaaagcgccgaaacgaatagaatgttaagaggtccgatgttactgagaacagacggaaatcagacgtcaacatacgatcaaagacttttagagtcaggcgcagatcacgcttggaagcatgcagatccgtggcgcgttctgagaatccagggagagttcgtggcaggatttgatgctctgagcgatctgcctaaggcggttacggtatacggctcagccagaacaaaagaggacacaccggaatatgcacagggcatcgatttaggtgcaaaattagttgagcatgactacgctgttgtgacaggcggtggcccaggcatcatggaggcggcgaatagaggcgcctatgaagctggaggtctttctgtgggcctgggcattgaacttccgtttgaacaaggtttaaacaaatatgtagatcttggaattaactttagatactttttcgcgagaaagacaatgttcctgaaatattcacaggcgtttgtgtgcctgccgggcggtatgggaacaatggatgagctgtttgaaatgctgtgtatggtgcaaacaggaaaagtgacaaactatccgattgtactgatcggcacagaatattggagcggcctggtagaatggatggctaatacactggtcgccgaaggctacatcaatccacaagatatggatctgttcctgctgacagacagcgtcgacgaagccgtcgaacatattgtcgcggctcataaagaaatgacagacgaaagagtctcaagactgaatcacgacgcataaattaattctagagtcgatccccgggttcgccagcaatgactaccggcagcccgcc

SEQ ID NO: 117 - DNA, 850 bp, Artificial Sequence
  Fragment-LOG9_SC
ttctttttgcgccaggtagccatagctggtcatatgatggccacaggcaacccggagggaaaaaaacgccctcctgaggaacaacgccttggccctgttcttagaagacgcggacaagttcaggagtctacaacagaccaaagactgcttgatgagagagccccgacagattgggtgcatacagatccatggagagttttaagaattcagtcagagtttattgagggatttggtacactggcggaactgccgccggccatttctgtttttggatcagcgagaacgccggccgactcaccggaatatgacgctggcgtcagactgggaagaggtctggtggaagcgggattcgccgtgatcacgggcggcggtccgggcgcaatggaagctgctaacaagggtgcgttagaagcgaaaggtacaagcgtcggtttaggaattgaacttccattcgaacaaggattaaatccttacgttgatatcggacttaatttcagatatttcttcgtaagaaagatgatgtttgtaaagtacgcacaaggatttgtagtgttaccaggcggcttaggcacacttgatgaactgtttgaagcactgacactggtacagacgcagaaagtcacgagatttccgatcgtcctgtttggcagcgaatattggggcggcctggtcgattggctgagaggcacactggttgctcagggcaaagcggctgaaaaagatctgatgctgtttcacgtcacagacgacgtagatgaagcagtggcactggtgtcaaaagaagcaggcagataaattaattctagagtcgatccccgggttcgccagcaatgactaccggcagcccgcc

SEQ ID NO: 118 - DNA, 739 bp, Artificial Sequence
  Fragment-LOG10_ATh
ttctttttgcgccaggtagccatagctggtcatatgatggagattaagggagagagcatgcaaaagtcaaagttcagacgcatttgtgtgttttgcggttcatcacagggcaaaaagtcttcatatcaagatgctgcagttgatctgggtaacgaattagtaagcagaaacatcgatcttgtctacggcggtggctctattggacttatgggcttagtgtctcaagcagtccatgacggcggtagacatgtcatcggcattattcctaaaacgttaatgccgcgcgagctgacaggcgaaacagtaggagaggtaagagcggtggccgacatgcatcaaagaaaagcggaaatggccaaacacagcgacgcatttattgctttaccgggcggctacggaacactggaagaacttcttgaggtcatcacgtgggcccagctgggcattcatgataaaccagtgggactgctgaatgtggatggctattataatagcctgctgtcatttattgataaagcggttgaagaaggattcatttcaccgacggcaagagaaatcattgtttcagcgcctacagctaaagaactggttaaaaaactggaagagtatgcgccgtgccatgaaagagttgctacaaaactgtgttgggaaatggagagaatcggatattcatcagaagaataaattaattctagagtcgatccccgggttcgccagcaatgactaccggcagcccgcc

SEQ ID NO: 119 - DNA, 1012 bp, Artificial Sequence
  Fragment-LOG11_ATh
ttctttttgcgccaggtagccatagctggtcatatgatgggtgtctcaatgctggattcttcttggtcatttcgcctttcaagcaagatgtctccaagcccgacgcaaaaattagccagctctctgacagaacgcgatccaatcagcttaaatctgcgcttcgaccattcaattagagcatatagcgcaagagctcttaagtgcagatctgtcaaatctgaagatttcgttgaaagatcatcaccatttgaagttaagaaggagctggaaatttgctttgatcttgtccatagattaggtagaggcattgtttaccttggcagcgctagaattccgccgaaccacagccattacctgcaggcacaggaattatcaagagaagcggcaacgttacttgactgcacaacgtggtcaggcgctggacctggccttatggatgcggtgacggaaggtgcccttgaggcggaaaagccggtaggtggcatcaaaattgaaaaagaagcgggcgagtggacggcgtcaaaattccacccgtatttacctccgcaaaattatcatacatgtagatttttctcagcgagaaaacatggccttgtcgatgccgtaattagaaataatgtttcagaaaaaacagctatcattgcactgccgggcggcattggaacattagatgagatgttcgaaatcctggctctgattcaactggagagaattggctcagctctgccggtgccttttatcgtaatgaactacgacgccttttattcaaaactgctggaatttatcgagacatgtgaaaatctgggaacagtttcaaaaggagaagtgtcagcactgtggaaagtgtgtaataacaactttgaggccctgacatatctggcggaattttatgacctgcctgatggatcagcaaaactggagacagaactgagaaaaacaacacctacaagaggataaattaattctagagtcgatccccgggttcgccagcaatgactaccggcagcccgcc

SEQ ID NO: 120 - DNA, 20 bp, Artificial Sequence
  primer for AmyE 0 + IPT1 amplification
gcggtagtggtgcttacgat

SEQ ID NO: 121 - DNA, 20 bp, Artificial Sequence
  primer for AmyE 0 + IPT1 amplification
gactcgttagccaggtcgtc

SEQ ID NO: 122 - DNA, 20 bp, Artificial Sequence
  primer for Amy 0 + IPT1 + LOG2-11  amplificatio n
cggtagtcattgctggcgaa

SEQ ID NO: 123 - DNA, 20 bp, Artificial Sequence
  primer for Amy 0 + IPT1 + LOG2-11  amplificatio n
tacgatgtacgacaggggga

SEQ ID NO: 124 - DNA, 25 bp, Artificial Sequence
  primer for erythromycin resistance cassette amp lification
aggatccatctacattccctttagt

SEQ ID NO: 125 - DNA, 20 bp, Artificial Sequence
  primer for erythromycin resistance cassette amp lification
cgcgtaacggttttcactgg

SEQ ID NO: 126 - DNA, 32 bp, Artificial Sequence; identical to SEQ ID NO: 77
  primer for EryR + AmyE END operon amplification

SEQ ID NO: 127 - DNA, 20 bp, Artificial Sequence
  primer for EryR + AmyE END operon amplification
caatggggaagagaaccgct

SEQ ID NO: 128 - DNA, 25 bp, Artificial Sequence
  primer for final operon amplification
atctacattccctttagtaacgtgt

SEQ ID NO: 129 - DNA, 2355 bp, Artificial Sequence
  operon AmyE 0 + IPT1
gcggtagtggtgcttacgatgtacgacagggggattccccatacattcttcgcttggctgaaaatgattcttctttttatcgtctgcggcggcgttctgtttctgcttcggtatgtgattgtgaagctggcttacagaagagcggtaaaagaagaaataaaaaagaaatcatcttttttgtttggaaagcgagggaagcgttcacagtttcgggcagctttttttataggaacattgatttgtattcactctgccaagttgttttgatagagtgattgtgataattttaaatgtaagcgttaacaaaattctccagtcttcacatcggtttgaaaggaggaagcggaagaatgaagtaagagggatttttgactccgaagtaagtcttcaaaaaatcaaataaggagtgtcaagaatgtttgcaaaacgattcaaaacctctttactgccgttattcgctggatttttattgctgtttcatttggttctggcaggaccggcggctgcgagtgctgaaacggcgaacaaatcgaatgagcttacagcaccgtcgatcaaaagcggaaccattcttcatgcatggaattggtcgttcaatacgttaaaacacaatatgaaggatattcatgatgcaggatatacagccattcagacatctccgattaaccaagtaaaggaagggaatcaaggagataaaagcatgtcgaactggtactggctgtatcagccgacatcgtatcaaattggcaaccgttacttaggtactgaacaagaatttaaagaaatgtgtgcagccgctgaagaatatggcataaaggtcattgttgacgcggtcatcaatcataccaccagtgattatgccgcgatttccaatgaggttaagagtattccaaactggacacatggaaacacacaaattaaaaactggtctgatcgatgggatgtcacgcagaattcattgctcgggctgtatgactggaatacacaaaatacacaagtacagtcctatctgaaacggttcttagacagggcattgaatgacggggcagacggttttcgatttgatgccgccaaacatatagagcttccagatgatggcagttacggcagtcaattttggccgaatatcacaaatacatctgcagagttccaatacggagaaatcctgcaggatagtgcctccagagatgctgcttatgcgaattatatggatgtgacagcgtctaactatgggcattccataaggtccgctttaaagaatcgtaatctgggcgtgtcgaatatctcccactatgcatctgatgtgtctgcggacaagctagtgacatgggtagagtcgcatgatacacgggtgcgcatgatcgtatggttcactgtccaccaaccaaaactgtgctcagtaccgccaatatttctcccttggggggtacaaagaggtgtccctagaagagatccacgctgtgtaaaaattttacaaaaaggtattgactttccctacagggtgtgtaataatttaattacaggcgggggcaaccccgctcagtacctagagcgtaaaagaggggagggaaacactagtatgttattacacctgatctacggtcctacatgtagtggtaaaactgatatggctatacaaatcgcgcaagaaacaggttggcccgtcgttgctcttgatcgtgtgcagtgctgtccacagattgcgacaggatccggcagacctctggaaagcgagttacaatctacccggcggatttatttagactcacgccctttgacagaaggcattcttgacgcggaatccgctcatcgccgtcttatattcgaggtagattggaggaaatcggaagaaggcttaatcctcgaaggagggtccatctcgcttctaaattgtatggctaagtctccattttggagatctggattccagtggcacgtcaagcgcctgagacttggggattcagatgcgtttctgacgcgagcaaaacagcgtgtggctgagatgtttgctattagagaagatcggccaagtttattggaggagctcgccgagctgtggaattacccggcagctcgcccgattttggaagacattgacggctatcgatgtgccattagatttgcaaggaaacacgatctcgccatatcacagctgccgaacatcgatgccggccggcatgttgaactgatcgaagccattgcaaacgaatatcttgaacatgcgctaagccaagaacgcgactttcctcaatggccggaggacggcgcaggacaaccagtatgcccggttaccttaacgagaatccgttgaaaaagaggggagggaaacattaatgacgacctggctaacgagtc

SEQ ID NO: 130 - DNA, 2966 bp, Artificial Sequence
  operon AmyE 0 + IPT1 + LOG2
gcggtagtggtgcttacgatgtacgacagggggattccccatacattcttcgcttggctgaaaatgattcttctttttatcgtctgcggcggcgttctgtttctgcttcggtatgtgattgtgaagctggcttacagaagagcggtaaaagaagaaataaaaaagaaatcatcttttttgtttggaaagcgagggaagcgttcacagtttcgggcagctttttttataggaacattgatttgtattcactctgccaagttgttttgatagagtgattgtgataattttaaatgtaagcgttaacaaaattctccagtcttcacatcggtttgaaaggaggaagcggaagaatgaagtaagagggatttttgactccgaagtaagtcttcaaaaaatcaaataaggagtgtcaagaatgtttgcaaaacgattcaaaacctctttactgccgttattcgctggatttttattgctgtttcatttggttctggcaggaccggcggctgcgagtgctgaaacggcgaacaaatcgaatgagcttacagcaccgtcgatcaaaagcggaaccattcttcatgcatggaattggtcgttcaatacgttaaaacacaatatgaaggatattcatgatgcaggatatacagccattcagacatctccgattaaccaagtaaaggaagggaatcaaggagataaaagcatgtcgaactggtactggctgtatcagccgacatcgtatcaaattggcaaccgttacttaggtactgaacaagaatttaaagaaatgtgtgcagccgctgaagaatatggcataaaggtcattgttgacgcggtcatcaatcataccaccagtgattatgccgcgatttccaatgaggttaagagtattccaaactggacacatggaaacacacaaattaaaaactggtctgatcgatgggatgtcacgcagaattcattgctcgggctgtatgactggaatacacaaaatacacaagtacagtcctatctgaaacggttcttagacagggcattgaatgacggggcagacggttttcgatttgatgccgccaaacatatagagcttccagatgatggcagttacggcagtcaattttggccgaatatcacaaatacatctgcagagttccaatacggagaaatcctgcaggatagtgcctccagagatgctgcttatgcgaattatatggatgtgacagcgtctaactatgggcattccataaggtccgctttaaagaatcgtaatctgggcgtgtcgaatatctcccactatgcatctgatgtgtctgcggacaagctagtgacatgggtagagtcgcatgatacacgggtgcgcatgatcgtatggttcactgtccaccaaccaaaactgtgctcagtaccgccaatatttctcccttggggggtacaaagaggtgtccctagaagagatccacgctgtgtaaaaattttacaaaaaggtattgactttccctacagggtgtgtaataatttaattacaggcgggggcaaccccgctcagtacctagagcgtaaaagaggggagggaaacactagtatgttattacacctgatctacggtcctacatgtagtggtaaaactgatatggctatacaaatcgcgcaagaaacaggttggcccgtcgttgctcttgatcgtgtgcagtgctgtccacagattgcgacaggatccggcagacctctggaaagcgagttacaatctacccggcggatttatttagactcacgccctttgacagaaggcattcttgacgcggaatccgctcatcgccgtcttatattcgaggtagattggaggaaatcggaagaaggcttaatcctcgaaggagggtccatctcgcttctaaattgtatggctaagtctccattttggagatctggattccagtggcacgtcaagcgcctgagacttggggattcagatgcgtttctgacgcgagcaaaacagcgtgtggctgagatgtttgctattagagaagatcggccaagtttattggaggagctcgccgagctgtggaattacccggcagctcgcccgattttggaagacattgacggctatcgatgtgccattagatttgcaaggaaacacgatctcgccatatcacagctgccgaacatcgatgccggccggcatgttgaactgatcgaagccattgcaaacgaatatcttgaacatgcgctaagccaagaacgcgactttcctcaatggccggaggacggcgcaggacaaccagtatgcccggttaccttaacgagaatccgttaaaaaagaggggagggaaacattatgatgaaaacaatctgtgtattcgctggcagcaaccctggcggtaatgaagcttacaagagaaaggccgctgaactgggtgtgtatatggcggaacagggaatcggattagtatacggtggctctcgcgtcggactgatgggcacgattgcagacgcaatcatggagaacggcggcacggcaattggagttatgccatctggattattttctggagaagttgtccatcagaatctgacagagctgattgaggtcaatggcatgcacgagagaaaagccaaaatgtcagagctggcggatggtttcatcagcatgcctggaggatttggaacatacgaggagctttttgaagttctttgctgggcgcaaattggcattcatcaaaaaccgattggcctgtataatgtgaacggctattttgaaccgatgatgaagatggtgaaatattcaattcaagaaggctttagcaatgaatcacatcttaaactgatccactcatcatcaagaccggatgaactgatcgaacaaatgcagaattattcatatccgattttagaaaaaaaatggacagaaatttaaattaattctagagtcgatccccgggttcgccagcaatgactaccggcagcccgcc

SEQ ID NO: 131 - DNA, 3161 bp, Artificial Sequence
  operon AmyE 0 + IPT1 + LOG3
gcggtagtggtgcttacgatgtacgacagggggattccccatacattcttcgcttggctgaaaatgattcttctttttatcgtctgcggcggcgttctgtttctgcttcggtatgtgattgtgaagctggcttacagaagagcggtaaaagaagaaataaaaaagaaatcatcttttttgtttggaaagcgagggaagcgttcacagtttcgggcagctttttttataggaacattgatttgtattcactctgccaagttgttttgatagagtgattgtgataattttaaatgtaagcgttaacaaaattctccagtcttcacatcggtttgaaaggaggaagcggaagaatgaagtaagagggatttttgactccgaagtaagtcttcaaaaaatcaaataaggagtgtcaagaatgtttgcaaaacgattcaaaacctctttactgccgttattcgctggatttttattgctgtttcatttggttctggcaggaccggcggctgcgagtgctgaaacggcgaacaaatcgaatgagcttacagcaccgtcgatcaaaagcggaaccattcttcatgcatggaattggtcgttcaatacgttaaaacacaatatgaaggatattcatgatgcaggatatacagccattcagacatctccgattaaccaagtaaaggaagggaatcaaggagataaaagcatgtcgaactggtactggctgtatcagccgacatcgtatcaaattggcaaccgttacttaggtactgaacaagaatttaaagaaatgtgtgcagccgctgaagaatatggcataaaggtcattgttgacgcggtcatcaatcataccaccagtgattatgccgcgatttccaatgaggttaagagtattccaaactggacacatggaaacacacaaattaaaaactggtctgatcgatgggatgtcacgcagaattcattgctcgggctgtatgactggaatacacaaaatacacaagtacagtcctatctgaaacggttcttagacagggcattgaatgacggggcagacggttttcgatttgatgccgccaaacatatagagcttccagatgatggcagttacggcagtcaattttggccgaatatcacaaatacatctgcagagttccaatacggagaaatcctgcaggatagtgcctccagagatgctgcttatgcgaattatatggatgtgacagcgtctaactatgggcattccataaggtccgctttaaagaatcgtaatctgggcgtgtcgaatatctcccactatgcatctgatgtgtctgcggacaagctagtgacatgggtagagtcgcatgatacacgggtgcgcatgatcgtatggttcactgtccaccaaccaaaactgtgctcagtaccgccaatatttctcccttggggggtacaaagaggtgtccctagaagagatccacgctgtgtaaaaattttacaaaaaggtattgactttccctacagggtgtgtaataatttaattacaggcgggggcaaccccgctcagtacctagagcgtaaaagaggggagggaaacactagtatgttattacacctgatctacggtcctacatgtagtggtaaaactgatatggctatacaaatcgcgcaagaaacaggttggcccgtcgttgctcttgatcgtgtgcagtgctgtccacagattgcgacaggatccggcagacctctggaaagcgagttacaatctacccggcggatttatttagactcacgccctttgacagaaggcattcttgacgcggaatccgctcatcgccgtcttatattcgaggtagattggaggaaatcggaagaaggcttaatcctcgaaggagggtccatctcgcttctaaattgtatggctaagtctccattttggagatctggattccagtggcacgtcaagcgcctgagacttggggattcagatgcgtttctgacgcgagcaaaacagcgtgtggctgagatgtttgctattagagaagatcggccaagtttattggaggagctcgccgagctgtggaattacccggcagctcgcccgattttggaagacattgacggctatcgatgtgccattagatttgcaaggaaacacgatctcgccatatcacagctgccgaacatcgatgccggccggcatgttgaactgatcgaagccattgcaaacgaatatcttgaacatgcgctaagccaagaacgcgactttcctcaatggccggaggacggcgcaggacaaccagtatgcccggttaccttaacgagaatccgttaaaaaagaggggagggaaacattatgatggcaccgaaacagacaccttctccagaaaaaaatagaaaccttgtgggtccagtgctgcaacgccgccaaacagagggcacgtttgaccagcgcctgcttgaaatgagagcagaccacaattggaagcacgcagacccgtggagagtgttaagaattcagtcagagtttgttgcaggatttgatgctctgcatgagatgccgaaggcggtcacagtgtttggaagcgcgagaattaaagaggatcatccttattataaagcaggtgttgagcttggagaaaaacttgttgccgctgattacgcggttgtaacgggcggaggccctggccttatggaagcaccgaataaaggagcgtcagaagccaatggcttatcagtcggtttaggtattgaacttcctcacgagcagcatttaaacccgtatgtggatctgggattaaattttagatacttttttgctagaaagacgatgtttctgaaatattcacaagcttttgtctgtctgccgggcggcttcggaacactggacgaactgttcgaagtgctgtgcatggtccaaacaggcaaagttacaaacttcccgattgtactgatcggcacagaattctgggctggcctggttgactggatcagacatagactggtcgaagaaggcatgattgatgaaaaagatgtagacagaatgctggtaacagatgatctggatcaagccgttaagttcatcgtcgatgcccatgccggcctggatgtagcgagaagacataactaaattaattctagagtcgatccccgggttcgccagcaatgactaccggcagcccgcc

SEQ ID NO: 132 - DNA, 2978 bp, Artificial Sequence
  operon AmyE 0 + IPT1 + LOG4
gcggtagtggtgcttacgatgtacgacagggggattccccatacattcttcgcttggctgaaaatgattcttctttttatcgtctgcggcggcgttctgtttctgcttcggtatgtgattgtgaagctggcttacagaagagcggtaaaagaagaaataaaaaagaaatcatcttttttgtttggaaagcgagggaagcgttcacagtttcgggcagctttttttataggaacattgatttgtattcactctgccaagttgttttgatagagtgattgtgataattttaaatgtaagcgttaacaaaattctccagtcttcacatcggtttgaaaggaggaagcggaagaatgaagtaagagggatttttgactccgaagtaagtcttcaaaaaatcaaataaggagtgtcaagaatgtttgcaaaacgattcaaaacctctttactgccgttattcgctggatttttattgctgtttcatttggttctggcaggaccggcggctgcgagtgctgaaacggcgaacaaatcgaatgagcttacagcaccgtcgatcaaaagcggaaccattcttcatgcatggaattggtcgttcaatacgttaaaacacaatatgaaggatattcatgatgcaggatatacagccattcagacatctccgattaaccaagtaaaggaagggaatcaaggagataaaagcatgtcgaactggtactggctgtatcagccgacatcgtatcaaattggcaaccgttacttaggtactgaacaagaatttaaagaaatgtgtgcagccgctgaagaatatggcataaaggtcattgttgacgcggtcatcaatcataccaccagtgattatgccgcgatttccaatgaggttaagagtattccaaactggacacatggaaacacacaaattaaaaactggtctgatcgatgggatgtcacgcagaattcattgctcgggctgtatgactggaatacacaaaatacacaagtacagtcctatctgaaacggttcttagacagggcattgaatgacggggcagacggttttcgatttgatgccgccaaacatatagagcttccagatgatggcagttacggcagtcaattttggccgaatatcacaaatacatctgcagagttccaatacggagaaatcctgcaggatagtgcctccagagatgctgcttatgcgaattatatggatgtgacagcgtctaactatgggcattccataaggtccgctttaaagaatcgtaatctgggcgtgtcgaatatctcccactatgcatctgatgtgtctgcggacaagctagtgacatgggtagagtcgcatgatacacgggtgcgcatgatcgtatggttcactgtccaccaaccaaaactgtgctcagtaccgccaatatttctcccttggggggtacaaagaggtgtccctagaagagatccacgctgtgtaaaaattttacaaaaaggtattgactttccctacagggtgtgtaataatttaattacaggcgggggcaaccccgctcagtacctagagcgtaaaagaggggagggaaacactagtatgttattacacctgatctacggtcctacatgtagtggtaaaactgatatggctatacaaatcgcgcaagaaacaggttggcccgtcgttgctcttgatcgtgtgcagtgctgtccacagattgcgacaggatccggcagacctctggaaagcgagttacaatctacccggcggatttatttagactcacgccctttgacagaaggcattcttgacgcggaatccgctcatcgccgtcttatattcgaggtagattggaggaaatcggaagaaggcttaatcctcgaaggagggtccatctcgcttctaaattgtatggctaagtctccattttggagatctggattccagtggcacgtcaagcgcctgagacttggggattcagatgcgtttctgacgcgagcaaaacagcgtgtggctgagatgtttgctattagagaagatcggccaagtttattggaggagctcgccgagctgtggaattacccggcagctcgcccgattttggaagacattgacggctatcgatgtgccattagatttgcaaggaaacacgatctcgccatatcacagctgccgaacatcgatgccggccggcatgttgaactgatcgaagccattgcaaacgaatatcttgaacatgcgctaagccaagaacgcgactttcctcaatggccggaggacggcgcaggacaaccagtatgcccggttaccttaacgagaatccgttaaaaaagaggggagggaaacattatgatgacgcttagatcagtctgtgtgttctgcggcgcgagcccgggagcttctccggtttatcaagaagctgctgtggcccttggtagacatttagcggaacgcggactgacgttagtatatggcggaggtgccgtaggactgatgggtacagtggccgacgccgctctggcggcgggcggagaagttattggtattatccctcaatcacttcaggaggccgaaattggccataaaggcttaacaagattagaagttgtagatggcatgcatgcacgcaaggctagaatggctgagttagctgatgcctttatcgcgcttccgggcggacttggaacacttgaggaactgtttgaggtctggacatggggccagttaggctaccacgcaaaaccactgggcctgctggaagttaatggcttctatgatcctctgctgacatttctggaccacctggtcgatgaaagatttgtgagagcggaacatagaggcatgctgcagagaggagcgtcacctgaagcactgctggacgcactggcagcatggacaccgtcagtcgcaccgaaatgggttgatagaacaccacaataaattaattctagagtcgatccccgggttcgccagcaatgactaccggcagcccgcc

SEQ ID NO: 133 - DNA, 3161 bp, Artificial Sequence
  operon AmyE 0 + IPT1 + LOG5
gcggtagtggtgcttacgatgtacgacagggggattccccatacattcttcgcttggctgaaaatgattcttctttttatcgtctgcggcggcgttctgtttctgcttcggtatgtgattgtgaagctggcttacagaagagcggtaaaagaagaaataaaaaagaaatcatcttttttgtttggaaagcgagggaagcgttcacagtttcgggcagctttttttataggaacattgatttgtattcactctgccaagttgttttgatagagtgattgtgataattttaaatgtaagcgttaacaaaattctccagtcttcacatcggtttgaaaggaggaagcggaagaatgaagtaagagggatttttgactccgaagtaagtcttcaaaaaatcaaataaggagtgtcaagaatgtttgcaaaacgattcaaaacctctttactgccgttattcgctggatttttattgctgtttcatttggttctggcaggaccggcggctgcgagtgctgaaacggcgaacaaatcgaatgagcttacagcaccgtcgatcaaaagcggaaccattcttcatgcatggaattggtcgttcaatacgttaaaacacaatatgaaggatattcatgatgcaggatatacagccattcagacatctccgattaaccaagtaaaggaagggaatcaaggagataaaagcatgtcgaactggtactggctgtatcagccgacatcgtatcaaattggcaaccgttacttaggtactgaacaagaatttaaagaaatgtgtgcagccgctgaagaatatggcataaaggtcattgttgacgcggtcatcaatcataccaccagtgattatgccgcgatttccaatgaggttaagagtattccaaactggacacatggaaacacacaaattaaaaactggtctgatcgatgggatgtcacgcagaattcattgctcgggctgtatgactggaatacacaaaatacacaagtacagtcctatctgaaacggttcttagacagggcattgaatgacggggcagacggttttcgatttgatgccgccaaacatatagagcttccagatgatggcagttacggcagtcaattttggccgaatatcacaaatacatctgcagagttccaatacggagaaatcctgcaggatagtgcctccagagatgctgcttatgcgaattatatggatgtgacagcgtctaactatgggcattccataaggtccgctttaaagaatcgtaatctgggcgtgtcgaatatctcccactatgcatctgatgtgtctgcggacaagctagtgacatgggtagagtcgcatgatacacgggtgcgcatgatcgtatggttcactgtccaccaaccaaaactgtgctcagtaccgccaatatttctcccttggggggtacaaagaggtgtccctagaagagatccacgctgtgtaaaaattttacaaaaaggtattgactttccctacagggtgtgtaataatttaattacaggcgggggcaaccccgctcagtacctagagcgtaaaagaggggagggaaacactagtatgttattacacctgatctacggtcctacatgtagtggtaaaactgatatggctatacaaatcgcgcaagaaacaggttggcccgtcgttgctcttgatcgtgtgcagtgctgtccacagattgcgacaggatccggcagacctctggaaagcgagttacaatctacccggcggatttatttagactcacgccctttgacagaaggcattcttgacgcggaatccgctcatcgccgtcttatattcgaggtagattggaggaaatcggaagaaggcttaatcctcgaaggagggtccatctcgcttctaaattgtatggctaagtctccattttggagatctggattccagtggcacgtcaagcgcctgagacttggggattcagatgcgtttctgacgcgagcaaaacagcgtgtggctgagatgtttgctattagagaagatcggccaagtttattggaggagctcgccgagctgtggaattacccggcagctcgcccgattttggaagacattgacggctatcgatgtgccattagatttgcaaggaaacacgatctcgccatatcacagctgccgaacatcgatgccggccggcatgttgaactgatcgaagccattgcaaacgaatatcttgaacatgcgctaagccaagaacgcgactttcctcaatggccggaggacggcgcaggacaaccagtatgcccggttaccttaacgagaatccgttaaaaaagaggggagggaaacattatgatgaacagcgttgacgaccctggttcatacgaacagattggcagcgagaagccggcaaagcataaaggctctgttgtactgcgcagaggcagaaatacggaaacatctacgtcagaccaacgcttattagatgaaagaggttcaggtaattggatccattctgatacgtggcgcgtcttacgcattcagagcgaatttgtggaaggctttggtgcgctggccgaagttcctagagctgtcacagtattcggcagcgccagaacgccagtcgatcacccggaatacgagagagctagagagttaggagccgcgcttgctagagaaggatttgcggtaattacaggcggtggaccaggcgtcatggagggagctaacagaggagcgtcagaggcggatggctattcaattggccttggcatcgagctgccgttcgaacaaggcttaaacgactgggtagatcttggagtgaatttcagatatttttttgtaagaaaaacaatgttcgtgaaatattcacaggcctttgtgtgccttccgggcggctttggaacactggacgaactgtttgaagcactgacacttgttcaaacagcaaaagtcacaagatttccgattgttctgctgggaaaggacttctggtcaggactgctggattggattaaaacaacactggttaaagagggcaaagtctcagaaaaagatctggatctgatccattgtacagattcagtggatgaagcagtgagaatcgttagagaagcacaacagtaaattaattctagagtcgatccccgggttcgccagcaatgactaccggcagcccgcc

SEQ ID NO: 134 - DNA, 2987 bp, Artificial Sequence
  operon AmyE 0 + IPT1 + LOG6
gcggtagtggtgcttacgatgtacgacagggggattccccatacattcttcgcttggctgaaaatgattcttctttttatcgtctgcggcggcgttctgtttctgcttcggtatgtgattgtgaagctggcttacagaagagcggtaaaagaagaaataaaaaagaaatcatcttttttgtttggaaagcgagggaagcgttcacagtttcgggcagctttttttataggaacattgatttgtattcactctgccaagttgttttgatagagtgattgtgataattttaaatgtaagcgttaacaaaattctccagtcttcacatcggtttgaaaggaggaagcggaagaatgaagtaagagggatttttgactccgaagtaagtcttcaaaaaatcaaataaggagtgtcaagaatgtttgcaaaacgattcaaaacctctttactgccgttattcgctggatttttattgctgtttcatttggttctggcaggaccggcggctgcgagtgctgaaacggcgaacaaatcgaatgagcttacagcaccgtcgatcaaaagcggaaccattcttcatgcatggaattggtcgttcaatacgttaaaacacaatatgaaggatattcatgatgcaggatatacagccattcagacatctccgattaaccaagtaaaggaagggaatcaaggagataaaagcatgtcgaactggtactggctgtatcagccgacatcgtatcaaattggcaaccgttacttaggtactgaacaagaatttaaagaaatgtgtgcagccgctgaagaatatggcataaaggtcattgttgacgcggtcatcaatcataccaccagtgattatgccgcgatttccaatgaggttaagagtattccaaactggacacatggaaacacacaaattaaaaactggtctgatcgatgggatgtcacgcagaattcattgctcgggctgtatgactggaatacacaaaatacacaagtacagtcctatctgaaacggttcttagacagggcattgaatgacggggcagacggttttcgatttgatgccgccaaacatatagagcttccagatgatggcagttacggcagtcaattttggccgaatatcacaaatacatctgcagagttccaatacggagaaatcctgcaggatagtgcctccagagatgctgcttatgcgaattatatggatgtgacagcgtctaactatgggcattccataaggtccgctttaaagaatcgtaatctgggcgtgtcgaatatctcccactatgcatctgatgtgtctgcggacaagctagtgacatgggtagagtcgcatgatacacgggtgcgcatgatcgtatggttcactgtccaccaaccaaaactgtgctcagtaccgccaatatttctcccttggggggtacaaagaggtgtccctagaagagatccacgctgtgtaaaaattttacaaaaaggtattgactttccctacagggtgtgtaataatttaattacaggcgggggcaaccccgctcagtacctagagcgtaaaagaggggagggaaacactagtatgttattacacctgatctacggtcctacatgtagtggtaaaactgatatggctatacaaatcgcgcaagaaacaggttggcccgtcgttgctcttgatcgtgtgcagtgctgtccacagattgcgacaggatccggcagacctctggaaagcgagttacaatctacccggcggatttatttagactcacgccctttgacagaaggcattcttgacgcggaatccgctcatcgccgtcttatattcgaggtagattggaggaaatcggaagaaggcttaatcctcgaaggagggtccatctcgcttctaaattgtatggctaagtctccattttggagatctggattccagtggcacgtcaagcgcctgagacttggggattcagatgcgtttctgacgcgagcaaaacagcgtgtggctgagatgtttgctattagagaagatcggccaagtttattggaggagctcgccgagctgtggaattacccggcagctcgcccgattttggaagacattgacggctatcgatgtgccattagatttgcaaggaaacacgatctcgccatatcacagctgccgaacatcgatgccggccggcatgttgaactgatcgaagccattgcaaacgaatatcttgaacatgcgctaagccaagaacgcgactttcctcaatggccggaggacggcgcaggacaaccagtatgcccggttaccttaacgagaatccgttaaaaaagaggggagggaaacattatgatgaatttacgcccgatgcctgccacgacggttagcgcccaagcaagaccaacgccaaagtcagttacggtgttctgcggagctatgccgggtagaggaacaaaatacggtcaacttgctgagggcatgggtagagctattgctagaagcaaattaagacttgtatatggaggagcgagagtcggccttatgggaacactggcaaacgcagccttagactctggcggtacagtcgttggagtcattcctgaatcatttacagctatccctgaagccgcccaccacggactgacagaactgcatgtagtgcacgatatgcatcaaagaaaagccttaatggctgagctgggcgatgcattcattgcacttcctggcggcgtcggcacagcggaggagttttttgaggtgctgacatggagccatctgggcctgcataacaaaccgtgcgttctgctgaatgataatgaatactatagaccgctgctgtcatatattgaacatgcagcggtcgaaggctttatcacacctgcgacaagatcaagagtaatcgtgtgtaaagacattgaaggcgcgattgcggcgattagatcaccgtaaattaattctagagtcgatccccgggttcgccagcaatgactaccggcagcccgcc

SEQ ID NO: 135 - DNA, 2954 bp, Artificial Sequence
  operon AmyE 0 + IPT1 + LOG7
gcggtagtggtgcttacgatgtacgacagggggattccccatacattcttcgcttggctgaaaatgattcttctttttatcgtctgcggcggcgttctgtttctgcttcggtatgtgattgtgaagctggcttacagaagagcggtaaaagaagaaataaaaaagaaatcatcttttttgtttggaaagcgagggaagcgttcacagtttcgggcagctttttttataggaacattgatttgtattcactctgccaagttgttttgatagagtgattgtgataattttaaatgtaagcgttaacaaaattctccagtcttcacatcggtttgaaaggaggaagcggaagaatgaagtaagagggatttttgactccgaagtaagtcttcaaaaaatcaaataaggagtgtcaagaatgtttgcaaaacgattcaaaacctctttactgccgttattcgctggatttttattgctgtttcatttggttctggcaggaccggcggctgcgagtgctgaaacggcgaacaaatcgaatgagcttacagcaccgtcgatcaaaagcggaaccattcttcatgcatggaattggtcgttcaatacgttaaaacacaatatgaaggatattcatgatgcaggatatacagccattcagacatctccgattaaccaagtaaaggaagggaatcaaggagataaaagcatgtcgaactggtactggctgtatcagccgacatcgtatcaaattggcaaccgttacttaggtactgaacaagaatttaaagaaatgtgtgcagccgctgaagaatatggcataaaggtcattgttgacgcggtcatcaatcataccaccagtgattatgccgcgatttccaatgaggttaagagtattccaaactggacacatggaaacacacaaattaaaaactggtctgatcgatgggatgtcacgcagaattcattgctcgggctgtatgactggaatacacaaaatacacaagtacagtcctatctgaaacggttcttagacagggcattgaatgacggggcagacggttttcgatttgatgccgccaaacatatagagcttccagatgatggcagttacggcagtcaattttggccgaatatcacaaatacatctgcagagttccaatacggagaaatcctgcaggatagtgcctccagagatgctgcttatgcgaattatatggatgtgacagcgtctaactatgggcattccataaggtccgctttaaagaatcgtaatctgggcgtgtcgaatatctcccactatgcatctgatgtgtctgcggacaagctagtgacatgggtagagtcgcatgatacacgggtgcgcatgatcgtatggttcactgtccaccaaccaaaactgtgctcagtaccgccaatatttctcccttggggggtacaaagaggtgtccctagaagagatccacgctgtgtaaaaattttacaaaaaggtattgactttccctacagggtgtgtaataatttaattacaggcgggggcaaccccgctcagtacctagagcgtaaaagaggggagggaaacactagtatgttattacacctgatctacggtcctacatgtagtggtaaaactgatatggctatacaaatcgcgcaagaaacaggttggcccgtcgttgctcttgatcgtgtgcagtgctgtccacagattgcgacaggatccggcagacctctggaaagcgagttacaatctacccggcggatttatttagactcacgccctttgacagaaggcattcttgacgcggaatccgctcatcgccgtcttatattcgaggtagattggaggaaatcggaagaaggcttaatcctcgaaggagggtccatctcgcttctaaattgtatggctaagtctccattttggagatctggattccagtggcacgtcaagcgcctgagacttggggattcagatgcgtttctgacgcgagcaaaacagcgtgtggctgagatgtttgctattagagaagatcggccaagtttattggaggagctcgccgagctgtggaattacccggcagctcgcccgattttggaagacattgacggctatcgatgtgccattagatttgcaaggaaacacgatctcgccatatcacagctgccgaacatcgatgccggccggcatgttgaactgatcgaagccattgcaaacgaatatcttgaacatgcgctaagccaagaacgcgactttcctcaatggccggaggacggcgcaggacaaccagtatgcccggttaccttaacgagaatccgttaaaaaagaggggagggaaacattatgatgaaatcagtagcgatttattgcggatcatcaatgggatctagccctagatatgcagaagtagctaaagagatgggagaggaacttgcaagacgcaatctgagagtagtgtatggaggaggaaacgtcggactgatgggtacagtggccgacgcagcgttagcggccggtggcgaggtggtaggcgtaattccaagacagttaattaaccgcgagatggcacatgccggtctgacagatttagaagtggtggacacaatggcgcagagaaaaacgagaatggaggaacttgccgatgcttttgtgtgtctgcctggcggtgttggcacgctggaagaagtcgttgaagttcttacaatgcaacaacttggccacgtccaaggcccggtcggcctggttaatgttgacggcttttgggaaccgtttgtcagaacactggaatcaatggtcgcttgcggcttcgttcagtcaagatacatcgatgctatcgcactgtcttcagatccggctgaaattctggatgaattcgcaggctggagcccgatcggcgcgaagtggagctaaattaattctagagtcgatccccgggttcgccagcaatgactaccggcagcccgcc

SEQ ID NO: 136 - DNA, 3218 bp, Artificial Sequence
  operon AmyE 0 + IPT1 + LOG8
gcggtagtggtgcttacgatgtacgacagggggattccccatacattcttcgcttggctgaaaatgattcttctttttatcgtctgcggcggcgttctgtttctgcttcggtatgtgattgtgaagctggcttacagaagagcggtaaaagaagaaataaaaaagaaatcatcttttttgtttggaaagcgagggaagcgttcacagtttcgggcagctttttttataggaacattgatttgtattcactctgccaagttgttttgatagagtgattgtgataattttaaatgtaagcgttaacaaaattctccagtcttcacatcggtttgaaaggaggaagcggaagaatgaagtaagagggatttttgactccgaagtaagtcttcaaaaaatcaaataaggagtgtcaagaatgtttgcaaaacgattcaaaacctctttactgccgttattcgctggatttttattgctgtttcatttggttctggcaggaccggcggctgcgagtgctgaaacggcgaacaaatcgaatgagcttacagcaccgtcgatcaaaagcggaaccattcttcatgcatggaattggtcgttcaatacgttaaaacacaatatgaaggatattcatgatgcaggatatacagccattcagacatctccgattaaccaagtaaaggaagggaatcaaggagataaaagcatgtcgaactggtactggctgtatcagccgacatcgtatcaaattggcaaccgttacttaggtactgaacaagaatttaaagaaatgtgtgcagccgctgaagaatatggcataaaggtcattgttgacgcggtcatcaatcataccaccagtgattatgccgcgatttccaatgaggttaagagtattccaaactggacacatggaaacacacaaattaaaaactggtctgatcgatgggatgtcacgcagaattcattgctcgggctgtatgactggaatacacaaaatacacaagtacagtcctatctgaaacggttcttagacagggcattgaatgacggggcagacggttttcgatttgatgccgccaaacatatagagcttccagatgatggcagttacggcagtcaattttggccgaatatcacaaatacatctgcagagttccaatacggagaaatcctgcaggatagtgcctccagagatgctgcttatgcgaattatatggatgtgacagcgtctaactatgggcattccataaggtccgctttaaagaatcgtaatctgggcgtgtcgaatatctcccactatgcatctgatgtgtctgcggacaagctagtgacatgggtagagtcgcatgatacacgggtgcgcatgatcgtatggttcactgtccaccaaccaaaactgtgctcagtaccgccaatatttctcccttggggggtacaaagaggtgtccctagaagagatccacgctgtgtaaaaattttacaaaaaggtattgactttccctacagggtgtgtaataatttaattacaggcgggggcaaccccgctcagtacctagagcgtaaaagaggggagggaaacactagtatgttattacacctgatctacggtcctacatgtagtggtaaaactgatatggctatacaaatcgcgcaagaaacaggttggcccgtcgttgctcttgatcgtgtgcagtgctgtccacagattgcgacaggatccggcagacctctggaaagcgagttacaatctacccggcggatttatttagactcacgccctttgacagaaggcattcttgacgcggaatccgctcatcgccgtcttatattcgaggtagattggaggaaatcggaagaaggcttaatcctcgaaggagggtccatctcgcttctaaattgtatggctaagtctccattttggagatctggattccagtggcacgtcaagcgcctgagacttggggattcagatgcgtttctgacgcgagcaaaacagcgtgtggctgagatgtttgctattagagaagatcggccaagtttattggaggagctcgccgagctgtggaattacccggcagctcgcccgattttggaagacattgacggctatcgatgtgccattagatttgcaaggaaacacgatctcgccatatcacagctgccgaacatcgatgccggccggcatgttgaactgatcgaagccattgcaaacgaatatcttgaacatgcgctaagccaagaacgcgactttcctcaatggccggaggacggcgcaggacaaccagtatgcccggttaccttaacgagaatccgttaaaaaagaggggagggaaacattatgatggcccctcttcgctcaaacaaagatggcaaacctagagttcctagaacgacggaaagcgccgaaacgaatagaatgttaagaggtccgatgttactgagaacagacggaaatcagacgtcaacatacgatcaaagacttttagagtcaggcgcagatcacgcttggaagcatgcagatccgtggcgcgttctgagaatccagggagagttcgtggcaggatttgatgctctgagcgatctgcctaaggcggttacggtatacggctcagccagaacaaaagaggacacaccggaatatgcacagggcatcgatttaggtgcaaaattagttgagcatgactacgctgttgtgacaggcggtggcccaggcatcatggaggcggcgaatagaggcgcctatgaagctggaggtctttctgtgggcctgggcattgaacttccgtttgaacaaggtttaaacaaatatgtagatcttggaattaactttagatactttttcgcgagaaagacaatgttcctgaaatattcacaggcgtttgtgtgcctgccgggcggtatgggaacaatggatgagctgtttgaaatgctgtgtatggtgcaaacaggaaaagtgacaaactatccgattgtactgatcggcacagaatattggagcggcctggtagaatggatggctaatacactggtcgccgaaggctacatcaatccacaagatatggatctgttcctgctgacagacagcgtcgacgaagccgtcgaacatattgtcgcggctcataaagaaatgacagacgaaagagtctcaagactgaatcacgacgcataaattaattctagagtcgatccccgggttcgccagcaatgactaccggcagcccgcc

SEQ ID NO: 137 - DNA, 3122 bp, Artificial Sequence
  operon AmyE 0 + IPT1 + LOG9
gcggtagtggtgcttacgatgtacgacagggggattccccatacattcttcgcttggctgaaaatgattcttctttttatcgtctgcggcggcgttctgtttctgcttcggtatgtgattgtgaagctggcttacagaagagcggtaaaagaagaaataaaaaagaaatcatcttttttgtttggaaagcgagggaagcgttcacagtttcgggcagctttttttataggaacattgatttgtattcactctgccaagttgttttgatagagtgattgtgataattttaaatgtaagcgttaacaaaattctccagtcttcacatcggtttgaaaggaggaagcggaagaatgaagtaagagggatttttgactccgaagtaagtcttcaaaaaatcaaataaggagtgtcaagaatgtttgcaaaacgattcaaaacctctttactgccgttattcgctggatttttattgctgtttcatttggttctggcaggaccggcggctgcgagtgctgaaacggcgaacaaatcgaatgagcttacagcaccgtcgatcaaaagcggaaccattcttcatgcatggaattggtcgttcaatacgttaaaacacaatatgaaggatattcatgatgcaggatatacagccattcagacatctccgattaaccaagtaaaggaagggaatcaaggagataaaagcatgtcgaactggtactggctgtatcagccgacatcgtatcaaattggcaaccgttacttaggtactgaacaagaatttaaagaaatgtgtgcagccgctgaagaatatggcataaaggtcattgttgacgcggtcatcaatcataccaccagtgattatgccgcgatttccaatgaggttaagagtattccaaactggacacatggaaacacacaaattaaaaactggtctgatcgatgggatgtcacgcagaattcattgctcgggctgtatgactggaatacacaaaatacacaagtacagtcctatctgaaacggttcttagacagggcattgaatgacggggcagacggttttcgatttgatgccgccaaacatatagagcttccagatgatggcagttacggcagtcaattttggccgaatatcacaaatacatctgcagagttccaatacggagaaatcctgcaggatagtgcctccagagatgctgcttatgcgaattatatggatgtgacagcgtctaactatgggcattccataaggtccgctttaaagaatcgtaatctgggcgtgtcgaatatctcccactatgcatctgatgtgtctgcggacaagctagtgacatgggtagagtcgcatgatacacgggtgcgcatgatcgtatggttcactgtccaccaaccaaaactgtgctcagtaccgccaatatttctcccttggggggtacaaagaggtgtccctagaagagatccacgctgtgtaaaaattttacaaaaaggtattgactttccctacagggtgtgtaataatttaattacaggcgggggcaaccccgctcagtacctagagcgtaaaagaggggagggaaacactagtatgttattacacctgatctacggtcctacatgtagtggtaaaactgatatggctatacaaatcgcgcaagaaacaggttggcccgtcgttgctcttgatcgtgtgcagtgctgtccacagattgcgacaggatccggcagacctctggaaagcgagttacaatctacccggcggatttatttagactcacgccctttgacagaaggcattcttgacgcggaatccgctcatcgccgtcttatattcgaggtagattggaggaaatcggaagaaggcttaatcctcgaaggagggtccatctcgcttctaaattgtatggctaagtctccattttggagatctggattccagtggcacgtcaagcgcctgagacttggggattcagatgcgtttctgacgcgagcaaaacagcgtgtggctgagatgtttgctattagagaagatcggccaagtttattggaggagctcgccgagctgtggaattacccggcagctcgcccgattttggaagacattgacggctatcgatgtgccattagatttgcaaggaaacacgatctcgccatatcacagctgccgaacatcgatgccggccggcatgttgaactgatcgaagccattgcaaacgaatatcttgaacatgcgctaagccaagaacgcgactttcctcaatggccggaggacggcgcaggacaaccagtatgcccggttaccttaacgagaatccgttaaaaaagaggggagggaaacattatgatgttattacatcttatttacggacctacatgttcaggaaaaacagacatggccattcaaattgctcaagaaacgggatggccggttgtggcgttagacagagtacagtgctgcccacagattgcaacgggctcaggtagaccgttagaatctgagttacaatcaacaagaagaatctatctggattcacgcccactgacagagggcatcctggacgcagagagcgcgcaccgccgccttatctttgaagtcgattggcgcaaatctgaggagggccttatccttgaaggaggaagcattagcctgcttaattgtatggcaaaatcaccgttttggagatctggcttccagtggcacgtaaagagactgagacttggcgattcagacgcatttctgacaagagcaaaacagagagtcgcagaaatgttcgcgattagagaagacagaccaagcctgctggaggaattagctgaactgtggaactatccggctgcgcgccctattctggaggatattgatggttatcgctgtgcgattagatttgctagaaagcatgatctggctatttcacaactgcctaatatcgatgcgggaagacatgttgaactgattgaagccatcgccaacgaatacctggaacatgccctgtcacaggaaagagattttcctcaatggccggaagatggtgccggccaaccggtttgcccggtgacactgacaagaattagataaattaattctagagtcgatccccgggttcgccagcaatgactaccggcagcccgcc

SEQ ID NO: 138 - DNA, 3038 bp, Artificial Sequence
  operon AmyE 0 + IPT1 + LOG10
gcggtagtggtgcttacgatgtacgacagggggattccccatacattcttcgcttggctgaaaatgattcttctttttatcgtctgcggcggcgttctgtttctgcttcggtatgtgattgtgaagctggcttacagaagagcggtaaaagaagaaataaaaaagaaatcatcttttttgtttggaaagcgagggaagcgttcacagtttcgggcagctttttttataggaacattgatttgtattcactctgccaagttgttttgatagagtgattgtgataattttaaatgtaagcgttaacaaaattctccagtcttcacatcggtttgaaaggaggaagcggaagaatgaagtaagagggatttttgactccgaagtaagtcttcaaaaaatcaaataaggagtgtcaagaatgtttgcaaaacgattcaaaacctctttactgccgttattcgctggatttttattgctgtttcatttggttctggcaggaccggcggctgcgagtgctgaaacggcgaacaaatcgaatgagcttacagcaccgtcgatcaaaagcggaaccattcttcatgcatggaattggtcgttcaatacgttaaaacacaatatgaaggatattcatgatgcaggatatacagccattcagacatctccgattaaccaagtaaaggaagggaatcaaggagataaaagcatgtcgaactggtactggctgtatcagccgacatcgtatcaaattggcaaccgttacttaggtactgaacaagaatttaaagaaatgtgtgcagccgctgaagaatatggcataaaggtcattgttgacgcggtcatcaatcataccaccagtgattatgccgcgatttccaatgaggttaagagtattccaaactggacacatggaaacacacaaattaaaaactggtctgatcgatgggatgtcacgcagaattcattgctcgggctgtatgactggaatacacaaaatacacaagtacagtcctatctgaaacggttcttagacagggcattgaatgacggggcagacggttttcgatttgatgccgccaaacatatagagcttccagatgatggcagttacggcagtcaattttggccgaatatcacaaatacatctgcagagttccaatacggagaaatcctgcaggatagtgcctccagagatgctgcttatgcgaattatatggatgtgacagcgtctaactatgggcattccataaggtccgctttaaagaatcgtaatctgggcgtgtcgaatatctcccactatgcatctgatgtgtctgcggacaagctagtgacatgggtagagtcgcatgatacacgggtgcgcatgatcgtatggttcactgtccaccaaccaaaactgtgctcagtaccgccaatatttctcccttggggggtacaaagaggtgtccctagaagagatccacgctgtgtaaaaattttacaaaaaggtattgactttccctacagggtgtgtaataatttaattacaggcgggggcaaccccgctcagtacctagagcgtaaaagaggggagggaaacactagtatgttattacacctgatctacggtcctacatgtagtggtaaaactgatatggctatacaaatcgcgcaagaaacaggttggcccgtcgttgctcttgatcgtgtgcagtgctgtccacagattgcgacaggatccggcagacctctggaaagcgagttacaatctacccggcggatttatttagactcacgccctttgacagaaggcattcttgacgcggaatccgctcatcgccgtcttatattcgaggtagattggaggaaatcggaagaaggcttaatcctcgaaggagggtccatctcgcttctaaattgtatggctaagtctccattttggagatctggattccagtggcacgtcaagcgcctgagacttggggattcagatgcgtttctgacgcgagcaaaacagcgtgtggctgagatgtttgctattagagaagatcggccaagtttattggaggagctcgccgagctgtggaattacccggcagctcgcccgattttggaagacattgacggctatcgatgtgccattagatttgcaaggaaacacgatctcgccatatcacagctgccgaacatcgatgccggccggcatgttgaactgatcgaagccattgcaaacgaatatcttgaacatgcgctaagccaagaacgcgactttcctcaatggccggaggacggcgcaggacaaccagtatgcccggttaccttaacgagaatccgttaaaaaagaggggagggaaacattatgatggagattaagggagagagcatgcaaaagtcaaagttcagacgcatttgtgtgttttgcggttcatcacagggcaaaaagtcttcatatcaagatgctgcagttgatctgggtaacgaattagtaagcagaaacatcgatcttgtctacggcggtggctctattggacttatgggcttagtgtctcaagcagtccatgacggcggtagacatgtcatcggcattattcctaaaacgttaatgccgcgcgagctgacaggcgaaacagtaggagaggtaagagcggtggccgacatgcatcaaagaaaagcggaaatggccaaacacagcgacgcatttattgctttaccgggcggctacggaacactggaagaacttcttgaggtcatcacgtgggcccagctgggcattcatgataaaccagtgggactgctgaatgtggatggctattataatagcctgctgtcatttattgataaagcggttgaagaaggattcatttcaccgacggcaagagaaatcattgtttcagcgcctacagctaaagaactggttaaaaaactggaagagtatgcgccgtgccatgaaagagttgctacaaaactgtgttgggaaatggagagaatcggatattcatcagaagaataaattaattctagagtcgatccccgggttcgccagcaatgactaccggcagcccgcc

SEQ ID NO: 139 - DNA, 3311 bp, Artificial Sequence
  operon AmyE 0 + IPT1 + LOG11
gcggtagtggtgcttacgatgtacgacagggggattccccatacattcttcgcttggctgaaaatgattcttctttttatcgtctgcggcggcgttctgtttctgcttcggtatgtgattgtgaagctggcttacagaagagcggtaaaagaagaaataaaaaagaaatcatcttttttgtttggaaagcgagggaagcgttcacagtttcgggcagctttttttataggaacattgatttgtattcactctgccaagttgttttgatagagtgattgtgataattttaaatgtaagcgttaacaaaattctccagtcttcacatcggtttgaaaggaggaagcggaagaatgaagtaagagggatttttgactccgaagtaagtcttcaaaaaatcaaataaggagtgtcaagaatgtttgcaaaacgattcaaaacctctttactgccgttattcgctggatttttattgctgtttcatttggttctggcaggaccggcggctgcgagtgctgaaacggcgaacaaatcgaatgagcttacagcaccgtcgatcaaaagcggaaccattcttcatgcatggaattggtcgttcaatacgttaaaacacaatatgaaggatattcatgatgcaggatatacagccattcagacatctccgattaaccaagtaaaggaagggaatcaaggagataaaagcatgtcgaactggtactggctgtatcagccgacatcgtatcaaattggcaaccgttacttaggtactgaacaagaatttaaagaaatgtgtgcagccgctgaagaatatggcataaaggtcattgttgacgcggtcatcaatcataccaccagtgattatgccgcgatttccaatgaggttaagagtattccaaactggacacatggaaacacacaaattaaaaactggtctgatcgatgggatgtcacgcagaattcattgctcgggctgtatgactggaatacacaaaatacacaagtacagtcctatctgaaacggttcttagacagggcattgaatgacggggcagacggttttcgatttgatgccgccaaacatatagagcttccagatgatggcagttacggcagtcaattttggccgaatatcacaaatacatctgcagagttccaatacggagaaatcctgcaggatagtgcctccagagatgctgcttatgcgaattatatggatgtgacagcgtctaactatgggcattccataaggtccgctttaaagaatcgtaatctgggcgtgtcgaatatctcccactatgcatctgatgtgtctgcggacaagctagtgacatgggtagagtcgcatgatacacgggtgcgcatgatcgtatggttcactgtccaccaaccaaaactgtgctcagtaccgccaatatttctcccttggggggtacaaagaggtgtccctagaagagatccacgctgtgtaaaaattttacaaaaaggtattgactttccctacagggtgtgtaataatttaattacaggcgggggcaaccccgctcagtacctagagcgtaaaagaggggagggaaacactagtatgttattacacctgatctacggtcctacatgtagtggtaaaactgatatggctatacaaatcgcgcaagaaacaggttggcccgtcgttgctcttgatcgtgtgcagtgctgtccacagattgcgacaggatccggcagacctctggaaagcgagttacaatctacccggcggatttatttagactcacgccctttgacagaaggcattcttgacgcggaatccgctcatcgccgtcttatattcgaggtagattggaggaaatcggaagaaggcttaatcctcgaaggagggtccatctcgcttctaaattgtatggctaagtctccattttggagatctggattccagtggcacgtcaagcgcctgagacttggggattcagatgcgtttctgacgcgagcaaaacagcgtgtggctgagatgtttgctattagagaagatcggccaagtttattggaggagctcgccgagctgtggaattacccggcagctcgcccgattttggaagacattgacggctatcgatgtgccattagatttgcaaggaaacacgatctcgccatatcacagctgccgaacatcgatgccggccggcatgttgaactgatcgaagccattgcaaacgaatatcttgaacatgcgctaagccaagaacgcgactttcctcaatggccggaggacggcgcaggacaaccagtatgcccggttaccttaacgagaatccgttaaaaaagaggggagggaaacattatgatgggtgtctcaatgctggattcttcttggtcatttcgcctttcaagcaagatgtctccaagcccgacgcaaaaattagccagctctctgacagaacgcgatccaatcagcttaaatctgcgcttcgaccattcaattagagcatatagcgcaagagctcttaagtgcagatctgtcaaatctgaagatttcgttgaaagatcatcaccatttgaagttaagaaggagctggaaatttgctttgatcttgtccatagattaggtagaggcattgtttaccttggcagcgctagaattccgccgaaccacagccattacctgcaggcacaggaattatcaagagaagcggcaacgttacttgactgcacaacgtggtcaggcgctggacctggccttatggatgcggtgacggaaggtgcccttgaggcggaaaagccggtaggtggcatcaaaattgaaaaagaagcgggcgagtggacggcgtcaaaattccacccgtatttacctccgcaaaattatcatacatgtagatttttctcagcgagaaaacatggccttgtcgatgccgtaattagaaataatgtttcagaaaaaacagctatcattgcactgccgggcggcattggaacattagatgagatgttcgaaatcctggctctgattcaactggagagaattggctcagctctgccggtgccttttatcgtaatgaactacgacgccttttattcaaaactgctggaatttatcgagacatgtgaaaatctgggaacagtttcaaaaggagaagtgtcagcactgtggaaagtgtgtaataacaactttgaggccctgacatatctggcggaattttatgacctgcctgatggatcagcaaaactggagacagaactgagaaaaacaacacctacaagaggataaattaattctagagtcgatccccgggttcgccagcaatgactaccggcagcccgcc

SEQ ID NO: 140 - DNA, 2093 bp, Artificial Sequence
  operon EryR + AmyE END
attaattctagaaattaagaaggagggattcgtcatgttggtattccaaatgcgttatgtagataaaacatctactgttttgaaacagactaaaaacagtgattacgcagataaataaatacgttagattaattcctaccagtgactaatcttatgactttttaaacagataactaaaattacaaacaaatcgtttaacttctgtatttgtttatagatgtaatcacttcaggagtgattacatgaacaaaaatataaaatattctcaaaactttttaacgagtgaaaaagtactcaaccaaataataaaacaattgaatttaaaagaaaccgataccgtttacgaaattggaacaggtaaagggcatttaacgacgaaactggctaaaataagtaaacaggtaacgtctattgaattagacagtcatctattcaacttatcgtcagaaaaattaaaactgaacattcgtgtcactttaattcaccaagatattctacagtttcaattccctaacaaacagaggtataaaattgttgggaatattccttaccatttaagcacacaaattattaaaaaagtggtttttgaaagccatgcgtctgacatctatctgattgttgaagaaggattctacaagcgtaccttggatattcaccgaacactagggttgctcttgcacactcaagtctcgattcagcaattgcttaagctgccagcggaatgctttcatcctaaaccaaaagtaaacagtgtcttaataaaacttacccgccataccacagatgttccagataaatattggaagctatatacgtactttgtttcaaaatgggtcaatcgagaatatcgtcaactgtttactaaaaatcagtttcatcaagcaatgaaacacgccaaagtaaacaatttaagtaccgttacttatgagcaagtattgtctatttttaatagttatctattatttaacgggaggaaataattctatgagtcgcttttgtaaatttggaaagttacacgttactaaagggaatgtagatggatcctgtatgccaatgatgatgaagagtcgacatggatgagcgatgatgatatccgtttaggctgggcggtgatagcttctcgttcaggcagtacgcctcttttcttttccagacctgagggaggcggaaatggtgtgaggttcccggggaaaagccaaataggcgatcgcgggagtgctttatttgaagatcaggctatcactgcggtcaatagatttcacaatgtgatggctggacagcctgaggaactctcgaacccgaatggaaacaaccagatatttatgaatcagcgcggctcacatggcgttgtgctggcaaatgcaggttcatcctctgtctctatcaatacggcaacaaaattgcctgatggcaggtatgacaataaagctggagcgggttcatttcaagtgaacgatggtaaactgacaggcacgatcaatgccaggtctgtagctgtgctttatcctgatgatattgcaaaagcgcctcatgttttccttgagaattacaaaacaggtgtaacacattctttcaatgatcaactgacgattaccttgcgtgcagatgcgaatacaacaaaagccgtttatcaaatcaataatggaccagagacggcgtttaaggatggagatcaattcacaatcggaaaaggagatccatttggcaaaacatacaccatcatgttaaaaggaacgaacagtgatggtgtaacgaggaccgagaaatacagttttgttaaaagagatccagcgtcggccaaaaccatcggctatcaaaatccgaatcattggagccaggtaaatgcttatatctataaacatgatgggagccgagtaattgaattgaccggatcttggcctggaaaaccaatgactaaaaatgcagacggaatttacacgctgacgctgcctgcggacacggatacaaccaacgcaaaagtgatttttaataatggcagcgcccaagtgcccggtcagaatcagcctggctttgattacgtgctaaatggtttatataatgactcgggcttaagcggttctcttccccattga

SEQ ID NO: 141 - DNA, 5003 bp, Artificial Sequence
  complete  AmyE 0 IPT1 LOG2 EryR AmyE END operon
gcggtagtggtgcttacgatgtacgacagggggattccccatacattcttcgcttggctgaaaatgattcttctttttatcgtctgcggcggcgttctgtttctgcttcggtatgtgattgtgaagctggcttacagaagagcggtaaaagaagaaataaaaaagaaatcatcttttttgtttggaaagcgagggaagcgttcacagtttcgggcagctttttttataggaacattgatttgtattcactctgccaagttgttttgatagagtgattgtgataattttaaatgtaagcgttaacaaaattctccagtcttcacatcggtttgaaaggaggaagcggaagaatgaagtaagagggatttttgactccgaagtaagtcttcaaaaaatcaaataaggagtgtcaagaatgtttgcaaaacgattcaaaacctctttactgccgttattcgctggatttttattgctgtttcatttggttctggcaggaccggcggctgcgagtgctgaaacggcgaacaaatcgaatgagcttacagcaccgtcgatcaaaagcggaaccattcttcatgcatggaattggtcgttcaatacgttaaaacacaatatgaaggatattcatgatgcaggatatacagccattcagacatctccgattaaccaagtaaaggaagggaatcaaggagataaaagcatgtcgaactggtactggctgtatcagccgacatcgtatcaaattggcaaccgttacttaggtactgaacaagaatttaaagaaatgtgtgcagccgctgaagaatatggcataaaggtcattgttgacgcggtcatcaatcataccaccagtgattatgccgcgatttccaatgaggttaagagtattccaaactggacacatggaaacacacaaattaaaaactggtctgatcgatgggatgtcacgcagaattcattgctcgggctgtatgactggaatacacaaaatacacaagtacagtcctatctgaaacggttcttagacagggcattgaatgacggggcagacggttttcgatttgatgccgccaaacatatagagcttccagatgatggcagttacggcagtcaattttggccgaatatcacaaatacatctgcagagttccaatacggagaaatcctgcaggatagtgcctccagagatgctgcttatgcgaattatatggatgtgacagcgtctaactatgggcattccataaggtccgctttaaagaatcgtaatctgggcgtgtcgaatatctcccactatgcatctgatgtgtctgcggacaagctagtgacatgggtagagtcgcatgatacacgggtgcgcatgatcgtatggttcactgtccaccaaccaaaactgtgctcagtaccgccaatatttctcccttggggggtacaaagaggtgtccctagaagagatccacgctgtgtaaaaattttacaaaaaggtattgactttccctacagggtgtgtaataatttaattacaggcgggggcaaccccgctcagtacctagagcgtaaaagaggggagggaaacactagtatgttattacacctgatctacggtcctacatgtagtggtaaaactgatatggctatacaaatcgcgcaagaaacaggttggcccgtcgttgctcttgatcgtgtgcagtgctgtccacagattgcgacaggatccggcagacctctggaaagcgagttacaatctacccggcggatttatttagactcacgccctttgacagaaggcattcttgacgcggaatccgctcatcgccgtcttatattcgaggtagattggaggaaatcggaagaaggcttaatcctcgaaggagggtccatctcgcttctaaattgtatggctaagtctccattttggagatctggattccagtggcacgtcaagcgcctgagacttggggattcagatgcgtttctgacgcgagcaaaacagcgtgtggctgagatgtttgctattagagaagatcggccaagtttattggaggagctcgccgagctgtggaattacccggcagctcgcccgattttggaagacattgacggctatcgatgtgccattagatttgcaaggaaacacgatctcgccatatcacagctgccgaacatcgatgccggccggcatgttgaactgatcgaagccattgcaaacgaatatcttgaacatgcgctaagccaagaacgcgactttcctcaatggccggaggacggcgcaggacaaccagtatgcccggttaccttaacgagaatccgttaaaaaagaggggagggaaacattatgatgaaaacaatctgtgtattcgctggcagcaaccctggcggtaatgaagcttacaagagaaaggccgctgaactgggtgtgtatatggcggaacagggaatcggattagtatacggtggctctcgcgtcggactgatgggcacgattgcagacgcaatcatggagaacggcggcacggcaattggagttatgccatctggattattttctggagaagttgtccatcagaatctgacagagctgattgaggtcaatggcatgcacgagagaaaagccaaaatgtcagagctggcggatggtttcatcagcatgcctggaggatttggaacatacgaggagctttttgaagttctttgctgggcgcaaattggcattcatcaaaaaccgattggcctgtataatgtgaacggctattttgaaccgatgatgaagatggtgaaatattcaattcaagaaggctttagcaatgaatcacatcttaaactgatccactcatcatcaagaccggatgaactgatcgaacaaatgcagaattattcatatccgattttagaaaaaaaatggacagaaatttaaattaattctagaaattaagaaggagggattcgtcatgttggtattccaaatgcgttatgtagataaaacatctactgttttgaaacagactaaaaacagtgattacgcagataaataaatacgttagattaattcctaccagtgactaatcttatgactttttaaacagataactaaaattacaaacaaatcgtttaacttctgtatttgtttatagatgtaatcacttcaggagtgattacatgaacaaaaatataaaatattctcaaaactttttaacgagtgaaaaagtactcaaccaaataataaaacaattgaatttaaaagaaaccgataccgtttacgaaattggaacaggtaaagggcatttaacgacgaaactggctaaaataagtaaacaggtaacgtctattgaattagacagtcatctattcaacttatcgtcagaaaaattaaaactgaacattcgtgtcactttaattcaccaagatattctacagtttcaattccctaacaaacagaggtataaaattgttgggaatattccttaccatttaagcacacaaattattaaaaaagtggtttttgaaagccatgcgtctgacatctatctgattgttgaagaaggattctacaagcgtaccttggatattcaccgaacactagggttgctcttgcacactcaagtctcgattcagcaattgcttaagctgccagcggaatgctttcatcctaaaccaaaagtaaacagtgtcttaataaaacttacccgccataccacagatgttccagataaatattggaagctatatacgtactttgtttcaaaatgggtcaatcgagaatatcgtcaactgtttactaaaaatcagtttcatcaagcaatgaaacacgccaaagtaaacaatttaagtaccgttacttatgagcaagtattgtctatttttaatagttatctattatttaacgggaggaaataattctatgagtcgcttttgtaaatttggaaagttacacgttactaaagggaatgtagatggatcctgtatgccaatgatgatgaagagtcgacatggatgagcgatgatgatatccgtttaggctgggcggtgatagcttctcgttcaggcagtacgcctcttttcttttccagacctgagggaggcggaaatggtgtgaggttcccggggaaaagccaaataggcgatcgcgggagtgctttatttgaagatcaggctatcactgcggtcaatagatttcacaatgtgatggctggacagcctgaggaactctcgaacccgaatggaaacaaccagatatttatgaatcagcgcggctcacatggcgttgtgctggcaaatgcaggttcatcctctgtctctatcaatacggcaacaaaattgcctgatggcaggtatgacaataaagctggagcgggttcatttcaagtgaacgatggtaaactgacaggcacgatcaatgccaggtctgtagctgtgctttatcctgatgatattgcaaaagcgcctcatgttttccttgagaattacaaaacaggtgtaacacattctttcaatgatcaactgacgattaccttgcgtgcagatgcgaatacaacaaaagccgtttatcaaatcaataatggaccagagacggcgtttaaggatggagatcaattcacaatcggaaaaggagatccatttggcaaaacatacaccatcatgttaaaaggaacgaacagtgatggtgtaacgaggaccgagaaatacagttttgttaaaagagatccagcgtcggccaaaaccatcggctatcaaaatccgaatcattggagccaggtaaatgcttatatctataaacatgatgggagccgagtaattgaattgaccggatcttggcctggaaaaccaatgactaaaaatgcagacggaatttacacgctgacgctgcctgcggacacggatacaaccaacgcaaaagtgatttttaataatggcagcgcccaagtgcccggtcagaatcagcctggctttgattacgtgctaaatggtttatataatgactcgggcttaagcggttctcttccccattg

SEQ ID NO: 142 - DNA, 5198 bp, Artificial Sequence
  complete  AmyE 0 IPT1 LOG3 EryR AmyE END operon
gcggtagtggtgcttacgatgtacgacagggggattccccatacattcttcgcttggctgaaaatgattcttctttttatcgtctgcggcggcgttctgtttctgcttcggtatgtgattgtgaagctggcttacagaagagcggtaaaagaagaaataaaaaagaaatcatcttttttgtttggaaagcgagggaagcgttcacagtttcgggcagctttttttataggaacattgatttgtattcactctgccaagttgttttgatagagtgattgtgataattttaaatgtaagcgttaacaaaattctccagtcttcacatcggtttgaaaggaggaagcggaagaatgaagtaagagggatttttgactccgaagtaagtcttcaaaaaatcaaataaggagtgtcaagaatgtttgcaaaacgattcaaaacctctttactgccgttattcgctggatttttattgctgtttcatttggttctggcaggaccggcggctgcgagtgctgaaacggcgaacaaatcgaatgagcttacagcaccgtcgatcaaaagcggaaccattcttcatgcatggaattggtcgttcaatacgttaaaacacaatatgaaggatattcatgatgcaggatatacagccattcagacatctccgattaaccaagtaaaggaagggaatcaaggagataaaagcatgtcgaactggtactggctgtatcagccgacatcgtatcaaattggcaaccgttacttaggtactgaacaagaatttaaagaaatgtgtgcagccgctgaagaatatggcataaaggtcattgttgacgcggtcatcaatcataccaccagtgattatgccgcgatttccaatgaggttaagagtattccaaactggacacatggaaacacacaaattaaaaactggtctgatcgatgggatgtcacgcagaattcattgctcgggctgtatgactggaatacacaaaatacacaagtacagtcctatctgaaacggttcttagacagggcattgaatgacggggcagacggttttcgatttgatgccgccaaacatatagagcttccagatgatggcagttacggcagtcaattttggccgaatatcacaaatacatctgcagagttccaatacggagaaatcctgcaggatagtgcctccagagatgctgcttatgcgaattatatggatgtgacagcgtctaactatgggcattccataaggtccgctttaaagaatcgtaatctgggcgtgtcgaatatctcccactatgcatctgatgtgtctgcggacaagctagtgacatgggtagagtcgcatgatacacgggtgcgcatgatcgtatggttcactgtccaccaaccaaaactgtgctcagtaccgccaatatttctcccttggggggtacaaagaggtgtccctagaagagatccacgctgtgtaaaaattttacaaaaaggtattgactttccctacagggtgtgtaataatttaattacaggcgggggcaaccccgctcagtacctagagcgtaaaagaggggagggaaacactagtatgttattacacctgatctacggtcctacatgtagtggtaaaactgatatggctatacaaatcgcgcaagaaacaggttggcccgtcgttgctcttgatcgtgtgcagtgctgtccacagattgcgacaggatccggcagacctctggaaagcgagttacaatctacccggcggatttatttagactcacgccctttgacagaaggcattcttgacgcggaatccgctcatcgccgtcttatattcgaggtagattggaggaaatcggaagaaggcttaatcctcgaaggagggtccatctcgcttctaaattgtatggctaagtctccattttggagatctggattccagtggcacgtcaagcgcctgagacttggggattcagatgcgtttctgacgcgagcaaaacagcgtgtggctgagatgtttgctattagagaagatcggccaagtttattggaggagctcgccgagctgtggaattacccggcagctcgcccgattttggaagacattgacggctatcgatgtgccattagatttgcaaggaaacacgatctcgccatatcacagctgccgaacatcgatgccggccggcatgttgaactgatcgaagccattgcaaacgaatatcttgaacatgcgctaagccaagaacgcgactttcctcaatggccggaggacggcgcaggacaaccagtatgcccggttaccttaacgagaatccgttaaaaaagaggggagggaaacattatgatggcaccgaaacagacaccttctccagaaaaaaatagaaaccttgtgggtccagtgctgcaacgccgccaaacagagggcacgtttgaccagcgcctgcttgaaatgagagcagaccacaattggaagcacgcagacccgtggagagtgttaagaattcagtcagagtttgttgcaggatttgatgctctgcatgagatgccgaaggcggtcacagtgtttggaagcgcgagaattaaagaggatcatccttattataaagcaggtgttgagcttggagaaaaacttgttgccgctgattacgcggttgtaacgggcggaggccctggccttatggaagcaccgaataaaggagcgtcagaagccaatggcttatcagtcggtttaggtattgaacttcctcacgagcagcatttaaacccgtatgtggatctgggattaaattttagatacttttttgctagaaagacgatgtttctgaaatattcacaagcttttgtctgtctgccgggcggcttcggaacactggacgaactgttcgaagtgctgtgcatggtccaaacaggcaaagttacaaacttcccgattgtactgatcggcacagaattctgggctggcctggttgactggatcagacatagactggtcgaagaaggcatgattgatgaaaaagatgtagacagaatgctggtaacagatgatctggatcaagccgttaagttcatcgtcgatgcccatgccggcctggatgtagcgagaagacataactaaattaattctagaaattaagaaggagggattcgtcatgttggtattccaaatgcgttatgtagataaaacatctactgttttgaaacagactaaaaacagtgattacgcagataaataaatacgttagattaattcctaccagtgactaatcttatgactttttaaacagataactaaaattacaaacaaatcgtttaacttctgtatttgtttatagatgtaatcacttcaggagtgattacatgaacaaaaatataaaatattctcaaaactttttaacgagtgaaaaagtactcaaccaaataataaaacaattgaatttaaaagaaaccgataccgtttacgaaattggaacaggtaaagggcatttaacgacgaaactggctaaaataagtaaacaggtaacgtctattgaattagacagtcatctattcaacttatcgtcagaaaaattaaaactgaacattcgtgtcactttaattcaccaagatattctacagtttcaattccctaacaaacagaggtataaaattgttgggaatattccttaccatttaagcacacaaattattaaaaaagtggtttttgaaagccatgcgtctgacatctatctgattgttgaagaaggattctacaagcgtaccttggatattcaccgaacactagggttgctcttgcacactcaagtctcgattcagcaattgcttaagctgccagcggaatgctttcatcctaaaccaaaagtaaacagtgtcttaataaaacttacccgccataccacagatgttccagataaatattggaagctatatacgtactttgtttcaaaatgggtcaatcgagaatatcgtcaactgtttactaaaaatcagtttcatcaagcaatgaaacacgccaaagtaaacaatttaagtaccgttacttatgagcaagtattgtctatttttaatagttatctattatttaacgggaggaaataattctatgagtcgcttttgtaaatttggaaagttacacgttactaaagggaatgtagatggatcctgtatgccaatgatgatgaagagtcgacatggatgagcgatgatgatatccgtttaggctgggcggtgatagcttctcgttcaggcagtacgcctcttttcttttccagacctgagggaggcggaaatggtgtgaggttcccggggaaaagccaaataggcgatcgcgggagtgctttatttgaagatcaggctatcactgcggtcaatagatttcacaatgtgatggctggacagcctgaggaactctcgaacccgaatggaaacaaccagatatttatgaatcagcgcggctcacatggcgttgtgctggcaaatgcaggttcatcctctgtctctatcaatacggcaacaaaattgcctgatggcaggtatgacaataaagctggagcgggttcatttcaagtgaacgatggtaaactgacaggcacgatcaatgccaggtctgtagctgtgctttatcctgatgatattgcaaaagcgcctcatgttttccttgagaattacaaaacaggtgtaacacattctttcaatgatcaactgacgattaccttgcgtgcagatgcgaatacaacaaaagccgtttatcaaatcaataatggaccagagacggcgtttaaggatggagatcaattcacaatcggaaaaggagatccatttggcaaaacatacaccatcatgttaaaaggaacgaacagtgatggtgtaacgaggaccgagaaatacagttttgttaaaagagatccagcgtcggccaaaaccatcggctatcaaaatccgaatcattggagccaggtaaatgcttatatctataaacatgatgggagccgagtaattgaattgaccggatcttggcctggaaaaccaatgactaaaaatgcagacggaatttacacgctgacgctgcctgcggacacggatacaaccaacgcaaaagtgatttttaataatggcagcgcccaagtgcccggtcagaatcagcctggctttgattacgtgctaaatggtttatataatgactcgggcttaagcggttctcttccccattg

SEQ ID NO: 143 - DNA, 5015 bp, Artificial Sequence
  complete  AmyE 0 IPT1 LOG4 EryR AmyE END operon
gcggtagtggtgcttacgatgtacgacagggggattccccatacattcttcgcttggctgaaaatgattcttctttttatcgtctgcggcggcgttctgtttctgcttcggtatgtgattgtgaagctggcttacagaagagcggtaaaagaagaaataaaaaagaaatcatcttttttgtttggaaagcgagggaagcgttcacagtttcgggcagctttttttataggaacattgatttgtattcactctgccaagttgttttgatagagtgattgtgataattttaaatgtaagcgttaacaaaattctccagtcttcacatcggtttgaaaggaggaagcggaagaatgaagtaagagggatttttgactccgaagtaagtcttcaaaaaatcaaataaggagtgtcaagaatgtttgcaaaacgattcaaaacctctttactgccgttattcgctggatttttattgctgtttcatttggttctggcaggaccggcggctgcgagtgctgaaacggcgaacaaatcgaatgagcttacagcaccgtcgatcaaaagcggaaccattcttcatgcatggaattggtcgttcaatacgttaaaacacaatatgaaggatattcatgatgcaggatatacagccattcagacatctccgattaaccaagtaaaggaagggaatcaaggagataaaagcatgtcgaactggtactggctgtatcagccgacatcgtatcaaattggcaaccgttacttaggtactgaacaagaatttaaagaaatgtgtgcagccgctgaagaatatggcataaaggtcattgttgacgcggtcatcaatcataccaccagtgattatgccgcgatttccaatgaggttaagagtattccaaactggacacatggaaacacacaaattaaaaactggtctgatcgatgggatgtcacgcagaattcattgctcgggctgtatgactggaatacacaaaatacacaagtacagtcctatctgaaacggttcttagacagggcattgaatgacggggcagacggttttcgatttgatgccgccaaacatatagagcttccagatgatggcagttacggcagtcaattttggccgaatatcacaaatacatctgcagagttccaatacggagaaatcctgcaggatagtgcctccagagatgctgcttatgcgaattatatggatgtgacagcgtctaactatgggcattccataaggtccgctttaaagaatcgtaatctgggcgtgtcgaatatctcccactatgcatctgatgtgtctgcggacaagctagtgacatgggtagagtcgcatgatacacgggtgcgcatgatcgtatggttcactgtccaccaaccaaaactgtgctcagtaccgccaatatttctcccttggggggtacaaagaggtgtccctagaagagatccacgctgtgtaaaaattttacaaaaaggtattgactttccctacagggtgtgtaataatttaattacaggcgggggcaaccccgctcagtacctagagcgtaaaagaggggagggaaacactagtatgttattacacctgatctacggtcctacatgtagtggtaaaactgatatggctatacaaatcgcgcaagaaacaggttggcccgtcgttgctcttgatcgtgtgcagtgctgtccacagattgcgacaggatccggcagacctctggaaagcgagttacaatctacccggcggatttatttagactcacgccctttgacagaaggcattcttgacgcggaatccgctcatcgccgtcttatattcgaggtagattggaggaaatcggaagaaggcttaatcctcgaaggagggtccatctcgcttctaaattgtatggctaagtctccattttggagatctggattccagtggcacgtcaagcgcctgagacttggggattcagatgcgtttctgacgcgagcaaaacagcgtgtggctgagatgtttgctattagagaagatcggccaagtttattggaggagctcgccgagctgtggaattacccggcagctcgcccgattttggaagacattgacggctatcgatgtgccattagatttgcaaggaaacacgatctcgccatatcacagctgccgaacatcgatgccggccggcatgttgaactgatcgaagccattgcaaacgaatatcttgaacatgcgctaagccaagaacgcgactttcctcaatggccggaggacggcgcaggacaaccagtatgcccggttaccttaacgagaatccgttaaaaaagaggggagggaaacattatgatgacgcttagatcagtctgtgtgttctgcggcgcgagcccgggagcttctccggtttatcaagaagctgctgtggcccttggtagacatttagcggaacgcggactgacgttagtatatggcggaggtgccgtaggactgatgggtacagtggccgacgccgctctggcggcgggcggagaagttattggtattatccctcaatcacttcaggaggccgaaattggccataaaggcttaacaagattagaagttgtagatggcatgcatgcacgcaaggctagaatggctgagttagctgatgcctttatcgcgcttccgggcggacttggaacacttgaggaactgtttgaggtctggacatggggccagttaggctaccacgcaaaaccactgggcctgctggaagttaatggcttctatgatcctctgctgacatttctggaccacctggtcgatgaaagatttgtgagagcggaacatagaggcatgctgcagagaggagcgtcacctgaagcactgctggacgcactggcagcatggacaccgtcagtcgcaccgaaatgggttgatagaacaccacaataaattaattctagaaattaagaaggagggattcgtcatgttggtattccaaatgcgttatgtagataaaacatctactgttttgaaacagactaaaaacagtgattacgcagataaataaatacgttagattaattcctaccagtgactaatcttatgactttttaaacagataactaaaattacaaacaaatcgtttaacttctgtatttgtttatagatgtaatcacttcaggagtgattacatgaacaaaaatataaaatattctcaaaactttttaacgagtgaaaaagtactcaaccaaataataaaacaattgaatttaaaagaaaccgataccgtttacgaaattggaacaggtaaagggcatttaacgacgaaactggctaaaataagtaaacaggtaacgtctattgaattagacagtcatctattcaacttatcgtcagaaaaattaaaactgaacattcgtgtcactttaattcaccaagatattctacagtttcaattccctaacaaacagaggtataaaattgttgggaatattccttaccatttaagcacacaaattattaaaaaagtggtttttgaaagccatgcgtctgacatctatctgattgttgaagaaggattctacaagcgtaccttggatattcaccgaacactagggttgctcttgcacactcaagtctcgattcagcaattgcttaagctgccagcggaatgctttcatcctaaaccaaaagtaaacagtgtcttaataaaacttacccgccataccacagatgttccagataaatattggaagctatatacgtactttgtttcaaaatgggtcaatcgagaatatcgtcaactgtttactaaaaatcagtttcatcaagcaatgaaacacgccaaagtaaacaatttaagtaccgttacttatgagcaagtattgtctatttttaatagttatctattatttaacgggaggaaataattctatgagtcgcttttgtaaatttggaaagttacacgttactaaagggaatgtagatggatcctgtatgccaatgatgatgaagagtcgacatggatgagcgatgatgatatccgtttaggctgggcggtgatagcttctcgttcaggcagtacgcctcttttcttttccagacctgagggaggcggaaatggtgtgaggttcccggggaaaagccaaataggcgatcgcgggagtgctttatttgaagatcaggctatcactgcggtcaatagatttcacaatgtgatggctggacagcctgaggaactctcgaacccgaatggaaacaaccagatatttatgaatcagcgcggctcacatggcgttgtgctggcaaatgcaggttcatcctctgtctctatcaatacggcaacaaaattgcctgatggcaggtatgacaataaagctggagcgggttcatttcaagtgaacgatggtaaactgacaggcacgatcaatgccaggtctgtagctgtgctttatcctgatgatattgcaaaagcgcctcatgttttccttgagaattacaaaacaggtgtaacacattctttcaatgatcaactgacgattaccttgcgtgcagatgcgaatacaacaaaagccgtttatcaaatcaataatggaccagagacggcgtttaaggatggagatcaattcacaatcggaaaaggagatccatttggcaaaacatacaccatcatgttaaaaggaacgaacagtgatggtgtaacgaggaccgagaaatacagttttgttaaaagagatccagcgtcggccaaaaccatcggctatcaaaatccgaatcattggagccaggtaaatgcttatatctataaacatgatgggagccgagtaattgaattgaccggatcttggcctggaaaaccaatgactaaaaatgcagacggaatttacacgctgacgctgcctgcggacacggatacaaccaacgcaaaagtgatttttaataatggcagcgcccaagtgcccggtcagaatcagcctggctttgattacgtgctaaatggtttatataatgactcgggcttaagcggttctcttccccattg

SEQ ID NO: 144 - DNA, 5198 bp, Artificial Sequence
  complete  AmyE 0 IPT1 LOG5 EryR AmyE END operon
gcggtagtggtgcttacgatgtacgacagggggattccccatacattcttcgcttggctgaaaatgattcttctttttatcgtctgcggcggcgttctgtttctgcttcggtatgtgattgtgaagctggcttacagaagagcggtaaaagaagaaataaaaaagaaatcatcttttttgtttggaaagcgagggaagcgttcacagtttcgggcagctttttttataggaacattgatttgtattcactctgccaagttgttttgatagagtgattgtgataattttaaatgtaagcgttaacaaaattctccagtcttcacatcggtttgaaaggaggaagcggaagaatgaagtaagagggatttttgactccgaagtaagtcttcaaaaaatcaaataaggagtgtcaagaatgtttgcaaaacgattcaaaacctctttactgccgttattcgctggatttttattgctgtttcatttggttctggcaggaccggcggctgcgagtgctgaaacggcgaacaaatcgaatgagcttacagcaccgtcgatcaaaagcggaaccattcttcatgcatggaattggtcgttcaatacgttaaaacacaatatgaaggatattcatgatgcaggatatacagccattcagacatctccgattaaccaagtaaaggaagggaatcaaggagataaaagcatgtcgaactggtactggctgtatcagccgacatcgtatcaaattggcaaccgttacttaggtactgaacaagaatttaaagaaatgtgtgcagccgctgaagaatatggcataaaggtcattgttgacgcggtcatcaatcataccaccagtgattatgccgcgatttccaatgaggttaagagtattccaaactggacacatggaaacacacaaattaaaaactggtctgatcgatgggatgtcacgcagaattcattgctcgggctgtatgactggaatacacaaaatacacaagtacagtcctatctgaaacggttcttagacagggcattgaatgacggggcagacggttttcgatttgatgccgccaaacatatagagcttccagatgatggcagttacggcagtcaattttggccgaatatcacaaatacatctgcagagttccaatacggagaaatcctgcaggatagtgcctccagagatgctgcttatgcgaattatatggatgtgacagcgtctaactatgggcattccataaggtccgctttaaagaatcgtaatctgggcgtgtcgaatatctcccactatgcatctgatgtgtctgcggacaagctagtgacatgggtagagtcgcatgatacacgggtgcgcatgatcgtatggttcactgtccaccaaccaaaactgtgctcagtaccgccaatatttctcccttggggggtacaaagaggtgtccctagaagagatccacgctgtgtaaaaattttacaaaaaggtattgactttccctacagggtgtgtaataatttaattacaggcgggggcaaccccgctcagtacctagagcgtaaaagaggggagggaaacactagtatgttattacacctgatctacggtcctacatgtagtggtaaaactgatatggctatacaaatcgcgcaagaaacaggttggcccgtcgttgctcttgatcgtgtgcagtgctgtccacagattgcgacaggatccggcagacctctggaaagcgagttacaatctacccggcggatttatttagactcacgccctttgacagaaggcattcttgacgcggaatccgctcatcgccgtcttatattcgaggtagattggaggaaatcggaagaaggcttaatcctcgaaggagggtccatctcgcttctaaattgtatggctaagtctccattttggagatctggattccagtggcacgtcaagcgcctgagacttggggattcagatgcgtttctgacgcgagcaaaacagcgtgtggctgagatgtttgctattagagaagatcggccaagtttattggaggagctcgccgagctgtggaattacccggcagctcgcccgattttggaagacattgacggctatcgatgtgccattagatttgcaaggaaacacgatctcgccatatcacagctgccgaacatcgatgccggccggcatgttgaactgatcgaagccattgcaaacgaatatcttgaacatgcgctaagccaagaacgcgactttcctcaatggccggaggacggcgcaggacaaccagtatgcccggttaccttaacgagaatccgttaaaaaagaggggagggaaacattatgatgaacagcgttgacgaccctggttcatacgaacagattggcagcgagaagccggcaaagcataaaggctctgttgtactgcgcagaggcagaaatacggaaacatctacgtcagaccaacgcttattagatgaaagaggttcaggtaattggatccattctgatacgtggcgcgtcttacgcattcagagcgaatttgtggaaggctttggtgcgctggccgaagttcctagagctgtcacagtattcggcagcgccagaacgccagtcgatcacccggaatacgagagagctagagagttaggagccgcgcttgctagagaaggatttgcggtaattacaggcggtggaccaggcgtcatggagggagctaacagaggagcgtcagaggcggatggctattcaattggccttggcatcgagctgccgttcgaacaaggcttaaacgactgggtagatcttggagtgaatttcagatatttttttgtaagaaaaacaatgttcgtgaaatattcacaggcctttgtgtgccttccgggcggctttggaacactggacgaactgtttgaagcactgacacttgttcaaacagcaaaagtcacaagatttccgattgttctgctgggaaaggacttctggtcaggactgctggattggattaaaacaacactggttaaagagggcaaagtctcagaaaaagatctggatctgatccattgtacagattcagtggatgaagcagtgagaatcgttagagaagcacaacagtaaattaattctagaaattaagaaggagggattcgtcatgttggtattccaaatgcgttatgtagataaaacatctactgttttgaaacagactaaaaacagtgattacgcagataaataaatacgttagattaattcctaccagtgactaatcttatgactttttaaacagataactaaaattacaaacaaatcgtttaacttctgtatttgtttatagatgtaatcacttcaggagtgattacatgaacaaaaatataaaatattctcaaaactttttaacgagtgaaaaagtactcaaccaaataataaaacaattgaatttaaaagaaaccgataccgtttacgaaattggaacaggtaaagggcatttaacgacgaaactggctaaaataagtaaacaggtaacgtctattgaattagacagtcatctattcaacttatcgtcagaaaaattaaaactgaacattcgtgtcactttaattcaccaagatattctacagtttcaattccctaacaaacagaggtataaaattgttgggaatattccttaccatttaagcacacaaattattaaaaaagtggtttttgaaagccatgcgtctgacatctatctgattgttgaagaaggattctacaagcgtaccttggatattcaccgaacactagggttgctcttgcacactcaagtctcgattcagcaattgcttaagctgccagcggaatgctttcatcctaaaccaaaagtaaacagtgtcttaataaaacttacccgccataccacagatgttccagataaatattggaagctatatacgtactttgtttcaaaatgggtcaatcgagaatatcgtcaactgtttactaaaaatcagtttcatcaagcaatgaaacacgccaaagtaaacaatttaagtaccgttacttatgagcaagtattgtctatttttaatagttatctattatttaacgggaggaaataattctatgagtcgcttttgtaaatttggaaagttacacgttactaaagggaatgtagatggatcctgtatgccaatgatgatgaagagtcgacatggatgagcgatgatgatatccgtttaggctgggcggtgatagcttctcgttcaggcagtacgcctcttttcttttccagacctgagggaggcggaaatggtgtgaggttcccggggaaaagccaaataggcgatcgcgggagtgctttatttgaagatcaggctatcactgcggtcaatagatttcacaatgtgatggctggacagcctgaggaactctcgaacccgaatggaaacaaccagatatttatgaatcagcgcggctcacatggcgttgtgctggcaaatgcaggttcatcctctgtctctatcaatacggcaacaaaattgcctgatggcaggtatgacaataaagctggagcgggttcatttcaagtgaacgatggtaaactgacaggcacgatcaatgccaggtctgtagctgtgctttatcctgatgatattgcaaaagcgcctcatgttttccttgagaattacaaaacaggtgtaacacattctttcaatgatcaactgacgattaccttgcgtgcagatgcgaatacaacaaaagccgtttatcaaatcaataatggaccagagacggcgtttaaggatggagatcaattcacaatcggaaaaggagatccatttggcaaaacatacaccatcatgttaaaaggaacgaacagtgatggtgtaacgaggaccgagaaatacagttttgttaaaagagatccagcgtcggccaaaaccatcggctatcaaaatccgaatcattggagccaggtaaatgcttatatctataaacatgatgggagccgagtaattgaattgaccggatcttggcctggaaaaccaatgactaaaaatgcagacggaatttacacgctgacgctgcctgcggacacggatacaaccaacgcaaaagtgatttttaataatggcagcgcccaagtgcccggtcagaatcagcctggctttgattacgtgctaaatggtttatataatgactcgggcttaagcggttctcttccccattg

SEQ ID NO: 145 - DNA, 5024 bp, Artificial Sequence
  complete  AmyE 0 IPT1 LOG6 EryR AmyE END operon
gcggtagtggtgcttacgatgtacgacagggggattccccatacattcttcgcttggctgaaaatgattcttctttttatcgtctgcggcggcgttctgtttctgcttcggtatgtgattgtgaagctggcttacagaagagcggtaaaagaagaaataaaaaagaaatcatcttttttgtttggaaagcgagggaagcgttcacagtttcgggcagctttttttataggaacattgatttgtattcactctgccaagttgttttgatagagtgattgtgataattttaaatgtaagcgttaacaaaattctccagtcttcacatcggtttgaaaggaggaagcggaagaatgaagtaagagggatttttgactccgaagtaagtcttcaaaaaatcaaataaggagtgtcaagaatgtttgcaaaacgattcaaaacctctttactgccgttattcgctggatttttattgctgtttcatttggttctggcaggaccggcggctgcgagtgctgaaacggcgaacaaatcgaatgagcttacagcaccgtcgatcaaaagcggaaccattcttcatgcatggaattggtcgttcaatacgttaaaacacaatatgaaggatattcatgatgcaggatatacagccattcagacatctccgattaaccaagtaaaggaagggaatcaaggagataaaagcatgtcgaactggtactggctgtatcagccgacatcgtatcaaattggcaaccgttacttaggtactgaacaagaatttaaagaaatgtgtgcagccgctgaagaatatggcataaaggtcattgttgacgcggtcatcaatcataccaccagtgattatgccgcgatttccaatgaggttaagagtattccaaactggacacatggaaacacacaaattaaaaactggtctgatcgatgggatgtcacgcagaattcattgctcgggctgtatgactggaatacacaaaatacacaagtacagtcctatctgaaacggttcttagacagggcattgaatgacggggcagacggttttcgatttgatgccgccaaacatatagagcttccagatgatggcagttacggcagtcaattttggccgaatatcacaaatacatctgcagagttccaatacggagaaatcctgcaggatagtgcctccagagatgctgcttatgcgaattatatggatgtgacagcgtctaactatgggcattccataaggtccgctttaaagaatcgtaatctgggcgtgtcgaatatctcccactatgcatctgatgtgtctgcggacaagctagtgacatgggtagagtcgcatgatacacgggtgcgcatgatcgtatggttcactgtccaccaaccaaaactgtgctcagtaccgccaatatttctcccttggggggtacaaagaggtgtccctagaagagatccacgctgtgtaaaaattttacaaaaaggtattgactttccctacagggtgtgtaataatttaattacaggcgggggcaaccccgctcagtacctagagcgtaaaagaggggagggaaacactagtatgttattacacctgatctacggtcctacatgtagtggtaaaactgatatggctatacaaatcgcgcaagaaacaggttggcccgtcgttgctcttgatcgtgtgcagtgctgtccacagattgcgacaggatccggcagacctctggaaagcgagttacaatctacccggcggatttatttagactcacgccctttgacagaaggcattcttgacgcggaatccgctcatcgccgtcttatattcgaggtagattggaggaaatcggaagaaggcttaatcctcgaaggagggtccatctcgcttctaaattgtatggctaagtctccattttggagatctggattccagtggcacgtcaagcgcctgagacttggggattcagatgcgtttctgacgcgagcaaaacagcgtgtggctgagatgtttgctattagagaagatcggccaagtttattggaggagctcgccgagctgtggaattacccggcagctcgcccgattttggaagacattgacggctatcgatgtgccattagatttgcaaggaaacacgatctcgccatatcacagctgccgaacatcgatgccggccggcatgttgaactgatcgaagccattgcaaacgaatatcttgaacatgcgctaagccaagaacgcgactttcctcaatggccggaggacggcgcaggacaaccagtatgcccggttaccttaacgagaatccgttaaaaaagaggggagggaaacattatgatgaatttacgcccgatgcctgccacgacggttagcgcccaagcaagaccaacgccaaagtcagttacggtgttctgcggagctatgccgggtagaggaacaaaatacggtcaacttgctgagggcatgggtagagctattgctagaagcaaattaagacttgtatatggaggagcgagagtcggccttatgggaacactggcaaacgcagccttagactctggcggtacagtcgttggagtcattcctgaatcatttacagctatccctgaagccgcccaccacggactgacagaactgcatgtagtgcacgatatgcatcaaagaaaagccttaatggctgagctgggcgatgcattcattgcacttcctggcggcgtcggcacagcggaggagttttttgaggtgctgacatggagccatctgggcctgcataacaaaccgtgcgttctgctgaatgataatgaatactatagaccgctgctgtcatatattgaacatgcagcggtcgaaggctttatcacacctgcgacaagatcaagagtaatcgtgtgtaaagacattgaaggcgcgattgcggcgattagatcaccgtaaattaattctagaaattaagaaggagggattcgtcatgttggtattccaaatgcgttatgtagataaaacatctactgttttgaaacagactaaaaacagtgattacgcagataaataaatacgttagattaattcctaccagtgactaatcttatgactttttaaacagataactaaaattacaaacaaatcgtttaacttctgtatttgtttatagatgtaatcacttcaggagtgattacatgaacaaaaatataaaatattctcaaaactttttaacgagtgaaaaagtactcaaccaaataataaaacaattgaatttaaaagaaaccgataccgtttacgaaattggaacaggtaaagggcatttaacgacgaaactggctaaaataagtaaacaggtaacgtctattgaattagacagtcatctattcaacttatcgtcagaaaaattaaaactgaacattcgtgtcactttaattcaccaagatattctacagtttcaattccctaacaaacagaggtataaaattgttgggaatattccttaccatttaagcacacaaattattaaaaaagtggtttttgaaagccatgcgtctgacatctatctgattgttgaagaaggattctacaagcgtaccttggatattcaccgaacactagggttgctcttgcacactcaagtctcgattcagcaattgcttaagctgccagcggaatgctttcatcctaaaccaaaagtaaacagtgtcttaataaaacttacccgccataccacagatgttccagataaatattggaagctatatacgtactttgtttcaaaatgggtcaatcgagaatatcgtcaactgtttactaaaaatcagtttcatcaagcaatgaaacacgccaaagtaaacaatttaagtaccgttacttatgagcaagtattgtctatttttaatagttatctattatttaacgggaggaaataattctatgagtcgcttttgtaaatttggaaagttacacgttactaaagggaatgtagatggatcctgtatgccaatgatgatgaagagtcgacatggatgagcgatgatgatatccgtttaggctgggcggtgatagcttctcgttcaggcagtacgcctcttttcttttccagacctgagggaggcggaaatggtgtgaggttcccggggaaaagccaaataggcgatcgcgggagtgctttatttgaagatcaggctatcactgcggtcaatagatttcacaatgtgatggctggacagcctgaggaactctcgaacccgaatggaaacaaccagatatttatgaatcagcgcggctcacatggcgttgtgctggcaaatgcaggttcatcctctgtctctatcaatacggcaacaaaattgcctgatggcaggtatgacaataaagctggagcgggttcatttcaagtgaacgatggtaaactgacaggcacgatcaatgccaggtctgtagctgtgctttatcctgatgatattgcaaaagcgcctcatgttttccttgagaattacaaaacaggtgtaacacattctttcaatgatcaactgacgattaccttgcgtgcagatgcgaatacaacaaaagccgtttatcaaatcaataatggaccagagacggcgtttaaggatggagatcaattcacaatcggaaaaggagatccatttggcaaaacatacaccatcatgttaaaaggaacgaacagtgatggtgtaacgaggaccgagaaatacagttttgttaaaagagatccagcgtcggccaaaaccatcggctatcaaaatccgaatcattggagccaggtaaatgcttatatctataaacatgatgggagccgagtaattgaattgaccggatcttggcctggaaaaccaatgactaaaaatgcagacggaatttacacgctgacgctgcctgcggacacggatacaaccaacgcaaaagtgatttttaataatggcagcgcccaagtgcccggtcagaatcagcctggctttgattacgtgctaaatggtttatataatgactcgggcttaagcggttctcttccccattg

SEQ ID NO: 146 - DNA, 4991 bp, Artificial Sequence
  complete  AmyE 0 IPT1 LOG7 EryR AmyE END operon
gcggtagtggtgcttacgatgtacgacagggggattccccatacattcttcgcttggctgaaaatgattcttctttttatcgtctgcggcggcgttctgtttctgcttcggtatgtgattgtgaagctggcttacagaagagcggtaaaagaagaaataaaaaagaaatcatcttttttgtttggaaagcgagggaagcgttcacagtttcgggcagctttttttataggaacattgatttgtattcactctgccaagttgttttgatagagtgattgtgataattttaaatgtaagcgttaacaaaattctccagtcttcacatcggtttgaaaggaggaagcggaagaatgaagtaagagggatttttgactccgaagtaagtcttcaaaaaatcaaataaggagtgtcaagaatgtttgcaaaacgattcaaaacctctttactgccgttattcgctggatttttattgctgtttcatttggttctggcaggaccggcggctgcgagtgctgaaacggcgaacaaatcgaatgagcttacagcaccgtcgatcaaaagcggaaccattcttcatgcatggaattggtcgttcaatacgttaaaacacaatatgaaggatattcatgatgcaggatatacagccattcagacatctccgattaaccaagtaaaggaagggaatcaaggagataaaagcatgtcgaactggtactggctgtatcagccgacatcgtatcaaattggcaaccgttacttaggtactgaacaagaatttaaagaaatgtgtgcagccgctgaagaatatggcataaaggtcattgttgacgcggtcatcaatcataccaccagtgattatgccgcgatttccaatgaggttaagagtattccaaactggacacatggaaacacacaaattaaaaactggtctgatcgatgggatgtcacgcagaattcattgctcgggctgtatgactggaatacacaaaatacacaagtacagtcctatctgaaacggttcttagacagggcattgaatgacggggcagacggttttcgatttgatgccgccaaacatatagagcttccagatgatggcagttacggcagtcaattttggccgaatatcacaaatacatctgcagagttccaatacggagaaatcctgcaggatagtgcctccagagatgctgcttatgcgaattatatggatgtgacagcgtctaactatgggcattccataaggtccgctttaaagaatcgtaatctgggcgtgtcgaatatctcccactatgcatctgatgtgtctgcggacaagctagtgacatgggtagagtcgcatgatacacgggtgcgcatgatcgtatggttcactgtccaccaaccaaaactgtgctcagtaccgccaatatttctcccttggggggtacaaagaggtgtccctagaagagatccacgctgtgtaaaaattttacaaaaaggtattgactttccctacagggtgtgtaataatttaattacaggcgggggcaaccccgctcagtacctagagcgtaaaagaggggagggaaacactagtatgttattacacctgatctacggtcctacatgtagtggtaaaactgatatggctatacaaatcgcgcaagaaacaggttggcccgtcgttgctcttgatcgtgtgcagtgctgtccacagattgcgacaggatccggcagacctctggaaagcgagttacaatctacccggcggatttatttagactcacgccctttgacagaaggcattcttgacgcggaatccgctcatcgccgtcttatattcgaggtagattggaggaaatcggaagaaggcttaatcctcgaaggagggtccatctcgcttctaaattgtatggctaagtctccattttggagatctggattccagtggcacgtcaagcgcctgagacttggggattcagatgcgtttctgacgcgagcaaaacagcgtgtggctgagatgtttgctattagagaagatcggccaagtttattggaggagctcgccgagctgtggaattacccggcagctcgcccgattttggaagacattgacggctatcgatgtgccattagatttgcaaggaaacacgatctcgccatatcacagctgccgaacatcgatgccggccggcatgttgaactgatcgaagccattgcaaacgaatatcttgaacatgcgctaagccaagaacgcgactttcctcaatggccggaggacggcgcaggacaaccagtatgcccggttaccttaacgagaatccgttaaaaaagaggggagggaaacattatgatgaaatcagtagcgatttattgcggatcatcaatgggatctagccctagatatgcagaagtagctaaagagatgggagaggaacttgcaagacgcaatctgagagtagtgtatggaggaggaaacgtcggactgatgggtacagtggccgacgcagcgttagcggccggtggcgaggtggtaggcgtaattccaagacagttaattaaccgcgagatggcacatgccggtctgacagatttagaagtggtggacacaatggcgcagagaaaaacgagaatggaggaacttgccgatgcttttgtgtgtctgcctggcggtgttggcacgctggaagaagtcgttgaagttcttacaatgcaacaacttggccacgtccaaggcccggtcggcctggttaatgttgacggcttttgggaaccgtttgtcagaacactggaatcaatggtcgcttgcggcttcgttcagtcaagatacatcgatgctatcgcactgtcttcagatccggctgaaattctggatgaattcgcaggctggagcccgatcggcgcgaagtggagctaaattaattctagaaattaagaaggagggattcgtcatgttggtattccaaatgcgttatgtagataaaacatctactgttttgaaacagactaaaaacagtgattacgcagataaataaatacgttagattaattcctaccagtgactaatcttatgactttttaaacagataactaaaattacaaacaaatcgtttaacttctgtatttgtttatagatgtaatcacttcaggagtgattacatgaacaaaaatataaaatattctcaaaactttttaacgagtgaaaaagtactcaaccaaataataaaacaattgaatttaaaagaaaccgataccgtttacgaaattggaacaggtaaagggcatttaacgacgaaactggctaaaataagtaaacaggtaacgtctattgaattagacagtcatctattcaacttatcgtcagaaaaattaaaactgaacattcgtgtcactttaattcaccaagatattctacagtttcaattccctaacaaacagaggtataaaattgttgggaatattccttaccatttaagcacacaaattattaaaaaagtggtttttgaaagccatgcgtctgacatctatctgattgttgaagaaggattctacaagcgtaccttggatattcaccgaacactagggttgctcttgcacactcaagtctcgattcagcaattgcttaagctgccagcggaatgctttcatcctaaaccaaaagtaaacagtgtcttaataaaacttacccgccataccacagatgttccagataaatattggaagctatatacgtactttgtttcaaaatgggtcaatcgagaatatcgtcaactgtttactaaaaatcagtttcatcaagcaatgaaacacgccaaagtaaacaatttaagtaccgttacttatgagcaagtattgtctatttttaatagttatctattatttaacgggaggaaataattctatgagtcgcttttgtaaatttggaaagttacacgttactaaagggaatgtagatggatcctgtatgccaatgatgatgaagagtcgacatggatgagcgatgatgatatccgtttaggctgggcggtgatagcttctcgttcaggcagtacgcctcttttcttttccagacctgagggaggcggaaatggtgtgaggttcccggggaaaagccaaataggcgatcgcgggagtgctttatttgaagatcaggctatcactgcggtcaatagatttcacaatgtgatggctggacagcctgaggaactctcgaacccgaatggaaacaaccagatatttatgaatcagcgcggctcacatggcgttgtgctggcaaatgcaggttcatcctctgtctctatcaatacggcaacaaaattgcctgatggcaggtatgacaataaagctggagcgggttcatttcaagtgaacgatggtaaactgacaggcacgatcaatgccaggtctgtagctgtgctttatcctgatgatattgcaaaagcgcctcatgttttccttgagaattacaaaacaggtgtaacacattctttcaatgatcaactgacgattaccttgcgtgcagatgcgaatacaacaaaagccgtttatcaaatcaataatggaccagagacggcgtttaaggatggagatcaattcacaatcggaaaaggagatccatttggcaaaacatacaccatcatgttaaaaggaacgaacagtgatggtgtaacgaggaccgagaaatacagttttgttaaaagagatccagcgtcggccaaaaccatcggctatcaaaatccgaatcattggagccaggtaaatgcttatatctataaacatgatgggagccgagtaattgaattgaccggatcttggcctggaaaaccaatgactaaaaatgcagacggaatttacacgctgacgctgcctgcggacacggatacaaccaacgcaaaagtgatttttaataatggcagcgcccaagtgcccggtcagaatcagcctggctttgattacgtgctaaatggtttatataatgactcgggcttaagcggttctcttccccattg

SEQ ID NO: 147 - DNA, 5255 bp, Artificial Sequence
  complete  AmyE 0 IPT1 LOG8 EryR AmyE END operon
gcggtagtggtgcttacgatgtacgacagggggattccccatacattcttcgcttggctgaaaatgattcttctttttatcgtctgcggcggcgttctgtttctgcttcggtatgtgattgtgaagctggcttacagaagagcggtaaaagaagaaataaaaaagaaatcatcttttttgtttggaaagcgagggaagcgttcacagtttcgggcagctttttttataggaacattgatttgtattcactctgccaagttgttttgatagagtgattgtgataattttaaatgtaagcgttaacaaaattctccagtcttcacatcggtttgaaaggaggaagcggaagaatgaagtaagagggatttttgactccgaagtaagtcttcaaaaaatcaaataaggagtgtcaagaatgtttgcaaaacgattcaaaacctctttactgccgttattcgctggatttttattgctgtttcatttggttctggcaggaccggcggctgcgagtgctgaaacggcgaacaaatcgaatgagcttacagcaccgtcgatcaaaagcggaaccattcttcatgcatggaattggtcgttcaatacgttaaaacacaatatgaaggatattcatgatgcaggatatacagccattcagacatctccgattaaccaagtaaaggaagggaatcaaggagataaaagcatgtcgaactggtactggctgtatcagccgacatcgtatcaaattggcaaccgttacttaggtactgaacaagaatttaaagaaatgtgtgcagccgctgaagaatatggcataaaggtcattgttgacgcggtcatcaatcataccaccagtgattatgccgcgatttccaatgaggttaagagtattccaaactggacacatggaaacacacaaattaaaaactggtctgatcgatgggatgtcacgcagaattcattgctcgggctgtatgactggaatacacaaaatacacaagtacagtcctatctgaaacggttcttagacagggcattgaatgacggggcagacggttttcgatttgatgccgccaaacatatagagcttccagatgatggcagttacggcagtcaattttggccgaatatcacaaatacatctgcagagttccaatacggagaaatcctgcaggatagtgcctccagagatgctgcttatgcgaattatatggatgtgacagcgtctaactatgggcattccataaggtccgctttaaagaatcgtaatctgggcgtgtcgaatatctcccactatgcatctgatgtgtctgcggacaagctagtgacatgggtagagtcgcatgatacacgggtgcgcatgatcgtatggttcactgtccaccaaccaaaactgtgctcagtaccgccaatatttctcccttggggggtacaaagaggtgtccctagaagagatccacgctgtgtaaaaattttacaaaaaggtattgactttccctacagggtgtgtaataatttaattacaggcgggggcaaccccgctcagtacctagagcgtaaaagaggggagggaaacactagtatgttattacacctgatctacggtcctacatgtagtggtaaaactgatatggctatacaaatcgcgcaagaaacaggttggcccgtcgttgctcttgatcgtgtgcagtgctgtccacagattgcgacaggatccggcagacctctggaaagcgagttacaatctacccggcggatttatttagactcacgccctttgacagaaggcattcttgacgcggaatccgctcatcgccgtcttatattcgaggtagattggaggaaatcggaagaaggcttaatcctcgaaggagggtccatctcgcttctaaattgtatggctaagtctccattttggagatctggattccagtggcacgtcaagcgcctgagacttggggattcagatgcgtttctgacgcgagcaaaacagcgtgtggctgagatgtttgctattagagaagatcggccaagtttattggaggagctcgccgagctgtggaattacccggcagctcgcccgattttggaagacattgacggctatcgatgtgccattagatttgcaaggaaacacgatctcgccatatcacagctgccgaacatcgatgccggccggcatgttgaactgatcgaagccattgcaaacgaatatcttgaacatgcgctaagccaagaacgcgactttcctcaatggccggaggacggcgcaggacaaccagtatgcccggttaccttaacgagaatccgttaaaaaagaggggagggaaacattatgatggcccctcttcgctcaaacaaagatggcaaacctagagttcctagaacgacggaaagcgccgaaacgaatagaatgttaagaggtccgatgttactgagaacagacggaaatcagacgtcaacatacgatcaaagacttttagagtcaggcgcagatcacgcttggaagcatgcagatccgtggcgcgttctgagaatccagggagagttcgtggcaggatttgatgctctgagcgatctgcctaaggcggttacggtatacggctcagccagaacaaaagaggacacaccggaatatgcacagggcatcgatttaggtgcaaaattagttgagcatgactacgctgttgtgacaggcggtggcccaggcatcatggaggcggcgaatagaggcgcctatgaagctggaggtctttctgtgggcctgggcattgaacttccgtttgaacaaggtttaaacaaatatgtagatcttggaattaactttagatactttttcgcgagaaagacaatgttcctgaaatattcacaggcgtttgtgtgcctgccgggcggtatgggaacaatggatgagctgtttgaaatgctgtgtatggtgcaaacaggaaaagtgacaaactatccgattgtactgatcggcacagaatattggagcggcctggtagaatggatggctaatacactggtcgccgaaggctacatcaatccacaagatatggatctgttcctgctgacagacagcgtcgacgaagccgtcgaacatattgtcgcggctcataaagaaatgacagacgaaagagtctcaagactgaatcacgacgcataaattaattctagaaattaagaaggagggattcgtcatgttggtattccaaatgcgttatgtagataaaacatctactgttttgaaacagactaaaaacagtgattacgcagataaataaatacgttagattaattcctaccagtgactaatcttatgactttttaaacagataactaaaattacaaacaaatcgtttaacttctgtatttgtttatagatgtaatcacttcaggagtgattacatgaacaaaaatataaaatattctcaaaactttttaacgagtgaaaaagtactcaaccaaataataaaacaattgaatttaaaagaaaccgataccgtttacgaaattggaacaggtaaagggcatttaacgacgaaactggctaaaataagtaaacaggtaacgtctattgaattagacagtcatctattcaacttatcgtcagaaaaattaaaactgaacattcgtgtcactttaattcaccaagatattctacagtttcaattccctaacaaacagaggtataaaattgttgggaatattccttaccatttaagcacacaaattattaaaaaagtggtttttgaaagccatgcgtctgacatctatctgattgttgaagaaggattctacaagcgtaccttggatattcaccgaacactagggttgctcttgcacactcaagtctcgattcagcaattgcttaagctgccagcggaatgctttcatcctaaaccaaaagtaaacagtgtcttaataaaacttacccgccataccacagatgttccagataaatattggaagctatatacgtactttgtttcaaaatgggtcaatcgagaatatcgtcaactgtttactaaaaatcagtttcatcaagcaatgaaacacgccaaagtaaacaatttaagtaccgttacttatgagcaagtattgtctatttttaatagttatctattatttaacgggaggaaataattctatgagtcgcttttgtaaatttggaaagttacacgttactaaagggaatgtagatggatcctgtatgccaatgatgatgaagagtcgacatggatgagcgatgatgatatccgtttaggctgggcggtgatagcttctcgttcaggcagtacgcctcttttcttttccagacctgagggaggcggaaatggtgtgaggttcccggggaaaagccaaataggcgatcgcgggagtgctttatttgaagatcaggctatcactgcggtcaatagatttcacaatgtgatggctggacagcctgaggaactctcgaacccgaatggaaacaaccagatatttatgaatcagcgcggctcacatggcgttgtgctggcaaatgcaggttcatcctctgtctctatcaatacggcaacaaaattgcctgatggcaggtatgacaataaagctggagcgggttcatttcaagtgaacgatggtaaactgacaggcacgatcaatgccaggtctgtagctgtgctttatcctgatgatattgcaaaagcgcctcatgttttccttgagaattacaaaacaggtgtaacacattctttcaatgatcaactgacgattaccttgcgtgcagatgcgaatacaacaaaagccgtttatcaaatcaataatggaccagagacggcgtttaaggatggagatcaattcacaatcggaaaaggagatccatttggcaaaacatacaccatcatgttaaaaggaacgaacagtgatggtgtaacgaggaccgagaaatacagttttgttaaaagagatccagcgtcggccaaaaccatcggctatcaaaatccgaatcattggagccaggtaaatgcttatatctataaacatgatgggagccgagtaattgaattgaccggatcttggcctggaaaaccaatgactaaaaatgcagacggaatttacacgctgacgctgcctgcggacacggatacaaccaacgcaaaagtgatttttaataatggcagcgcccaagtgcccggtcagaatcagcctggctttgattacgtgctaaatggtttatataatgactcgggcttaagcggttctcttccccattg

SEQ ID NO: 148 - DNA, 5159 bp, Artificial Sequence
  complete  AmyE 0 IPT1 LOG9 EryR AmyE END operon
gcggtagtggtgcttacgatgtacgacagggggattccccatacattcttcgcttggctgaaaatgattcttctttttatcgtctgcggcggcgttctgtttctgcttcggtatgtgattgtgaagctggcttacagaagagcggtaaaagaagaaataaaaaagaaatcatcttttttgtttggaaagcgagggaagcgttcacagtttcgggcagctttttttataggaacattgatttgtattcactctgccaagttgttttgatagagtgattgtgataattttaaatgtaagcgttaacaaaattctccagtcttcacatcggtttgaaaggaggaagcggaagaatgaagtaagagggatttttgactccgaagtaagtcttcaaaaaatcaaataaggagtgtcaagaatgtttgcaaaacgattcaaaacctctttactgccgttattcgctggatttttattgctgtttcatttggttctggcaggaccggcggctgcgagtgctgaaacggcgaacaaatcgaatgagcttacagcaccgtcgatcaaaagcggaaccattcttcatgcatggaattggtcgttcaatacgttaaaacacaatatgaaggatattcatgatgcaggatatacagccattcagacatctccgattaaccaagtaaaggaagggaatcaaggagataaaagcatgtcgaactggtactggctgtatcagccgacatcgtatcaaattggcaaccgttacttaggtactgaacaagaatttaaagaaatgtgtgcagccgctgaagaatatggcataaaggtcattgttgacgcggtcatcaatcataccaccagtgattatgccgcgatttccaatgaggttaagagtattccaaactggacacatggaaacacacaaattaaaaactggtctgatcgatgggatgtcacgcagaattcattgctcgggctgtatgactggaatacacaaaatacacaagtacagtcctatctgaaacggttcttagacagggcattgaatgacggggcagacggttttcgatttgatgccgccaaacatatagagcttccagatgatggcagttacggcagtcaattttggccgaatatcacaaatacatctgcagagttccaatacggagaaatcctgcaggatagtgcctccagagatgctgcttatgcgaattatatggatgtgacagcgtctaactatgggcattccataaggtccgctttaaagaatcgtaatctgggcgtgtcgaatatctcccactatgcatctgatgtgtctgcggacaagctagtgacatgggtagagtcgcatgatacacgggtgcgcatgatcgtatggttcactgtccaccaaccaaaactgtgctcagtaccgccaatatttctcccttggggggtacaaagaggtgtccctagaagagatccacgctgtgtaaaaattttacaaaaaggtattgactttccctacagggtgtgtaataatttaattacaggcgggggcaaccccgctcagtacctagagcgtaaaagaggggagggaaacactagtatgttattacacctgatctacggtcctacatgtagtggtaaaactgatatggctatacaaatcgcgcaagaaacaggttggcccgtcgttgctcttgatcgtgtgcagtgctgtccacagattgcgacaggatccggcagacctctggaaagcgagttacaatctacccggcggatttatttagactcacgccctttgacagaaggcattcttgacgcggaatccgctcatcgccgtcttatattcgaggtagattggaggaaatcggaagaaggcttaatcctcgaaggagggtccatctcgcttctaaattgtatggctaagtctccattttggagatctggattccagtggcacgtcaagcgcctgagacttggggattcagatgcgtttctgacgcgagcaaaacagcgtgtggctgagatgtttgctattagagaagatcggccaagtttattggaggagctcgccgagctgtggaattacccggcagctcgcccgattttggaagacattgacggctatcgatgtgccattagatttgcaaggaaacacgatctcgccatatcacagctgccgaacatcgatgccggccggcatgttgaactgatcgaagccattgcaaacgaatatcttgaacatgcgctaagccaagaacgcgactttcctcaatggccggaggacggcgcaggacaaccagtatgcccggttaccttaacgagaatccgttaaaaaagaggggagggaaacattatgatgttattacatcttatttacggacctacatgttcaggaaaaacagacatggccattcaaattgctcaagaaacgggatggccggttgtggcgttagacagagtacagtgctgcccacagattgcaacgggctcaggtagaccgttagaatctgagttacaatcaacaagaagaatctatctggattcacgcccactgacagagggcatcctggacgcagagagcgcgcaccgccgccttatctttgaagtcgattggcgcaaatctgaggagggccttatccttgaaggaggaagcattagcctgcttaattgtatggcaaaatcaccgttttggagatctggcttccagtggcacgtaaagagactgagacttggcgattcagacgcatttctgacaagagcaaaacagagagtcgcagaaatgttcgcgattagagaagacagaccaagcctgctggaggaattagctgaactgtggaactatccggctgcgcgccctattctggaggatattgatggttatcgctgtgcgattagatttgctagaaagcatgatctggctatttcacaactgcctaatatcgatgcgggaagacatgttgaactgattgaagccatcgccaacgaatacctggaacatgccctgtcacaggaaagagattttcctcaatggccggaagatggtgccggccaaccggtttgcccggtgacactgacaagaattagataaattaattctagaaattaagaaggagggattcgtcatgttggtattccaaatgcgttatgtagataaaacatctactgttttgaaacagactaaaaacagtgattacgcagataaataaatacgttagattaattcctaccagtgactaatcttatgactttttaaacagataactaaaattacaaacaaatcgtttaacttctgtatttgtttatagatgtaatcacttcaggagtgattacatgaacaaaaatataaaatattctcaaaactttttaacgagtgaaaaagtactcaaccaaataataaaacaattgaatttaaaagaaaccgataccgtttacgaaattggaacaggtaaagggcatttaacgacgaaactggctaaaataagtaaacaggtaacgtctattgaattagacagtcatctattcaacttatcgtcagaaaaattaaaactgaacattcgtgtcactttaattcaccaagatattctacagtttcaattccctaacaaacagaggtataaaattgttgggaatattccttaccatttaagcacacaaattattaaaaaagtggtttttgaaagccatgcgtctgacatctatctgattgttgaagaaggattctacaagcgtaccttggatattcaccgaacactagggttgctcttgcacactcaagtctcgattcagcaattgcttaagctgccagcggaatgctttcatcctaaaccaaaagtaaacagtgtcttaataaaacttacccgccataccacagatgttccagataaatattggaagctatatacgtactttgtttcaaaatgggtcaatcgagaatatcgtcaactgtttactaaaaatcagtttcatcaagcaatgaaacacgccaaagtaaacaatttaagtaccgttacttatgagcaagtattgtctatttttaatagttatctattatttaacgggaggaaataattctatgagtcgcttttgtaaatttggaaagttacacgttactaaagggaatgtagatggatcctgtatgccaatgatgatgaagagtcgacatggatgagcgatgatgatatccgtttaggctgggcggtgatagcttctcgttcaggcagtacgcctcttttcttttccagacctgagggaggcggaaatggtgtgaggttcccggggaaaagccaaataggcgatcgcgggagtgctttatttgaagatcaggctatcactgcggtcaatagatttcacaatgtgatggctggacagcctgaggaactctcgaacccgaatggaaacaaccagatatttatgaatcagcgcggctcacatggcgttgtgctggcaaatgcaggttcatcctctgtctctatcaatacggcaacaaaattgcctgatggcaggtatgacaataaagctggagcgggttcatttcaagtgaacgatggtaaactgacaggcacgatcaatgccaggtctgtagctgtgctttatcctgatgatattgcaaaagcgcctcatgttttccttgagaattacaaaacaggtgtaacacattctttcaatgatcaactgacgattaccttgcgtgcagatgcgaatacaacaaaagccgtttatcaaatcaataatggaccagagacggcgtttaaggatggagatcaattcacaatcggaaaaggagatccatttggcaaaacatacaccatcatgttaaaaggaacgaacagtgatggtgtaacgaggaccgagaaatacagttttgttaaaagagatccagcgtcggccaaaaccatcggctatcaaaatccgaatcattggagccaggtaaatgcttatatctataaacatgatgggagccgagtaattgaattgaccggatcttggcctggaaaaccaatgactaaaaatgcagacggaatttacacgctgacgctgcctgcggacacggatacaaccaacgcaaaagtgatttttaataatggcagcgcccaagtgcccggtcagaatcagcctggctttgattacgtgctaaatggtttatataatgactcgggcttaagcggttctcttccccattg

SEQ ID NO: 149 - DNA, 5075 bp, Artificial Sequence
  complete  AmyE 0 IPT1 LOG10 EryR AmyE END opero n
gcggtagtggtgcttacgatgtacgacagggggattccccatacattcttcgcttggctgaaaatgattcttctttttatcgtctgcggcggcgttctgtttctgcttcggtatgtgattgtgaagctggcttacagaagagcggtaaaagaagaaataaaaaagaaatcatcttttttgtttggaaagcgagggaagcgttcacagtttcgggcagctttttttataggaacattgatttgtattcactctgccaagttgttttgatagagtgattgtgataattttaaatgtaagcgttaacaaaattctccagtcttcacatcggtttgaaaggaggaagcggaagaatgaagtaagagggatttttgactccgaagtaagtcttcaaaaaatcaaataaggagtgtcaagaatgtttgcaaaacgattcaaaacctctttactgccgttattcgctggatttttattgctgtttcatttggttctggcaggaccggcggctgcgagtgctgaaacggcgaacaaatcgaatgagcttacagcaccgtcgatcaaaagcggaaccattcttcatgcatggaattggtcgttcaatacgttaaaacacaatatgaaggatattcatgatgcaggatatacagccattcagacatctccgattaaccaagtaaaggaagggaatcaaggagataaaagcatgtcgaactggtactggctgtatcagccgacatcgtatcaaattggcaaccgttacttaggtactgaacaagaatttaaagaaatgtgtgcagccgctgaagaatatggcataaaggtcattgttgacgcggtcatcaatcataccaccagtgattatgccgcgatttccaatgaggttaagagtattccaaactggacacatggaaacacacaaattaaaaactggtctgatcgatgggatgtcacgcagaattcattgctcgggctgtatgactggaatacacaaaatacacaagtacagtcctatctgaaacggttcttagacagggcattgaatgacggggcagacggttttcgatttgatgccgccaaacatatagagcttccagatgatggcagttacggcagtcaattttggccgaatatcacaaatacatctgcagagttccaatacggagaaatcctgcaggatagtgcctccagagatgctgcttatgcgaattatatggatgtgacagcgtctaactatgggcattccataaggtccgctttaaagaatcgtaatctgggcgtgtcgaatatctcccactatgcatctgatgtgtctgcggacaagctagtgacatgggtagagtcgcatgatacacgggtgcgcatgatcgtatggttcactgtccaccaaccaaaactgtgctcagtaccgccaatatttctcccttggggggtacaaagaggtgtccctagaagagatccacgctgtgtaaaaattttacaaaaaggtattgactttccctacagggtgtgtaataatttaattacaggcgggggcaaccccgctcagtacctagagcgtaaaagaggggagggaaacactagtatgttattacacctgatctacggtcctacatgtagtggtaaaactgatatggctatacaaatcgcgcaagaaacaggttggcccgtcgttgctcttgatcgtgtgcagtgctgtccacagattgcgacaggatccggcagacctctggaaagcgagttacaatctacccggcggatttatttagactcacgccctttgacagaaggcattcttgacgcggaatccgctcatcgccgtcttatattcgaggtagattggaggaaatcggaagaaggcttaatcctcgaaggagggtccatctcgcttctaaattgtatggctaagtctccattttggagatctggattccagtggcacgtcaagcgcctgagacttggggattcagatgcgtttctgacgcgagcaaaacagcgtgtggctgagatgtttgctattagagaagatcggccaagtttattggaggagctcgccgagctgtggaattacccggcagctcgcccgattttggaagacattgacggctatcgatgtgccattagatttgcaaggaaacacgatctcgccatatcacagctgccgaacatcgatgccggccggcatgttgaactgatcgaagccattgcaaacgaatatcttgaacatgcgctaagccaagaacgcgactttcctcaatggccggaggacggcgcaggacaaccagtatgcccggttaccttaacgagaatccgttaaaaaagaggggagggaaacattatgatggagattaagggagagagcatgcaaaagtcaaagttcagacgcatttgtgtgttttgcggttcatcacagggcaaaaagtcttcatatcaagatgctgcagttgatctgggtaacgaattagtaagcagaaacatcgatcttgtctacggcggtggctctattggacttatgggcttagtgtctcaagcagtccatgacggcggtagacatgtcatcggcattattcctaaaacgttaatgccgcgcgagctgacaggcgaaacagtaggagaggtaagagcggtggccgacatgcatcaaagaaaagcggaaatggccaaacacagcgacgcatttattgctttaccgggcggctacggaacactggaagaacttcttgaggtcatcacgtgggcccagctgggcattcatgataaaccagtgggactgctgaatgtggatggctattataatagcctgctgtcatttattgataaagcggttgaagaaggattcatttcaccgacggcaagagaaatcattgtttcagcgcctacagctaaagaactggttaaaaaactggaagagtatgcgccgtgccatgaaagagttgctacaaaactgtgttgggaaatggagagaatcggatattcatcagaagaataaattaattctagaaattaagaaggagggattcgtcatgttggtattccaaatgcgttatgtagataaaacatctactgttttgaaacagactaaaaacagtgattacgcagataaataaatacgttagattaattcctaccagtgactaatcttatgactttttaaacagataactaaaattacaaacaaatcgtttaacttctgtatttgtttatagatgtaatcacttcaggagtgattacatgaacaaaaatataaaatattctcaaaactttttaacgagtgaaaaagtactcaaccaaataataaaacaattgaatttaaaagaaaccgataccgtttacgaaattggaacaggtaaagggcatttaacgacgaaactggctaaaataagtaaacaggtaacgtctattgaattagacagtcatctattcaacttatcgtcagaaaaattaaaactgaacattcgtgtcactttaattcaccaagatattctacagtttcaattccctaacaaacagaggtataaaattgttgggaatattccttaccatttaagcacacaaattattaaaaaagtggtttttgaaagccatgcgtctgacatctatctgattgttgaagaaggattctacaagcgtaccttggatattcaccgaacactagggttgctcttgcacactcaagtctcgattcagcaattgcttaagctgccagcggaatgctttcatcctaaaccaaaagtaaacagtgtcttaataaaacttacccgccataccacagatgttccagataaatattggaagctatatacgtactttgtttcaaaatgggtcaatcgagaatatcgtcaactgtttactaaaaatcagtttcatcaagcaatgaaacacgccaaagtaaacaatttaagtaccgttacttatgagcaagtattgtctatttttaatagttatctattatttaacgggaggaaataattctatgagtcgcttttgtaaatttggaaagttacacgttactaaagggaatgtagatggatcctgtatgccaatgatgatgaagagtcgacatggatgagcgatgatgatatccgtttaggctgggcggtgatagcttctcgttcaggcagtacgcctcttttcttttccagacctgagggaggcggaaatggtgtgaggttcccggggaaaagccaaataggcgatcgcgggagtgctttatttgaagatcaggctatcactgcggtcaatagatttcacaatgtgatggctggacagcctgaggaactctcgaacccgaatggaaacaaccagatatttatgaatcagcgcggctcacatggcgttgtgctggcaaatgcaggttcatcctctgtctctatcaatacggcaacaaaattgcctgatggcaggtatgacaataaagctggagcgggttcatttcaagtgaacgatggtaaactgacaggcacgatcaatgccaggtctgtagctgtgctttatcctgatgatattgcaaaagcgcctcatgttttccttgagaattacaaaacaggtgtaacacattctttcaatgatcaactgacgattaccttgcgtgcagatgcgaatacaacaaaagccgtttatcaaatcaataatggaccagagacggcgtttaaggatggagatcaattcacaatcggaaaaggagatccatttggcaaaacatacaccatcatgttaaaaggaacgaacagtgatggtgtaacgaggaccgagaaatacagttttgttaaaagagatccagcgtcggccaaaaccatcggctatcaaaatccgaatcattggagccaggtaaatgcttatatctataaacatgatgggagccgagtaattgaattgaccggatcttggcctggaaaaccaatgactaaaaatgcagacggaatttacacgctgacgctgcctgcggacacggatacaaccaacgcaaaagtgatttttaataatggcagcgcccaagtgcccggtcagaatcagcctggctttgattacgtgctaaatggtttatataatgactcgggcttaagcggttctcttccccattg

SEQ ID NO: 150 - DNA, 5348 bp, Artificial Sequence
  complete  AmyE 0 IPT1 LOG11 EryR AmyE END opero n
gcggtagtggtgcttacgatgtacgacagggggattccccatacattcttcgcttggctgaaaatgattcttctttttatcgtctgcggcggcgttctgtttctgcttcggtatgtgattgtgaagctggcttacagaagagcggtaaaagaagaaataaaaaagaaatcatcttttttgtttggaaagcgagggaagcgttcacagtttcgggcagctttttttataggaacattgatttgtattcactctgccaagttgttttgatagagtgattgtgataattttaaatgtaagcgttaacaaaattctccagtcttcacatcggtttgaaaggaggaagcggaagaatgaagtaagagggatttttgactccgaagtaagtcttcaaaaaatcaaataaggagtgtcaagaatgtttgcaaaacgattcaaaacctctttactgccgttattcgctggatttttattgctgtttcatttggttctggcaggaccggcggctgcgagtgctgaaacggcgaacaaatcgaatgagcttacagcaccgtcgatcaaaagcggaaccattcttcatgcatggaattggtcgttcaatacgttaaaacacaatatgaaggatattcatgatgcaggatatacagccattcagacatctccgattaaccaagtaaaggaagggaatcaaggagataaaagcatgtcgaactggtactggctgtatcagccgacatcgtatcaaattggcaaccgttacttaggtactgaacaagaatttaaagaaatgtgtgcagccgctgaagaatatggcataaaggtcattgttgacgcggtcatcaatcataccaccagtgattatgccgcgatttccaatgaggttaagagtattccaaactggacacatggaaacacacaaattaaaaactggtctgatcgatgggatgtcacgcagaattcattgctcgggctgtatgactggaatacacaaaatacacaagtacagtcctatctgaaacggttcttagacagggcattgaatgacggggcagacggttttcgatttgatgccgccaaacatatagagcttccagatgatggcagttacggcagtcaattttggccgaatatcacaaatacatctgcagagttccaatacggagaaatcctgcaggatagtgcctccagagatgctgcttatgcgaattatatggatgtgacagcgtctaactatgggcattccataaggtccgctttaaagaatcgtaatctgggcgtgtcgaatatctcccactatgcatctgatgtgtctgcggacaagctagtgacatgggtagagtcgcatgatacacgggtgcgcatgatcgtatggttcactgtccaccaaccaaaactgtgctcagtaccgccaatatttctcccttggggggtacaaagaggtgtccctagaagagatccacgctgtgtaaaaattttacaaaaaggtattgactttccctacagggtgtgtaataatttaattacaggcgggggcaaccccgctcagtacctagagcgtaaaagaggggagggaaacactagtatgttattacacctgatctacggtcctacatgtagtggtaaaactgatatggctatacaaatcgcgcaagaaacaggttggcccgtcgttgctcttgatcgtgtgcagtgctgtccacagattgcgacaggatccggcagacctctggaaagcgagttacaatctacccggcggatttatttagactcacgccctttgacagaaggcattcttgacgcggaatccgctcatcgccgtcttatattcgaggtagattggaggaaatcggaagaaggcttaatcctcgaaggagggtccatctcgcttctaaattgtatggctaagtctccattttggagatctggattccagtggcacgtcaagcgcctgagacttggggattcagatgcgtttctgacgcgagcaaaacagcgtgtggctgagatgtttgctattagagaagatcggccaagtttattggaggagctcgccgagctgtggaattacccggcagctcgcccgattttggaagacattgacggctatcgatgtgccattagatttgcaaggaaacacgatctcgccatatcacagctgccgaacatcgatgccggccggcatgttgaactgatcgaagccattgcaaacgaatatcttgaacatgcgctaagccaagaacgcgactttcctcaatggccggaggacggcgcaggacaaccagtatgcccggttaccttaacgagaatccgttaaaaaagaggggagggaaacattatgatgggtgtctcaatgctggattcttcttggtcatttcgcctttcaagcaagatgtctccaagcccgacgcaaaaattagccagctctctgacagaacgcgatccaatcagcttaaatctgcgcttcgaccattcaattagagcatatagcgcaagagctcttaagtgcagatctgtcaaatctgaagatttcgttgaaagatcatcaccatttgaagttaagaaggagctggaaatttgctttgatcttgtccatagattaggtagaggcattgtttaccttggcagcgctagaattccgccgaaccacagccattacctgcaggcacaggaattatcaagagaagcggcaacgttacttgactgcacaacgtggtcaggcgctggacctggccttatggatgcggtgacggaaggtgcccttgaggcggaaaagccggtaggtggcatcaaaattgaaaaagaagcgggcgagtggacggcgtcaaaattccacccgtatttacctccgcaaaattatcatacatgtagatttttctcagcgagaaaacatggccttgtcgatgccgtaattagaaataatgtttcagaaaaaacagctatcattgcactgccgggcggcattggaacattagatgagatgttcgaaatcctggctctgattcaactggagagaattggctcagctctgccggtgccttttatcgtaatgaactacgacgccttttattcaaaactgctggaatttatcgagacatgtgaaaatctgggaacagtttcaaaaggagaagtgtcagcactgtggaaagtgtgtaataacaactttgaggccctgacatatctggcggaattttatgacctgcctgatggatcagcaaaactggagacagaactgagaaaaacaacacctacaagaggataaattaattctagaaattaagaaggagggattcgtcatgttggtattccaaatgcgttatgtagataaaacatctactgttttgaaacagactaaaaacagtgattacgcagataaataaatacgttagattaattcctaccagtgactaatcttatgactttttaaacagataactaaaattacaaacaaatcgtttaacttctgtatttgtttatagatgtaatcacttcaggagtgattacatgaacaaaaatataaaatattctcaaaactttttaacgagtgaaaaagtactcaaccaaataataaaacaattgaatttaaaagaaaccgataccgtttacgaaattggaacaggtaaagggcatttaacgacgaaactggctaaaataagtaaacaggtaacgtctattgaattagacagtcatctattcaacttatcgtcagaaaaattaaaactgaacattcgtgtcactttaattcaccaagatattctacagtttcaattccctaacaaacagaggtataaaattgttgggaatattccttaccatttaagcacacaaattattaaaaaagtggtttttgaaagccatgcgtctgacatctatctgattgttgaagaaggattctacaagcgtaccttggatattcaccgaacactagggttgctcttgcacactcaagtctcgattcagcaattgcttaagctgccagcggaatgctttcatcctaaaccaaaagtaaacagtgtcttaataaaacttacccgccataccacagatgttccagataaatattggaagctatatacgtactttgtttcaaaatgggtcaatcgagaatatcgtcaactgtttactaaaaatcagtttcatcaagcaatgaaacacgccaaagtaaacaatttaagtaccgttacttatgagcaagtattgtctatttttaatagttatctattatttaacgggaggaaataattctatgagtcgcttttgtaaatttggaaagttacacgttactaaagggaatgtagatggatcctgtatgccaatgatgatgaagagtcgacatggatgagcgatgatgatatccgtttaggctgggcggtgatagcttctcgttcaggcagtacgcctcttttcttttccagacctgagggaggcggaaatggtgtgaggttcccggggaaaagccaaataggcgatcgcgggagtgctttatttgaagatcaggctatcactgcggtcaatagatttcacaatgtgatggctggacagcctgaggaactctcgaacccgaatggaaacaaccagatatttatgaatcagcgcggctcacatggcgttgtgctggcaaatgcaggttcatcctctgtctctatcaatacggcaacaaaattgcctgatggcaggtatgacaataaagctggagcgggttcatttcaagtgaacgatggtaaactgacaggcacgatcaatgccaggtctgtagctgtgctttatcctgatgatattgcaaaagcgcctcatgttttccttgagaattacaaaacaggtgtaacacattctttcaatgatcaactgacgattaccttgcgtgcagatgcgaatacaacaaaagccgtttatcaaatcaataatggaccagagacggcgtttaaggatggagatcaattcacaatcggaaaaggagatccatttggcaaaacatacaccatcatgttaaaaggaacgaacagtgatggtgtaacgaggaccgagaaatacagttttgttaaaagagatccagcgtcggccaaaaccatcggctatcaaaatccgaatcattggagccaggtaaatgcttatatctataaacatgatgggagccgagtaattgaattgaccggatcttggcctggaaaaccaatgactaaaaatgcagacggaatttacacgctgacgctgcctgcggacacggatacaaccaacgcaaaagtgatttttaataatggcagcgcccaagtgcccggtcagaatcagcctggctttgattacgtgctaaatggtttatataatgactcgggcttaagcggttctcttccccattg

SEQ ID NO: 151 - DNA, 842 bp, Artificial Sequence
  Fragment IPT6_ Bsu
cgcagcatacgcagcgaaatcagcatcaccggagaatcccaacgaagccaactagtatgatcctgcatcttatttatggtccgacgtgcagcggcaaaacggacatggccattcagattgcgcaggagacaggatggccagtagtagctctggatagagtccagtgttgtccacaaattgctacaggctctggccgcccgctggagtctgagttacagagcacacgcagaatctatctggatagcagaccactgacagaaggtattctggatgcagaatcagcacatagacgcttaatcttcgaagtcgattggagaaagtctgaggacggacttattcttgaaggaggctcaatttctcttttaaactgtatggcaaaatcacctttctggagatcaggctttcagtggcacgttaaaagattaagacttggcgactcagatgcgtttctgacaagagcaaagcaaagagttgcagaaatgtttgcgattcgcgaggatagaccgtcacttttagaagaactggcagaactgtggaactatcctgccgcgcgccctatcttagaagacattgatggatacagatgcgccatcagatttgctagaaaacacgatctggccatttcacaactgcctaatatcgacgccggaagacatgttgaactgattgaagcgatcgcgaatgaatatctggaacatgctctgtctcaagaaagagattttccgcaatggccggaagatggcgctggtcaacctgtgtgcccggtgacactgacaagaattagataaaaaagaggggagggaaacattaatgacgacctggctaacgagtctcgccgatct

SEQ ID NO: 152 - DNA, 827 bp, Artificial Sequence
  Frgament IPT7_Bsu
cgcagcatacgcagcgaaatcagcatcaccggagaatcccaacgaagccaactagtatgccggtcagactgtatctgatttggggcgctacaacaacgggcaagacagcacaatctgtggcactggctagatcagcaggtgctcctgtaatttcactggacagagttcaatgttgccatgaactggccgttggttctggccgcccatcaccgagcgagctgctgggaacgagaagagaatatttatgtgagcgcgaagtgtcaagaggagtggttagcgccgccgaagcgaatcagctgctgctggataaagtggcgcgctatgccacacaggaacgcgcgctgattcttgaaggaggatcagtctcactgatcaacgctatgatcagagatgcgagatggagcgaaagaggcgaatggatcttaagaagaatcccggttccgggcagagccgcatttatggccgccgcgagaaaaagagttcgcgaaatgcttgacccgcctccaggccaggcaggaattttagatgagcttcaaggtctttggggctatccgagaaatcacgccgtcttagaggatattgacggctacagacagattattagatacgccaatgcattacaggtccctatctgcagaatcacatctattgaccctaacgccaaagctctgcttattgagcgcattgcacaggaatactgggaacatgctttatggcaagagcaagagttcctgggcattccggcaagctggatgagagctgatgatgcgtaaaaaagaggggagggaaacattaatgacgacctggctaacgagtctcgccgatct

SEQ ID NO: 153 - DNA, 842 bp, Artificial Sequence
  Fragment IPT9_Bsu
cgcagcatacgcagcgaaatcagcatcaccggagaatcccaacgaagccaactagtatgttattacatcttatttacggacctacatgttcaggaaaaacagacatggccattcaaattgctcaagaaacgggatggccggttgtggcgttagacagagtacagtgctgcccacagattgcaacgggctcaggtagaccgttagaatctgagttacaatcaacaagaagaatctatctggattcacgcccactgacagagggcatcctggacgcagagagcgcgcaccgccgccttatctttgaagtcgattggcgcaaatctgaggagggccttatccttgaaggaggaagcattagcctgcttaattgtatggcaaaatcaccgttttggagatctggcttccagtggcacgtaaagagactgagacttggcgattcagacgcatttctgacaagagcaaaacagagagtcgcagaaatgttcgcgattagagaagacagaccaagcctgctggaggaattagctgaactgtggaactatccggctgcgcgccctattctggaggatattgatggttatcgctgtgcgattagatttgctagaaagcatgatctggctatttcacaactgcctaatatcgatgcgggaagacatgttgaactgattgaagccatcgccaacgaatacctggaacatgccctgtcacaggaaagagattttcctcaatggccggaagatggtgccggccaaccggtttgcccggtgacactgacaagaattagataaaaaagaggggagggaaacattaatgacgacctggctaacgagtctcgccgatct

SEQ ID NO: 154 - DNA, 2365 bp, Artificial Sequence
  operon AmyE 0 + IPT6
gcggtagtggtgcttacgatgtacgacagggggattccccatacattcttcgcttggctgaaaatgattcttctttttatcgtctgcggcggcgttctgtttctgcttcggtatgtgattgtgaagctggcttacagaagagcggtaaaagaagaaataaaaaagaaatcatcttttttgtttggaaagcgagggaagcgttcacagtttcgggcagctttttttataggaacattgatttgtattcactctgccaagttgttttgatagagtgattgtgataattttaaatgtaagcgttaacaaaattctccagtcttcacatcggtttgaaaggaggaagcggaagaatgaagtaagagggatttttgactccgaagtaagtcttcaaaaaatcaaataaggagtgtcaagaatgtttgcaaaacgattcaaaacctctttactgccgttattcgctggatttttattgctgtttcatttggttctggcaggaccggcggctgcgagtgctgaaacggcgaacaaatcgaatgagcttacagcaccgtcgatcaaaagcggaaccattcttcatgcatggaattggtcgttcaatacgttaaaacacaatatgaaggatattcatgatgcaggatatacagccattcagacatctccgattaaccaagtaaaggaagggaatcaaggagataaaagcatgtcgaactggtactggctgtatcagccgacatcgtatcaaattggcaaccgttacttaggtactgaacaagaatttaaagaaatgtgtgcagccgctgaagaatatggcataaaggtcattgttgacgcggtcatcaatcataccaccagtgattatgccgcgatttccaatgaggttaagagtattccaaactggacacatggaaacacacaaattaaaaactggtctgatcgatgggatgtcacgcagaattcattgctcgggctgtatgactggaatacacaaaatacacaagtacagtcctatctgaaacggttcttagacagggcattgaatgacggggcagacggttttcgatttgatgccgccaaacatatagagcttccagatgatggcagttacggcagtcaattttggccgaatatcacaaatacatctgcagagttccaatacggagaaatcctgcaggatagtgcctccagagatgctgcttatgcgaattatatggatgtgacagcgtctaactatgggcattccataaggtccgctttaaagaatcgtaatctgggcgtgtcgaatatctcccactatgcatctgatgtgtctgcggacaagctagtgacatgggtagagtcgcatgatacacgggtgcgcatgatcgtatggttcactgtccaccaaccaaaactgtgctcagtaccgccaatatttctcccttggggggtacaaagaggtgtccctagaagagatccacgctgtgtaaaaattttacaaaaaggtattgactttccctacagggtgtgtaataatttaattacaggcgggggcaaccccgctcagtacctagagcgtaaaagaggggagggaaacactagtatgatcctgcatcttatttatggtccgacgtgcagcggcaaaacggacatggccattcagattgcgcaggagacaggatggccagtagtagctctggatagagtccagtgttgtccacaaattgctacaggctctggccgcccgctggagtctgagttacagagcacacgcagaatctatctggatagcagaccactgacagaaggtattctggatgcagaatcagcacatagacgcttaatcttcgaagtcgattggagaaagtctgaggacggacttattcttgaaggaggctcaatttctcttttaaactgtatggcaaaatcacctttctggagatcaggctttcagtggcacgttaaaagattaagacttggcgactcagatgcgtttctgacaagagcaaagcaaagagttgcagaaatgtttgcgattcgcgaggatagaccgtcacttttagaagaactggcagaactgtggaactatcctgccgcgcgccctatcttagaagacattgatggatacagatgcgccatcagatttgctagaaaacacgatctggccatttcacaactgcctaatatcgacgccggaagacatgttgaactgattgaagcgatcgcgaatgaatatctggaacatgctctgtctcaagaaagagattttccgcaatggccggaagatggcgctggtcaacctgtgtgcccggtgacactgacaagaattagataaaaaagaggggagggaaacattaatgacgacctggctaacgagtctcgccgatct

SEQ ID NO: 155 - DNA, 2350 bp, Artificial Sequence
  operon AmyE 0 + IPT7
gcggtagtggtgcttacgatgtacgacagggggattccccatacattcttcgcttggctgaaaatgattcttctttttatcgtctgcggcggcgttctgtttctgcttcggtatgtgattgtgaagctggcttacagaagagcggtaaaagaagaaataaaaaagaaatcatcttttttgtttggaaagcgagggaagcgttcacagtttcgggcagctttttttataggaacattgatttgtattcactctgccaagttgttttgatagagtgattgtgataattttaaatgtaagcgttaacaaaattctccagtcttcacatcggtttgaaaggaggaagcggaagaatgaagtaagagggatttttgactccgaagtaagtcttcaaaaaatcaaataaggagtgtcaagaatgtttgcaaaacgattcaaaacctctttactgccgttattcgctggatttttattgctgtttcatttggttctggcaggaccggcggctgcgagtgctgaaacggcgaacaaatcgaatgagcttacagcaccgtcgatcaaaagcggaaccattcttcatgcatggaattggtcgttcaatacgttaaaacacaatatgaaggatattcatgatgcaggatatacagccattcagacatctccgattaaccaagtaaaggaagggaatcaaggagataaaagcatgtcgaactggtactggctgtatcagccgacatcgtatcaaattggcaaccgttacttaggtactgaacaagaatttaaagaaatgtgtgcagccgctgaagaatatggcataaaggtcattgttgacgcggtcatcaatcataccaccagtgattatgccgcgatttccaatgaggttaagagtattccaaactggacacatggaaacacacaaattaaaaactggtctgatcgatgggatgtcacgcagaattcattgctcgggctgtatgactggaatacacaaaatacacaagtacagtcctatctgaaacggttcttagacagggcattgaatgacggggcagacggttttcgatttgatgccgccaaacatatagagcttccagatgatggcagttacggcagtcaattttggccgaatatcacaaatacatctgcagagttccaatacggagaaatcctgcaggatagtgcctccagagatgctgcttatgcgaattatatggatgtgacagcgtctaactatgggcattccataaggtccgctttaaagaatcgtaatctgggcgtgtcgaatatctcccactatgcatctgatgtgtctgcggacaagctagtgacatgggtagagtcgcatgatacacgggtgcgcatgatcgtatggttcactgtccaccaaccaaaactgtgctcagtaccgccaatatttctcccttggggggtacaaagaggtgtccctagaagagatccacgctgtgtaaaaattttacaaaaaggtattgactttccctacagggtgtgtaataatttaattacaggcgggggcaaccccgctcagtacctagagcgtaaaagaggggagggaaacactagtatgccggtcagactgtatctgatttggggcgctacaacaacgggcaagacagcacaatctgtggcactggctagatcagcaggtgctcctgtaatttcactggacagagttcaatgttgccatgaactggccgttggttctggccgcccatcaccgagcgagctgctgggaacgagaagagaatatttatgtgagcgcgaagtgtcaagaggagtggttagcgccgccgaagcgaatcagctgctgctggataaagtggcgcgctatgccacacaggaacgcgcgctgattcttgaaggaggatcagtctcactgatcaacgctatgatcagagatgcgagatggagcgaaagaggcgaatggatcttaagaagaatcccggttccgggcagagccgcatttatggccgccgcgagaaaaagagttcgcgaaatgcttgacccgcctccaggccaggcaggaattttagatgagcttcaaggtctttggggctatccgagaaatcacgccgtcttagaggatattgacggctacagacagattattagatacgccaatgcattacaggtccctatctgcagaatcacatctattgaccctaacgccaaagctctgcttattgagcgcattgcacaggaatactgggaacatgctttatggcaagagcaagagttcctgggcattccggcaagctggatgagagctgatgatgcgtaaaaaagaggggagggaaacattaatgacgacctggctaacgagtctcgccgatct

SEQ ID NO: 156 - DNA, 2365 bp, Artificial Sequence
  operon AmyE 0 + IPT9
gcggtagtggtgcttacgatgtacgacagggggattccccatacattcttcgcttggctgaaaatgattcttctttttatcgtctgcggcggcgttctgtttctgcttcggtatgtgattgtgaagctggcttacagaagagcggtaaaagaagaaataaaaaagaaatcatcttttttgtttggaaagcgagggaagcgttcacagtttcgggcagctttttttataggaacattgatttgtattcactctgccaagttgttttgatagagtgattgtgataattttaaatgtaagcgttaacaaaattctccagtcttcacatcggtttgaaaggaggaagcggaagaatgaagtaagagggatttttgactccgaagtaagtcttcaaaaaatcaaataaggagtgtcaagaatgtttgcaaaacgattcaaaacctctttactgccgttattcgctggatttttattgctgtttcatttggttctggcaggaccggcggctgcgagtgctgaaacggcgaacaaatcgaatgagcttacagcaccgtcgatcaaaagcggaaccattcttcatgcatggaattggtcgttcaatacgttaaaacacaatatgaaggatattcatgatgcaggatatacagccattcagacatctccgattaaccaagtaaaggaagggaatcaaggagataaaagcatgtcgaactggtactggctgtatcagccgacatcgtatcaaattggcaaccgttacttaggtactgaacaagaatttaaagaaatgtgtgcagccgctgaagaatatggcataaaggtcattgttgacgcggtcatcaatcataccaccagtgattatgccgcgatttccaatgaggttaagagtattccaaactggacacatggaaacacacaaattaaaaactggtctgatcgatgggatgtcacgcagaattcattgctcgggctgtatgactggaatacacaaaatacacaagtacagtcctatctgaaacggttcttagacagggcattgaatgacggggcagacggttttcgatttgatgccgccaaacatatagagcttccagatgatggcagttacggcagtcaattttggccgaatatcacaaatacatctgcagagttccaatacggagaaatcctgcaggatagtgcctccagagatgctgcttatgcgaattatatggatgtgacagcgtctaactatgggcattccataaggtccgctttaaagaatcgtaatctgggcgtgtcgaatatctcccactatgcatctgatgtgtctgcggacaagctagtgacatgggtagagtcgcatgatacacgggtgcgcatgatcgtatggttcactgtccaccaaccaaaactgtgctcagtaccgccaatatttctcccttggggggtacaaagaggtgtccctagaagagatccacgctgtgtaaaaattttacaaaaaggtattgactttccctacagggtgtgtaataatttaattacaggcgggggcaaccccgctcagtacctagagcgtaaaagaggggagggaaacactagtatgttattacatcttatttacggacctacatgttcaggaaaaacagacatggccattcaaattgctcaagaaacgggatggccggttgtggcgttagacagagtacagtgctgcccacagattgcaacgggctcaggtagaccgttagaatctgagttacaatcaacaagaagaatctatctggattcacgcccactgacagagggcatcctggacgcagagagcgcgcaccgccgccttatctttgaagtcgattggcgcaaatctgaggagggccttatccttgaaggaggaagcattagcctgcttaattgtatggcaaaatcaccgttttggagatctggcttccagtggcacgtaaagagactgagacttggcgattcagacgcatttctgacaagagcaaaacagagagtcgcagaaatgttcgcgattagagaagacagaccaagcctgctggaggaattagctgaactgtggaactatccggctgcgcgccctattctggaggatattgatggttatcgctgtgcgattagatttgctagaaagcatgatctggctatttcacaactgcctaatatcgatgcgggaagacatgttgaactgattgaagccatcgccaacgaatacctggaacatgccctgtcacaggaaagagattttcctcaatggccggaagatggtgccggccaaccggtttgcccggtgacactgacaagaattagataaaaaagaggggagggaaacattaatgacgacctggctaacgagtctcgccgatct

SEQ ID NO: 157 - DNA, 3218 bp, Artificial Sequence
  operon AmyE 0 + IPT6 + LOG8
gcggtagtggtgcttacgatgtacgacagggggattccccatacattcttcgcttggctgaaaatgattcttctttttatcgtctgcggcggcgttctgtttctgcttcggtatgtgattgtgaagctggcttacagaagagcggtaaaagaagaaataaaaaagaaatcatcttttttgtttggaaagcgagggaagcgttcacagtttcgggcagctttttttataggaacattgatttgtattcactctgccaagttgttttgatagagtgattgtgataattttaaatgtaagcgttaacaaaattctccagtcttcacatcggtttgaaaggaggaagcggaagaatgaagtaagagggatttttgactccgaagtaagtcttcaaaaaatcaaataaggagtgtcaagaatgtttgcaaaacgattcaaaacctctttactgccgttattcgctggatttttattgctgtttcatttggttctggcaggaccggcggctgcgagtgctgaaacggcgaacaaatcgaatgagcttacagcaccgtcgatcaaaagcggaaccattcttcatgcatggaattggtcgttcaatacgttaaaacacaatatgaaggatattcatgatgcaggatatacagccattcagacatctccgattaaccaagtaaaggaagggaatcaaggagataaaagcatgtcgaactggtactggctgtatcagccgacatcgtatcaaattggcaaccgttacttaggtactgaacaagaatttaaagaaatgtgtgcagccgctgaagaatatggcataaaggtcattgttgacgcggtcatcaatcataccaccagtgattatgccgcgatttccaatgaggttaagagtattccaaactggacacatggaaacacacaaattaaaaactggtctgatcgatgggatgtcacgcagaattcattgctcgggctgtatgactggaatacacaaaatacacaagtacagtcctatctgaaacggttcttagacagggcattgaatgacggggcagacggttttcgatttgatgccgccaaacatatagagcttccagatgatggcagttacggcagtcaattttggccgaatatcacaaatacatctgcagagttccaatacggagaaatcctgcaggatagtgcctccagagatgctgcttatgcgaattatatggatgtgacagcgtctaactatgggcattccataaggtccgctttaaagaatcgtaatctgggcgtgtcgaatatctcccactatgcatctgatgtgtctgcggacaagctagtgacatgggtagagtcgcatgatacacgggtgcgcatgatcgtatggttcactgtccaccaaccaaaactgtgctcagtaccgccaatatttctcccttggggggtacaaagaggtgtccctagaagagatccacgctgtgtaaaaattttacaaaaaggtattgactttccctacagggtgtgtaataatttaattacaggcgggggcaaccccgctcagtacctagagcgtaaaagaggggagggaaacactagtatgatcctgcatcttatttatggtccgacgtgcagcggcaaaacggacatggccattcagattgcgcaggagacaggatggccagtagtagctctggatagagtccagtgttgtccacaaattgctacaggctctggccgcccgctggagtctgagttacagagcacacgcagaatctatctggatagcagaccactgacagaaggtattctggatgcagaatcagcacatagacgcttaatcttcgaagtcgattggagaaagtctgaggacggacttattcttgaaggaggctcaatttctcttttaaactgtatggcaaaatcacctttctggagatcaggctttcagtggcacgttaaaagattaagacttggcgactcagatgcgtttctgacaagagcaaagcaaagagttgcagaaatgtttgcgattcgcgaggatagaccgtcacttttagaagaactggcagaactgtggaactatcctgccgcgcgccctatcttagaagacattgatggatacagatgcgccatcagatttgctagaaaacacgatctggccatttcacaactgcctaatatcgacgccggaagacatgttgaactgattgaagcgatcgcgaatgaatatctggaacatgctctgtctcaagaaagagattttccgcaatggccggaagatggcgctggtcaacctgtgtgcccggtgacactgacaagaattagataaaaaagaggggagggaaacattatgatggcccctcttcgctcaaacaaagatggcaaacctagagttcctagaacgacggaaagcgccgaaacgaatagaatgttaagaggtccgatgttactgagaacagacggaaatcagacgtcaacatacgatcaaagacttttagagtcaggcgcagatcacgcttggaagcatgcagatccgtggcgcgttctgagaatccagggagagttcgtggcaggatttgatgctctgagcgatctgcctaaggcggttacggtatacggctcagccagaacaaaagaggacacaccggaatatgcacagggcatcgatttaggtgcaaaattagttgagcatgactacgctgttgtgacaggcggtggcccaggcatcatggaggcggcgaatagaggcgcctatgaagctggaggtctttctgtgggcctgggcattgaacttccgtttgaacaaggtttaaacaaatatgtagatcttggaattaactttagatactttttcgcgagaaagacaatgttcctgaaatattcacaggcgtttgtgtgcctgccgggcggtatgggaacaatggatgagctgtttgaaatgctgtgtatggtgcaaacaggaaaagtgacaaactatccgattgtactgatcggcacagaatattggagcggcctggtagaatggatggctaatacactggtcgccgaaggctacatcaatccacaagatatggatctgttcctgctgacagacagcgtcgacgaagccgtcgaacatattgtcgcggctcataaagaaatgacagacgaaagagtctcaagactgaatcacgacgcataaattaattctagagtcgatccccgggttcgccagcaatgactaccggcagcccgcc

SEQ ID NO: 158 - DNA, 3203 bp, Artificial Sequence
  operon AmyE 0 + IPT7 + LOG8
gcggtagtggtgcttacgatgtacgacagggggattccccatacattcttcgcttggctgaaaatgattcttctttttatcgtctgcggcggcgttctgtttctgcttcggtatgtgattgtgaagctggcttacagaagagcggtaaaagaagaaataaaaaagaaatcatcttttttgtttggaaagcgagggaagcgttcacagtttcgggcagctttttttataggaacattgatttgtattcactctgccaagttgttttgatagagtgattgtgataattttaaatgtaagcgttaacaaaattctccagtcttcacatcggtttgaaaggaggaagcggaagaatgaagtaagagggatttttgactccgaagtaagtcttcaaaaaatcaaataaggagtgtcaagaatgtttgcaaaacgattcaaaacctctttactgccgttattcgctggatttttattgctgtttcatttggttctggcaggaccggcggctgcgagtgctgaaacggcgaacaaatcgaatgagcttacagcaccgtcgatcaaaagcggaaccattcttcatgcatggaattggtcgttcaatacgttaaaacacaatatgaaggatattcatgatgcaggatatacagccattcagacatctccgattaaccaagtaaaggaagggaatcaaggagataaaagcatgtcgaactggtactggctgtatcagccgacatcgtatcaaattggcaaccgttacttaggtactgaacaagaatttaaagaaatgtgtgcagccgctgaagaatatggcataaaggtcattgttgacgcggtcatcaatcataccaccagtgattatgccgcgatttccaatgaggttaagagtattccaaactggacacatggaaacacacaaattaaaaactggtctgatcgatgggatgtcacgcagaattcattgctcgggctgtatgactggaatacacaaaatacacaagtacagtcctatctgaaacggttcttagacagggcattgaatgacggggcagacggttttcgatttgatgccgccaaacatatagagcttccagatgatggcagttacggcagtcaattttggccgaatatcacaaatacatctgcagagttccaatacggagaaatcctgcaggatagtgcctccagagatgctgcttatgcgaattatatggatgtgacagcgtctaactatgggcattccataaggtccgctttaaagaatcgtaatctgggcgtgtcgaatatctcccactatgcatctgatgtgtctgcggacaagctagtgacatgggtagagtcgcatgatacacgggtgcgcatgatcgtatggttcactgtccaccaaccaaaactgtgctcagtaccgccaatatttctcccttggggggtacaaagaggtgtccctagaagagatccacgctgtgtaaaaattttacaaaaaggtattgactttccctacagggtgtgtaataatttaattacaggcgggggcaaccccgctcagtacctagagcgtaaaagaggggagggaaacactagtatgccggtcagactgtatctgatttggggcgctacaacaacgggcaagacagcacaatctgtggcactggctagatcagcaggtgctcctgtaatttcactggacagagttcaatgttgccatgaactggccgttggttctggccgcccatcaccgagcgagctgctgggaacgagaagagaatatttatgtgagcgcgaagtgtcaagaggagtggttagcgccgccgaagcgaatcagctgctgctggataaagtggcgcgctatgccacacaggaacgcgcgctgattcttgaaggaggatcagtctcactgatcaacgctatgatcagagatgcgagatggagcgaaagaggcgaatggatcttaagaagaatcccggttccgggcagagccgcatttatggccgccgcgagaaaaagagttcgcgaaatgcttgacccgcctccaggccaggcaggaattttagatgagcttcaaggtctttggggctatccgagaaatcacgccgtcttagaggatattgacggctacagacagattattagatacgccaatgcattacaggtccctatctgcagaatcacatctattgaccctaacgccaaagctctgcttattgagcgcattgcacaggaatactgggaacatgctttatggcaagagcaagagttcctgggcattccggcaagctggatgagagctgatgatgcgtaaaaaagaggggagggaaacattatgatggcccctcttcgctcaaacaaagatggcaaacctagagttcctagaacgacggaaagcgccgaaacgaatagaatgttaagaggtccgatgttactgagaacagacggaaatcagacgtcaacatacgatcaaagacttttagagtcaggcgcagatcacgcttggaagcatgcagatccgtggcgcgttctgagaatccagggagagttcgtggcaggatttgatgctctgagcgatctgcctaaggcggttacggtatacggctcagccagaacaaaagaggacacaccggaatatgcacagggcatcgatttaggtgcaaaattagttgagcatgactacgctgttgtgacaggcggtggcccaggcatcatggaggcggcgaatagaggcgcctatgaagctggaggtctttctgtgggcctgggcattgaacttccgtttgaacaaggtttaaacaaatatgtagatcttggaattaactttagatactttttcgcgagaaagacaatgttcctgaaatattcacaggcgtttgtgtgcctgccgggcggtatgggaacaatggatgagctgtttgaaatgctgtgtatggtgcaaacaggaaaagtgacaaactatccgattgtactgatcggcacagaatattggagcggcctggtagaatggatggctaatacactggtcgccgaaggctacatcaatccacaagatatggatctgttcctgctgacagacagcgtcgacgaagccgtcgaacatattgtcgcggctcataaagaaatgacagacgaaagagtctcaagactgaatcacgacgcataaattaattctagagtcgatccccgggttcgccagcaatgactaccggcagcccgcc

SEQ ID NO: 159 - DNA, 3218 bp, Artificial Sequence
  operon AmyE 0 + IPT9 + LOG8
gcggtagtggtgcttacgatgtacgacagggggattccccatacattcttcgcttggctgaaaatgattcttctttttatcgtctgcggcggcgttctgtttctgcttcggtatgtgattgtgaagctggcttacagaagagcggtaaaagaagaaataaaaaagaaatcatcttttttgtttggaaagcgagggaagcgttcacagtttcgggcagctttttttataggaacattgatttgtattcactctgccaagttgttttgatagagtgattgtgataattttaaatgtaagcgttaacaaaattctccagtcttcacatcggtttgaaaggaggaagcggaagaatgaagtaagagggatttttgactccgaagtaagtcttcaaaaaatcaaataaggagtgtcaagaatgtttgcaaaacgattcaaaacctctttactgccgttattcgctggatttttattgctgtttcatttggttctggcaggaccggcggctgcgagtgctgaaacggcgaacaaatcgaatgagcttacagcaccgtcgatcaaaagcggaaccattcttcatgcatggaattggtcgttcaatacgttaaaacacaatatgaaggatattcatgatgcaggatatacagccattcagacatctccgattaaccaagtaaaggaagggaatcaaggagataaaagcatgtcgaactggtactggctgtatcagccgacatcgtatcaaattggcaaccgttacttaggtactgaacaagaatttaaagaaatgtgtgcagccgctgaagaatatggcataaaggtcattgttgacgcggtcatcaatcataccaccagtgattatgccgcgatttccaatgaggttaagagtattccaaactggacacatggaaacacacaaattaaaaactggtctgatcgatgggatgtcacgcagaattcattgctcgggctgtatgactggaatacacaaaatacacaagtacagtcctatctgaaacggttcttagacagggcattgaatgacggggcagacggttttcgatttgatgccgccaaacatatagagcttccagatgatggcagttacggcagtcaattttggccgaatatcacaaatacatctgcagagttccaatacggagaaatcctgcaggatagtgcctccagagatgctgcttatgcgaattatatggatgtgacagcgtctaactatgggcattccataaggtccgctttaaagaatcgtaatctgggcgtgtcgaatatctcccactatgcatctgatgtgtctgcggacaagctagtgacatgggtagagtcgcatgatacacgggtgcgcatgatcgtatggttcactgtccaccaaccaaaactgtgctcagtaccgccaatatttctcccttggggggtacaaagaggtgtccctagaagagatccacgctgtgtaaaaattttacaaaaaggtattgactttccctacagggtgtgtaataatttaattacaggcgggggcaaccccgctcagtacctagagcgtaaaagaggggagggaaacactagtatgttattacatcttatttacggacctacatgttcaggaaaaacagacatggccattcaaattgctcaagaaacgggatggccggttgtggcgttagacagagtacagtgctgcccacagattgcaacgggctcaggtagaccgttagaatctgagttacaatcaacaagaagaatctatctggattcacgcccactgacagagggcatcctggacgcagagagcgcgcaccgccgccttatctttgaagtcgattggcgcaaatctgaggagggccttatccttgaaggaggaagcattagcctgcttaattgtatggcaaaatcaccgttttggagatctggcttccagtggcacgtaaagagactgagacttggcgattcagacgcatttctgacaagagcaaaacagagagtcgcagaaatgttcgcgattagagaagacagaccaagcctgctggaggaattagctgaactgtggaactatccggctgcgcgccctattctggaggatattgatggttatcgctgtgcgattagatttgctagaaagcatgatctggctatttcacaactgcctaatatcgatgcgggaagacatgttgaactgattgaagccatcgccaacgaatacctggaacatgccctgtcacaggaaagagattttcctcaatggccggaagatggtgccggccaaccggtttgcccggtgacactgacaagaattagataaaaaagaggggagggaaacattatgatggcccctcttcgctcaaacaaagatggcaaacctagagttcctagaacgacggaaagcgccgaaacgaatagaatgttaagaggtccgatgttactgagaacagacggaaatcagacgtcaacatacgatcaaagacttttagagtcaggcgcagatcacgcttggaagcatgcagatccgtggcgcgttctgagaatccagggagagttcgtggcaggatttgatgctctgagcgatctgcctaaggcggttacggtatacggctcagccagaacaaaagaggacacaccggaatatgcacagggcatcgatttaggtgcaaaattagttgagcatgactacgctgttgtgacaggcggtggcccaggcatcatggaggcggcgaatagaggcgcctatgaagctggaggtctttctgtgggcctgggcattgaacttccgtttgaacaaggtttaaacaaatatgtagatcttggaattaactttagatactttttcgcgagaaagacaatgttcctgaaatattcacaggcgtttgtgtgcctgccgggcggtatgggaacaatggatgagctgtttgaaatgctgtgtatggtgcaaacaggaaaagtgacaaactatccgattgtactgatcggcacagaatattggagcggcctggtagaatggatggctaatacactggtcgccgaaggctacatcaatccacaagatatggatctgttcctgctgacagacagcgtcgacgaagccgtcgaacatattgtcgcggctcataaagaaatgacagacgaaagagtctcaagactgaatcacgacgcataaattaattctagagtcgatccccgggttcgccagcaatgactaccggcagcccgcc

SEQ ID NO: 160 - DNA, 5255 bp, Artificial Sequence
  complete  AmyE 0 IPT6 LOG8 EryR AmyE END operon
gcggtagtggtgcttacgatgtacgacagggggattccccatacattcttcgcttggctgaaaatgattcttctttttatcgtctgcggcggcgttctgtttctgcttcggtatgtgattgtgaagctggcttacagaagagcggtaaaagaagaaataaaaaagaaatcatcttttttgtttggaaagcgagggaagcgttcacagtttcgggcagctttttttataggaacattgatttgtattcactctgccaagttgttttgatagagtgattgtgataattttaaatgtaagcgttaacaaaattctccagtcttcacatcggtttgaaaggaggaagcggaagaatgaagtaagagggatttttgactccgaagtaagtcttcaaaaaatcaaataaggagtgtcaagaatgtttgcaaaacgattcaaaacctctttactgccgttattcgctggatttttattgctgtttcatttggttctggcaggaccggcggctgcgagtgctgaaacggcgaacaaatcgaatgagcttacagcaccgtcgatcaaaagcggaaccattcttcatgcatggaattggtcgttcaatacgttaaaacacaatatgaaggatattcatgatgcaggatatacagccattcagacatctccgattaaccaagtaaaggaagggaatcaaggagataaaagcatgtcgaactggtactggctgtatcagccgacatcgtatcaaattggcaaccgttacttaggtactgaacaagaatttaaagaaatgtgtgcagccgctgaagaatatggcataaaggtcattgttgacgcggtcatcaatcataccaccagtgattatgccgcgatttccaatgaggttaagagtattccaaactggacacatggaaacacacaaattaaaaactggtctgatcgatgggatgtcacgcagaattcattgctcgggctgtatgactggaatacacaaaatacacaagtacagtcctatctgaaacggttcttagacagggcattgaatgacggggcagacggttttcgatttgatgccgccaaacatatagagcttccagatgatggcagttacggcagtcaattttggccgaatatcacaaatacatctgcagagttccaatacggagaaatcctgcaggatagtgcctccagagatgctgcttatgcgaattatatggatgtgacagcgtctaactatgggcattccataaggtccgctttaaagaatcgtaatctgggcgtgtcgaatatctcccactatgcatctgatgtgtctgcggacaagctagtgacatgggtagagtcgcatgatacacgggtgcgcatgatcgtatggttcactgtccaccaaccaaaactgtgctcagtaccgccaatatttctcccttggggggtacaaagaggtgtccctagaagagatccacgctgtgtaaaaattttacaaaaaggtattgactttccctacagggtgtgtaataatttaattacaggcgggggcaaccccgctcagtacctagagcgtaaaagaggggagggaaacactagtatgatcctgcatcttatttatggtccgacgtgcagcggcaaaacggacatggccattcagattgcgcaggagacaggatggccagtagtagctctggatagagtccagtgttgtccacaaattgctacaggctctggccgcccgctggagtctgagttacagagcacacgcagaatctatctggatagcagaccactgacagaaggtattctggatgcagaatcagcacatagacgcttaatcttcgaagtcgattggagaaagtctgaggacggacttattcttgaaggaggctcaatttctcttttaaactgtatggcaaaatcacctttctggagatcaggctttcagtggcacgttaaaagattaagacttggcgactcagatgcgtttctgacaagagcaaagcaaagagttgcagaaatgtttgcgattcgcgaggatagaccgtcacttttagaagaactggcagaactgtggaactatcctgccgcgcgccctatcttagaagacattgatggatacagatgcgccatcagatttgctagaaaacacgatctggccatttcacaactgcctaatatcgacgccggaagacatgttgaactgattgaagcgatcgcgaatgaatatctggaacatgctctgtctcaagaaagagattttccgcaatggccggaagatggcgctggtcaacctgtgtgcccggtgacactgacaagaattagataaaaaagaggggagggaaacattatgatggcccctcttcgctcaaacaaagatggcaaacctagagttcctagaacgacggaaagcgccgaaacgaatagaatgttaagaggtccgatgttactgagaacagacggaaatcagacgtcaacatacgatcaaagacttttagagtcaggcgcagatcacgcttggaagcatgcagatccgtggcgcgttctgagaatccagggagagttcgtggcaggatttgatgctctgagcgatctgcctaaggcggttacggtatacggctcagccagaacaaaagaggacacaccggaatatgcacagggcatcgatttaggtgcaaaattagttgagcatgactacgctgttgtgacaggcggtggcccaggcatcatggaggcggcgaatagaggcgcctatgaagctggaggtctttctgtgggcctgggcattgaacttccgtttgaacaaggtttaaacaaatatgtagatcttggaattaactttagatactttttcgcgagaaagacaatgttcctgaaatattcacaggcgtttgtgtgcctgccgggcggtatgggaacaatggatgagctgtttgaaatgctgtgtatggtgcaaacaggaaaagtgacaaactatccgattgtactgatcggcacagaatattggagcggcctggtagaatggatggctaatacactggtcgccgaaggctacatcaatccacaagatatggatctgttcctgctgacagacagcgtcgacgaagccgtcgaacatattgtcgcggctcataaagaaatgacagacgaaagagtctcaagactgaatcacgacgcataaattaattctagaaattaagaaggagggattcgtcatgttggtattccaaatgcgttatgtagataaaacatctactgttttgaaacagactaaaaacagtgattacgcagataaataaatacgttagattaattcctaccagtgactaatcttatgactttttaaacagataactaaaattacaaacaaatcgtttaacttctgtatttgtttatagatgtaatcacttcaggagtgattacatgaacaaaaatataaaatattctcaaaactttttaacgagtgaaaaagtactcaaccaaataataaaacaattgaatttaaaagaaaccgataccgtttacgaaattggaacaggtaaagggcatttaacgacgaaactggctaaaataagtaaacaggtaacgtctattgaattagacagtcatctattcaacttatcgtcagaaaaattaaaactgaacattcgtgtcactttaattcaccaagatattctacagtttcaattccctaacaaacagaggtataaaattgttgggaatattccttaccatttaagcacacaaattattaaaaaagtggtttttgaaagccatgcgtctgacatctatctgattgttgaagaaggattctacaagcgtaccttggatattcaccgaacactagggttgctcttgcacactcaagtctcgattcagcaattgcttaagctgccagcggaatgctttcatcctaaaccaaaagtaaacagtgtcttaataaaacttacccgccataccacagatgttccagataaatattggaagctatatacgtactttgtttcaaaatgggtcaatcgagaatatcgtcaactgtttactaaaaatcagtttcatcaagcaatgaaacacgccaaagtaaacaatttaagtaccgttacttatgagcaagtattgtctatttttaatagttatctattatttaacgggaggaaataattctatgagtcgcttttgtaaatttggaaagttacacgttactaaagggaatgtagatggatcctgtatgccaatgatgatgaagagtcgacatggatgagcgatgatgatatccgtttaggctgggcggtgatagcttctcgttcaggcagtacgcctcttttcttttccagacctgagggaggcggaaatggtgtgaggttcccggggaaaagccaaataggcgatcgcgggagtgctttatttgaagatcaggctatcactgcggtcaatagatttcacaatgtgatggctggacagcctgaggaactctcgaacccgaatggaaacaaccagatatttatgaatcagcgcggctcacatggcgttgtgctggcaaatgcaggttcatcctctgtctctatcaatacggcaacaaaattgcctgatggcaggtatgacaataaagctggagcgggttcatttcaagtgaacgatggtaaactgacaggcacgatcaatgccaggtctgtagctgtgctttatcctgatgatattgcaaaagcgcctcatgttttccttgagaattacaaaacaggtgtaacacattctttcaatgatcaactgacgattaccttgcgtgcagatgcgaatacaacaaaagccgtttatcaaatcaataatggaccagagacggcgtttaaggatggagatcaattcacaatcggaaaaggagatccatttggcaaaacatacaccatcatgttaaaaggaacgaacagtgatggtgtaacgaggaccgagaaatacagttttgttaaaagagatccagcgtcggccaaaaccatcggctatcaaaatccgaatcattggagccaggtaaatgcttatatctataaacatgatgggagccgagtaattgaattgaccggatcttggcctggaaaaccaatgactaaaaatgcagacggaatttacacgctgacgctgcctgcggacacggatacaaccaacgcaaaagtgatttttaataatggcagcgcccaagtgcccggtcagaatcagcctggctttgattacgtgctaaatggtttatataatgactcgggcttaagcggttctcttccccattg

SEQ ID NO: 161 - DNA, 5240 bp, Artificial Sequence
  complete  AmyE 0 IPT7 LOG8 EryR AmyE END operon
gcggtagtggtgcttacgatgtacgacagggggattccccatacattcttcgcttggctgaaaatgattcttctttttatcgtctgcggcggcgttctgtttctgcttcggtatgtgattgtgaagctggcttacagaagagcggtaaaagaagaaataaaaaagaaatcatcttttttgtttggaaagcgagggaagcgttcacagtttcgggcagctttttttataggaacattgatttgtattcactctgccaagttgttttgatagagtgattgtgataattttaaatgtaagcgttaacaaaattctccagtcttcacatcggtttgaaaggaggaagcggaagaatgaagtaagagggatttttgactccgaagtaagtcttcaaaaaatcaaataaggagtgtcaagaatgtttgcaaaacgattcaaaacctctttactgccgttattcgctggatttttattgctgtttcatttggttctggcaggaccggcggctgcgagtgctgaaacggcgaacaaatcgaatgagcttacagcaccgtcgatcaaaagcggaaccattcttcatgcatggaattggtcgttcaatacgttaaaacacaatatgaaggatattcatgatgcaggatatacagccattcagacatctccgattaaccaagtaaaggaagggaatcaaggagataaaagcatgtcgaactggtactggctgtatcagccgacatcgtatcaaattggcaaccgttacttaggtactgaacaagaatttaaagaaatgtgtgcagccgctgaagaatatggcataaaggtcattgttgacgcggtcatcaatcataccaccagtgattatgccgcgatttccaatgaggttaagagtattccaaactggacacatggaaacacacaaattaaaaactggtctgatcgatgggatgtcacgcagaattcattgctcgggctgtatgactggaatacacaaaatacacaagtacagtcctatctgaaacggttcttagacagggcattgaatgacggggcagacggttttcgatttgatgccgccaaacatatagagcttccagatgatggcagttacggcagtcaattttggccgaatatcacaaatacatctgcagagttccaatacggagaaatcctgcaggatagtgcctccagagatgctgcttatgcgaattatatggatgtgacagcgtctaactatgggcattccataaggtccgctttaaagaatcgtaatctgggcgtgtcgaatatctcccactatgcatctgatgtgtctgcggacaagctagtgacatgggtagagtcgcatgatacacgggtgcgcatgatcgtatggttcactgtccaccaaccaaaactgtgctcagtaccgccaatatttctcccttggggggtacaaagaggtgtccctagaagagatccacgctgtgtaaaaattttacaaaaaggtattgactttccctacagggtgtgtaataatttaattacaggcgggggcaaccccgctcagtacctagagcgtaaaagaggggagggaaacactagtatgccggtcagactgtatctgatttggggcgctacaacaacgggcaagacagcacaatctgtggcactggctagatcagcaggtgctcctgtaatttcactggacagagttcaatgttgccatgaactggccgttggttctggccgcccatcaccgagcgagctgctgggaacgagaagagaatatttatgtgagcgcgaagtgtcaagaggagtggttagcgccgccgaagcgaatcagctgctgctggataaagtggcgcgctatgccacacaggaacgcgcgctgattcttgaaggaggatcagtctcactgatcaacgctatgatcagagatgcgagatggagcgaaagaggcgaatggatcttaagaagaatcccggttccgggcagagccgcatttatggccgccgcgagaaaaagagttcgcgaaatgcttgacccgcctccaggccaggcaggaattttagatgagcttcaaggtctttggggctatccgagaaatcacgccgtcttagaggatattgacggctacagacagattattagatacgccaatgcattacaggtccctatctgcagaatcacatctattgaccctaacgccaaagctctgcttattgagcgcattgcacaggaatactgggaacatgctttatggcaagagcaagagttcctgggcattccggcaagctggatgagagctgatgatgcgtaaaaaagaggggagggaaacattatgatggcccctcttcgctcaaacaaagatggcaaacctagagttcctagaacgacggaaagcgccgaaacgaatagaatgttaagaggtccgatgttactgagaacagacggaaatcagacgtcaacatacgatcaaagacttttagagtcaggcgcagatcacgcttggaagcatgcagatccgtggcgcgttctgagaatccagggagagttcgtggcaggatttgatgctctgagcgatctgcctaaggcggttacggtatacggctcagccagaacaaaagaggacacaccggaatatgcacagggcatcgatttaggtgcaaaattagttgagcatgactacgctgttgtgacaggcggtggcccaggcatcatggaggcggcgaatagaggcgcctatgaagctggaggtctttctgtgggcctgggcattgaacttccgtttgaacaaggtttaaacaaatatgtagatcttggaattaactttagatactttttcgcgagaaagacaatgttcctgaaatattcacaggcgtttgtgtgcctgccgggcggtatgggaacaatggatgagctgtttgaaatgctgtgtatggtgcaaacaggaaaagtgacaaactatccgattgtactgatcggcacagaatattggagcggcctggtagaatggatggctaatacactggtcgccgaaggctacatcaatccacaagatatggatctgttcctgctgacagacagcgtcgacgaagccgtcgaacatattgtcgcggctcataaagaaatgacagacgaaagagtctcaagactgaatcacgacgcataaattaattctagaaattaagaaggagggattcgtcatgttggtattccaaatgcgttatgtagataaaacatctactgttttgaaacagactaaaaacagtgattacgcagataaataaatacgttagattaattcctaccagtgactaatcttatgactttttaaacagataactaaaattacaaacaaatcgtttaacttctgtatttgtttatagatgtaatcacttcaggagtgattacatgaacaaaaatataaaatattctcaaaactttttaacgagtgaaaaagtactcaaccaaataataaaacaattgaatttaaaagaaaccgataccgtttacgaaattggaacaggtaaagggcatttaacgacgaaactggctaaaataagtaaacaggtaacgtctattgaattagacagtcatctattcaacttatcgtcagaaaaattaaaactgaacattcgtgtcactttaattcaccaagatattctacagtttcaattccctaacaaacagaggtataaaattgttgggaatattccttaccatttaagcacacaaattattaaaaaagtggtttttgaaagccatgcgtctgacatctatctgattgttgaagaaggattctacaagcgtaccttggatattcaccgaacactagggttgctcttgcacactcaagtctcgattcagcaattgcttaagctgccagcggaatgctttcatcctaaaccaaaagtaaacagtgtcttaataaaacttacccgccataccacagatgttccagataaatattggaagctatatacgtactttgtttcaaaatgggtcaatcgagaatatcgtcaactgtttactaaaaatcagtttcatcaagcaatgaaacacgccaaagtaaacaatttaagtaccgttacttatgagcaagtattgtctatttttaatagttatctattatttaacgggaggaaataattctatgagtcgcttttgtaaatttggaaagttacacgttactaaagggaatgtagatggatcctgtatgccaatgatgatgaagagtcgacatggatgagcgatgatgatatccgtttaggctgggcggtgatagcttctcgttcaggcagtacgcctcttttcttttccagacctgagggaggcggaaatggtgtgaggttcccggggaaaagccaaataggcgatcgcgggagtgctttatttgaagatcaggctatcactgcggtcaatagatttcacaatgtgatggctggacagcctgaggaactctcgaacccgaatggaaacaaccagatatttatgaatcagcgcggctcacatggcgttgtgctggcaaatgcaggttcatcctctgtctctatcaatacggcaacaaaattgcctgatggcaggtatgacaataaagctggagcgggttcatttcaagtgaacgatggtaaactgacaggcacgatcaatgccaggtctgtagctgtgctttatcctgatgatattgcaaaagcgcctcatgttttccttgagaattacaaaacaggtgtaacacattctttcaatgatcaactgacgattaccttgcgtgcagatgcgaatacaacaaaagccgtttatcaaatcaataatggaccagagacggcgtttaaggatggagatcaattcacaatcggaaaaggagatccatttggcaaaacatacaccatcatgttaaaaggaacgaacagtgatggtgtaacgaggaccgagaaatacagttttgttaaaagagatccagcgtcggccaaaaccatcggctatcaaaatccgaatcattggagccaggtaaatgcttatatctataaacatgatgggagccgagtaattgaattgaccggatcttggcctggaaaaccaatgactaaaaatgcagacggaatttacacgctgacgctgcctgcggacacggatacaaccaacgcaaaagtgatttttaataatggcagcgcccaagtgcccggtcagaatcagcctggctttgattacgtgctaaatggtttatataatgactcgggcttaagcggttctcttccccattg

SEQ ID NO: 162 - DNA, 5255 bp, Artificial Sequence
  complete  AmyE 0 IPT9 LOG8 EryR AmyE END operon
gcggtagtggtgcttacgatgtacgacagggggattccccatacattcttcgcttggctgaaaatgattcttctttttatcgtctgcggcggcgttctgtttctgcttcggtatgtgattgtgaagctggcttacagaagagcggtaaaagaagaaataaaaaagaaatcatcttttttgtttggaaagcgagggaagcgttcacagtttcgggcagctttttttataggaacattgatttgtattcactctgccaagttgttttgatagagtgattgtgataattttaaatgtaagcgttaacaaaattctccagtcttcacatcggtttgaaaggaggaagcggaagaatgaagtaagagggatttttgactccgaagtaagtcttcaaaaaatcaaataaggagtgtcaagaatgtttgcaaaacgattcaaaacctctttactgccgttattcgctggatttttattgctgtttcatttggttctggcaggaccggcggctgcgagtgctgaaacggcgaacaaatcgaatgagcttacagcaccgtcgatcaaaagcggaaccattcttcatgcatggaattggtcgttcaatacgttaaaacacaatatgaaggatattcatgatgcaggatatacagccattcagacatctccgattaaccaagtaaaggaagggaatcaaggagataaaagcatgtcgaactggtactggctgtatcagccgacatcgtatcaaattggcaaccgttacttaggtactgaacaagaatttaaagaaatgtgtgcagccgctgaagaatatggcataaaggtcattgttgacgcggtcatcaatcataccaccagtgattatgccgcgatttccaatgaggttaagagtattccaaactggacacatggaaacacacaaattaaaaactggtctgatcgatgggatgtcacgcagaattcattgctcgggctgtatgactggaatacacaaaatacacaagtacagtcctatctgaaacggttcttagacagggcattgaatgacggggcagacggttttcgatttgatgccgccaaacatatagagcttccagatgatggcagttacggcagtcaattttggccgaatatcacaaatacatctgcagagttccaatacggagaaatcctgcaggatagtgcctccagagatgctgcttatgcgaattatatggatgtgacagcgtctaactatgggcattccataaggtccgctttaaagaatcgtaatctgggcgtgtcgaatatctcccactatgcatctgatgtgtctgcggacaagctagtgacatgggtagagtcgcatgatacacgggtgcgcatgatcgtatggttcactgtccaccaaccaaaactgtgctcagtaccgccaatatttctcccttggggggtacaaagaggtgtccctagaagagatccacgctgtgtaaaaattttacaaaaaggtattgactttccctacagggtgtgtaataatttaattacaggcgggggcaaccccgctcagtacctagagcgtaaaagaggggagggaaacactagtatgttattacatcttatttacggacctacatgttcaggaaaaacagacatggccattcaaattgctcaagaaacgggatggccggttgtggcgttagacagagtacagtgctgcccacagattgcaacgggctcaggtagaccgttagaatctgagttacaatcaacaagaagaatctatctggattcacgcccactgacagagggcatcctggacgcagagagcgcgcaccgccgccttatctttgaagtcgattggcgcaaatctgaggagggccttatccttgaaggaggaagcattagcctgcttaattgtatggcaaaatcaccgttttggagatctggcttccagtggcacgtaaagagactgagacttggcgattcagacgcatttctgacaagagcaaaacagagagtcgcagaaatgttcgcgattagagaagacagaccaagcctgctggaggaattagctgaactgtggaactatccggctgcgcgccctattctggaggatattgatggttatcgctgtgcgattagatttgctagaaagcatgatctggctatttcacaactgcctaatatcgatgcgggaagacatgttgaactgattgaagccatcgccaacgaatacctggaacatgccctgtcacaggaaagagattttcctcaatggccggaagatggtgccggccaaccggtttgcccggtgacactgacaagaattagataaaaaagaggggagggaaacattatgatggcccctcttcgctcaaacaaagatggcaaacctagagttcctagaacgacggaaagcgccgaaacgaatagaatgttaagaggtccgatgttactgagaacagacggaaatcagacgtcaacatacgatcaaagacttttagagtcaggcgcagatcacgcttggaagcatgcagatccgtggcgcgttctgagaatccagggagagttcgtggcaggatttgatgctctgagcgatctgcctaaggcggttacggtatacggctcagccagaacaaaagaggacacaccggaatatgcacagggcatcgatttaggtgcaaaattagttgagcatgactacgctgttgtgacaggcggtggcccaggcatcatggaggcggcgaatagaggcgcctatgaagctggaggtctttctgtgggcctgggcattgaacttccgtttgaacaaggtttaaacaaatatgtagatcttggaattaactttagatactttttcgcgagaaagacaatgttcctgaaatattcacaggcgtttgtgtgcctgccgggcggtatgggaacaatggatgagctgtttgaaatgctgtgtatggtgcaaacaggaaaagtgacaaactatccgattgtactgatcggcacagaatattggagcggcctggtagaatggatggctaatacactggtcgccgaaggctacatcaatccacaagatatggatctgttcctgctgacagacagcgtcgacgaagccgtcgaacatattgtcgcggctcataaagaaatgacagacgaaagagtctcaagactgaatcacgacgcataaattaattctagaaattaagaaggagggattcgtcatgttggtattccaaatgcgttatgtagataaaacatctactgttttgaaacagactaaaaacagtgattacgcagataaataaatacgttagattaattcctaccagtgactaatcttatgactttttaaacagataactaaaattacaaacaaatcgtttaacttctgtatttgtttatagatgtaatcacttcaggagtgattacatgaacaaaaatataaaatattctcaaaactttttaacgagtgaaaaagtactcaaccaaataataaaacaattgaatttaaaagaaaccgataccgtttacgaaattggaacaggtaaagggcatttaacgacgaaactggctaaaataagtaaacaggtaacgtctattgaattagacagtcatctattcaacttatcgtcagaaaaattaaaactgaacattcgtgtcactttaattcaccaagatattctacagtttcaattccctaacaaacagaggtataaaattgttgggaatattccttaccatttaagcacacaaattattaaaaaagtggtttttgaaagccatgcgtctgacatctatctgattgttgaagaaggattctacaagcgtaccttggatattcaccgaacactagggttgctcttgcacactcaagtctcgattcagcaattgcttaagctgccagcggaatgctttcatcctaaaccaaaagtaaacagtgtcttaataaaacttacccgccataccacagatgttccagataaatattggaagctatatacgtactttgtttcaaaatgggtcaatcgagaatatcgtcaactgtttactaaaaatcagtttcatcaagcaatgaaacacgccaaagtaaacaatttaagtaccgttacttatgagcaagtattgtctatttttaatagttatctattatttaacgggaggaaataattctatgagtcgcttttgtaaatttggaaagttacacgttactaaagggaatgtagatggatcctgtatgccaatgatgatgaagagtcgacatggatgagcgatgatgatatccgtttaggctgggcggtgatagcttctcgttcaggcagtacgcctcttttcttttccagacctgagggaggcggaaatggtgtgaggttcccggggaaaagccaaataggcgatcgcgggagtgctttatttgaagatcaggctatcactgcggtcaatagatttcacaatgtgatggctggacagcctgaggaactctcgaacccgaatggaaacaaccagatatttatgaatcagcgcggctcacatggcgttgtgctggcaaatgcaggttcatcctctgtctctatcaatacggcaacaaaattgcctgatggcaggtatgacaataaagctggagcgggttcatttcaagtgaacgatggtaaactgacaggcacgatcaatgccaggtctgtagctgtgctttatcctgatgatattgcaaaagcgcctcatgttttccttgagaattacaaaacaggtgtaacacattctttcaatgatcaactgacgattaccttgcgtgcagatgcgaatacaacaaaagccgtttatcaaatcaataatggaccagagacggcgtttaaggatggagatcaattcacaatcggaaaaggagatccatttggcaaaacatacaccatcatgttaaaaggaacgaacagtgatggtgtaacgaggaccgagaaatacagttttgttaaaagagatccagcgtcggccaaaaccatcggctatcaaaatccgaatcattggagccaggtaaatgcttatatctataaacatgatgggagccgagtaattgaattgaccggatcttggcctggaaaaccaatgactaaaaatgcagacggaatttacacgctgacgctgcctgcggacacggatacaaccaacgcaaaagtgatttttaataatggcagcgcccaagtgcccggtcagaatcagcctggctttgattacgtgctaaatggtttatataatgactcgggcttaagcggttctcttccccattg

SEQ ID NO: 163 - DNA, 45 bp, Artificial Sequence
  primer ipt1_RBS_F (pBBR1)
tttaagaaggagatatacatatgttattacacctgatctacggtc

SEQ ID NO: 164 - DNA, 48 bp, Artificial Sequence
  primer ipt1_RBS_R(pBBR1)
tcatatgtatatctccttcttttaacggattctcgttaaggtaaccgg

SEQ ID NO: 165 - DNA, 42 bp, Artificial Sequence
  primer log1_RBS_F(pBBR1)
taaaagaaggagatatacatatgacatcgctttttgacgcgc

SEQ ID NO: 166 - DNA, 40 bp, Artificial Sequence
  primer log1_RBS_R(pBBR1)
ccttactcgagtttggatccttaccattttggggctggag

SEQ ID NO: 167 - DNA, 20 bp, Artificial Sequence
  primer BB_pBBR1_F
ggatccaaactcgagtaagg

SEQ ID NO: 168 - DNA, 27 bp, Artificial Sequence
  primer BB_pBBR1_R
atgtatatctccttcttaaagttaaac

SEQ ID NO: 169 - DNA, 50 bp, Artificial Sequence
  primer dxs_F(p15A)
acaataataatggagtcatgaacatatggacttgttgtccattcaagacc

SEQ ID NO: 170 - DNA, 50 bp, Artificial Sequence
  primer dxs_R (p15A)
gaaggtgagccagtgtgactctagtttatgagccgatgcccttatgtgtc

SEQ ID NO: 171 - DNA, 27 bp, Artificial Sequence
  primer BB_15a_R
atgttcatgactccattattattgtac

SEQ ID NO: 172 - DNA, 25 bp, Artificial Sequence
  primer BB_p15a_F
actagagtcacactggctcaccttc

SEQ ID NO: 173 - DNA, 67 bp, Artificial Sequence
  primer for IPT1 LOG1 amplification
atggaattcgagctcggtacccggggaaaggaggcccttcagatgttattacacctgatctacggtc

SEQ ID NO: 174 - DNA, 49 bp, Artificial Sequence
  primer for IPT1 LOG1 amplification
gcatgcctgcaggtcgactctagaggatcttaccattttggggctggag

SEQ ID NO: 175 - DNA, 67 bp, Artificial Sequence
  atggaattcgagctcggtacccggggaAAGGAGGCCCTTCAGATGGA CTTGTTGTCCATTCAAGA CC
atggaattcgagctcggtacccggggaaaggaggcccttcagatggacttgttgtccattcaagacc

SEQ ID NO: 176 - DNA, 54 bp, Artificial Sequence
  primer for dxs amplification
gcatgcctgcaggtcgactctagaggatcttatgagccgatgcccttatgtgtc

SEQ ID NO: 177 - DNA, 56 bp, Artificial Sequence
  primer for IPT1 amplification
gcatgcctgcaggtcgactctagaggatcttaacggattctcgttaaggtaaccgg

SEQ ID NO: 178 - DNA, 1415 bp, Artificial Sequence
  IPT1-LOG1 Coryneb.
atggaattcgagctcggtacccggggaaaggaggcccttcagatgttattacacctgatctacggtcctacatgtagtggtaaaactgatatggctatacaaatcgcgcaagaaacaggttggcccgtcgttgctcttgatcgtgtgcagtgctgtccacagattgcgacaggatccggcagacctctggaaagcgagttacaatctacccggcggatttatttagactcacgccctttgacagaaggcattcttgacgcggaatccgctcatcgccgtcttatattcgaggtagattggaggaaatcggaagaaggcttaatcctcgaaggagggtccatctcgcttctaaattgtatggctaagtctccattttggagatctggattccagtggcacgtcaagcgcctgagacttggggattcagatgcgtttctgacgcgagcaaaacagcgtgtggctgagatgtttgctattagagaagatcggccaagtttattggaggagctcgccgagctgtggaattacccggcagctcgcccgattttggaagacattgacggctatcgatgtgccattagatttgcaaggaaacacgatctcgccatatcacagctgccgaacatcgatgccggccggcatgttgaactgatcgaagccattgcaaacgaatatcttgaacatgcgctaagccaagaacgcgactttcctcaatggccggaggacggcgcaggacaaccagtatgcccggttaccttaacgagaatccgttaaaaaagaggggagggaaacattatgatgacatcgctttttgacgcgcctacgcttcaacgcgtaacggttttcactgggagcgccttgggctcctctagtctttatactcaggctgctcaaaccctggctaaaaccgcggtagaccgagggattgatctggtttatggcggtggaaaagtcggcctcatgggtattgttgctgatgcattccttgaaagcgggggtgaggcattcggcgtgataacagagtcactgatgaaaggagaattaggccatgagaaactgacggagttggaaatcgtgcccgacatgcatattcgtaaaaggcggatggcggaacttggggatggattcattgcgatgcctggaggagccggcaccctggaagaactgtttgaagtctggacatggcagcagttaggaattcatcaaaagccggttgcactctatgacgtcgatggcttctggcaaccgttattggaaatgttggaacaaatgacacagagaggatttatcaagagagatttttttgaatgcctcatcgtcgaatcagatccgcacgcattattaaaggccatgcagacatggacacctccagccccaaaatggtaagatcctctagagtcgacctgcaggcatgc

SEQ ID NO: 179 - DNA, 842 bp, Artificial Sequence
  Fragment IPT1
cgcagcatacgcagcgaaatcagcatcaccggagaatcccaacgaagccaactagtatgttattacacctgatctacggtcctacatgtagtggtaaaactgatatggctatacaaatcgcgcaagaaacaggttggcccgtcgttgctcttgatcgtgtgcagtgctgtccacagattgcgacaggatccggcagacctctggaaagcgagttacaatctacccggcggatttatttagactcacgccctttgacagaaggcattcttgacgcggaatccgctcatcgccgtcttatattcgaggtagattggaggaaatcggaagaaggcttaatcctcgaaggagggtccatctcgcttctaaattgtatggctaagtctccattttggagatctggattccagtggcacgtcaagcgcctgagacttggggattcagatgcgtttctgacgcgagcaaaacagcgtgtggctgagatgtttgctattagagaagatcggccaagtttattggaggagctcgccgagctgtggaattacccggcagctcgcccgattttggaagacattgacggctatcgatgtgccattagatttgcaaggaaacacgatctcgccatatcacagctgccgaacatcgatgccggccggcatgttgaactgatcgaagccattgcaaacgaatatcttgaacatgcgctaagccaagaacgcgactttcctcaatggccggaggacggcgcaggacaaccagtatgcccggttaccttaacgagaatccgttgaaaaagaggggagggaaacattaatgacgacctggctaacgagtctcgccgatct

SEQ ID NO: 180 - DNA, 833 bp, Artificial Sequence
  IPT1 Coryneb.
atggaattcgagctcggtacccggggaaaggaggcccttcagatgttattacacctgatctacggtcctacatgtagtggtaaaactgatatggctatacaaatcgcgcaagaaacaggttggcccgtcgttgctcttgatcgtgtgcagtgctgtccacagattgcgacaggatccggcagacctctggaaagcgagttacaatctacccggcggatttatttagactcacgccctttgacagaaggcattcttgacgcggaatccgctcatcgccgtcttatattcgaggtagattggaggaaatcggaagaaggcttaatcctcgaaggagggtccatctcgcttctaaattgtatggctaagtctccattttggagatctggattccagtggcacgtcaagcgcctgagacttggggattcagatgcgtttctgacgcgagcaaaacagcgtgtggctgagatgtttgctattagagaagatcggccaagtttattggaggagctcgccgagctgtggaattacccggcagctcgcccgattttggaagacattgacggctatcgatgtgccattagatttgcaaggaaacacgatctcgccatatcacagctgccgaacatcgatgccggccggcatgttgaactgatcgaagccattgcaaacgaatatcttgaacatgcgctaagccaagaacgcgactttcctcaatggccggaggacggcgcaggacaaccagtatgcccggttaccttaacgagaatccgttaagatcctctagagtcgacctgcaggcatgccgagctcgaattcactggccgtcgttttac

SEQ ID NO: 181 - DNA, 1973 bp, Artificial Sequence
  DXS Coryneb.
atggaattcgagctcggtacccggggaaaggaggcccttcagatggacttgttgtccattcaagacccgtctttcctaaagaatatgagcattgacgagcttgaaaaattgtccgatgagatccgccaatttctcatcacatctctatcggcctcgggcggacacattggaccgaatctgggggtcgtggaactgacagtagccctccacaaggagttcaactcacccaaagacaaatttctctgggacgtggggcaccaaagctacgttcataaattgcttactggtcgtggcaaagagttcgccacattgcgccagtacaaaggactctgcggattccctaaacgctcggaatccgagcacgatgtgtgggaaacaggtcattcttctacttctctcagcggcgccatgggaatggccgctgcacgagatattaaagggactgatgagtacataatcccaataataggtgacggcgctttaacaggaggaatggccctagaggccttgaaccacataggggatgagaagaaagatatgatagtcattttgaatgataatgagatgtctattgctcccaacgttggagcaatacatagcatgttagggcggttacgtactgcaggaaagtaccagtgggtaaaagatgaacttgaatacctcttcaagaagatacccgctgtaggaggcaagcttgcagccacggccgagagagtgaaggattctcttaaatatatgttggtttcagggatgttctttgaagaattgggattcacgtaccttggtccggttgacggccattcttaccacgagttaattgaaaatttacaatacgcgaaaaaaacgaaggggcctgttctgctgcacgttattaccaaaaaaggaaaaggttataaacctgcggaaaccgatacaattggcacgtggcacggcaccgggccttataaaatcaacacgggggactttgtcaagccgaaagctgccgcgcctagttggtccggcctggttagtgggacggtacagcgaatggcccgggaagatgggaggattgtcgctatcacaccggcaatgccagtaggtagcaagttagaaggttttgcgaaagaatttccggataggatgtttgacgtaggtattgctgagcagcatgcggcgacgatggctgcggcaatggcaatgcagggcatgaagccttttttagctatttattcaacatttctgcaaagagcgtatgaccaagtggtacatgacatttgtcggcaaaatgctaatgtctttattgggattgatcgcgcagggttagtgggtgcagacggggaaacgcatcaaggagtgtttgacatcgcgtttatgcgacatatcccaaatatggtccttatgatgccaaaagacgaaaacgagggtcaacacatggtgcatacagctcttagttatgacgaaggacctatcgcgatgagatttccgagagggaatggattaggcgtcaaaatggatgagcagcttaaaacaattccgattggaacatgggaagtcttacgtccaggaaatgacgcggttatccttacatttggcactaccattgagatggcgattgaggcagcagaagaactccagaaggagggcctgtcagtccgtgttgttaatgcgagatttattaaaccgattgacgaaaagatgatgaaaagtatcctgaaagaaggcctgccgatacttaccatcgaggaagctgttctggaaggaggatttggctcatcaatccttgaatttgcacatgatcagggcgaatatcacacaccgatcgatcgaatgggcatcccggatcgatttatcgagcatggctccgttacagcactgcttgaagagatcggcttaacgaaacagcaggtggcaaacagaatcaggttacttatgccgccgaagacacataagggcatcggctcataagatcctctagagtcgacctgcaggcatgc

SEQ ID NO: 182 - DNA, 47 bp, Artificial Sequence
  primer ipt1_R(pBBR1)
ccttactcgagtttggatccttaacggattctcgttaaggtaaccgg

SEQ ID NO: 183 - DNA, 33 bp, Artificial Sequence
  primer ipt1_R_XbaI
atcattctagatcaacggattctcgttaaggta

SEQ ID NO: 184 - DNA, 4397 bp, Artificial Sequence
  complete AmyE IPT1 EryR construct
gcggtagtggtgcttacgatgtacgacagggggattccccatacattcttcgcttggctgaaaatgattcttctttttatcgtctgcggcggcgttctgtttctgcttcggtatgtgattgtgaagctggcttacagaagagcggtaaaagaagaaataaaaaagaaatcatcttttttgtttggaaagcgagggaagcgttcacagtttcgggcagctttttttataggaacattgatttgtattcactctgccaagttgttttgatagagtgattgtgataattttaaatgtaagcgttaacaaaattctccagtcttcacatcggtttgaaaggaggaagcggaagaatgaagtaagagggatttttgactccgaagtaagtcttcaaaaaatcaaataaggagtgtcaagaatgtttgcaaaacgattcaaaacctctttactgccgttattcgctggatttttattgctgtttcatttggttctggcaggaccggcggctgcgagtgctgaaacggcgaacaaatcgaatgagcttacagcaccgtcgatcaaaagcggaaccattcttcatgcatggaattggtcgttcaatacgttaaaacacaatatgaaggatattcatgatgcaggatatacagccattcagacatctccgattaaccaagtaaaggaagggaatcaaggagataaaagcatgtcgaactggtactggctgtatcagccgacatcgtatcaaattggcaaccgttacttaggtactgaacaagaatttaaagaaatgtgtgcagccgctgaagaatatggcataaaggtcattgttgacgcggtcatcaatcataccaccagtgattatgccgcgatttccaatgaggttaagagtattccaaactggacacatggaaacacacaaattaaaaactggtctgatcgatgggatgtcacgcagaattcattgctcgggctgtatgactggaatacacaaaatacacaagtacagtcctatctgaaacggttcttagacagggcattgaatgacggggcagacggttttcgatttgatgccgccaaacatatagagcttccagatgatggcagttacggcagtcaattttggccgaatatcacaaatacatctgcagagttccaatacggagaaatcctgcaggatagtgcctccagagatgctgcttatgcgaattatatggatgtgacagcgtctaactatgggcattccataaggtccgctttaaagaatcgtaatctgggcgtgtcgaatatctcccactatgcatctgatgtgtctgcggacaagctagtgacatgggtagagtcgcatgatacacgggtgcgcatgatcgtatggttcactgtccaccaaccaaaactgtgctcagtaccgccaatatttctcccttggggggtacaaagaggtgtccctagaagagatccacgctgtgtaaaaattttacaaaaaggtattgactttccctacagggtgtgtaataatttaattacaggcgggggcaaccccgctcagtacctagagcgtaaaagaggggagggaaacactagtatgttattacacctgatctacggtcctacatgtagtggtaaaactgatatggctatacaaatcgcgcaagaaacaggttggcccgtcgttgctcttgatcgtgtgcagtgctgtccacagattgcgacaggatccggcagacctctggaaagcgagttacaatctacccggcggatttatttagactcacgccctttgacagaaggcattcttgacgcggaatccgctcatcgccgtcttatattcgaggtagattggaggaaatcggaagaaggcttaatcctcgaaggagggtccatctcgcttctaaattgtatggctaagtctccattttggagatctggattccagtggcacgtcaagcgcctgagacttggggattcagatgcgtttctgacgcgagcaaaacagcgtgtggctgagatgtttgctattagagaagatcggccaagtttattggaggagctcgccgagctgtggaattacccggcagctcgcccgattttggaagacattgacggctatcgatgtgccattagatttgcaaggaaacacgatctcgccatatcacagctgccgaacatcgatgccggccggcatgttgaactgatcgaagccattgcaaacgaatatcttgaacatgcgctaagccaagaacgcgactttcctcaatggccggaggacggcgcaggacaaccagtatgcccggttaccttaacgagaatccgttaatctagaaattaagaaggagggattcgtcatgttggtattccaaatgcgttatgtagataaaacatctactgttttgaaacagactaaaaacagtgattacgcagataaataaatacgttagattaattcctaccagtgactaatcttatgactttttaaacagataactaaaattacaaacaaatcgtttaacttctgtatttgtttatagatgtaatcacttcaggagtgattacatgaacaaaaatataaaatattctcaaaactttttaacgagtgaaaaagtactcaaccaaataataaaacaattgaatttaaaagaaaccgataccgtttacgaaattggaacaggtaaagggcatttaacgacgaaactggctaaaataagtaaacaggtaacgtctattgaattagacagtcatctattcaacttatcgtcagaaaaattaaaactgaacattcgtgtcactttaattcaccaagatattctacagtttcaattccctaacaaacagaggtataaaattgttgggaatattccttaccatttaagcacacaaattattaaaaaagtggtttttgaaagccatgcgtctgacatctatctgattgttgaagaaggattctacaagcgtaccttggatattcaccgaacactagggttgctcttgcacactcaagtctcgattcagcaattgcttaagctgccagcggaatgctttcatcctaaaccaaaagtaaacagtgtcttaataaaacttacccgccataccacagatgttccagataaatattggaagctatatacgtactttgtttcaaaatgggtcaatcgagaatatcgtcaactgtttactaaaaatcagtttcatcaagcaatgaaacacgccaaagtaaacaatttaagtaccgttacttatgagcaagtattgtctatttttaatagttatctattatttaacgggaggaaataattctatgagtcgcttttgtaaatttggaaagttacacgttactaaagggaatgtagatggatcctgtatgccaatgatgatgaagagtcgacatggatgagcgatgatgatatccgtttaggctgggcggtgatagcttctcgttcaggcagtacgcctcttttcttttccagacctgagggaggcggaaatggtgtgaggttcccggggaaaagccaaataggcgatcgcgggagtgctttatttgaagatcaggctatcactgcggtcaatagatttcacaatgtgatggctggacagcctgaggaactctcgaacccgaatggaaacaaccagatatttatgaatcagcgcggctcacatggcgttgtgctggcaaatgcaggttcatcctctgtctctatcaatacggcaacaaaattgcctgatggcaggtatgacaataaagctggagcgggttcatttcaagtgaacgatggtaaactgacaggcacgatcaatgccaggtctgtagctgtgctttatcctgatgatattgcaaaagcgcctcatgttttccttgagaattacaaaacaggtgtaacacattctttcaatgatcaactgacgattaccttgcgtgcagatgcgaatacaacaaaagccgtttatcaaatcaataatggaccagagacggcgtttaaggatggagatcaattcacaatcggaaaaggagatccatttggcaaaacatacaccatcatgttaaaaggaacgaacagtgatggtgtaacgaggaccgagaaatacagttttgttaaaagagatccagcgtcggccaaaaccatcggctatcaaaatccgaatcattggagccaggtaaatgcttatatctataaacatgatgggagccgagtaattgaattgaccggatcttggcctggaaaaccaatgactaaaaatgcagacggaatttacacgctgacgctgcctgcggacacggatacaaccaacgcaaaagtgatttttaataatggcagcgcccaagtgcccggtcagaatcagcctggctttgattacgtgctaaatggtttatataatgactcgggcttaagcggttctcttccccattg